(19) United States
(12) Patent Application Publication
(10) Pub. No.: US 2025/0018046 A1
(43) Pub. Date: Jan. 16, 2025

(54) COMPOUNDS FOR TARGETING DEGRADATION OF IRAK4 PROTEINS

(71) Applicants: BIOGEN MA INC, Cambridge, MA (US); C4 THERAPEUTICS, INC., Watertown, MA (US)

(72) Inventors: Kevin M. Guckian, Northborough, MA (US); Emily Anne Peterson, Belmont, MA (US); Fang Gao, Somerville, MA (US); Ryan Evans, Somerville, MA (US); Eric Stefan, Watertown, MA (US); Jeremy L. Yap, Sudbury, MA (US); Corey Don Anderson, Brighton, MA (US); Morgan Weizel O'Shea, Waltham, MA (US); Jae Young M. Ahn, Somerville, MA (US); Christopher G. Nasveschuk, Stoneham, MA (US); James A. Henderson, Weston, MA (US)

(21) Appl. No.: 18/576,933

(22) PCT Filed: Jul. 7, 2022
(86) PCT No.: PCT/US2022/036409

§ 371 Date: Jan. 5, 2024

Related U.S. Application Data
(60) Provisional application No. 63/354,020, filed on Jun. 21, 2022.
(60) Provisional application No. 63/219,167, filed on Jul. 7, 2021.

(51) Int. Cl. A61K 47/55; A61K 47/54

(52) U.S. Cl. CPC A61K 47/55; A61K 47/545

(57) ABSTRACT

This disclosure relates to compounds of Formula (A): Formula (A), or a pharmaceutically acceptable salt thereof, wherein DSM is a degradation signaling moiety that is covalently attached to the linker L, L is a linker that covalently attaches IRAK to DSM; and IRAK is an IRAK4 binding moiety represented by Formula (I) that is covalently attached to linker L; Formula (I) in which all of the variables are as defined in the application. Compounds or pharmaceutically acceptable salts thereof as described herein are capable of activating the selective ubiqitination of IRAK4 proteins via the ubiquitin-proteasome pathways (UPP) and cause degradation of IRAK4 proteins. The present disclosure also provides methods of treating disorders responsive to modulation of IRAK4 activity and/or degradation of IRAK4 with at least one compound described herein.

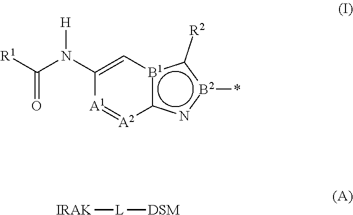

### CROSS-REFERENCE TO RELATED APPLICATIONS

This application claims priority to U.S. Provisional Patent Application Nos. 63/219,167, filed Jul. 7, 2021 and 63/354,020, filed Jun. 21, 2022. The entire contents of each of the foregoing applications are expressly incorporated herein by reference.

### TECHNICAL FIELD

Provided are certain agents that target the degradation of interleukin-1 receptor-associated kinase 4 (IRAK4), and methods of making and using such agents.

### BACKGROUND

Protein degradation is a highly regulated and essential process that maintains cellular homeostasis. The selective identification and removal of damaged, misfolded, or excess proteins is achieved via the ubiquitin-proteasome pathway (UPP). The UPP is central to the regulation of almost all cellular processes, including antigen processing, apoptosis, biogenesis of organelles, cell cycling, DNA transcription and repair, differentiation and development, immune response and inflammation, neural and muscular degeneration, morphogenesis of neural networks, modulation of cell surface receptors, ion channels and the secretory pathway, the response to stress and extracellular modulators, ribosome biogenesis and viral infection.

Covalent attachment of multiple ubiquitin molecules by an E3 ubiquitin ligase to a terminal lysine residue marks the protein for proteasome degradation, where the protein is digested into small peptides and eventually into its constituent amino acids that serve as building blocks for new proteins. There are over 600 E3 ubiquitin ligases which facilitate the ubiquitination of different proteins in vivo, which can be divided into four families: HECT-domain E3s, U-box E3s, monomeric RING E3s and multi-subunit E3s.

It is known that the ubiquitin-proteasome pathway (UPP) can be harnessed for therapeutic intervention by using chimeric compounds capable of activating the ubiquitination of a Target Protein, where the chimeric compound comprises a Target Protein binding element that is covalently linked to ubiquitination recognition element. Such chimeric compounds that are capable of binding a Target Protein and a ubiquitin ligase may cause the Target Protein to be selectively degraded via the UPP. The discovery, for example, that thalidomide binds to the cereblon E3 ubiquitin ligase has led to research investigating the incorporation of thalidomide and certain derivatives into chimeric compounds for the targeted destruction of proteins.

Protein kinases are a large multigene family consisting of more than 500 proteins which play a critical role in the development and treatment of a number of human diseases in oncology, neurology and immunology. Kinases catalyze the phosphorylation of proteins, lipids, sugars, nucleosides and other cellular metabolites and play key roles in all aspects of eukaryotic cell physiology. Especially, protein kinases and lipid kinases participate in the signaling events which control the activation, growth, differentiation and survival of cells in response to extracellular mediators or stimuli such as growth factors, cytokines or chemokines. In general, protein kinases are classified in two groups, those that preferentially phosphorylate tyrosine residues and those that preferentially phosphorylate serine and/or threonine residues.

Kinases are important therapeutic targets for the development of anti-inflammatory drugs (Cohen, 2009. Current Opinion in Cell Biology 21, 1-8), for example kinases that are involved in the orchestration of adaptive and innate immune responses. Many diseases are associated with abnormal cellular responses triggered by kinase-mediated events. Kinase targets of particular interest are members of the IRAK family.

The interleukin-1 receptor-associated kinases (IRAKs) are critically involved in the regulation of intracellular signaling networks controlling inflammation (Ringwood and Li, 2008. Cytokine 42, 1-7). IRAKs are expressed in many cell types and can mediate signals from various cell receptors including toll-like receptors (TLRs).

IRAK1 was first identified through biochemical purification of the IL-1 dependent kinase activity that co-immunoprecipitates with the IL-1 type 1 receptor (Cao et al., 1996. Science 271(5252): 1128-31). IRAK2 was identified by the search of the human expressed sequence tag (EST) database for sequences homologous to IRAK1 (Muzio et al., 1997. Science 278(5343): 1612-5). IRAK3 (also called IRAKM) was identified using a murine EST sequence encoding a polypeptide with significant homology to IRAK1 to screen a human phytohemagglutinin-activated peripheral blood leukocyte (PBL) cDNA library (Wesche et al., 1999. J. Biol. Chem. 274(27): 19403-10). IRAK4 was identified by database searching for IRAK-like sequences and PCR of a universal cDNA library (Li et al., 2002. Proc. Natl. Acad. Sci. USA 99(8):5567-5572).

IRAK4 is thought to be the initial protein kinase activated downstream of the interleukin-1 (IL-1) receptor and all toll-like-receptors (TLRs) except TLR3, and initiates signaling in the innate immune system via the rapid activation of IRAK1 and slower activation of IRAK2.

Given that IRAK4 plays an important role in signaling networks controlling inflammation, there is a great need to develop chimeric compounds capable of activating the ubiquitination and degradation of IRAK4 proteins. It is an object of the present disclosure to provide new compounds, methods, compositions and methods of manufacture that are useful for the selective degradation of IRAK4 protein in vivo via the ubiquitin-proteasome pathway (UPP).

### SUMMARY

In a first aspect, the present disclosure is a compound of formula (A):

IRAK-L-DSM (A),

or a pharmaceutically acceptable salt thereof, wherein:
DSM is a degradation signaling moiety that is covalently attached to the linker L,
L is a linker that covalently attaches IRAK to DSM; and
IRAK is an IRAK4 binding moiety represented by Formula (I) that is covalently attached to linker L;

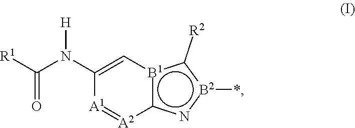

wherein:
A¹ is selected from N, CH and CR³, and A² is selected from N, CH and CR⁴, provided only one of A¹ or A² may be N;
one of B¹ and B² is N, and the other is C;
R¹ is selected from:
   i. phenyl optionally substituted with 1 to 3 R⁵,
   ii. a 5 or 6 membered heteroaryl having 1 to 3 heteroatoms independently selected from nitrogen, oxygen and sulfur, said heteroaryl is optionally substituted with 1 to 3 R⁵,
   iii. a 5 or 6 membered partially or fully saturated heterocycle having 1 to 2 heteroatoms independently selected from oxygen and nitrogen, said heterocycle may be optionally substituted with 1 to 3 R⁵,
   iv. a partially or fully saturated C₃₋₆ cycloalkyl which may be optionally substituted with 1 to 3 R⁵,
   v. a 7 to 10 membered fused heterobicyclic ring system having 1, 2 or 3 heteroatoms independently selected from nitrogen and oxygen, said heterobicylic ring system is optionally substituted with 1 to 3 R⁵, and
   vi. a 7 to 10 membered fused carbobicyclic ring system, said carbobicyclic ring system is optionally substituted with 1 to 3 R⁵;
R² is hydrogen, C₁₋₄ alkyl or halogen;
R³ and R⁴ are each independently selected from halogen, C₁₋₄alkyl, nitrile and —OR⁶, wherein the C₁₋₄alkyl is optionally substituted with C₁₋₄alkoxy or at least one halogen;
R⁵ for each occurrence, is independently selected from CN, hydroxyl, C₁₋₄ alkyl, oxo, halogen, —NR⁸R⁹, C₁₋₄ alkoxy, —O—C₁₋₄ alkyl, C₃₋₆cycloalkyl, —C₁₋₄alkyl-C₃₋₆cycloalkyl, C(O)NR¹⁰R¹¹, a C₄₋₇ heterocycle, and a 5 or 6 membered heteroaryl having 1 to 2 heteroatoms independently selected from nitrogen, oxygen and sulfur, said C₁₋₄ alkyl is optionally substituted with one or more substituents independently selected from CN, halo, C₁₋₄alkoxy, and hydroxyl, said C₃₋₆cycloalkyl and heteroaryl is optionally substituted with 1 to 2 substituents independently selected from the group consisting of C₁₋₄ alkyl, hydroxyl and halogen; or two R⁵ groups together with the intervening atoms can form a ring selected from phenyl, C₄₋₆ carbocycle, C₄₋₆ heterocycle, or a 7-membered bridged ring system optionally having 1 heteroatom selected from nitrogen and oxygen, wherein said phenyl, C₄₋₆ carbocycle and C₄₋₆ heterocycle are each optionally substituted with 1 to 2 C₁₋₄ alkyl, halogen or C₁₋₄ haloalkyl;
R⁶ is hydrogen, C₁₋₅alkyl, C₃₋₆cycloalkyl, a 4 to 7 membered partially or fully saturated heterocycle containing 1 or 2 heteroatoms selected from nitrogen and oxygen, a 5 to 10 membered spiro carbocyclic ring and a 4 to 10 membered heterocycle having 1 to 2 heteroatoms independently selected from nitrogen and oxygen; wherein the C₁₋₅alkyl represented by R⁶ is optionally substituted with 1 to 3 substituents R^{6a} independently selected from halogen, hydroxyl, C₁₋₅alkyl, C₁₋₄alkoxy, C₁₋₄ haloalkoxy, C₃₋₆cycloalkyl, phenyl, a 4 to 7 membered partially or fully saturated heterocycle containing 1 or 2 heteroatoms selected from nitrogen and oxygen, and a fully saturated 5 to 8 membered bridged-heterocyclic ring system having 1 to 2 heteroatoms independently selected from nitrogen and oxygen; wherein the C₃₋₆cycloalkyl represented by R⁶ is optionally substituted with 1 to 3 substituents R^{6b} independently selected from halogen, C₁₋₄alky, C₁₋₄ haloalkyl, and C₁₋₄alkoxy; wherein the 4 to 7 membered partially or fully saturated heterocycle, the 5 to 10 membered spiro carbocyclic ring and 5 to 10 membered spiro heterobicyclic ring system represented by R⁶ is optionally substituted with 1 to 3 substituents R⁶, independently selected from C₁₋₄alky and oxo; and wherein said C₃₋₆cycloalkyl, phenyl, 4 to 7 membered partially or fully saturated heterocycle represented by R^{6a} are optionally substituted with 1 to 3 R⁷;
each R⁷ is independently selected from oxo, halogen, C₁₋₄ haloalkyl and C₁₋₄ alkyl;
R⁸ and R⁹ are each independently selected from hydrogen, —C(O)C₁₋₄ alkyl and C₁₋₄ alkyl; or R⁸ and R⁹ may combine to form a 4 to 6 membered saturated ring optionally containing one additional heteroatom selected from nitrogen or oxygen wherein said additional nitrogen may be optionally substituted with C₁₋₄ alkyl;
R¹⁰ and R¹¹ are each independently selected from hydrogen and C₁₋₄ alkyl; and
—* represents a bond to the linker L.

In another aspect, the present disclosure provides methods of treating a disorder responsive to modulation of IRAK4 activity and/or degradation of IRAK4 in a subject comprising administering to the subject an effective amount of at least one compound described herein. The present disclosure also includes the use of at least one compound described herein, or a pharmaceutically acceptable salt thereof, for the manufacture of a medicament for the treatment of a disorder responsive to modulation of IRAK4 activity and/or degradation of IRAK4. Also provided are compounds described herein, or pharmaceutically acceptable salts thereof, for use in treating a disorder responsive to modulation of IRAK4 activity and/or degradation of IRAK4. Methods of making the compounds described herein and any synthetic intermediates are also included in the present disclosure.

Other features or advantages will be apparent from the following detailed description of several embodiments, and also from the appended claims.

### DETAILED DESCRIPTION

Compounds or pharmaceutically acceptable salts thereof as described herein are capable of activating the selective ubiqitination of IRAK4 proteins via the ubiquitin-proteasome pathways (UPP) and cause degradation of IRAK4 proteins. In some embodiments, compounds or pharmaceutically acceptable salts thereof as described herein can modulate IRAK4 activities.

Compounds of the present disclosure, and pharmaceutical formulations thereof, may be useful in the treatment or prevention of conditions and/or disorders through mediation of IRAK4 function such as, for example, autoimmune disease, an inflammatory disease, bone diseases, metabolic diseases, neurological and neurodegenerative diseases, Alzheimer's disease, Ischemic stroke, Cerebral Ischemia, hypoxia, TBI (Traumatic Brain Injury), CTE (Chronic Traumatic Encephalopathy), epilepsy, Parkinson's disease (PD), Multiple Sclerosis (MS) and Amyotrophic Lateral Sclerosis (ALS).

### I. Definitions

Compounds are described using standard nomenclature. Unless defined otherwise, all technical and scientific terms used herein have the same meaning as is commonly understood by one of ordinary skill in the relevant art.

The terms “a” and “an” do not denote a limitation of quantity, but rather denote the presence of at least one of the referenced item. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the disclosure and does not pose a limitation on the scope of the disclosure unless otherwise claimed.

As used herein, the term “alkyl” refers to a fully saturated branched or unbranched hydrocarbon moiety. In some embodiments, the alkyl comprises 1 to 20 carbon atoms, 1 to 10 carbon atoms, 1 to 8 carbon atoms, 1 to 6 carbon atoms, or 1 to 4 carbon atoms. In some embodiments, an alkyl comprises from 6 to 20 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, or n-hexyl. Similarly, the alkyl portion (i.e., alkyl moiety) of an alkoxy or a haloalkyl have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls).

As used herein, the term “alkoxy” refers to a fully saturated branched or unbranched alkyl moiety attached through an oxygen bridge (i.e., a —O—C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. Preferably, alkoxy groups have about 1-4 carbons, more preferably about 1-2 carbons.

As used herein, the term “aryl” refers to a carbocyclic (all carbon) aromatic monocyclic or bicyclic ring system containing 6-10 carbon atoms. Examples of 6-10 membered aryl groups include phenyl and naphthyl. In some embodiments, the aryl is phenyl.

The term “bridged ring system”, as used herein, is a ring system where two non-adjacent atoms of the ring are connected (bridged) by one or more (preferably from one to three) atoms selected from C, N, O, and S. In one embodiment, a bridged ring system have from 6 to 8 ring members.

The term “fused ring system”, as used herein, is a ring system that has two ring structures sharing two adjacent ring atoms. In one embodiment, a fused ring system have from 8 to 12 ring members.

The term “spiro ring system,” as used herein, is a ring system that has two ring structures having one ring atom in common. In one embodiment, spiro ring systems have from 5 to 8 ring members.

The term “cycloalkyl” refers to partially or fully saturated monocyclic or bicyclic or spiro hydrocarbon groups of 3-7 carbon atoms, 3-6 carbon atoms, or 5-7 carbon atoms. In some embodiments, cycloalkyl is a 3- to 6-membered fully saturated monocyclic cycloalkyl (e.g., cyclopropyl, cyclobutyl, cyclopentyl, and cyclohexyl).

As used herein, the terms “carbocycle” and “carbocyclic ring” refer to saturated or partially unsaturated (i.e., non-aromatic) monocyclic or bicyclic hydrocarbon groups of, for example, 3-10, 3-8, 3-7, 3-5, 3-6, 4-6, 5-7 or 7-10 carbon atoms. 3 to 7 membered monocyclic carbocycles include, but are not limited to, cyclopropyl, cyclobutyl, cyclopentyl, cyclohexyl, cycloheptyl, cyclopropenyl, cyclobutenyl, cyclopenentyl, cyclohexenyl, cycloheptenyl, cyclobutadienyl, cyclopentadienyl, cyclohexadienyl, cycloheptadienyl and cycloheptatrienyl. Bicyclic carbocycles include, but are not limited to, bicyclo[2.1.1]hexyl, bicyclo[2.2.1]heptyl, bicyclo[2.2.1]heptenyl, 6,6-dimethylbicyclo-[3.1.1]heptyl, 2,6,6-trimethylbicyclo[3.1.1]heptyl, spiro[2.2]pentanyl and spiro[3.3]heptanyl. 7 to 10 membered bicyclic carbocycles include, but are not limited to, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, spiro[3.3]heptanyl, spiro[2.5]octanyl, bicyclo[3.3.0]octanyl, bicyclo[2.2.2]octanyl, bicyclo[3.3.1]nonanyl, bicyclo[3.3.2]decanyl and decalinyl.

As used herein the term “bridged-carbocyclic ring” refers to a cyclic moiety connected at two non-adjacent ring atoms of the carbocycle (e.g. bicyclo[1.1.1]pentane, bicyclo[2.2.1] heptane and bicyclo[3.2.1] octane).

As used herein the term “fused bicyclic ring system” or “fused carbobicyclic ring system” refers to a carbocycle connected at two non-adjacent ring atoms of the carbocycle.

Fused bicyclic ring systems include, but are not limited to, 1,2,3,4-tetrahydronaphthalene, (1S,5R)-1-methylbicyclo[3.1.0]hexane, bicyclo[3.1.0]hexane, bicyclo[4.1.0]heptane and 2,3-dihydro-1H-indene.

As used herein the term “spiro carbocyclic ring” means a two-ring system wherein both rings share one common carbon atom. Examples of spiro carbocyclic rings include spiro[2.5]octane, spiro[2.3]hexane, spiro[2.4]heptane, spiro[3.4]octane and the like.

“Halogen” or “halo” may be fluorine, chlorine, bromine or iodine (preferred halogens as substituents are fluorine and chlorine).

As used herein, the term “haloalkyl” or “halo-substituted alkyl” or refers to an alkyl group as defined herein, wherein at least one of the hydrogen atoms is replaced by a halo atom. The haloalkyl group can be monohalo-alkyl, dihaloalkyl or polyhaloalkyl including perhaloalkyl. A monohaloalkyl can have one iodo, bromo, chloro or fluoro within the alkyl group. Dihaloalkyl and polyhaloalkyl groups can have two or more of the same halo atoms or a combination of different halo groups within the alkyl. Typically the polyhaloalkyl group contains up to 9, or 8, or 7, or 6, or 5, or 4, or 3, or 2 halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl group refers to an alkyl group having all hydrogen atoms replaced with halo atoms.

As used herein, the term “haloalkoxy” refers to a fully saturated branched or unbranched haloalkyl moiety attached through an oxygen bridge (i.e., a —O—C₁₋₄ haloalkyl group wherein C₁₋₄ haloalkyl is as defined herein).

As used herein, the term “heteroaryl” refers to an aromatic 5- to 6-membered monocyclic or an 8- to 10-membered bicyclic ring system, having 1 to 4 heteroatoms independently selected from O, N and S, and wherein N can be oxidized (e.g., N(O)) or quaternized, and S can be optionally oxidized to sulfoxide and sulfone.

Examples of “5 or 6 membered heteroaryl” or “5- to 6-membered monocyclic heteroaryl” include, but are not limited to, pyrrolyl, furanyl, thiophenyl (or thienyl), imidazolyl, pyrazolyl, oxazolyl, isoxazolyl, thiazolyl, isothiazolyl, furazanyl, oxadiazolyl, thiadiazolyl, dithiazolyl, triazolyl, tetrazolyl, pyridinyl, pyrazinyl, pyrimidinyl, pyridazinyl, triazinyl, tetrazinyl, and the like. In some embodiments, a 5 to 6 membered heteroaryl is selected from pyrrolyl, pyridyl, pyrazolyl, thienyl, furanyl, oxazolyl, isoxazolyl, isothiazolyl, thiazolyl, imidazolyl, tetrazolyl, triazinyl, pyrimidyl, pyrazinyl, and thiazolyl. In some embodiments, a 5 to 6 membered heteroaryl is selected from pyridinyl, pyrimidinyl, 2H-1,2,3-triazolyl, isoxazolyl, isothiazolyl, thiazolyl, pyrazolyl and thienyl.

Examples of a 5-membered heteroaryl include, but are not limited to, pyrazolyl, imidazolyl, oxazolyl, isoxazolyl, 1,2,3-oxadiazolyl, 1,3,4-oxadiazolyl, 1,2,4-oxadizolyl, 1,2,3-thiadiazolyl, 1,3,4-thiadiazolyl, 1,2,4-thiadiazolyl, 1,2,3-triazolyl, 1,2,4-triazolyl, and tetrazolyl. Examples of 8- to 10-membered bicyclic heteroaryls include, but are not limited to, imidazolthiazolyl, imidazopyridinyl, imidazo[1,2-a]pyridinyl, imidazo[2,1-b]thiazolyl, indazolyl, 2H-indazolyl, indolyl, isoindolyl, 2λ²-isoindolinyl, benzimidazolyl, benzofuranyl, benzothiophenyl, benzothiazolyl, quinolinyl, isoquinolinyl, quinazolinyl, purinyl, thienopyridinyl and thieno[3,2-b]pyridinyl. Examples of 9- to 10-membered bicyclic heteroaryls include, but are not imitated to, imidazopyridinyl, imidazo[1,2-a]pyridinyl, indazolyl, 2H-indazolyl, indolyl, isoindolyl, 2λ²-isoindolinyl, benzimidazolyl, benzofuranyl, benzothiophenyl, benzothiazolyl, quinolinyl, isoquinolinyl, quinazolinyl, purinyl, thienopyridinyl and thieno[3,2-b]pyridinyl.

In some embodiments, a 5-membered heteroaryl is selected from

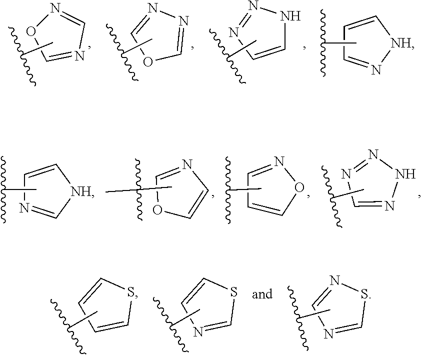

In some embodiments, a 6-membered heteroaryl is selected from

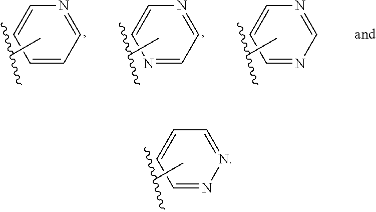

Examples of 9 to 10 membered heteroaryls include indolyl, indazolyl, benzofuranyl, quinoxalinyl, pyrazolo[1,5-a]pyridinyl, [1,2,4]triazolo[4,3-a]pyridinyl, isothiazolo[4,3-b]pyridinyl, pyrazolo[1,5-a]pyrimidinyl, pyrido[3,2-d]pyrimidinyl, imidazo[1,2-b]pyridazinyl, thieno[2,3-b]pyrazinyl, 1H-benzo[d]imidazolyl, benzo[d]thiazolyl, 1,6-naphthyridinyl, and 1,5-naphthyridinyl. In some embodiments, a 9 to 10 membered heteroaryl is selected from pyrazolo[1,5-a]pyridinyl, [1,2,4]triazolo[4,3-a]pyridinyl, isothiazolo[4,3-b]pyridinyl, pyrazolo[1,5-a]pyrimidinyl, pyrido[3,2-d]pyrimidinyl, imidazo[1,2-b]pyridazinyl, thieno[2,3-b]pyrazinyl, 1H-benzo[d]imidazolyl, benzo[d]thiazolyl, 1,6-naphthyridinyl, 1,5-naphthyridinyl, and 2H-indazolyl.

In some embodiments, a heteroaryl is an 8- to 9-membered bicyclic heteroaryl selected from:

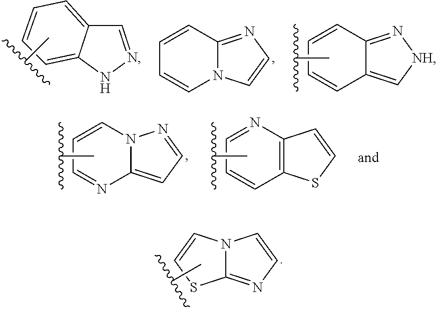

The term “heterocycle” or “monocyclic heterocycle” refers to a monocyclic ring which is partially or fully saturated and contains 1 to 2 heteroatoms, independently selected from sulfur, oxygen and/or nitrogen. Monocyclic heterocycles include, but are not limited to, oxtanyl, tetrahydrofuranyl, dihydrofuranyl, 1,4-dioxanyl, morpholinyl, 1,4-dithianyl, piperazinyl, piperidinyl, 1,3-dioxolanyl, pyrrolinyl, pyrrolidinyl, tetrahydropyranyl, oxathiolanyl, dithiolanyl, 1,3-dioxanyl, 1,3-dithianyl, oxathianyl, thiomorpholinyl, thiomorpholinyl 1,1 dioxide, tetrahydro-thiopyran 1,1-dioxide, 1,4-diazepanyl.

In some embodiments, a monocyclic heterocycle is selected from:

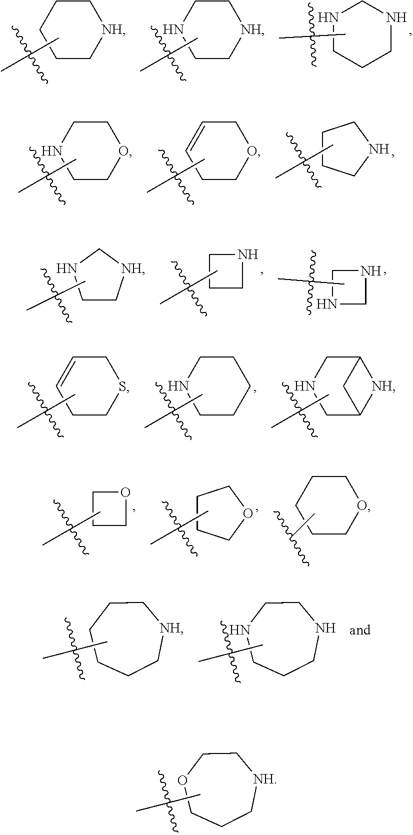

The term “bicyclic heterocycle” refers to a bicyclic ring which is partially or fully saturated and contains 1 to 2 heteroatoms, independently selected from sulfur, oxygen and/or nitrogen. Bicyclic heterocycles include, but are not limited to, 2,6-diazaspiro[3.3]heptane and pyrazolo[1,5-a]pyrimidine.

As used herein, the term “spiro bicyclic heterocycle” refers to a fully saturated bicyclic heterocycle ring system having two ring structures with one ring atom in common. In one embodiment, a spiro bicyclic heterocycle has from 7 to 11 ring members.

The term “partially or fully saturated heterocycle” refers to a nonaromatic ring that is either partially or fully saturated and may exist as a single ring, bicyclic ring (including fused heterocyclic rings) or a spiro ring. Unless specified otherwise, the heterocyclic ring is generally a 3 to 7 membered ring containing 1 to 3 heteroatoms (preferably 1, 2 or 3 heteroatoms) independently selected from sulfur, oxygen and/or nitrogen. Partially saturated or fully saturated heterocyclic rings include groups such as epoxy, aziridinyl, azetidinyl, tetrahydrofuranyl, dihydrofuranyl, dihydropyridinyl, pyrrolidinyl, imidazolidinyl, imidazolinyl, 1H-dihydroimidazolyl, hexahydropyrimidinyl, piperidinyl, piperazinyl, pyrazolidinyl, 2H-pyranyl, 4H-pyranyl, oxazinyl, morpholino, thiomorpholino, tetrahydrothienyl, tetrahydrothienyl 1,1-dioxide, oxazolidinyl, thiazolidinyl, 7-oxabicyclo[2.2.1]heptane, and the like. Partially saturated heterocycles include, but are not limited to, pyridin-2(1H)-one. A partially saturated heterocyclic ring also includes groups wherein a heterocyclic ring is fused to an aryl or heteroaryl ring (e.g., 2,3-dihydrobenzo furanyl, indolinyl (or 2,3-dihydroindolyl), 2,3-dihydrobenzothiophenyl, 2,3-dihydro benzothiazolyl, 1,3-dihydro-2H-benzo[d]imidazol-2-one, 1,2,3,4-tetrahydro quinolinyl, 1,2,3,4-tetrahydroisoquinolinyl, 5,6,7,8-tetrahydropyrido[3,4-b]pyrazinyl).

In some embodiments, a partially or fully saturated heterocycle is selected from:

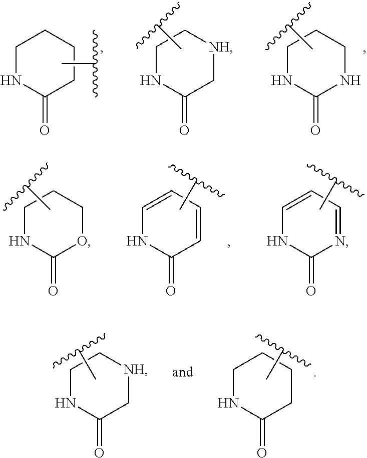

As used herein the term “bridged-heterocyclic ring system” refers to a 5 to 10 membered heterobicyclic moiety connected at two non-adjacent ring atoms of the heterocycle containing at least one heteroatom (e.g., oxygen, sulfur, nitrogen or combinations thereof) within a 5 to 10 membered cyclic ring system. Examples of the “bridged-heterocyclic ring system” include, but are not limited to, 2-oxabicyclo[2.1.1]hexane, 3-oxabicyclo[4.1.0]heptane, 2-oxabicyclo[2.2.1]heptane, 2-oxabicyclo[2.2.2]octane, 8-oxabicyclo[3.2.1]octane, and 2,6-dioxabicyclo[3.2.1]octane.

As used herein the term “fused heterobicyclic ring system” refers to two ring systems that share two adjacent ring atoms and at least one of the rings containing a ring atom that is a heteroatom selected from O, N and S. Examples of fused heterobicylic ring systems include, but are not limited to, 1,3-dihydroisobenzofuran, 4-methyl-3,4-dihydro-2H-benzo[b][1,4]oxazine, pyrazolo[1,5-a]pyrimidine, 5,6-dihydro-4H-pyrrolo[1,2-b]pyrazole, 6,7-dihydro-5H-cyclopenta[b]pyridine, 2-oxabicyclo[2.1.0]pentane, indolin-2-one, 2,3-dihydrobenzofuran, 1-methyl-2-oxo-1,2,3,4-tetrahydroquinoline, 3,4-dihydroquinolin-2(1H)-one, chromane, isochromane, 4,5,6,7-tetrahydro-3H-imidazo[4,5-c]pyridine, 8-azabicyclo [3.2.1]octan-3-ol, octahydropyrrolo[1,2-a]pyrazine, 5,6,7,8-tetrahydroimidazo[1,2-a]pyrazine, 3,8 diazabicyclo[3.2.1]octane, 8-oxa-3-azabicyclo[3.2.1]octane, 7-oxabicyclo [2.2.1]heptane, 1H-pyrazole, 2,5-diazabicyclo[2.2.1]heptane, 5,6,7,8-tetrahydro-[1,2,4]triazolo[4,3-a]pyrazine, 3-oxabicyclo[3.1.0]hexane, or 3-azabicyclo[3.1.0]hexane. A partially saturated heterocyclic ring also includes groups wherein the heterocyclic ring is fused to an aryl or heteroaryl ring (e.g., 2,3-dihydrobenzofuranyl, indolinyl (or 2,3-dihydro indolyl), 2,3-dihydrobenzothiophenyl, 2,3-dihydrobenzothiazolyl, 1,2,3,4-tetrahydro quinolinyl, 1,2,3,4-tetrahydroisoquinolinyl, 5,6,7,8-tetrahydropyrido[3,4-b]pyrazinyl, 6,7-dihydro-5H-pyrazolo[5,1-b][1,3]oxazine, and the like. In some embodiments, “fused heterobicyclic ring system” refers fused bicyclic heteroaryl.

In some embodiments, the term “7 to 10 membered fused heterobicyclic ring system” is limited to a 7 to 10 membered bicyclic heteroaryl, such as pyrazolo[1,5-a]pyrimidine, pyrazolo[1,5-a]pyridine, [1,2,4]triazolo[4,3-a]pyridine, [1,2,4]triazolo[1,5-a]pyridine, isothiazolo[4,3-b]pyridine, pyrrolo[1,2-a]pyrimidine, pyrido[3,2-d]pyrimidine, imidazo[1,2-b]pyridazine, thieno[2,3-b]pyrazine, 1H-benzo[d]imidazole, benzo[d]thiazole, 1,6-naphthyridine and 1,5-naphthyridine.

As used herein the term “spiro heterobicyclic ring system” means a two-ring system wherein both rings share one common atom. Examples of spiro heterobicyclic ring systems include oxaspiro[2.4]heptanyl, 5-oxaspiro[2.4]heptanyl, 4-oxaspiro[2.4]heptane, 4-oxaspiro[2.5]octanyl, 6-oxaspiro[2.5]octanyl, oxaspiro[2.5]octanyl, oxaspiro[3.4]octanyl, oxaspiro[bicyclo[2.1.1]hexane-2,3′-oxetan]-1-yl, oxaspiro[bicyclo[3.2.0]heptane-6,1′-cyclobutan]-7-yl, 2,6-diazaspiro[3.3]heptanyl, -oxa-6-azaspiro[3.3]heptane, 2,2,6-diazaspiro[3.3]heptane, 3-azaspiro[5.5]undecanyl, 3,9-diazaspiro[5.5]undecanyl, 7-azaspiro[3.5]nonane, 2,6-diazaspiro[3.4]octane, 8-azaspiro[4.5]decane, 1,6-diazaspiro[3.3]heptane, 5-azaspiro[2.5]octane, 4,7-diazaspiro[2.5]octane, 5-oxa-2-azaspiro[3.4]octane, 6-oxa-1-azaspiro[3.3]heptane, 3-azaspiro[5.5]undecanyl, 3,9-diazaspiro[5.5]undecanyl, and the like.

As used herein “Hydroxyl” or “Hydroxy” refers to the group —OH.

The term “oxo” (═O) refers to an oxygen atom connected to a carbon or sulfur atom by a double bond. Examples include carbonyl, sulfinyl, or sulfonyl groups (—C(O)—, —S(O)— or —S(O)₂—) such as, a ketone, aldehyde, or part of an acid, ester, amide, lactone, or lactam group and the like.

As used herein, when a group/variable (e.g., L, Z¹, Z² etc.) is defined as “bond”, it means that the two moieties attached to the group/variable are connected directly to each other. For example, when L in Formula (A) is a bond, it means that the IRAK moiety and the DSM moiety are connected directly.

IRAK-L-DSM (A).

As used herein, the phrase “optionally substituted” is used interchangeably with the phrase “substituted or unsubstituted.” In general the term “optionally substituted” refers to the replacement of hydrogen radicals in a given structure with the radical of a specified substituent. Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position.

Unless specified otherwise, the term “compounds of the present disclosure” refers to compounds of formula (A), as well as all stereoisomers (including diastereoisomers and enantiomers), rotamers, tautomers, isotopically labeled compounds (including deuterium substitutions), and inherently formed moieties (e.g., polymorphs, solvates and/or hydrates). When a moiety is present that is capable of forming a salt, then salts are included as well, in particular pharmaceutically acceptable salts.

The compounds and intermediates described herein may be isolated and used as the compound per se. Alternatively, when a moiety is present that is capable of forming a salt, the compound or intermediate may be isolated and used as its corresponding salt. As used herein, the terms “salt” or “salts” refers to an acid addition or base addition salt of a compound of the disclosure. “Salts” include in particular “pharmaceutical acceptable salts”.

The term “pharmaceutically acceptable salts” refers to salts that retain the biological effectiveness and properties of the compounds of this disclosure and, which typically are not biologically or otherwise undesirable. In many cases, the compounds of the present disclosure are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The salts can be synthesized by conventional chemical methods from a compound containing a basic or acidic moiety. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two.

Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

In some embodiments, the disclosure provides deuterated compounds in which any or more positions occupied by hydrogen can include enrichment by deuterium above the natural abundance of deuterium. For example, one or more hydrogen atoms are replaced with deuterium at an abundance that is at least 3340 times greater than the natural abundance of deuterium, which is 0.015% (i.e., at least 50.1% incorporation of deuterium), at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). In one embodiment, hydrogen is present at all positions at its natural abundance.

Isotopically-labeled compounds of formula (A) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the disclosure include those wherein the solvent of crystallization may be isotopically substituted, e.g. D₂O, d₆-acetone, d₆-DMSO.

It will be recognized by those skilled in the art that the compounds of the present disclosure may contain chiral centers and as such may exist in different stereoisomeric forms.

As used herein, the term “an optical isomer” or “a stereoisomer” refers to any of the various stereo isomeric configurations which may exist for a given compound of the present disclosure. It is understood that a substituent may be attached at a chiral center of a carbon atom. Therefore, the disclosure includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term is used to designate a racemic mixture where appropriate. When designating the stereochemistry for the compounds of the present disclosure, a single stereoisomer with known relative and absolute configuration of the two chiral centers is designated using the conventional RS system (e.g., (1S,2S)); a single stereoisomer with known relative configuration but unknown absolute configuration is designated with stars (e.g., (1R*,2R*)); and a racemate with two letters (e.g, (1RS,2RS) as a racemic mixture of (1R,2R) and (1S,2S); (1RS,2SR) as a racemic mixture of (1R,2S) and (1S,2R)).

“Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)— or (S)—.

Unless specified otherwise, the compounds of the present disclosure are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or HPLC chromatography columns, such as CHIRALPAK® and CHIRALCEL® available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the term “inhibit”, “inhibition” or “inhibiting” refers to the reduction or suppression of a given condition, symptom, or disorder, or disease, or a significant decrease in the baseline activity of a biological activity or process.

As used herein, a “patient,” “subject” or “individual” are used interchangeably and refer to either a human or non-human animal. The term includes mammals such as humans.

Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. Preferably, the subject is a human.

The phrase “pharmaceutically acceptable” indicates that the substance, composition or dosage form must be compatible chemically and/or toxicologically, with the other ingredients comprising a formulation, and/or the mammal being treated therewith.

As used herein, the term “treat”, “treating” or “treatment” of any disease or disorder, refers to the management and care of a patient for the purpose of combating the disease, condition, or disorder and includes the administration of a compound of the present disclosure to prevent the onset of the symptoms or complications, alleviating the symptoms or complications, or eliminating the disease, condition or disorder.

As used herein the term “stroke” has the meaning normally accepted in the art. The term can broadly refer to the development of neurological deficits associated with the impaired blood flow regardless of cause. Potential causes include, but are not limited to, thrombosis, hemorrhage and embolism. The term “ischemic stroke” refers more specifically to a type of stroke that is of limited extent and caused due to a blockage of blood flow.

As used herein, a subject is “in need of” a treatment if such subject would benefit biologically, medically or in quality of life from such treatment (preferably, a human).

As used herein the term “co-administer” refers to the presence of two active agents in the blood of an individual. Active agents that are co-administered can be concurrently or sequentially delivered.

The term “combination therapy” or “in combination with” or “pharmaceutical combination” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent being administered prior to, concurrent with, or sequentially to each other with no specific time limits. In each case, the treatment regimen will provide beneficial effects of the drug combination in treating the conditions or disorders described herein.

### II. Compounds of the Disclosure

The compounds of the present disclosure comprise a degradation signaling moiety (DSM) that can bind to an E3 ligase (e.g., the cereblon protein), an IRAK4 binding or targeting moiety and optionally a Linker that covalently links the DSM to the IRAK4 binding or targeting moiety.

In a first embodiment, the compound of the present disclosure is a compound of Formula (A):

IRAK-L-DSM (A)

or a pharmaceutically acceptable salt thereof, wherein the IRAK, L and DSM portions in Formula (A) as described in the first aspect above. In some embodiments, the DSM, IRAK and Linker portions in Formula (A) are as described below.

### A. IRAK4 Binding or Targeting Moiety

In a second embodiment of the present disclosure, for the compound of formula (A), IRAK is an IRAK4 binding moiety represented by Formula (IA) or (IB):

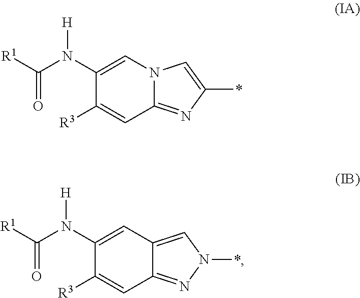

or a pharmaceutically acceptable salt thereof; and the definitions for the other variables are as defined in the first embodiment.

In a third embodiment of the present disclosure, for the compound of formula (A), or a pharmaceutically acceptable salt thereof, IRAK is an IRAK4 binding moiety represented by Formula (I), (IA) or (IB), wherein R¹ is selected from phenyl optionally substituted with 1 to 3 R⁵; 5 or 6 membered heteroaryl having 1 to 2 nitrogen atoms, said heteroaryl is optionally substituted with 1 to 3 R⁵; and 9 to 10 membered bicyclic heteroaryl having 1, 2 or 3 nitrogen atoms, said ring system is optionally substituted with 1 to 3 R⁵; and the definitions for the other variables are as defined in the first embodiment.

In a fourth embodiment of the present disclosure, for the compound of formula (A), or a pharmaceutically acceptable salt thereof, IRAK is an IRAK4 binding moiety represented by Formula (I), (IA) or (IB), wherein R¹ is selected from oxazole optionally substituted with 1 to 2 R⁵; phenyl optionally substituted with 1 to 2 R⁵; pyrazole optionally substituted with 1 to 2 R⁵; pyridine optionally substituted with 1 to 2 R⁵; pyridone optionally substituted with 1 to 2 R⁵; pyrimidine optionally substituted with 1 to 2 R⁵; and pyrazolo[1,5-a]pyrimidine optionally substituted with 1 to 2 R⁵; and the definitions for the other variables are as defined in the first embodiment. In some embodiments, R¹ is selected from oxazole optionally substituted with 1 to 2 R⁵; phenyl optionally substituted with 1 to 2 R⁵; pyrazole optionally substituted with 1 to 2 R⁵; pyridine optionally substituted with 1 to 2 R⁵; pyrimidine optionally substituted with 1 to 2 R⁵; and pyrazolo[1,5-a]pyrimidine optionally substituted with 1 to 2 R⁵; and the definitions for the other variables are as defined in the first embodiment.

In a fifth embodiment of the present disclosure, for the compound of formula (A), or a pharmaceutically acceptable salt thereof, IRAK is an IRAK4 binding moiety represented by Formula (I), (IA) or (IB), wherein R¹ is represented by one of the following formulae:

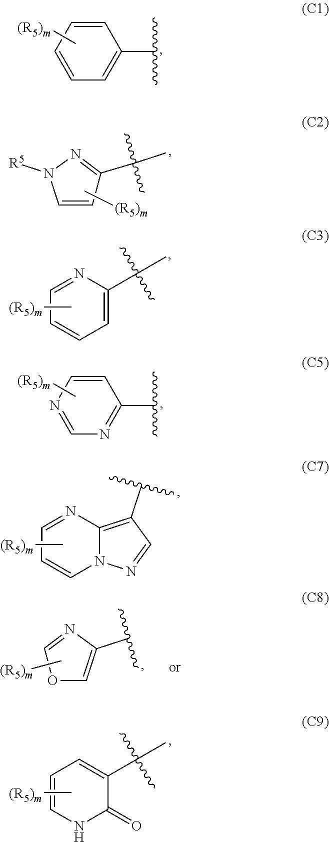

wherein m is 0, 1 or 2; and the definitions for the other variables are as defined in the first embodiment. In some embodiments, R¹ is represented by formula (C1), (C2), (C3), (C5), (C7) or (C8) and the definitions for the other variables are as defined in the first embodiment.

In a sixth embodiment of the present disclosure, for the compound of formula (A), or a pharmaceutically acceptable salt thereof, IRAK is an IRAK4 binding moiety represented by Formula (I), (IA) or (IB), wherein R¹ is represented by one of the following formulae:

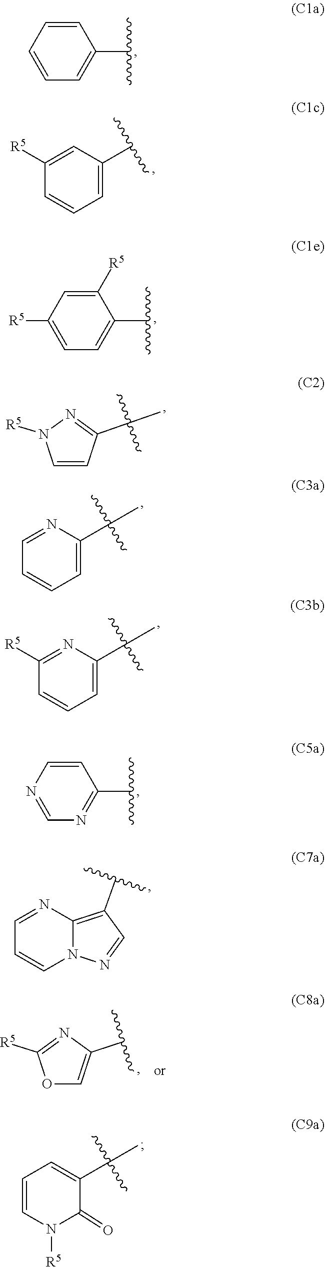

and the definitions for the other variables are as defined in the first embodiment. In some embodiments, R¹ is represented by formula (C1a), (C1c), (C1e), (C2), (C3a), (C3b), (C5a), (C7a) or (C8a) and the definitions for the other variables are as defined in the first embodiment.

In a seventh embodiment of the present disclosure, for the compound of formula (A), or a pharmaceutically acceptable salt thereof, IRAK is an IRAK4 binding moiety represented by Formula (I), (IA) or (IB), wherein R¹ is represented by the following formula:

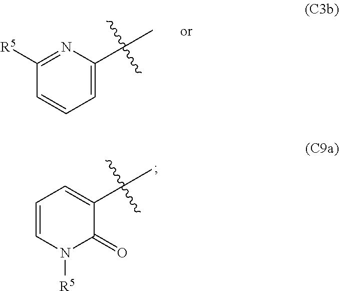

and the definitions for the other variables are as defined in the first embodiment. In some embodiment, R¹ is represented by formula (C3b) and the definitions for the other variables are as defined in the first embodiment.

In an eighth embodiment of the present disclosure, for the compound of formula (A), or a pharmaceutically acceptable salt thereof, IRAK is an IRAK4 binding moiety represented by Formula (I), (IA) or (IB), wherein R² is hydrogen; and the definitions for the other variables are as defined in the first, second, third, fourth, fifth, sixth or seventh embodiment.

In a ninth embodiment of the present disclosure, for the compound of formula (A), or a pharmaceutically acceptable salt thereof, IRAK is an IRAK4 binding moiety represented by one of the following formulae:

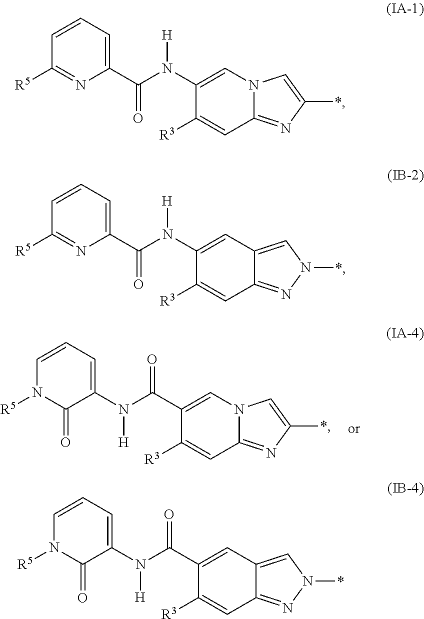

and the definitions for the other variables are as defined in the first embodiment. In some embodiments, the IRAK4 binding moiety is represented by formula (IA-1) or (IB-2) and the definitions for the other variables are as defined in the first embodiment.

In a tenth embodiment of the present disclosure, for the compound of formula (A), or a pharmaceutically acceptable salt thereof, IRAK is an IRAK4 binding moiety represented by one of Formula (I), (IA), (IB), (IA-1) or (IB-2), wherein R³ is C₁₋₄alkyl or —OR⁶, wherein the C₁₋₄alkyl is optionally substituted with at least one halogen; and R⁶ is C₁₋₅alkyl, C₃₋₆cycloalkyl or a 4 to 7 membered fully saturated heterocycle containing 1 or 2 heteroatoms selected from nitrogen and oxygen, wherein the C₁₋₅alkyl represented by R⁶ is optionally substituted with one to three halogens, and the C₃₋₆cycloalkyl represented by R⁶ is optionally substituted with 1 to 3 substituents R^{6b} independently selected from halogen, C₁₋₄alky, C₁₋₄ haloalkyl, and C₁₋₄alkoxy. In some embodiments, R³ is C₁₋₄alkyl or —OR⁶, wherein the C₁₋₄alkyl is optionally substituted with at least one halogen; and R⁶ is C₁₋₅alkyl; and the definitions for the other variables are as defined in the first, third, fourth, fifth, sixth, seventh or eighth embodiment.

In an eleventh embodiment of the present disclosure, for the compound of formula (A), or a pharmaceutically acceptable salt thereof, IRAK is an IRAK4 binding moiety represented by one of Formula (I), (IA), (IB), (IA-1) or (IB-2), wherein R³ is —CF₃ or —O—CH(CH₃)₂; and the definitions for the other variables are as defined in the first, third, fourth, fifth, sixth, seventh or eighth embodiment.

In a twelfth embodiment of the present disclosure, for the compound of formula (A), or a pharmaceutically acceptable salt thereof, IRAK is an IRAK4 binding moiety represented by one of Formula (I), (IA), (IB), (IA-1) or (IB-2), wherein R³ is —O—CH(CH₃)₂; and the definitions for the other variables are as defined in the first, third, fourth, fifth, sixth, seventh or eighth embodiment.

In a thirteenth embodiment of the present disclosure, for the compound of formula (A), or a pharmaceutically acceptable salt thereof, IRAK is an IRAK4 binding moiety represented by one of Formula (I), (IA), (IB), (IA-1) or (IB-2), wherein R⁵ for each occurrence, is independently selected from C₁₋₄ alkyl, halogen, C₁₋₄haloalkyl and C₃₋₄cycloalkyl, and wherein said C₃₋₄cycloalkyl is optionally substituted with 1 halo; and the definitions for the other variables are as defined in the first, third, fourth, fifth, sixth, seventh, eighth, tenth, eleventh or twelfth embodiment. In some embodiments, R⁵ for each occurrence, is independently selected from C₁₋₄ alkyl, halogen and C₁₋₄haloalkyl; and the definitions for the other variables are as defined in the first, third, fourth, fifth, sixth, seventh, eighth, tenth, eleventh or twelfth embodiment.

In a fourteenth embodiment of the present disclosure, for the compound of formula (A), or a pharmaceutically acceptable salt thereof, IRAK is an IRAK4 binding moiety represented by one of Formula (I), (IA), (IB), (IA-1) or (IB-2), wherein R⁵ for each occurrence, is independently selected from —CH₃, —CHF₂, —CF₃, F, cyclopropyl, and

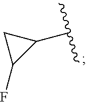 [SMILES: *C1CC1F]

and the definitions for the other variables are as defined in the first, third, fourth, fifth, sixth, seventh, eighth, tenth, eleventh or twelfth embodiment. In some embodiments, R⁵ for each occurrence, is independently selected from —CH₃, —CHF₂, —CF₃ and F; and the definitions for the other variables are as defined in the first, third, fourth, fifth, sixth, seventh, eighth, tenth, eleventh or twelfth embodiment.

In a fifteenth embodiment of the present disclosure, for the compound of formula (A), or a pharmaceutically acceptable salt thereof, IRAK is an IRAK4 binding moiety represented by one of the following formulae:

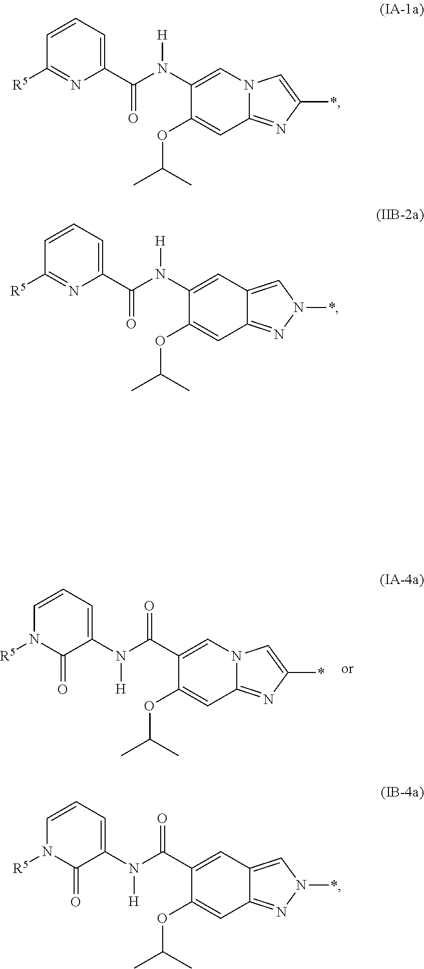

wherein R⁵ is C₁₋₃ alkyl, C₁₋₃ haloalkyl, C₃₋₄cycloalkyl, and wherein said C₃₋₄cycloalkyl is optionally substituted with 1 halo; and the definitions for the other variables are as defined in the first embodiment. In some embodiments, the IRAK4 binding moiety represented formula (IA-1a) or (IIB-2a), R⁵ is C₁₋₃ alkyl or C₁₋₃ haloalkyl; and the definitions for the other variables are as defined in the first embodiment.
In a sixteenth embodiment of the present disclosure, for the compound of formula (A), or a pharmaceutically acceptable salt thereof, IRAK is an IRAK4 binding moiety represented by one of Formula (IA-1a) or (JIB-2a), wherein R⁵ is CH₃, CHF₂, CF₃, cyclopropyl, or

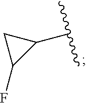 [SMILES: *C1CC1F]

and the definitions for the other variables are as defined in the fifteenth embodiment. In some embodiments, R⁵ is CH₃, CHF₂, or CF₃, and the definitions for the other variables are as defined in the fifteenth embodiment.

### B. Degradation Signaling Moiety (DSM)

The degradation signaling moiety (DSM) in compounds of formula (A) or a pharmaceutically acceptable salt thereof can be a suitable moiety that binds to an E3 ubiquitin ligase (e.g., the cereblon protein), for example, a degron or E3 ubiquitin ligase binding or targeting moiety described in WO2020/210630 titled “Tricyclic Degraders of Ikaros and Aiolos”; WO2020/181232 titled “Heterocyclic Compounds for Medical Treatment”; WO2020/132561 titled “Targeted Protein Degradation”; WO2019/204354 titled “Spirocyclic Compounds”; WO2019/099868 titled “Degraders and Degrons for Targeted Protein Degradation”; WO2018/237026 titled “N/O-Linked Degrons and Degronimers for Protein Degradation”; WO2017/197051 titled “Amine-Linked C3-Glutarimide Degronimers for Target Protein Degradation”; WO2017/197055 titled “Heterocyclic Degronimers for Target Protein Degradation”; WO2017/197036 titled “Spirocyclic Degronimers for Target Protein Degradation”; WO2017/197046 titled “C3-Carbon Linked Glutarimide Degronimers for Target Protein Degradation”; and WO2017/197056 titled “Bromodomain Targeting Degronimers for Target Protein Degradation”. Other degradation signaling moiety or E3 ubiquitin ligase binding or targeting moiety that can be used are those described in WO2015/160845; WO2016/105518; WO2016/118666; WO2016/149668; WO2016/197032; WO2016/197114; WO2017/007612; WO2017/011371; WO2017/011590; WO2017/030814; WO2017/046036; WO2017/176708; WO2017/176957; WO2017/180417; WO2018/053354; WO2018/071606; WO2018/102067; WO2018/102725; WO2018/118598; WO2018/119357; WO2018/119441; WO2018/119448; WO2018/140809; WO2018/144649; WO2018/119448; WO2018/226542; WO2019/023553, WO2019/195201, WO2019/199816, and WO2019/099926. The entire teachings of the above-referenced PCT publications are incorporated herein by reference.

In a seventeenth embodiment of the present disclosure, for the compound of formula (A), or a pharmaceutically acceptable salt thereof, DSM is a degradation signaling moiety of formula (D):

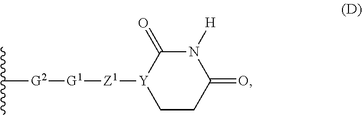

wherein [symbol] represents a bond to the linker L; Y is CR^{D1} or N; Z¹ is selected from bond, —NR^{D2}—, —O— and —CH₂—; G¹ is selected from 6- to 10-membered aryl, 5- to 10-membered heteroaryl and partially saturated 4- to 11-membered heterocycle; wherein the 6- to 10-membered aryl, 5- to 10-membered heteroaryl and partially saturated 4- to 11-membered heterocycle represented by G¹ are each optionally substituted with one or more R^{D3}; G² is selected from Het₁, *—NR^{D4}-Het₁-_{*}^{*}, *—NR^{D4}-Het₁-C₁₋₄ alkyl-_{*}^{*}, *—C₁₋₄ alkyl-C(R^{D1})=Het₁-_{*}^{*}, *—C(O)—C₁₋₄ alkyl-Het₁-_{*}^{*}, *-Het₁-C₁₋₆ alkyl-_{*}^{*}, *-Het₁-O—_{*}^{*}, *—C(O)—C₁₋₄ alkyl-Het₁-C(O)—_{*}^{*}, —C(O)-Het₁-C(O)—_{*}^{*}, *—C(O)-phenyl-C₁₋₄ alkyl-NHC(O)—_{*}^{*}; wherein *— represents a bond to the linker L, and _{*}^{*}— represents a bond to G¹; Het₁ is 4- to 7-membered monocyclic heterocycle or 7- to 11-membered bicyclic heterocycle, each of which is optionally substituted with one or more R^{D5}; R^{D1} is selected from H, C₁₋₆ alkyl or halogen; R^{D2} is H or C₁₋₃ alkyl; R^{D3} is, for each occurrence, independently selected from H, halogen, C₁₋₄ alkyl and C₁₋₄haloalkyl; R^{D4} is H or C₁₋₃ alkyl; and R^{D5} is, for each occurrence, independently selected from H, halogen, hydroxyl, C₁₋₄ alkyl, C₁₋₄haloalkyl and C₁₋₄ alkoxy; and the definitions for the other variables are as defined in the first, second, third, fourth, fifth, sixth, seventh, eighth, ninth, tenth, eleventh, twelfth, thirteenth, fourteenth, fifteenth or sixteenth embodiment.

In an eighteenth embodiment of the present disclosure, for the compound of formula (A), or a pharmaceutically acceptable salt thereof, DSM is a degradation signaling moiety of formula (D), wherein Het₁ is a 4 to 7 membered monocyclic saturated heterocycle containing 1 or 2 nitrogen atoms or a 7 to 11 membered saturated spiro bicyclic heterocycle containing 1 or 2 heteroatoms selected from N and O, each of which is optionally substituted with 1 or 2 R^{D5}; and the definitions for the other variables are as defined in the seventeenth embodiment.

In a nineteenth embodiment of the present disclosure, for the compound of formula (A), or a pharmaceutically acceptable salt thereof, DSM is a degradation signaling moiety of formula (D), wherein Het₁ is piperidine, piperazine, 2-azaspiro[3.3]heptane, 2,6-diazaspiro[3.3]heptane, 5-azaspiro[3.4]octane or 9-oxa-9-azaspiro[5.5]undecane, each of which is optionally substituted with 1 or 2 R^{D5}; and the definitions for the other variables are as defined in the seventeenth embodiment.

In a twentieth embodiment of the present disclosure, for the compound of formula (A), or a pharmaceutically acceptable salt thereof, DSM is a degradation signaling moiety of formula (D-I) or (D-II):

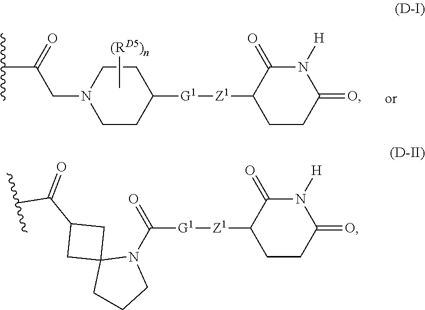

wherein: [symbol] represents a bond to the linker L; Z¹ is selected from bond, —NR^{D2}— and —O—; G¹ is selected from 6- to 10-membered aryl, 5- to 10-membered heteroaryl and partially saturated 4- to 11-membered heterocycle; wherein the 6- to 10-membered aryl, 5- to 10-membered heteroaryl and partially saturated 4- to 11-membered heterocycle represented by G¹ are each optionally substituted with one or more R^{D3}; R^{D2} is H or C₁₋₃ alkyl; R^{D3} is, for each occurrence, independently selected from H, halogen and C₁₋₄ alkyl; R^{D4} is C₁₋₃ alkyl; R^{D5} is halogen; and n is 0, 1 or 2; and the definitions for the other variables are as defined in the seventeenth embodiment.

In a twenty-first embodiment of the present disclosure, for the compound of formula (A), or a pharmaceutically acceptable salt thereof, DSM is a degradation signaling moiety of formula (D), (D-I) or (D-II), wherein G¹ is 6- to 10-membered aryl, 5- to 10-membered heteroaryl or partially saturated 4- to 11-membered heterocycle; wherein the 6- to 10-membered aryl, 5- to 10-membered heteroaryl and partially saturated 4- to 11-membered heterocycle represented by G¹ are each optionally substituted with 1 or 2 R^{D3}; and the definitions for the other variables are as defined in the seventeenth, eighteenth, nineteenth or twentieth embodiment.

In a twenty-second embodiment of the present disclosure, for the compound of formula (A), or a pharmaceutically acceptable salt thereof, DSM is a degradation signaling moiety of formula (D), (D-I) or (D-II), wherein G¹ is selected from phenyl, pyrazolyl, pyridinyl and pyrimidinyl, 1,3-dihydro-2H-benzo[d]imidazol-2-one, indazolyl, and indolyl, each of which is optionally substituted with 1 or 2 R^{D3}; and the definitions for the other variables are as defined in the seventeenth, eighteenth, nineteenth or twentieth embodiment.

In a twenty-third embodiment of the present disclosure, for the compound of formula (A), or a pharmaceutically acceptable salt thereof, DSM is a degradation signaling moiety of formula (D), (D-I) or (D-II), wherein G¹ is represented by any one of the following formulae:

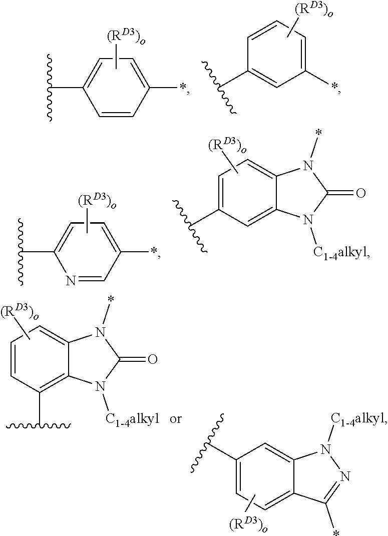

wherein: o is 0, 1 or 2, [symbol] represents a bond to G², and —* represents a bond to Z¹; and the definitions for the other variables are as defined in the seventeenth, eighteenth, nineteenth or twentieth embodiment.

In a twenty-fourth embodiment of the present disclosure, for the compound of formula (A), or a pharmaceutically acceptable salt thereof, DSM is a degradation signaling moiety of formula (D), (D-I) or (D-II), wherein G¹ is 6- to 10-membered aryl or 5- to 10-membered heteroaryl; wherein the 6- to 10-membered aryl and 5- to 10-membered heteroaryl represented by G¹ are each optionally substituted with 1 or 2 R^{D3}; and the definitions for the other variables are as defined in the seventeenth, eighteenth, nineteenth or twentieth embodiment.

In a twenty-fifth embodiment of the present disclosure, for the compound of formula (A), or a pharmaceutically acceptable salt thereof, DSM is a degradation signaling moiety of formula (D), (D-I) or (D-II), wherein G¹ is selected from phenyl, pyrazolyl, pyridinyl and pyrimidinyl, indazolyl, and indolyl, each of which is optionally substituted with 1 or 2 R^{D3}; and the definitions for the other variables are as defined in the seventeenth, eighteenth, nineteenth or twentieth embodiment.

In a twenty-sixth embodiment of the present disclosure, for the compound of formula (A), or a pharmaceutically acceptable salt thereof, DSM is a degradation signaling moiety of formula (D), (D-I) or (D-II), wherein G¹ is represented by any one of the following formulae:

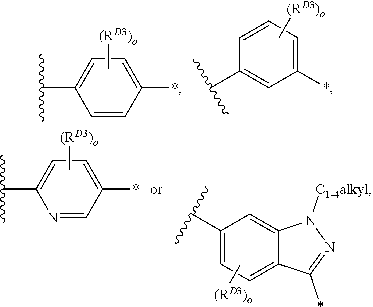

wherein: o is 0, 1 or 2, [symbol] represents a bond to G², and —* represents a bond to Z¹; and the [symbol] definitions for the other variables are as defined in the seventeenth, eighteenth, nineteenth or twentieth embodiment.

In a twenty-seventh embodiment of the present disclosure, for the compound of formula (A), or a pharmaceutically acceptable salt thereof, DSM is a degradation signaling moiety of formula (D), (D-I) or (D-II), wherein R^{D1} is H, —CH₃ or F; and the definitions for the other variables are as defined in the seventeenth, eighteenth, nineteenth, twentieth, twenty-first, twenty-second, twenty-third, twenty-fourth, twenty-fifth or twenty-sixth embodiment.

In a twenty-eighth embodiment of the present disclosure, for the compound of formula (A), or a pharmaceutically acceptable salt thereof, DSM is a degradation signaling moiety of formula (D), (D-I) or (D-II), wherein R^{D2} is H; and the definitions for the other variables are as defined in the seventeenth, eighteenth, nineteenth, twentieth, twenty-first, twenty-second, twenty-third, twenty-fourth, twenty-fifth, twenty-sixth or twenty-seventh embodiment.

In a twenty-ninth embodiment of the present disclosure, for the compound of formula (A), or a pharmaceutically acceptable salt thereof, DSM is a degradation signaling moiety of formula (D), (D-I) or (D-II), wherein R^{D3} is, for each occurrence, independently selected from H, Cl, F and —CH₃; and the definitions for the other variables are as defined in the seventeenth, eighteenth, nineteenth, twentieth, twenty-first, twenty-second, twenty-third, twenty-fourth, twenty-fifth, twenty-sixth, twenty-seventh or twenty-eighth embodiment.

In a thirtieth embodiment of the present disclosure, for the compound of formula (A), or a pharmaceutically acceptable salt thereof, DSM is a degradation signaling moiety of formula (D), (D-I) or (D-II), wherein R^{D4} is —CH₃; and the definitions for the other variables are as defined in the seventeenth, eighteenth, nineteenth, twentieth, twenty-first, twenty-second, twenty-third, twenty-fourth, twenty-fifth, twenty-sixth, twenty-seventh, twenty-eighth or twenty-ninth embodiment.

In a thirty-first embodiment of the present disclosure, for the compound of formula (A), or a pharmaceutically acceptable salt thereof, DSM is a degradation signaling moiety of formula (D), (D-I) or (D-II), wherein R^{D5} for each occurrence, is independently F or OH; and the definitions for the other variables are as defined in the seventeenth, eighteenth, nineteenth, twentieth, twenty-first, twenty-second, twenty-third, twenty-fourth, twenty-fifth, twenty-sixth, twenty-seventh, twenty-eighth, twenty-ninth or thirtieth embodiment.

In a thirty-second embodiment of the present disclosure, for the compound of formula (A), or a pharmaceutically acceptable salt thereof, DSM represents any one of the following attached to L:

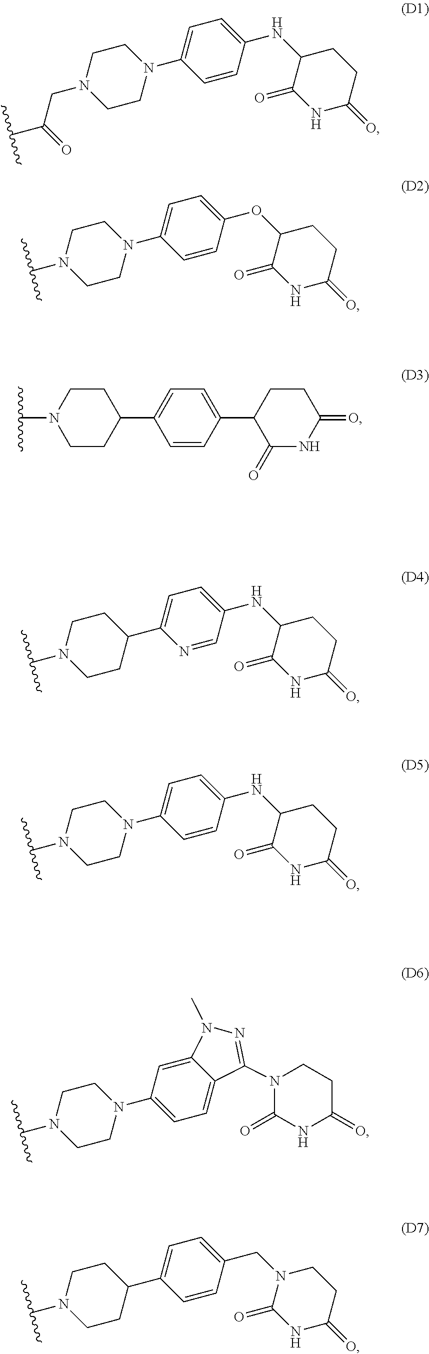

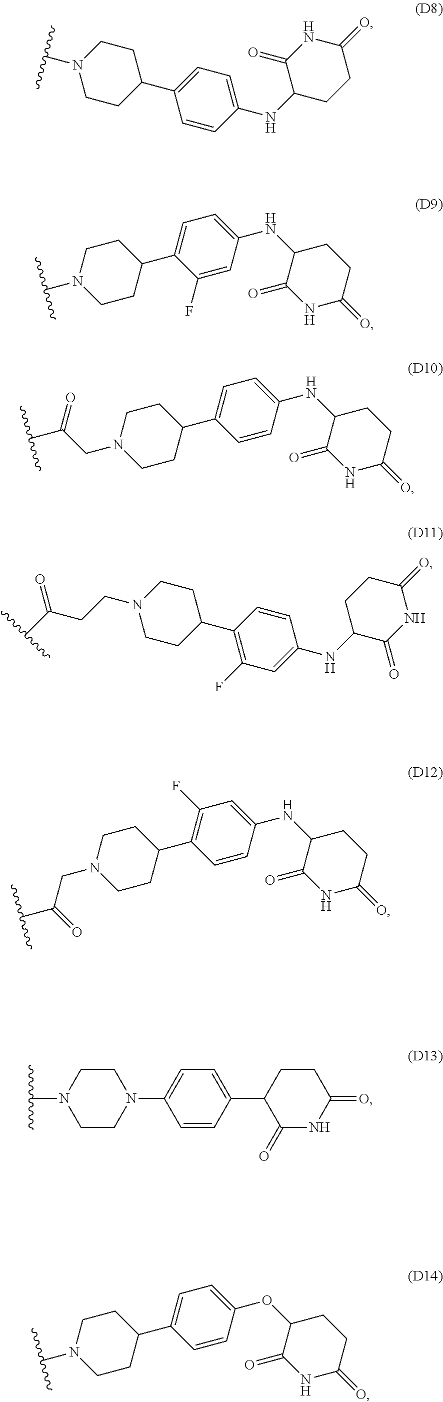

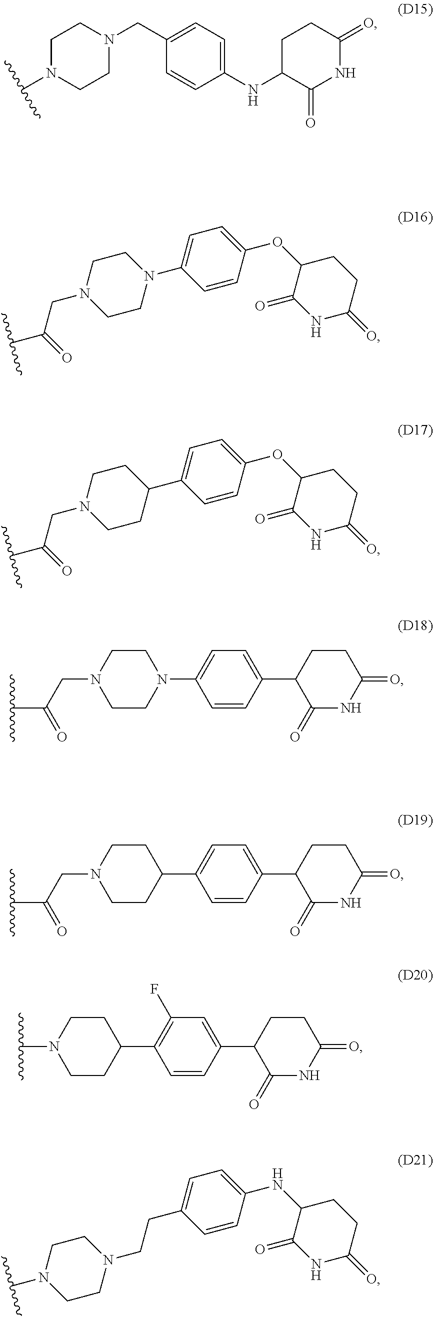

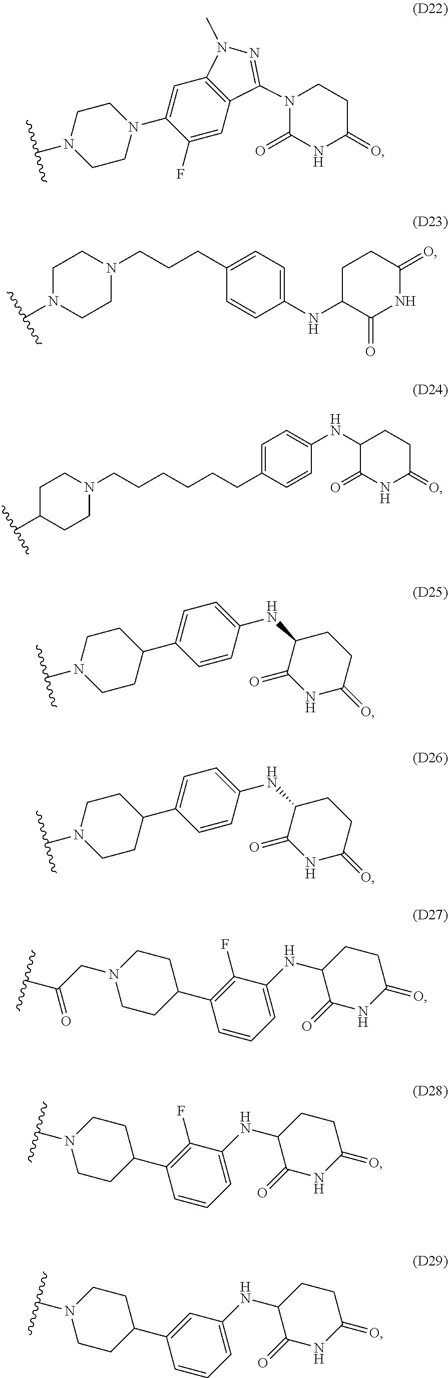

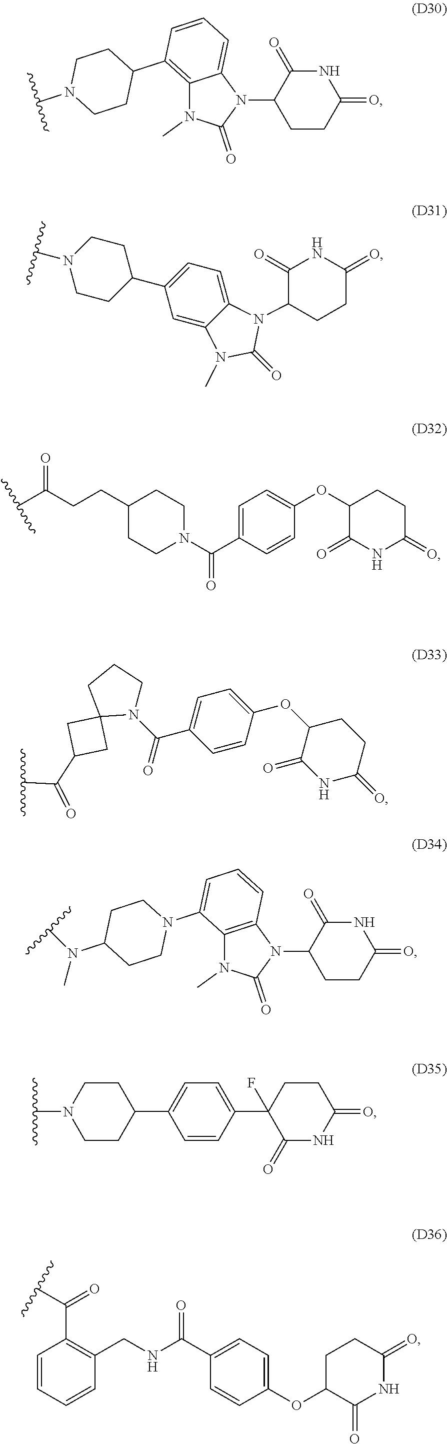

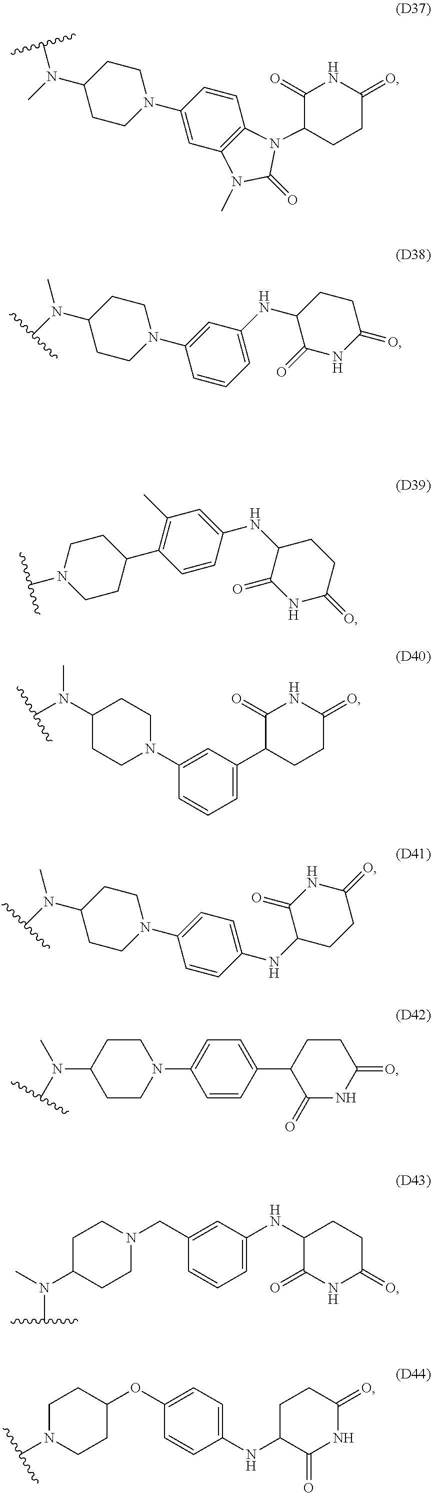

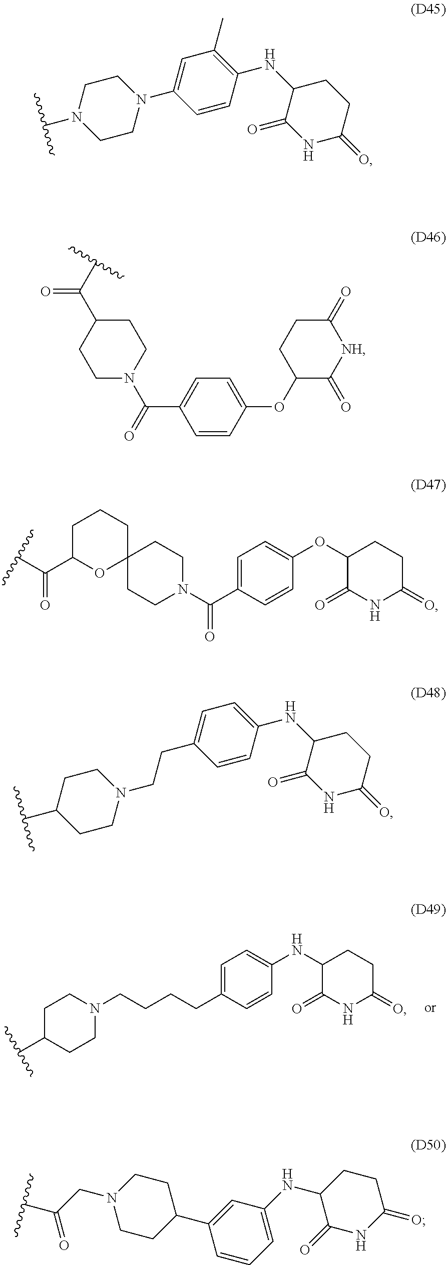

and the definitions for the other variables are as defined in the first, second, third, fourth, fifth, sixth, seventh, eighth, ninth, tenth, eleventh, twelfth, thirteenth, fourteenth, fifteenth or sixteenth embodiment.

### C. Linker

In a thirty-third embodiment of the present disclosure, for the compound of formula (A), or a pharmaceutically acceptable salt thereof, L is a bond, C₁₋₈ alkyl or is represented by formula (L-1), (L-2) or (L-3):

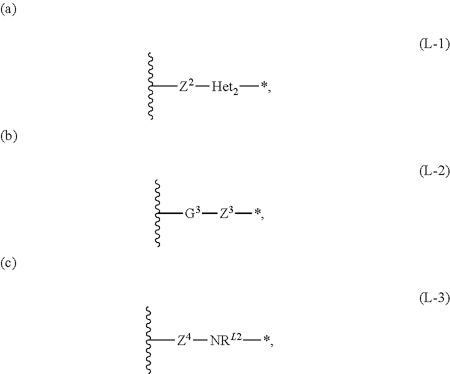

wherein: Z² is bond or C₁₋₄ alkyl optionally substituted with one or more halogen; Het₂ is 4- to 7-membered heterocycle optionally substituted by one or more R^{L1}; G³ is C₃₋₇ cycloalkyl or 4- to 7-membered heterocycle; wherein the C₃₋₇ cycloalkyl and 4- to 7-membered heterocycle represented by G³ are each optionally substituted with one or more R^{L3}; Z³ is C₁₋₄ alkyl or _{*}^{*}—C₁₋₄ alkyl-C(O)—*, wherein _{*}^{*}— represents a bond connected to G³; —* is a bond connected to the DSM; and the C₁₋₄ alkyl is optionally substituted with one or more halogen; Z⁴ is C₁₋₄ alkyl optionally substituted by R^{L}; R^{U} is, for each occurrence, independently selected from H, halogen, C₁₋₄ alkyl and C₁₋₄haloalkyl; R^{L2} is H or C₁₋₄ alkyl; R^{L3} is, for each occurrence, independently selected from H, halogen, C₁₋₄ alkyl and C₁₋₄haloalkyl; R^{L} is halo, —OR^{L5}, or C₁₋₄ alkyl optionally substituted by halogen, C₃₋₇ cycloalkyl, phenyl, 4- to 7-membered monocyclic saturated heterocycle, or 5- to 6-membered heteroaryl, wherein the C₃₋₇ cycloalkyl, phenyl, 4- to 7-membered monocyclic saturated heterocycle, and 5- to 6-membered heteroaryl are each optionally substituted with one to three substituents independently selected from halogen, C₁₋₄ alkyl, C₁₋₄ haloalkyl, C₁₋₄ alkoxy and C₁₋₄ [symbol] haloalkoxy; R^{L5} is H, C₁₋₄ alkyl or C₁₋₄ haloalkyl; [symbol] represents a bond to the IRAK4 binding moiety; and —* represents a bond to the degradation signaling moiety DSM; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first or thirty-second embodiment.

In a thirty-fourth embodiment of the present disclosure, for the compound of formula (A), or a pharmaceutically acceptable salt thereof, L is a bond; and the definitions for the other variables are as defined in the thirty-third embodiment.

In a thirty-fifth embodiment of the present disclosure, for the compound of formula (A), or a pharmaceutically acceptable salt thereof, L is C₁₋₈ alkyl; and the definitions for the other variables are as defined in the thirty-third embodiment.

In a thirty-sixth embodiment of the present disclosure, for the compound of formula (A), or a pharmaceutically acceptable salt thereof, L is represented by formula (L-1), (L-2) or (L-3), wherein Het₂ is selected from azetidinyl, piperidinyl and pyrrolidinyl; wherein the azetidinyl, piperidinyl and pyrrolidinyl represented by Het₂ are each optionally substituted by one or more R^{L1}; and G³ is azetidinyl, cyclohexyl or piperidinyl; wherein the cyclohexyl and piperidinyl represented by G³ are each optionally substituted with one or more R^{L3}; and the definitions for the other variables are as defined in the thirty-third embodiment.

In a thirty-seventh embodiment of the present disclosure, for the compound of formula (A), or a pharmaceutically acceptable salt thereof, L is represented by formula (L-1), (L-2) or (L-3), wherein Z² is bond or —CH₂—; Z³ is —CH₂—, —CH₂—CH₂—, _{*}^{*}—CH₂—C(O)—* or _{*}^{*}—CH₂—CH₂—C(O)—*; and Z⁴ is —CH(CH₂Ph)- or —CH₂—CH₂—CH₂—; and the definitions for the other variables are as defined in the thirty-third or thirty-sixth embodiment.

In a thirty-eighth embodiment of the present disclosure, for the compound of formula (A), or a pharmaceutically acceptable salt thereof, L is represented by formula (L-1), (L-2) or (L-3), wherein R^{L1} is H; R^{L2} is H; R^{L3} is H; and R^{L4} is benzyl; and the definitions for the other variables are as defined in the thirty-third, thirty-sixth or thirty-seventh embodiment.

In a thirty-ninth embodiment of the present disclosure, for the compound of formula (A), or a pharmaceutically acceptable salt thereof, L is represented by formula (L-1) and Het₂ is represented by one of the formulae:

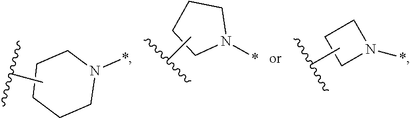

wherein: [symbol] represents a bond to Z²; and —* represents a bond to the degradation signaling moiety DSM; and the definitions for the other variables are as defined in the thirty-third, thirty-seventh or thirty-eighth embodiment.

In a fortieth embodiment of the present disclosure, for the compound of formula (A), or a pharmaceutically acceptable salt thereof, L is represented by formula (L-2) and G³ is represented by one of the formulae:

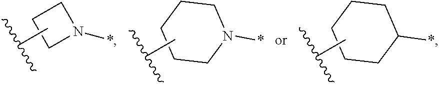

wherein: [symbol] represents a bond to the IRAK4 binding moiety; and —* represents a bond to Z³; and the definitions for the other variables are as defined in the thirty-third, thirty-seventh or thirty-eighth embodiment.

In a forty-first embodiment of the present disclosure, for the compound of formula (A), or a pharmaceutically acceptable salt thereof, L is represented by formula (L-1) and Het₂ is:

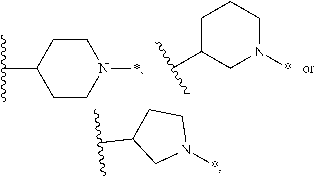

wherein [symbol] represents a bond to Z²; and —* represents a bond to the degradation signaling [symbol] moiety DSM; and the definitions for the other variables are as defined in the thirty-third, thirty-seventh or thirty-eighth embodiment.

In a forty-second embodiment of the present disclosure, for the compound of formula (A), or a pharmaceutically acceptable salt thereof, L is represented by formula (L-3); Z⁴ is C₁₋₄ alkyl optionally substituted by benzyl; and R^{L2} is H; and the definitions for the other variables are as defined in the thirty-third embodiment.

In a forty-third embodiment of the present disclosure, for the compound of formula (A), or a pharmaceutically acceptable salt thereof, L is represented by any one of the following formulae:

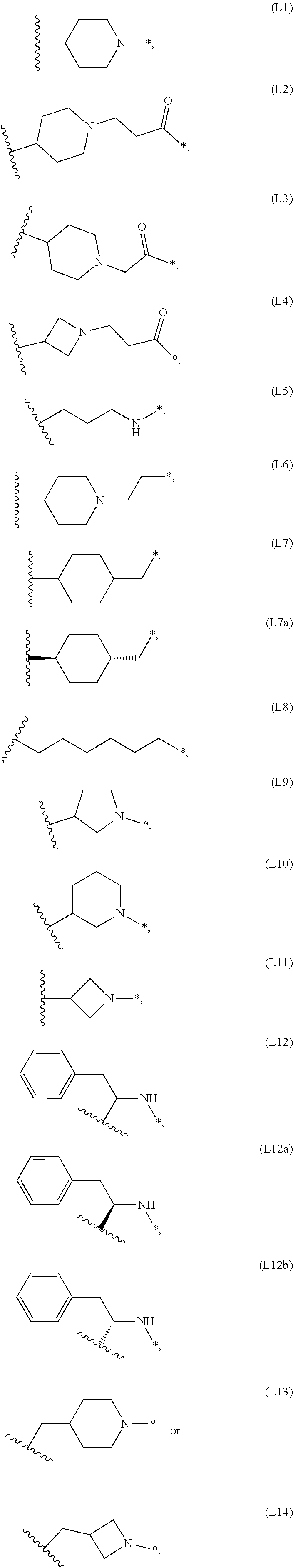

wherein: [symbol] represents a bond to the IRAK4 binding moiety; and —* represents a bond to the degradation signaling moiety DSM; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first or thirty-second embodiment.

In a forty-fourth embodiment of the present disclosure, the compound of formula (A), or a pharmaceutically acceptable salt thereof, is a compound of any one of Examples 1-87 or a pharmaceutically acceptable salt thereof.

### III. Pharmaceutical Compositions and Methods of Uses

Another aspect of the present disclosure is a pharmaceutical composition comprising at least one compound described herein (e.g., a compound or a pharmaceutically acceptable salt thereof described in any of the embodiments described above), and at least one pharmaceutically acceptable carrier.

The compounds of the present disclosure are typically used as a pharmaceutical composition (e.g., a compound of the present disclosure and at least one pharmaceutically acceptable carrier). As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this disclosure, solvates and hydrates are considered pharmaceutical compositions comprising a compound of the present disclosure and a solvent (i.e., solvate) or water (i.e., hydrate).

Compounds of the present disclosure have been found to modulate IRAK4 activity and may be beneficial for the treatment of neurological, neurodegenerative and other additional diseases

In some embodiments, the compounds described herein (e.g., a compound or a pharmaceutically acceptable salt thereof described in any of the embodiments described above) can be used to cause the degradation of IRAK4 proteins. In some embodiments, the compounds described herein (e.g., a compound or a pharmaceutically acceptable salt thereof described in any of the embodiments described above) can be used to modulate (e.g., decrease) the level of IRAK4 proteins. In some embodiments, the compounds or pharmaceutically acceptable salts thereof described herein (e.g., a compound or a pharmaceutically acceptable salt thereof described in any of the embodiments described above) can be used to modulate (e.g., decrease) the activity of IRAK4, or to otherwise affect the properties and/or behavior of IRAK4, e.g., stability, phosphorylation, kinase activity, interactions with other proteins, etc.

In some embodiments, the present disclosure provides methods of decreasing protein levels of IRAK4 and/or IRAK4 enzymatic activity. In some embodiments, such methods include contacting a cell with an effective amount of a compound described herein (e.g., a compound or a pharmaceutically acceptable salt thereof described in any of the embodiments described above).

One aspect of the present disclosure includes a method of treating a disorder responsive to degradation of IRAK4 and/or inhibition of IRAK4 activity in a subject comprising administering to the subject an effective amount of at least one compound described herein (e.g., a compound or a pharmaceutically acceptable salt thereof described in any of the embodiments described above), or a pharmaceutical composition described herein.

One embodiment of the disclosure includes a method for treating an autoimmune disease, cancer, cardiovascular disease, a disease of the central nervous system, a disease of the skin, an ophthalmic disease and condition, and bone disease in a subject, the method comprising administering to the patient a therapeutically effective amount of a compound disclosed herein, or a pharmaceutically acceptable salt thereof, thereby treating the autoimmune disease, cancer, cardiovascular disease, disease of the central nervous system, disease of the skin, ophthalmic disease and condition, and bone disease in the subject.

In one embodiment, the cardiovascular disease is selected from stroke and atherosclerosis. In one embodiment, the disease of the central nervous system is a neurodegenerative disease. In one embodiment, the disease of the skin is selected from rash, contact dermatitis, psoriasis, Hidradenitis Suppurativa and atopic dermatitis. In one embodiment, the bone disease is selected from osteoporosis and osteoarthritis.

In one embodiment, the present disclosure provides methods of treating autoimmune disorders, inflammatory disorders, and cancers in a subject in need thereof comprising administering to the subject an effective amount of at least one compound described herein (e.g., a compound or a pharmaceutically acceptable salt thereof described in any of the embodiments described above), or a pharmaceutical composition described herein.

The term “autoimmune disorders” includes diseases or disorders involving inappropriate immune response against native antigens, such as acute disseminated encephalomyelitis (ADEM), Addison's disease, alopecia areata, antiphospholipid antibody syndrome (APS), autoimmune hemolytic anemia, autoimmune hepatitis, bullous pemphigoid (BP), Coeliac disease, dermatomyositis, diabetes mellitus type 1, Goodpasture's syndrome, Graves' disease, Guillain-Barre syndrome (GBS), Hashimoto's disease, idiopathic thrombocytopenic purpura, lupus erythematosus, cutaneous lupus erythematosus (CLE), neuromyelitis optica (NMO), mixed connective tissue disease, multiple sclerosis, myasthenia gravis, pemphigus vulgaris, pernicious anaemia, polymyositis, primary biliary cirrhosis, Sjogren's syndrome, temporal arteritis, and Wegener's granulomatosis.

In one embodiment, the autoimmune disease is selected from rheumatoid arthritis, systemic lupus erythematosus, multiple sclerosis, diabetes, systemic sclerosis, and Sjogren's syndrome. In one embodiment, the autoimmune disease is type 1 diabetes.

The term “inflammatory disorders” includes diseases or disorders involving acute or chronic inflammation such as allergies, asthma, prostatitis, glomerulonephritis, pelvic inflammatory disease (PID), inflammatory bowel disease (IBD, e.g., Crohn's disease, ulcerative colitis), reperfusion injury, rheumatoid arthritis, transplant rejection, and vasculitis. In some embodiments, the present disclosure provides a method of treating rheumatoid arthritis or lupus. In some embodiments, the present disclosure provides a method of treating multiple sclerosis. In some embodiments, the present disclosure provides a method of treating systemic lupus erythematosus or atopic dermatitis.

One embodiment of the disclosure includes a method for treating an inflammatory disease in a subject, the method comprising administering to the patient a therapeutically effective amount of a compound described herein, or a pharmaceutically acceptable salt thereof, thereby treating the inflammatory disease in the subject.

In one embodiment, the inflammatory disease is a pulmonary disease or a disease of the airway. In one embodiment, the pulmonary disease and disease of the airway is selected from Adult Respiratory Disease Syndrome (ARDS), Chronic Obstructive Pulmonary Disease (COPD), pulmonary fibrosis, interstitial lung disease, asthma, chronic cough, and allergic rhinitis.

In one embodiment, the inflammatory disease is selected from transplant rejection, CD14 mediated sepsis, non-CD14 mediated sepsis, inflammatory bowel disease, Behcet's syndrome, ankylosing spondylitis, sarcoidosis, and gout. In one embodiment, the inflammatory bowel disease is selected from Crohn's disease and ulcerative colitis.

One embodiment of the disclosure includes a method for treating an ischemic fibrotic disease, the method comprising administering to the patient a therapeutically effective amount of a compound described herein, or a pharmaceutically acceptable salt thereof, thereby treating the ischemic fibrotic disease in the subject. In one embodiment, the ischemic fibrotic disease is selected from stroke, acute lung injury, acute kidney injury, ischemic cardiac injury, acute liver injury, and ischemic skeletal muscle injury.

One embodiment of the disclosure includes a method for treating post-organ transplantation fibrosis, the method comprising administering to the patient a therapeutically effective amount of a compound described herein, or a pharmaceutically acceptable salt thereof, thereby treating post-organ transplantation fibrosis in the subject.

One embodiment of the disclosure includes a method for treating hypertensive or diabetic end organ disease, the method comprising administering to the patient a therapeutically effective amount of a compound described herein, or a pharmaceutically acceptable salt thereof, thereby treating hypertensive or diabetic end organ disease in the subject.

One embodiment of the disclosure includes a method for treating hypertensive kidney disease, the method comprising administering to the patient a therapeutically effective amount of a compound described herein, or a pharmaceutically acceptable salt thereof, thereby treating hypertensive kidney disease in the subject.

One embodiment of the disclosure includes a method for treating idiopathic pulmonary fibrosis (IPF), the method comprising administering to the patient a therapeutically effective amount of a compound described herein, or a pharmaceutically acceptable salt thereof, thereby treating IPF in the subject.

One embodiment of the disclosure includes a method for treating scleroderma or systemic sclerosis, the method comprising administering to the patient a therapeutically effective amount of a compound described herein, or a pharmaceutically acceptable salt thereof, thereby treating scleroderma or systemic sclerosis in the subject.

One embodiment of the disclosure includes a method for treating liver cirrhosis, the method comprising administering to the patient a therapeutically effective amount of a compound described herein, or a pharmaceutically acceptable salt thereof, thereby treating liver cirrhosis in the subject.

One embodiment of the disclosure includes a method for treating fibrotic diseases wherein tissue injury and/or inflammation are present, the method comprising administering to the patient a therapeutically effective amount of a compound described herein, or a pharmaceutically acceptable salt thereof, thereby treating fibrotic diseases where tissue injury and/or inflammation are present in the subject. The fibrotic diseases include, for example, pancreatitis, peritonitis, burns, glomerulonephritis, complications of drug toxicity, and scarring following infections.

Scarring of the internal organs is a major global health problem, which is the consequence of subclinical injury to the organ over a period of time or as the sequela of acute severe injury or inflammation. All organs may be affected by scarring and currently there are few therapies the specifically target the evolution of scarring. Increasing evidence indicates that scarring per se provokes further decline in organ function, inflammation and tissue ischemia. This may be directly due the deposition of the fibrotic matrix which impairs function such as in contractility and relaxation of the heart and vasculature or impaired inflation and deflation of lungs, or by increasing the space between microvasculature and vital cells of the organ that are deprived of nutrients and distorting normal tissue architecture. However recent studies have shown that myofibroblasts themselves are inflammatory cells, generating cytokines, chemokines and radicals that promote injury; and myofibroblasts appear as a result of a transition from cells that normally nurse and maintain the microvasculature, known as pericytes. The consequence of this transition of phenotype is an unstable microvasculature that leads to aberrant angiogenesis, or rarefaction.

The present disclosure relates to methods and compositions for treating, preventing, and/or reducing scarring in organs. More particularly, the present disclosure relates to methods and composition for treating, preventing, and/or reducing scarring in kidneys. Some non-limiting examples of organs include: kidney, hearts, lungs, stomach, liver, pancreas, hypothalamus, stomach, uterus, bladder, diaphragm, pancreas, intestines, colon, and so forth.

It is contemplated that the present disclosure, methods and compositions described herein can be used as an antifibrotic, or used to treat, prevent, and/or reduce the severity and damage from fibrosis. It is additionally contemplated that the present disclosure, methods and compositions described herein can be used to treat, prevent, and/or reduce the severity and damage from fibrosis.

The compounds of the present disclosure (e.g., a compound or a pharmaceutically acceptable salt thereof described in any of the embodiments described above) may be useful in the treatment of cancer, for example a cancer selected from solid tumor cancers and hematopoietic cancers.

The term “cancer” includes diseases or disorders involving abnormal cell growth and/or proliferation, such as glioma, thyroid carcinoma, breast carcinoma, lung cancer (e.g. small-cell lung carcinoma, non-small-cell lung carcinoma), gastric carcinoma, gastrointestinal stromal tumors, pancreatic carcinoma, bile duct carcinoma, ovarian carcinoma, endometrial carcinoma, prostate carcinoma, renal cell carcinoma, lymphoma (e.g., anaplastic large-cell lymphoma), leukemia (e.g. acute myeloid leukemia, T-cell leukemia, chronic lymphocytic leukemia), multiple myeloma, malignant mesothelioma, malignant melanoma, and colon cancer (e.g. microsatellite instability-high colorectal cancer). In some embodiments, the present disclosure provides a method of treating leukemia or lymphoma.

Examples of solid tumor cancers include central nervous system cancer, brain cancer, breast cancer, head and neck cancer, lung cancer; esophageal and esophagogastric junction cancer, gastric cancer, colorectal cancer, rectal cancer, anal cancer, hepatobiliary cancer, pancreatic cancer, non-melanoma skin cancer, melanoma, renal cancer, prostate cancer, bladder cancer, uterine cancer, cervical cancer, ovarian cancer, bone cancer, neuroendocrine cancer, mesothelioma cancer, testicular cancer, thymoma and thymic carcinoma, and thyroid cancer.

Examples of hematopoietic cancers include B-cell neoplasms (including rare B-cell malignancies), Hodgkin lymphoma, non-Hodgkin lymphoma, post-transplant lymphoproliferative disorder, hairy cell leukemia, histiocytic and dendritic neoplasms.

Examples of B-cell neoplasms include chronic lymphocytic leukemia (CLL), mantle cell lymphoma (MCL), small lymphocytic lymphoma (SLL), Waldenstrom's macroglobulinemia, diffuse large B-cell lymphoma (DLBCL), follicular lymphoma, Burkitt lymphoma, Marginal Zone Lymphoma, immunoblastic large cell lymphoma, Richter Syndrome, and precursor B-lymphoblastic lymphoma, primary and secondary multiple myeloma, B-cell prolymphocytic leukemia, lymphoplasmacytic lymphoma, splenic marginal zone lymphoma, plasma cell myeloma, plasmacytoma, extranodal marginal zone B-cell lymphoma, nodal marginal zone B-cell lymphoma, mediastinal (thymic) large B-cell lymphoma, intravascular large B-cell lymphoma, primary effusion lymphoma, lymphomatoid granulomatosis, and acute lymphoblastic leukemia.

In some embodiments, the cancer is selected from chronic lymphocytic leukemia (CLL), diffuse large B-cell lymphoma (DLBCL), mantle cell lymphoma (MCL), small lymphocytic lymphoma (SLL), and Waldenstrom's macroglobulinemia. In one embodiment, the cancer is chronic lymphocytic leukemia (CLL). In another embodiment, the cancer is diffuse large B-cell lymphoma (DLBCL).

In certain embodiments, the present disclosure relates to the aforementioned methods, wherein said subject is a mammal. In certain embodiments, the present disclosure relates to the aforementioned methods, wherein said subject is a primate. In certain embodiments, the present disclosure relates to the aforementioned methods, wherein said subject is a human.

According to the disclosure an “effective dose” or an “effective amount” of the compound or pharmaceutical composition is that amount effective for treating or lessening the severity of one or more of the diseases, disorders or conditions as recited above. The effective dose of a compound provided herein, or a pharmaceutically acceptable salt thereof, administered to a subject can be 10 μg-500 mg.

The formulations may be prepared using conventional dissolution and mixing procedures. For example, the bulk drug substance (i.e., compound of the present disclosure or stabilized form of the compound (e.g., complex with a cyclodextrin derivative or other known complexation agent)) is dissolved in a suitable solvent in the presence of one or more of the excipients described above. The compound of the present disclosure is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present disclosure is generally formulated for use as a parenteral or oral administration or alternatively suppositories.

For example, the pharmaceutical oral compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethylene glycol; for tablets also c) binders, e.g., magnesium aluminum silicate, starch paste, gelatin, tragacanth, methylcellulose, sodium carboxymethylcellulose and/or polyvinylpyrrolidone; if desired d) disintegrants, e.g., starches, agar, alginic acid or its sodium salt, or effervescent mixtures; and/or e) absorbents, colorants, flavors and sweeteners. Tablets may be either film coated or enteric coated according to methods known in the art.

Suitable compositions for oral administration include a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations.

Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compounds and compositions, according to the methods of the present disclosure, may be administered using any amount and any route of administration effective for treating or lessening the severity of one or more of the diseases, disorders or conditions recited above.

Administering a compound described herein, or a pharmaceutically acceptable salt thereof, to a mammal comprises any suitable delivery method. Administering a compound described herein, or a pharmaceutically acceptable salt thereof, to a mammal includes administering a compound described herein, or a pharmaceutically acceptable salt thereof, topically, enterally, parenterally, transdermally, transmucosally, via inhalation, intracisternally, epidurally, intravaginally, intravenously, intramuscularly, subcutaneously, intradermally or intravitreally to the mammal. Administering a compound described herein, or a pharmaceutically acceptable salt thereof, to a mammal also includes administering topically, enterally, parenterally, transdermally, transmucosally, via inhalation, intracisternally, epidurally, intravaginally, intravenously, intramuscularly, subcutaneously, intradermally or intravitreally to a mammal a compound that metabolizes within or on a surface of the body of the mammal to a compound described herein, or a pharmaceutically acceptable salt thereof.

The compound of the present disclosure or pharmaceutical composition thereof for use in a subject (e.g., human) is typically administered orally or parenterally at a therapeutic dose of less than or equal to about 100 mg/kg, 75 mg/kg, 50 mg/kg, 25 mg/kg, 10 mg/kg, 7.5 mg/kg, 5.0 mg/kg, 3.0 mg/kg, 1.0 mg/kg, 0.5 mg/kg, 0.05 mg/kg or 0.01 mg/kg, but preferably not less than about 0.0001 mg/kg. When administered intravenously via infusion, the dosage may depend upon the infusion rate at which an IV formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

Thus, a compound or pharmaceutically acceptable salt thereof as described herein, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the compound or pharmaceutically acceptable salt thereof as described herein may be combined with one or more excipients and used in the form of ingestible tablets, buccal tablets, troches, capsules, elixirs, suspensions, syrups, or wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions can be such that an effective dosage level will be obtained.

The tablets, troches, pills, capsules, and the like can include the following: binders such as gum tragacanth, acacia, corn starch or gelatin; excipients such as dicalcium phosphate; a disintegrating agent such as corn starch, potato starch, alginic acid and the like; a lubricant such as magnesium stearate; or a sweetening agent such as sucrose, fructose, lactose or aspartame or a flavoring agent.

In certain embodiments, the present disclosure relates to the aforementioned methods, wherein said compound is administered intramuscularly, intravenously, subcutaneously, orally, pulmonary, rectally, intrathecally, topically or intranasally. The active compound may also be administered intravenously or intraperitoneally by infusion or injection. Solutions of the active compound or its salts can be prepared in water, optionally mixed with a nontoxic surfactant. In certain embodiments, the present disclosure relates to the aforementioned methods, wherein said compound is administered parenterally. In certain embodiments, the present disclosure relates to the aforementioned methods, wherein said compound is administered systemically.

Exemplary pharmaceutical dosage forms for injection or infusion can include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid and stable under the conditions of manufacture and storage.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation can be vacuum drying and the freeze drying techniques, which can yield a powder of the active ingredient plus any additional desired ingredient present in the previously sterile-filtered solutions.

Exemplary solid carriers can include finely divided solids such as talc, clay, microcrystalline cellulose, silica, alumina and the like. Useful liquid carriers include water, alcohols or glycols or water-alcohol/glycol blends, in which the compounds or pharmaceutically acceptable salts thereof as described herein can be dissolved or dispersed at effective levels, optionally with the aid of non-toxic surfactants.

Useful dosages of a compound or pharmaceutically acceptable salt thereof as described herein can be determined by comparing their in vitro activity, and in vivo activity in animal models. Methods for the extrapolation of effective dosages in mice, and other animals, to humans are known to the art; for example, see U.S. Pat. No. 4,938,949, which is incorporated by reference in its entirety.

The amount of a compound or pharmaceutically acceptable salt thereof as described herein, required for use in treatment can vary not only with the particular salt selected but also with the route of administration, the nature of the condition being treated and the age and condition of the patient and can be ultimately at the discretion of the attendant physician or clinician. In general, however, a dose can be in the range of from about 0.1 to about 10 mg/kg of body weight per day.

The compound or pharmaceutically acceptable salt thereof as described herein can be conveniently administered in unit dosage form; for example, containing 0.01 to 10 mg, or 0.05 to 1 mg, of active ingredient per unit dosage form. In some embodiments, a dose of 5 mg/kg or less can be suitable.

The desired dose may conveniently be presented in a single dose or as divided doses administered at appropriate intervals.

The disclosed method can include a kit comprising a compound or pharmaceutically acceptable salt thereof as described herein and instructional material which can describe administering a compound or pharmaceutically acceptable salt thereof as described herein or a composition comprising a compound or pharmaceutically acceptable salt thereof as described herein to a cell or a subject. This should be construed to include other embodiments of kits that are known to those skilled in the art, such as a kit comprising a (such as sterile) solvent for dissolving or suspending a compound or pharmaceutically acceptable salt thereof as described herein or composition prior to administering a compound or pharmaceutically acceptable salt thereof as described herein or composition to a cell or a subject. In some embodiments, the subject can be a human.

### IV. Exemplifications

### A. Abbreviations and Acronyms Used Herein Include the Following

ACN: means acetonitrile (CH₃CN);
AcOH: means Acetic acid;
Aq. or aq.: means aqueous;
Ar: means argon;
br: means broad;
tBuXPhos Pd G3: means [(2-Di-tert-butylphosphino-2′,4′,6′-triisopropyl-1,1′-biphenyl)-2-(2′-amino-1,1′-biphenyl)] palladium(II) methanesulfonate;
° C.: means degrees Celsius;
CAN means ceric ammonium nitrate [(NH₄)₂Ce(NO₃)₆];
CDCl₃: means deutero-chloroform;
CDI: means 1,1′-carbonyldiimidazole;
CH₂Cl₂: means methylene chloride;
CaCl₂): means Calcium chloride;
Cs₂CO₃: means cesium carbonate;
d: means doublet;
dd: means double doublet;
δ: means chemical shift;
D₂O: means deuterated water;
DCM: dichloromethane;
Dess-Martin Periodinane: means 3-Oxo-1λ⁵,2-benziodoxole-1,1,1(3H)-triyl triacetate;
DIPEA: diisopropyl ethylamine;
DMF: dimethylformamide;
DMSO: means dimethylsulfoxide;
DMSO-d₆: means hexadeuterodimethyl sulfoxide;
ESI: electrospray ionization;
Et: means ethyl;
Et₃N: means triethylamine;
EtOH: ethanol;
EtOAc: means ethyl acetate;
g: means gram;
h: means hour;
HATU: means 1-[bis(dimethylamino)methylene]-1H-1,2,3-triazolo[4,5-b]pyridinium 3-oxid hexafluorophosphate;
HBr: means hydrogen bromide;
HCl: means hydrochloric acid;
HPLC: means high pressure liquid chromatography;
¹H NMR: means proton nuclear magnetic resonance;
H₂O: means water;
IPA: means isopropyl alcohol;
K₂CO₃: means potassium carbonate;
KOH: means potassium hydroxide;
L: means litre;
LC: means liquid chromatography;
LC-MS: means liquid chromatography mass spectrometry;
LDA means lithium diisopropylamide;
m: means multiplet;
M: means molar;
mins: means minutes;
mL: means millilitres;
μL: means micro litres;
mmol: means millimole;
m/z: mass to charge ratio;
mg: means milligram;
Me: means methyl;
MeCN: means acetonitrile;
MeOH: means methanol;
MHz: means mega Hertz;
Min(s): minute(s);
MS m/z: means mass spectrum peak;
MTBE: means tert-butyl methyl ether;
M/V: means Mass volume ratio;
N₂ or N₂: means nitrogen;
NH₄Cl: means ammonium chloride;
Na: means sodium;
NaH: means sodium hydride;
NaHCO₃: means sodium bicarbonate;
NaOH: means sodium hydroxide;
NaOCN: means sodium cyanate;
Na₂SO₄: means sodium sulfate;
NH₄Cl: means ammonium chloride;
NMP: is N-methyl-2-pyrrolidone;
2-picoline borane complex: is 2-methylpyridine-borane complex;
Pd(dppf)Cl₂: means [1,1′-bis(diphenylphosphino)ferrocene]dichloropalladium(II);
Pd-PEPPSI-IHeptCl: means Dichloro[1,3-bis(2,6-di-4-heptylphenyl)imidazol-2-ylidene](3-chloropyridyl)palladium(II);
Pd(t-Bu₃P)₂: means Bis(tri-tert-butylphosphine)palladium(0);
PE or Pet ether: means petroleum ether;
Psi: means pounds per square inch;
q: means quartet;
R_{f}: means retention factor;
RT: or means room temperature;
s: means singlet;
sat.: means saturated;
soln.: means solution;
SFC: means supercritical fluid chromatography;
t: means triplet;
TEA: means triethylamine;
TFA: means trifluoroacetic acid;
THF: means tetrahydrofuran;
TLC: means thin layer chromatography;
μmol: means micromole;
UPLC: means ultra performance liquid chromatography;
V: volumes;
XPhos: means 2-dicyclohexylphosphino-2′,4′,6′-triisopropylbiphenyl.

### B. General Methods

The compounds of the Examples were analyzed or purified according to one of the purification methods referred to below unless otherwise described.

Where preparative TLC or silica gel chromatography have been used, one skilled in the art may choose any combination of solvents to purify the desired compound. Silica gel column chromatography was performed using 20-40 μM (particle size), 100-200 mesh, 250-400 mesh, or 400-632 mesh silica gel using either a Teledyne ISCO Combiflash® RF, a Biotage® Isolera One 3.3.0, a Biotage® Flash Isolera Prime, a Grace Reveleris X2 with ELSD purification, a Gilson-281 with ELSD purification systems or using pressurized nitrogen (˜10-15 psi) to drive solvent through the column (“flash chromatography”).

Except where otherwise noted, reactions were run under an atmosphere of nitrogen. Where indicated, solutions and reaction mixtures were concentrated by rotary evaporation under vacuum.

### C. Analytical Methods

### NMR

### Instrument Specifications:

Bruker AVANCE III 400
Bruker AVANCE III HD 400
Bruker AVANCE NEO 400
Bruker AVANCE DRX 500
Varian UNITYplus 400

### LC/MS

### Instrument Specifications:

Agilent 1200 Series LC/MSD system with DAD and Agilent LCMS G6110A, mass-spectrometer.
Agilent(Degasser: 1200; Pump: 1260; Hip-ALS: 1200; TCC: 1200; DAD: 1100)
Series LC/MS system with DAD\ELSD and Agilent LCMS G6110A, mass-spectrometer.
Agilent(Degasser: 1200; Pump: 1260; Hip-ALS: 1100; TCC: 1260; DAD: 1100)
Series LC/MS system with DAD and Agilent LCMS G1956A, mass-spectrometer.
Agilent(Degasser: 1200; Pump: 1200; Hip-ALS: 1100; TCC: 1200; DAD: 1200)
Series LC/MS system with DAD and Agilent LCMS G1956A, mass-spectrometer.
Agilent 1200 Series LC/MSD system with DAD\ELSD Alltech 3300 and Agilent LC\MSD G6130A, G6120B mass-spectrometer.
Agilent Technologies 1260 Infinity LC/MSD system with DAD\ELSD Alltech 3300 and Agilent LC\MSD G6120B mass-spectrometer.
Agilent Technologies 1260 Infinity II LC/MSD system with DAD\ELSD G7102A 1290 Infinity II and Agilent LC\MSD G6120B mass-spectrometer.
Agilent 1260 Series LC/MSD system with DAD\ELSD and Agilent LC\MSD (G6120B) mass-spectrometer.
UHPLC Agilent 1290 Series LC/MSD system with DAD\ELSD and Agilent LC\MSD (G6125B) mass-spectrometer.
Agilent 1290 Infinity II-6130 Quadrupole MS (single Quad)
SHIMADZU LC-20AD Series LC/MS system with SPD-M20A and SHIMADZU LCMS LCMS-2020, mass-spectrometer.
SHIMADZU LC-20AD Series LC/MS system with SPD-M20A\ELSD and SHIMADZU LCMS LCMS-2020, mass-spectrometer
SHIMADZU LC-20AD Series LC/MS system with SPD-M40 and SHIMADZU LCMS LCMS-2020, mass-spectrometer.
SHIMADZU LC-20AB Series LC/MS system with SPD-M20A and SHIMADZU LCMS LCMS-2020, mass-spectrometer.
SHIMADZU LC-20AB Series LC/MS system with SPD-M20A\ELSD and SHIMADZU LCMS LCMS-2020, mass-spectrometer.
Waters Acquity UPLC H-Class-SQ Detector 2
Ultima 3000 Dionex UHPLC-Thermo LCQ fleet ion trap

### HPLC

### Instrument Specifications:

SHIMADZU LC-20AD Series LC system with SPD-M20A
SHIMADZU LC-20AB Series LC system with SPD-M40
SHIMADZU LC-20AB Series LC system with SPD-M20A
Waters Acquity HPLC (binary/Quaternary Pump)
Agilent 1260 Infinity II LC system with PDA detector

### Prep-HPLC

### Instrument Specifications:

Shimadzu Nexera Prep-Pump-LC-20 AP with auto sampler and auto fraction collector
Gilson 331/332 HPLC pump system
Waters-MS prep-QDA

### SFC

### Instrument Specifications:

Waters 150/200 purification system
Waters investigator
Waters UPC2
Sepiatec screening system

### D. LC-MS Methods

### Method 1

0.1% Formic acid in water (Aqueous phase)
100% Acetonitrile (Organic Phase)
Mode: gradient % B (5 to 95 in 3.7 minute)
Run Time: 4.8 minute
Column: Acquity UPLC BEH/X-Bridge BEH C18, 1.7 μm/2.5 μm, 2.1×50 mm
Flow rate: 0.5 mL/0.6 mL per minute
Temp: 40° C.

### Method 2

10 mM Ammonium Acetate in water (Aqueous phase)
100% Acetonitrile (Organic Phase)
Mode: gradient % B (5 to 95 in 3.7 minute)
Run Time: 4.8 minute
Column: Acquity UPLC BEH/X-Bridge BEH C18, 1.7 μm/2.5 μm, 2.1×50 mm
Flow rate: 0.5 mL/0.6 mL per minute
Temp: 40° C.

### Method 3

0.1% TFA in water (Aqueous phase)
100% Acetonitrile (Organic Phase)
Mode: gradient % B (5 to 95 in 3.7 minute)
Run Time: 4.8 minute
Column: Acquity UPLC BEH/X-Bridge BEH C18, 1.7 μm/2.5 μm, 2.1×50 mm
Flow rate: 0.5 mL/0.6 mL per minute
Temp: 40° C.

### Method 4

10 mM Ammonium Bicarbonate in water (Aqueous phase)
100% Acetonitrile (Organic Phase)
Mode: gradient % B (5 to 95 in 3.7 minute)
Run Time: 4.8 minute
Column: Acquity UPLC BEH/X-Bridge BEH C18, 1.7 μm/2.5 μm, 2.1×50 mm
Flow rate: 0.5 mL/0.6 mL per minute
Temp: 40° C.

### Method 5

Mobile phase: A: 0.0375% TFA in H₂O v/v
   B: 0.01875% TFA in ACN, v/v
Column: Kinetex EVO C18 30*2.1 mm, 5 μm
Flow rate: 1.5 mL/min
Temp: 50° C.
Gradient: 5-95% B, 0-60% B, 30-90% B, or 50-100% B in 1.55 min

### Method 6

Mobile phase: A: 0.025% NH₃ H₂O in H₂O, v/v
   B: ACN
Column: Kinetex EVO C18 30*2.1 mm, 5 μm
Flow rate: 1.5 mL/min
Temp: 50° C.
Gradient: 5-95% B, 0-60% B, 30-90% B, or 50-100% B in 1.55 min

### Method 7

Inject volume: 0.5 μl
Column Temperature: 60° C.
UV scan: 207-223 nM
   246-262 nM
   272-288 nM
Agilent Poroshell 120 SB-C18 4.6×30 mm 2.7 μm
with UHPLC Guard Infinity Lab Poroshell 120 SB-C18 4.6×5 mm 2.7 μm
Mobile phase A: 0.1% FA in Water
Mobile phase B: 0.1% FA in Acetonitrile

### Details of Elution

| Time (min) | Flow (mL/min) | % A | % B |
|---|---|---|---|
| 0.00 | 3.00 | 99 | 1 |
| 0.01 | 3.00 | 99 | 1 |
| 1.5 | 3.00 | 0 | 100 |
| 1.73 | 3.00 | 0 | 100 |
| 1.74 | 3.00 | 99 | 1 |

### Method 8

Inject volume: 0.5 μl
Column Temperature: 60° C.
UV scan: 207-223 nM
   246-262 nM
   272-288 nM
Agilent Poroshell 120 SB-C18 4.6×30 mm 2.7 μm
with UHPLC Guard Infinity Lab Poroshell 120 SB-C18 4.6×5 mm 2.7 μm
Mobile phase A: 0.1% FA in Water
Mobile phase B: 0.1% FA in Acetonitrile

### Details of Elution

| Time (min) | Flow (mL/min) | % A | % B |
|---|---|---|---|
| 0.00 | 1.5 | 99 | 1 |
| 0.01 | 1.5 | 99 | 1 |
| 5.00 | 1.5 | 0 | 100 |
| 5.99 | 1.5 | 0 | 100 |
| 6.00 | 1.5 | 99 | 1 |

### E. Synthesis of Degradation Signaling Moieties (DSMs)

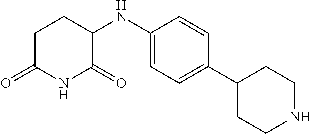 [SMILES: O=C1CCC(Nc2ccc(C3CCNCC3)cc2)C(=O)N1]

Intermediate 3-((4-(piperidin-4-yl)phenyl)amino)piperidine-2,6-dione was prepared according to the method described in page 267 of WO2018237026A1.

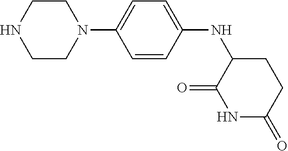 [SMILES: O=C1CCC(Nc2ccc(N3CCNCC3)cc2)C(=O)N1]

Intermediate 3-((4-(piperazin-1-yl)phenyl)amino)piperidine-2,6-dione was prepared according to the method described in page 268 of WO2018237026A1.

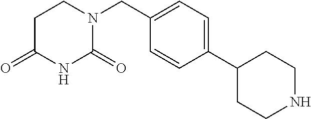 [SMILES: O=C1CCN(Cc2ccc(C3CCNCC3)cc2)C(=O)N1]

Intermediate 3-((4-(piperazin-1-yl)phenyl)amino)piperidine-2,6-dione was prepared according to the method described in page 353 of WO2020132561A1.

### Synthesis of 3-[4-(4-piperidyl)anilino]piperidine-2,6-dione Isomer 1 and 2

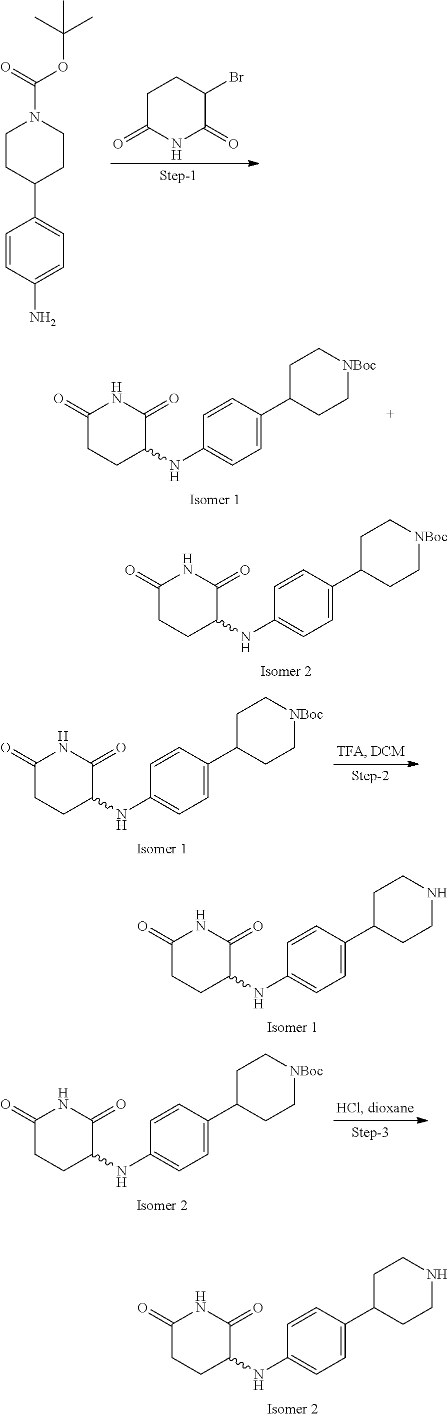

### Step-1

To a stirred solution of tert-butyl 4-(4-aminophenyl)piperidine-1-carboxylate (2 g, 7.24 mmol) and 3-bromopiperidine-2,6-dione (4.17 g, 21.71 mmol) in DMF (20 mL) was added sodium bicarbonate (6.08 g, 72.37 mmol) and the reaction mixture was stirred at 80° C. for 16 h. After consumption of the starting material, the reaction mixture was diluted with water, the precipitate filtered, and the solid dried under vacuum. SFC purification gave tert-butyl 4-[4-[[(3S)-2,6-dioxo-3-piperidyl]amino]phenyl]piperidine-1-carboxylate (0.2 g, 491.39 μmol, 6.79% yield) and tert-butyl 4-[4-[[(3R)-2,6-dioxo-3-piperidyl]amino]phenyl]piperidine-1-carboxylate (0.2 g, 491.39 μmol, 6.79% yield)

### Preparative SFC Conditions

Column/dimensions: Chiralcel—OJ-H (21*250) mm, 5 μm
% CO₂: 70%
% Co solvent: 30% (ACN)
Total Flow: 60 g/min
Back Pressure: 100 bar
Temperature: 30 OC
UV: 243 nm
Solubility: ACN
No of injections: 86
Total purification time: 10 Hrs
Instrument details: Make/Model: SFC-150-II
The first eluted peak during SFC separation was assigned as Isomer 1 (elution time=1.993 min) and the second eluted peak was assigned as Isomer 2 (elution time=2.905 min).
Isomer 1: LC-MS (ES⁻): m/z 386.39 [M−H]⁻
Isomer 2: LC-MS (ES⁻): m/z 386.39 [M−H]⁻

### Step-2

To a stirred solution of tert-butyl 4-[4-[[-2,6-dioxo-3-piperidyl]amino]phenyl]piperidine-1-carboxylate Isomer 1 (0.2 g, 516.17 μmol) in DCM (5 mL) at 0° C. was added TFA (1.48 g, 12.98 mmol, 1 mL) over 2 minutes. The reaction mixture was stirred at 25° C. for 3 h. The reaction mixture was concentrated and co-distilled with toluene (10 ml) and diethyl ether (2×50 ml) to afford 3-[4-(4-piperidyl)anilino]piperidine-2,6-dione Isomer 1 TFA salt (0.15 g, 358.76 μmol, 69.51% yield) as a gummy green solid.

LC-MS (ES⁺): m/z 288.28 [M+H]⁺.

### Step-3

To a solution of tert-butyl 4-[4-[(2,6-dioxo-3-piperidyl)amino]phenyl]piperidine-1-carboxylate Isomer 2 (0.15 g, 387.13 μmol) in DCM (10 mL) at 0° C. was added 4N HCl in 1,4-dioxane (13.55 mg, 376.37 μmol, 1.5 mL). The resulting reaction mixture was stirred at 0° C. to room temperature over 3 hr. DCM was removed under reduced pressure. To the crude residue was added ether and the resulting mixture was stirred for 5 minutes to separate the desired product as the HCl salt. The solid was filtered and washed with ether (2×10 mL) to afford 3-[4-(4-piperidyl)anilino]piperidine-2,6-dione Isomer 2 HCl salt (0.13 g, 385.40 μmol, 99.56% yield) and was directly used for the next step without further purification.

LC-MS (ES⁺): m/z 288.20 [M+H]⁺.

### Synthesis of 3-[3-fluoro-4-(4-piperidyl)anilino]piperidine-2,6-dione

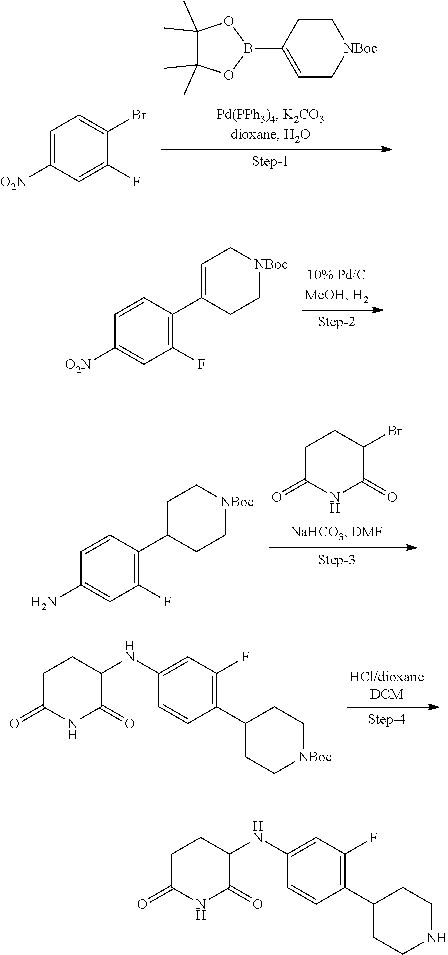

### Step-1

A solution of 1-bromo-2-fluoro-4-nitro-benzene (6 g, 27.27 mmol) and tert-butyl 4-(4,4,5,5-tetramethyl-1,3,2-dioxaborolan-2-yl)-3,6-dihydro-2H-pyridine-1-carboxylate (8.43 g, 27.27 mmol) in dioxane (60 mL) and water (15 mL) in a round bottom flask was purged with argon gas for 10 minutes, followed by the addition of potassium carbonate, granular (11.31 g, 81.82 mmol). The solution was purged with argon gas for another 20 minutes before palladium; triphenylphosphane (1.58 g, 1.36 mmol) was added and the reaction was stirred at 90° C. for 16 hours. The progress of the reaction was monitored by TLC and LC-MS. After completion of the reaction, the reaction mixture was filtered through celite bed and washed with ethyl acetate. The filtrate was concentrated under reduced pressure and the crude product was diluted with water and extracted with ethyl acetate (2×150 ml). The combined organic layer was concentrated in vacuo and purified by normal phase column chromatography (Davisil silica, 5% ethyl acetate in pet ether) to obtain tert-butyl 4-(2-fluoro-4-nitro-phenyl)-3,6-dihydro-2H-pyridine-1-carboxylate (5.95 g, 18.27 mmol, 67.01% yield) as a light-yellow solid. LC-MS (ES⁺): m/z 267.15 [M-tBu+H]⁺.

### Step-2

To a stirred solution of tert-butyl 4-(2-fluoro-4-nitro-phenyl)-3,6-dihydro-2H-pyridine-1-carboxylate (3 g, 9.31 mmol) in methanol (70 mL) was added palladium, 10% on carbon, type 487, dry (3 g, 28.19 mmol) at room temperature. The reaction mixture was stirred for 6 hours at this temperature under hydrogen atmosphere, and the reaction progress was monitored by LC-MS. After completion of reaction, the reaction mixture was filtered through celite and the filtrate was concentrated under reduced pressure to afford compound tert-butyl 4-(4-amino-2-fluoro-phenyl)piperidine-1-carboxylate (2.5 g, 5.95 mmol, 63.88% yield) as purple solid, which was taken to the next step without purification. LC-MS (ES⁺): m/z 239.30 [M-tBu+H]⁺.

### Step-3

In a sealed tube, a solution of tert-butyl 4-(4-amino-2-fluoro-phenyl)piperidine-1-carboxylate (2.5 g, 8.49 mmol) and 3-bromopiperidine-2,6-dione (4.08 g, 21.23 mmol) in DMF (40 mL) was stirred for 10 minutes before sodium bicarbonate (3.57 g, 42.46 mmol) was added and the reaction was heated at 60° C. for 16 hours. The progress of reaction was monitored by LC-MS and TLC. After completion of the reaction, the reaction mixture was filtered and concentrated in vacuo. The crude product was purified by column chromatography (Devisil silica, 0-30% ethyl acetate in pet ether) to furnish tert-butyl 4-[4-[(2,6-dioxo-3-piperidyl)amino]-2-fluoro-phenyl]piperidine-1-carboxylate (1.8 g, 3.64 mmol, 42.86% yield) as a brown solid. LC-MS (ES⁻): m/z 404.3 [M−H]⁻.

### Step-4

To a solution of tert-butyl 4-(4-((2,6-dioxopiperidin-3-yl)amino)-2-fluorophenyl)piperidine-1-carboxylate (100 mg, 246.63 μmol) in DCM (1 mL) was added HCl/dioxane (2 mL). The mixture was stirred at 25° C. for 0.5 hour. After completion of the reaction as confirmed by LC-MS, the solvent was removed and the residue was dissolved in MeCN (30 mL), adjusted to pH=7 with NaHCO₃, and filtered. The filtrate was concentrated in vacuo and used in the next step directly. Compound 3-[3-fluoro-4-(4-piperidyl)anilino]piperidine-2,6-dione (75 mg, 233.34 μmol, 94.61% yield) was obtained as a white solid. LC-MS (ES⁺): m/z 306.2 [M+H]⁺.

### Synthesis of 3-[3-(4-piperidyl)anilino]piperidine-2,6-dione

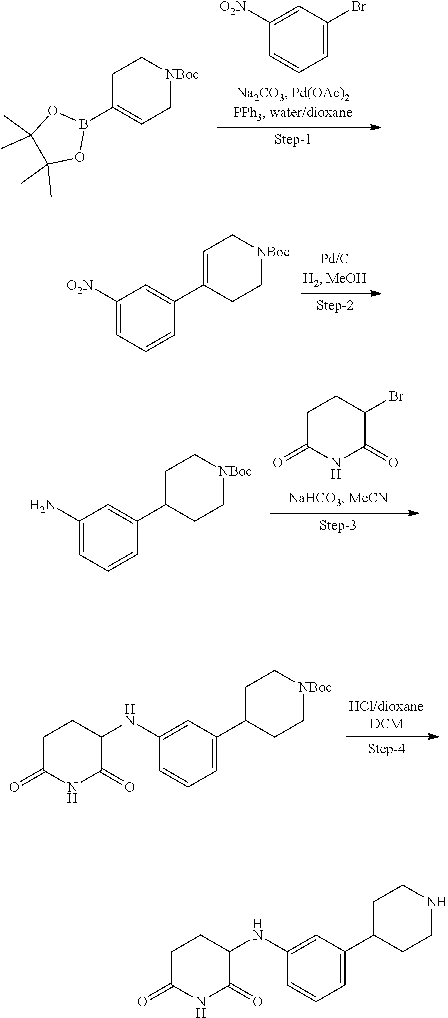

### Step-1

To a solution of 1-bromo-3-nitrobenzene (5 g, 24.75 mmol) and tert-butyl 4-(4,4,5,5-tetramethyl-1,3,2-dioxaborolan-2-yl)-5,6-dihydropyridine-1(2H)-carboxylate (8.42 g, 27.23 mmol) in water (15 mL) and dioxane (50 mL) were added sodium carbonate (7.87 g, 74.26 mmol) and palladium acetate (555.70 mg, 2.48 mmol). The mixture was stirred at 90° C. for 12 hours. After LC-MS showed consumption of the reactant, the reaction mixture was diluted with water (80 mL) and extracted with ethyl acetate (50 mL×3). The combined organic layers were washed with brine (100 mL), dried over Na₂SO₄, filtered, and concentrated under reduced pressure. The residue was purified by column chromatography (silica gel, petroleum ether/ethyl acetate=1/0 to 1/1). Compound tert-butyl 4-(3-nitrophenyl)-5,6-dihydropyridine-1(2H)-carboxylate (6.5 g, 16.87 mmol, 68.17% yield) was obtained as a yellow solid. LC-MS (ES⁺): m/z 249.1 [M-tBu+H]⁺.

### Step-2

To a solution of tert-butyl 4-(3-nitrophenyl)-3,6-dihydro-2H-pyridine-1-carboxylate (4 g, 13.14 mmol) in methanol (50 mL) was added 10 wt. % Pd/C (400 mg). The mixture was stirred at 25° C. for 5 hours under H₂ atmosphere (15 psi) and the reaction was monitored by TLC. Upon completion of the reaction, the reaction mixture was filtered, and the filtrate was concentrated in vacuo. Compound tert-butyl 4-(3-aminophenyl)piperidine-1-carboxylate (3.5 g, 12.66 mmol, 96.35% yield) was obtained as a white solid. ¹H NMR (400 MHz, DMSO-d₆) δ=6.91 (t, J=7.7 Hz, 1H), 6.51-6.22 (m, 3H), 4.92 (s, 2H), 4.03 (br d, J=12.1 Hz, 2H), 2.90-2.64 (m, 2H), 2.49-2.43 (m, 1H), 1.68 (br d, J=12.6 Hz, 2H), 1.40 (s, 10H).

### Step-3

To a solution of tert-butyl 4-(3-aminophenyl)piperidine-1-carboxylate (2.5 g, 9.05 mmol) and 3-bromopiperidine-2,6-dione (1.74 g, 9.05 mmol) in MeCN (3 mL) was added NaHCO₃ (2.28 g, 27.14 mmol) and the mixture was stirred at 90° C. for 12 hours. After 73% of the desired product was detected by LC-MS, the reaction mixture was concentrated under reduced pressure to remove the solvent. The residue was diluted with water (20 mL) and extracted with ethyl acetate (20 mL×3). The combined organic layers were washed with brine (50 mL), dried over Na₂SO₄, filtered and concentrated under reduced pressure. The residue was purified by column chromatography (silica gel, petroleum ether/ethyl acetate=1/0 to 0/1). Compound tert-butyl 4-(3-((2,6-dioxopiperidin-3-yl)amino)phenyl)piperidine-1-carboxylate (2.5 g, 6.45 mmol, 71.33% yield) was obtained as a yellow solid. LC-MS (ES⁺): m/z 332.0 [M-tBu+H]⁺.

### Step-4

To stirred solution of tert-butyl 4-[3-[(2,6-dioxo-3-piperidyl)amino]phenyl]piperidine-1-carboxylate (160 mg, 412.93 μmol) in DCM (2 mL) was added 4M HCl in dioxane (4 M, 1.03 mL) at 0° C. and the reaction was stirred at room temperature for 3 hours. After completion of the reaction, the solvent was removed under reduced pressure. The residue was washed with MTBE (10 mL×2) and then dried under reduced pressure to afford crude 3-[3-(4-piperidyl)anilino]piperidine-2,6-dione HCl salt (120 mg, 351.24 μmol, 85.06% yield) as a pale yellow solid. LC-MS (ES⁺): m/z 288.4 [M+H]⁺.

### Synthesis of 3-((6-(piperidin-4-yl)pyridin-3-yl)amino)piperidine-2,6-dione

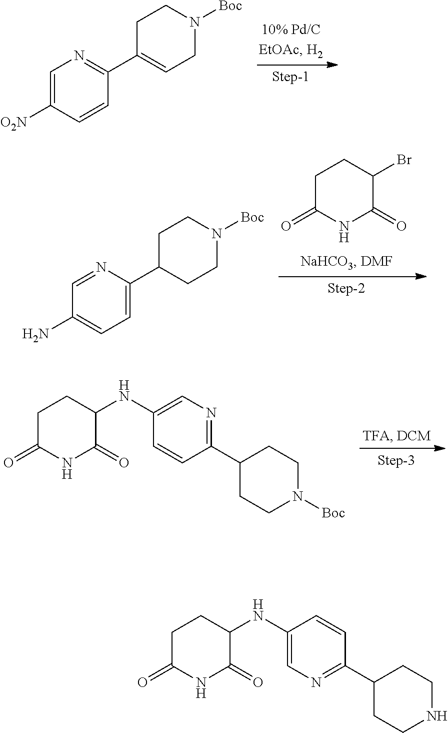

### Step-1

To a stirred solution of tert-butyl 4-(5-nitro-2-pyridyl)-3,6-dihydro-2H-pyridine-1-carboxylate (10 g, 32.75 mmol) in ethyl acetate (100 mL) was added 10 wt. % palladium on carbon, type 487, dry (3.49 g, 32.75 mmol) and the reaction was stirred under hydrogen atmosphere for 16 hours. The reaction progress was monitored by TLC and LC-MS. After completion, the reaction mixture was filtered through a pad of celite and the filtrate was concentrate to dryness. The resulting crude product was purified by column chromatography (silica gel 60-120 mesh, 0-30% ethyl acetate in pet ether) to afford tert-butyl 4-(5-amino-2-pyridyl)piperidine-1-carboxylate (7 g, 23.47 mmol, 71.66% yield). LC-MS (ES⁻): m/z 276.24 [M−H]⁻.

### Step-2

To a stirred solution of tert-butyl 4-(5-amino-2-pyridyl)piperidine-1-carboxylate (6.5 g, 23.44 mmol) and 3-bromopiperidine-2,6-dione (13.50 g, 70.31 mmol) in DMF (40 mL) was added sodium bicarbonate (19.69 g, 234.35 mmol) in a sealed tube. The reaction mixture was stirred at 85° C. for 16 hours. The progress of the reaction was monitored by TLC and LC-MS. Upon completion of the reaction, the reaction mixture was poured into ice water and the product was extracted with ethyl acetate. The organic layer was washed with cold brine solution, dried over anhydrous sodium sulfate and concentrated to give the crude product, which was purified by column chromatography (silica gel 230-400 mesh, 0-100% ethyl acetate in pet ether) to afford tert-butyl 4-[5-[(2,6-dioxo-3-piperidyl)amino]-2-pyridyl]piperidine-1-carboxylate (2.84 g, 6.40 mmol, 27.32% yield) as a light green solid. LC-MS (ES⁻): m/z 387.28 [M−H]⁻.

### Step-3

To a stirred solution of tert-butyl 4-[5-[(2,6-dioxo-3-piperidyl)amino]-2-pyridyl]piperidine-1-carboxylate (1 g, 2.57 mmol) in DCM (10 mL) was added TFA (5.92 g, 51.92 mmol, 4 mL) at 0° C. The reaction mixture was stirred for 3 hours and the reaction progress was monitored by TLC and LC-MS. Upon completion of the reaction, the reaction mixture was evaporated to obtain the crude product, which was triturated with diethyl ether and concentrated in vacuo to afford 3-[[6-(4-piperidyl)-3-pyridyl]amino]piperidine-2,6-dione (700 mg, 2.03 mmol, 78.74% yield) as a green solid. LC-MS (ES⁺): m/z 289.46 [M+H]⁺.

### Synthesis of 3-[4-(4-piperidyl)phenoxy]piperidine-2,6-dione

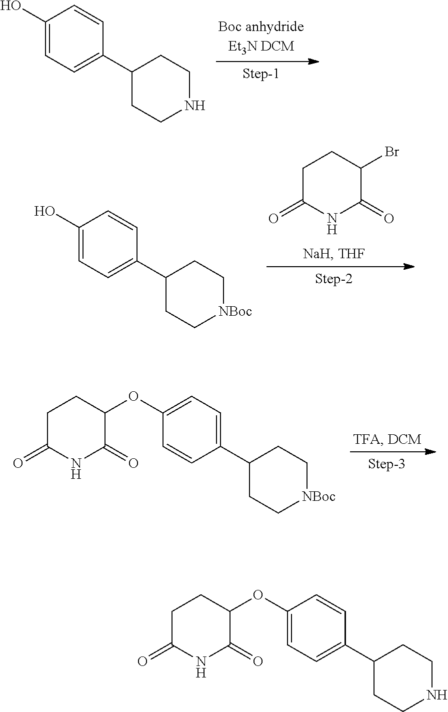 [SMILES: Oc1ccc(C2CCNCC2)cc1 ; CC(C)(C)OC(=O)N1CCC(c2ccc(O)cc2)CC1 ; O=C1CCC(Br)C(=O)N1 ; CC(C)(C)OC(=O)N1CCC(c2ccc(OC3CCC(=O)NC3=O)cc2)CC1 ; O=C1CCC(Oc2ccc(C3CCNCC3)cc2)C(=O)N1]

### Step-1

A solution of 4-(4-piperidyl)phenol HBr salt (2.00 g, 7.75 mmol) in DCM (20 mL) was added into a 100 mL round bottom flask. Tert-butoxycarbonyl tert-butyl carbonate (2.03 g, 9.30 mmol, 2.13 mL) and triethylamine (3.92 g, 38.74 mmol, 5.40 mL) were added and the resulting mixture was stirred at room temperature for 2 hours. After completion of the reaction (confirmed by TLC), the reaction mixture was diluted with ethyl acetate (50 mL), and consecutively washed with water (20 mL) and brine (20 mL). The organic layer was dried over anhydrous sodium sulfate, filtered and concentrated to obtain the crude product, which was purified by flash column chromatography (silica gel 230-400 mesh, 0-80% ethyl acetate in pet ether) to afford tert-butyl 4-(4-hydroxyphenyl)piperidine-1-carboxylate (1.8 g, 6.45 mmol, 83.22% yield) as a white solid. LC-MS (ES⁺): m/z 178.2 [M-Boc+H]⁺.

### Step-2

Sodium hydride (93.78 mg, 3.61 mmol) was added slowly to a stirred solution of tert-butyl 4-(4-hydroxyphenyl)piperidine-1-carboxylate (1.0 g, 3.61 mmol) in THF (10 mL) at 0° C. After addition, the reaction mixture was heated at 70° C. for 30 minutes. It was cooled 0° C. again before 3-bromopiperidine-2,6-dione (553.83 mg, 2.88 mmol) was added very slowly, after which the reaction mixture was heated at 70° C. for 2 hours. Progress of the reaction was monitored by TLC. Upon completion, the reaction was quenched by ammonium chloride and extracted with ethyl acetate, concentrated under reduce pressure to give the crude product, which was purified by column chromatography (silica gel 230-400 mesh, 0-50% ethyl acetate in pet-ether) to afford tert-butyl 4-[4-[(2,6-dioxo-3-piperidyl)oxy]phenyl]piperidine-1-carboxylate (0.5 g, 1.05 mmol, 29.17% yield). LC-MS (ES⁺): m/z 411.41 [M+Na]⁺.

### Step-3

To a solution of tert-butyl 4-[4-[(2,6-dioxo-3-piperidyl)oxy]phenyl]piperidine-1-carboxylate (0.55 g, 1.42 mmol) in DCM (5 mL) was added. 2,2,2-trifluoroacetic acid (161.44 mg, 1.42 mmol, 109.08 p L) at 0° C. and the reaction mixture was stirred at room temperature for 1 hour. The reaction mixture was then concentrated in vacuo to give the crude product, which was triturated with diethyl ether (20 mL) to afford 3-[4-(4-piperidyl)phenoxy]piperidine-2,6-dione TFA salt (0.5 g, 1.13 mmol, 80.02% yield) as a white solid. LC-MS (ES⁺): m/z 289.28 [M+H]⁺.

### Synthesis of 3-[4-(4-piperidyl)phenyl]piperidine-2,6-dione

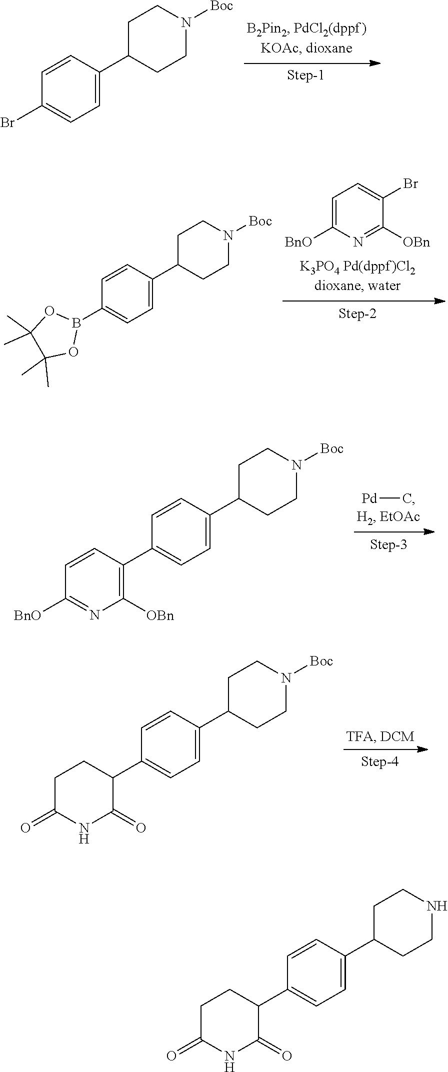

### Step-1

To a 500 mL round bottom flask was added a solution of tert-butyl 4-(4-bromophenyl)piperidine-1-carboxylate (10 g, 29.39 mmol) in 1,4 dioxane (100 mL) was added 4,4,5,5-tetramethyl-2-(4,4,5,5-tetramethyl-1,3,2-dioxaborolan-2-yl)-1,3,2-dioxaborolane (11.19 g, 44.08 mmol) followed by the addition of potassium acetate (8.65 g, 88.17 mmol) at room temperature under argon atmosphere. The reaction mixture was degassed with argon for 20 minutes, after which cyclopentyl(diphenyl)phosphane; dichloromethane; dichloropalladium; iron (2.40 g, 2.94 mmol) was added and the reaction was heated at 100° C. for 6 hours while monitoring with TLC and LC-MS. After completion of the reaction, the volatiles were removed under reduced pressure and the residue was extracted with ethyl acetate (200 mL×3) and water (200 mL). The combined organic layers were washed with brine solution (200 mL), dried over anhydrous sodium sulfate, filtered, and concentrated under reduced pressure. The crude product was purified by column chromatography (silica gel 100-200 mesh, 0-30% EtOAc in pet-ether) to afford tert-butyl 4-[4-(4,4,5,5-tetramethyl-1,3,2-dioxaborolan-2-yl)phenyl]piperidine-1-carboxylate (10 g, 24.27 mmol, 82.58% yield) as a pale yellow solid. LC-MS (ES⁺): m/z 332.41 [M-56+H]⁺.

### Step-2

To a 500 mL round bottom flask was added a solution of tert-butyl 4-[4-(4,4,5,5-tetramethyl-1,3,2-dioxaborolan-2-yl)phenyl]piperidine-1-carboxylate (10 g, 25.82 mmol) in 1,4 dioxane (120 mL) and water (30 mL), followed by the addition of 2,6-dibenzyloxy-3-bromo-pyridine (10.04 g, 27.11 mmol) and potassium phosphate tribasic anhydrous (16.44 g, 77.46 mmol) at room temperature under argon atmosphere. The reaction mixture was degassed with argon for 20 minutes, after which cyclopentyl(diphenyl) phosphane; dichloropalladium; iron (1.89 g, 2.58 mmol) was added and the reaction was heated at 110° C. for 16 hours while monitoring with TLC and LC-MS. Upon completion of the reaction, the catalyst was filtered off through celite bed and washed with ethyl acetate (100 mL×3). The filtrate was washed with water (100 mL) and brine solution (100 mL). The combined organic layers were dried over anhydrous sodium sulfate, filtered, and concentrated under reduced pressure. The crude product was purified by column chromatography (silica gel 230-400 mesh, 0-40% ethyl acetate in pet-ether) to afford the desired product as a yellow thick liquid, which was triturated with pet ether to furnish pure tert-butyl 4-[4-(2,6-dibenzyloxy-3-pyridyl)phenyl]piperidine-1-carboxylate (7 g, 11.57 mmol, 44.80% yield) as a white color solid. LC-MS (ES⁺): m/z 551.43 [M+H]⁺.

### Step-3

A solution of tert-butyl 4-[4-(2,6-dibenzyloxy-3-pyridyl)phenyl]piperidine-1-carboxylate (14 g, 25.42 mmol) in ethyl acetate (420 mL) was added 10% wt. palladium on charcoal (14 g, 25.42 mmol), and the reaction was stirred under hydrogen pressure (70 psi) at room temperature for 16 hours. The reaction progress was monitored by TLC and LC-MS. After the reaction was complete, the catalyst was filtered off through celite and washed with ethyl acetate (200 mL). The filtrate was concentrated under reduced pressure and the residue was triturated in pentane (100 mL) and diethyl ether (100 mL), dried, and concentrated under reduced pressure to afford tert-butyl 4-[4-(2,6-dioxo-3-piperidyl)phenyl]piperidine-1-carboxylate (8.6 g, 23.05 mmol, 90.65% yield) as a white solid. LC-MS (ES⁻): m/z 371.23 [M−H]⁻.

### Step-4

To a stirred solution of tert-butyl 4-[4-(2,6-dioxo-3-piperidyl)phenyl]piperidine-1-carboxylate (250 mg, 671.22 μmol) in DCM (5 mL) was added TFA (5.92 g, 51.92 mmol, 4 mL) at 0° C. The reaction was stirred for 2 hours, and the reaction progress was monitored by LC-MS and TLC. Upon completion, the reaction mixture was concentrated in vacuo to yield the crude product, which was triturated with diethyl ether to obtained the desired product 3-[4-(4-piperidyl)phenyl]piperidine-2,6-dione TFA salt (250 mg, 404.22 μmol, 60.22% yield) as a brown liquid. LC-MS (ES⁻): m/z 371.23 [M−H]⁻.

### Synthesis of 3-(3-fluoro-4-(piperidin-4-yl)phenyl)piperidine-2,6-dione

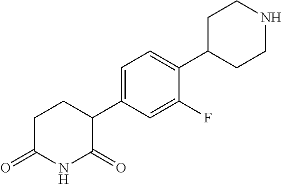 [SMILES: O=C1CCC(c2ccc(C3CCNCC3)c(F)c2)C(=O)N1]

The procedures were substantially similar to those of 3-[4-(4-piperidyl)phenyl]piperidine-2,6-dione, except the synthesis started with tert-butyl 4-(4-bromo-2-fluoro-phenyl)piperidine-1-carboxylate instead of tert-butyl 4-(4-bromophenyl)piperidine-1-carboxylate and palladium hydroxide was used instead of palladium for step-3. LC-MS (ES⁺): m/z 291.37 [M+H]⁺.

### Synthesis of 3-fluoro-3-[4-(4-piperidyl)phenyl]piperidine-2,6-dione

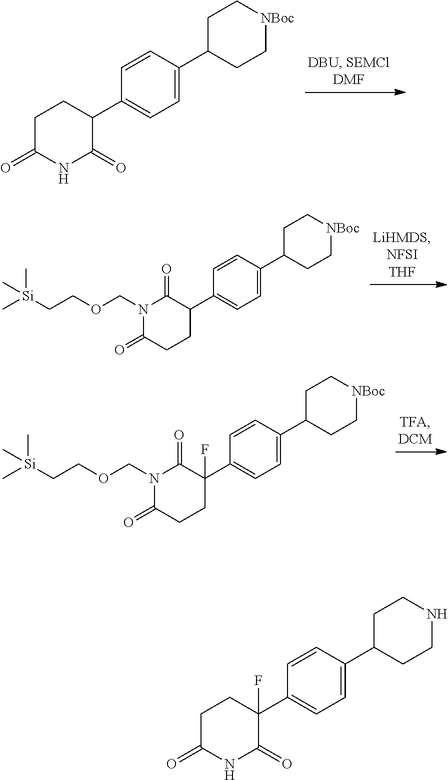 [SMILES: CC(C)(C)OC(=O)N1CCC(c2ccc(C3CCC(=O)NC3=O)cc2)CC1 ; CC(C)(C)OC(=O)N1CCC(c2ccc(C3CCC(=O)N(COCC[Si](C)(C)C)C3=O)cc2)CC1 ; CC(C)(C)OC(=O)N1CCC(c2ccc(C3(F)CCC(=O)N(COCC[Si](C)(C)C)C3=O)cc2)CC1 ; O=C1CCC(F)(c2ccc(C3CCNCC3)cc2)C(=O)N1]

### Step-1

To a stirred solution of tert-butyl 4-[4-(2,6-dioxo-3-piperidyl)phenyl]piperidine-1-carboxylate (8.4 g, 22.55 mmol) in DMF (10 mL) were added 1,8-diazabicyclo[5.4.0]undec-7-ene (6.87 g, 45.11 mmol, 6.73 mL) and 2-(trimethylsilyl)ethoxymethyl chloride (5.64 g, 33.83 mmol, 5.99 mL) at 0° C. The reaction mixture was stirred at 25° C. for 16 hours. The progress of the reaction was monitored by TLC and LC-MS. After completion of the reaction, it was cooled to room temperature, diluted with water, and extracted with ethyl acetate. The combined organic phase was dried over anhydrous sodium sulfate, filtered and concentrated under reduced pressure. The crude product was purified by column chromatography (silica gel 100-200 mesh, 0-50% EtOAc in pet ether) to afford tert-butyl 4-(4-(2,6-dioxo-1-((2-(trimethylsilyl)ethoxy)methyl)piperidin-3-yl)phenyl)piperidine-1-carboxylate (6.2 g, 11.59 mmol, 51.40% yield) as yellow color gummy liquid. LC-MS (ES⁻): m/z 501.36 [M−H]⁻.

### Step-2

To a stirred solution of tert-butyl 4-(4-(2,6-dioxo-1-((2-(trimethylsilyl)ethoxy)methyl) piperidin-3-yl)phenyl)piperidine-1-carboxylate (6.0 g, 11.94 mmol) in THF (120 mL) was added lithium bis(trimethylsilyl)amide (3.99 g, 23.87 mmol) and N-fluorobenzenesulfon imide (3.76 g, 11.94 mmol) at 0° C. The reaction mixture was stirred at −78° C. for 20 minutes. The progress of the reaction was monitored by TLC and LC-MS. After completion of the reaction, the mixture was cooled to room temperature, quenched with NH₄Cl solution (200 mL), and extracted with ethyl acetate (500 mL). The combined organic phase was dried over anhydrous sodium sulfate, filtered, and concentrated under reduced pressure. The crude product was purified by prep-HPLC to afford tert-butyl 4-(4-(3-fluoro-2,6-dioxo-1-((2-(trimethylsilyl)ethoxy)methyl)piperidin-3-yl)phenyl)piperidine-1-carboxylate (1.34 g, 1.78 mmol, 14.88% yield) as yellow color gummy liquid. LC-MS (ES⁻): m/z 519.29 [M−H]⁻.

### Step-3

To a solution of tert-butyl 4-[4-[3-fluoro-2,6-dioxo-1-(2-trimethylsilylethoxymethyl)-3-piperidyl]phenyl]piperidine-1-carboxylate (0.580 g, 1.11 mmol) in DCM (6 mL) was added TFA (1.27 g, 11.14 mmol, 858.13 μL) at 0° C. and the reaction mixture was stirred at room temperature for 16 hours. The reaction mixture was concentrated in vacuo to give the crude product, which was triturated with diethyl ether (50 mL) to afford 3-fluoro-3-[4-(4-piperidyl)phenyl]piperidine-2,6-dione TFA salt (0.580 g, 1.00 mmol, 90.14% yield) as an off-white semi solid. LC-MS (ES⁺): m/z 291.22 [M+H]⁺.

### Synthesis of 1-(1-methyl-6-piperazin-1-yl-indazol-3-yl)hexahydropyrimidine-2,4-dione

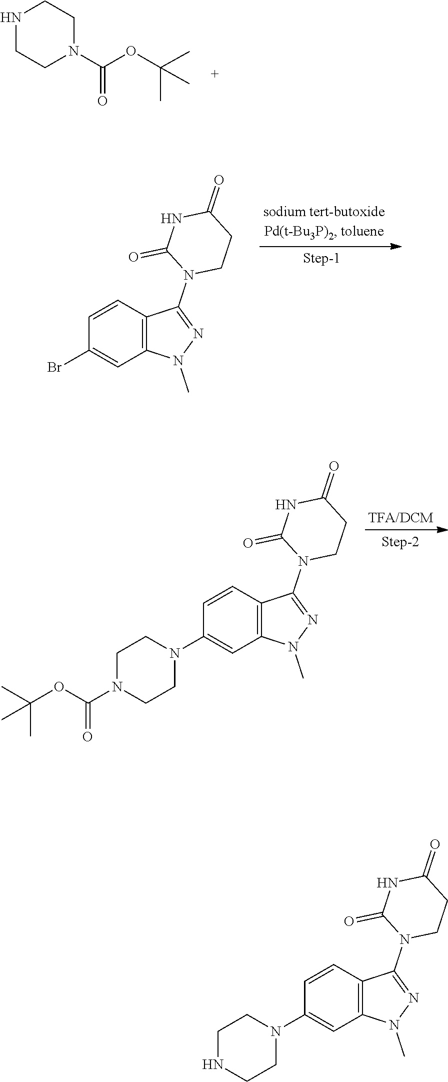 [SMILES: CC(C)(C)OC(=O)N1CCNCC1 ; Cn1nc(N2CCC(=O)NC2=O)c2ccc(Br)cc21 ; Cn1nc(N2CCC(=O)NC2=O)c2ccc(N3CCN(C(=O)OC(C)(C)C)CC3)cc21 ; Cn1nc(N2CCC(=O)NC2=O)c2ccc(N3CCNCC3)cc21]

### Step-1

In a 100 mL round bottom flask, to a stirred solution of 1-(6-bromo-1-methyl-indazol-3-yl)hexahydropyrimidine-2,4-dione (0.5 g, 1.55 mmol) in toluene (10 mL) was added tert-butyl piperazine-1-carboxylate (288.18 mg, 1.55 mmol) and sodium tert-butoxide (297.40 mg, 3.09 mmol) at room temperature. The reaction mixture was degassed with argon for 10 minutes, then Pd(t-Bu₃P)₂ (79.07 mg, 154.73 μmol) was added, then again degassed for 5 minutes. It was stirred at 110° C. for 16 hours, while the progress of reaction was monitored by LC-MS. The reaction mixture was evaporated to give a residue, which was poured into water (20 mL), and the resulting solution was extracted with DCM (2×50 mL). The combined organic layers were washed with brine (50 mL), dried over anhydrous Na₂SO₄, and evaporated to afford the crude product, which was triturated with diethyl ether (30 mL) to afford tert-butyl 4-[3-(2,4-dioxohexahydropyrimidin-1-yl)-1-methyl-indazol-6-yl]piperazine-1-carboxylate (0.310 g, 614.96 μmol, 39.74% yield) as a pale yellow solid. LC-MS (ES⁺): m/z 429.50 [M+H]⁺.

### Step-2

To a stirred solution of tert-butyl 4-[3-(2,4-dioxohexahydropyrimidin-1-yl)-1-methyl-indazol-6-yl]piperazine-1-carboxylate (0.3 g, 700.14 μmol) in DCM (10 mL) was added TFA (1.48 g, 12.98 mmol, 1 mL) at 0° C. and stirring was continued for 6 hours at room temperature. The reaction progress was monitored by LC-MS. After the completion of reaction, the solvent was evaporated under vacuum to obtain the crude product. The crude was triturated in diethyl ether (20 mL) and the solid was filtered and dried to afford 1-(1-methyl-6-piperazin-1-yl-indazol-3-yl)hexahydropyrimidine-2,4-dione (0.280 g, 591.28 μmol, 84.45% yield) as a pale-yellow solid. LC-MS (ES⁺): m/z 329.30 [M+H]⁺.

### Synthesis of 1-(5-fluoro-1-methyl-6-piperazin-1-yl-indazol-3-yl)hexahydropyrimidine-2,4-dione

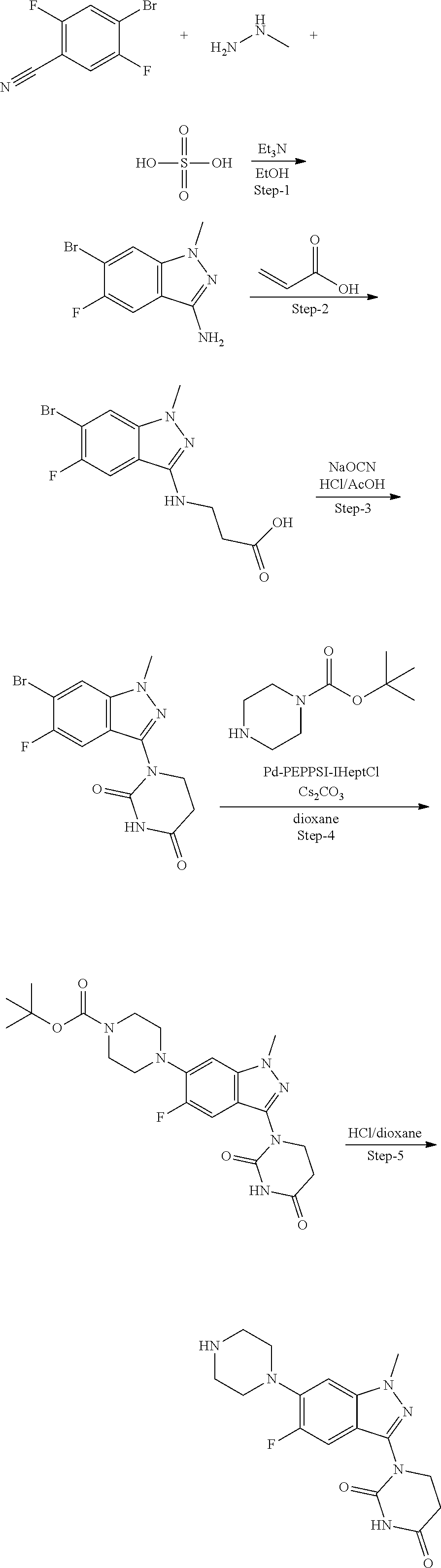

### Step-1

To a solution of 4-bromo-2,5-difluoro-benzonitrile (10 g, 45.87 mmol) in EtOH (30 mL) was added methylhydrazine sulfuric acid (19.84 g, 137.62 mmol) and Et₃N (18.57 g, 183.49 mmol, 25.61 mL). The mixture was stirred at 80° C. for 12 hours. LC-MS showed starting material was consumed completely and one main peak with desired mass was detected. The mixture was cooled down to 30° C., water (300 mL) was added. The mixture was filtered, and the filter cake was washed with water (5 mL×2), and then concentrated at 40° C. under vacuum to afford 6-bromo-5-fluoro-1-methyl-indazol-3-amine (6.5 g, 25.30 mmol, 55.16% yield) as a yellow solid. ¹H-NMR (400 MHz, DMSO-d₆) δ=7.46 (d, J=8.4 Hz, 1H), 7.06-7.04 (m, 1H), 5.68 (s, 2H), 3.83 (d, J=0.8 Hz, 3H). LC-MS (ES⁺): m/z 245 [M+H]⁺.

### Step-2

To a solution of 6-bromo-5-fluoro-1-methyl-indazol-3-amine (22 g, 90.14 mmol) and acrylic acid (9.74 g, 135.21 mmol, 9.28 mL) in 2 M aq. HCl (220 mL) was added tetrabutylammonium bromide (2.91 g, 9.01 mmol). The mixture was stirred at 100° C. for 12 hours. LC-MS showed complete consumption and one main peak with desired mass was detected. All the reaction mixture was basified with a saturated solution of NaHCO₃ until pH=8. The solution was acidified with acetic acid to pH=5. A white solid was precipitated, filtered, and washed with water (250 ml), then dried under reduced pressure to afford 3-[(6-bromo-5-fluoro-1-methyl-indazol-3-yl)amino] propanoic acid (28 g, 88.57 mmol, 98.26% yield) as a white solid. LC-MS (ES⁺): m/z 318.2 [M+H]⁺.

### Step-3

To a solution of 3-[(6-bromo-5-fluoro-1-methyl-indazol-3-yl)amino]propanoic acid (26 g, 82.25 mmol) in AcOH (260 mL) was added NaOCN (11.36 g, 164.49 mmol). The mixture was stirred at 60° C. for 16 hours. To the mixture was added HCl (260 mL). The mixture was stirred at 60° C. for another 3 hours. LC-MS showed starting material was consumed completely and one main peak with desired mass was detected. The reaction mixture was cooled down to room temperature and stirred for 1 hour, filtered and washed with water (250 mL). The cake was dried under vacuum to afford 1-(6-bromo-5-fluoro-1-methyl-indazol-3-yl)hexahydropyrimidine-2,4-dione (18 g, 47.63 mmol, 57.91% yield) as a white solid. ¹H-NMR (400 MHz, DMSO-d₆) δ=10.59 (s, 1H), 8.16 (d, J=5.6 Hz, 1H), 7.62 (d, J=9.2 Hz, 1H), 4.00 (s, 3H), 3.93-3.90 (m, 2H), 2.77-2.73 (m, 2H).

### Step-4

To a solution of 1-(6-bromo-5-fluoro-1-methyl-indazol-3-yl)hexahydropyrimidine-2,4-dione (5 g, 14.66 mmol) and tert-butyl piperazine-1-carboxylate (8.19 g, 43.97 mmol) in dioxane (50 mL) was added Pd-PEPPSI-IHeptCl (427.40 mg, 439.71 μmol) and Cs₂CO₃ (14.33 g, 43.97 mmol) at 25° C. under N₂ atmosphere. The reaction mixture was stirred at 100° C. under N₂ for 16 hours. LC-MS showed complete consumption and desired mass detected. The reaction mixture was diluted with water (200 mL), extract with EtOAc (100 mL×3). The combined organic layer was dried over Na₂SO₄, filtered, and concentrated to a residue which was triturated by EtOAc:MTBE (1:5). The suspension was filtered and dried to afford tert-butyl 4-[3-(2,4-dioxohexahydropyrimidin-1-yl)-5-fluoro-1-methyl-indazol-6-yl]piperazine-1-carboxylate (3.4 g, 6.85 mmol, 46.76% yield) as a grey solid. ¹H-NMR (400 MHz, DMSO-d₆) δ=10.53 (s, 1H), 7.38 (d, J=12.8 Hz, 1H), 7.16 (d, J=6.8 Hz, 1H), 3.94 (s, 3H), 3.89-3.87 (m, 2H), 3.52 (br s, 4H), 3.06-2.98 (m, 4H), 2.75-2.73 (m, 2H), 1.43 (s, 9H).

### Step-5

A solution of tert-butyl 4-[3-(2,4-dioxohexahydropyrimidin-1-yl)-5-fluoro-1-methyl-indazol-6-yl]piperazine-1-carboxylate (2.4 g, 5.38 mmol) in 4M HCl/dioxane (30 mL) was stirred at 25° C. for 2 hours. TLC showed reactant was consumed and a new spot was formed. The reaction mixture was concentrated to a residue which was triturated with MTBE (200 mL), filtered, and the filter cake was dried under vacuum to afford 1-(5-fluoro-1-methyl-6-piperazin-1-yl-indazol-3-yl)hexahydropyrimidine-2,4-dione (2 g, 4.70 mmol, 87.47% yield) as a grey solid. ¹H-NMR (400 MHz, DMSO-d₆) δ=10.54 (s, 1H), 9.22 (br s, 2H), 7.41 (d, J=12.4 Hz, 1H), 7.23 (d, J=7.2 Hz, 1H), 3.97 (s, 3H), 3.91-3.88 (m, 2H), 3.31 (br s, 8H), 2.76-2.72 (m, 2H).

### Synthesis of 3-[3-methyl-2-oxo-5-(4-piperidyl)benzimidazol-1-yl]piperidine-2,6-dione

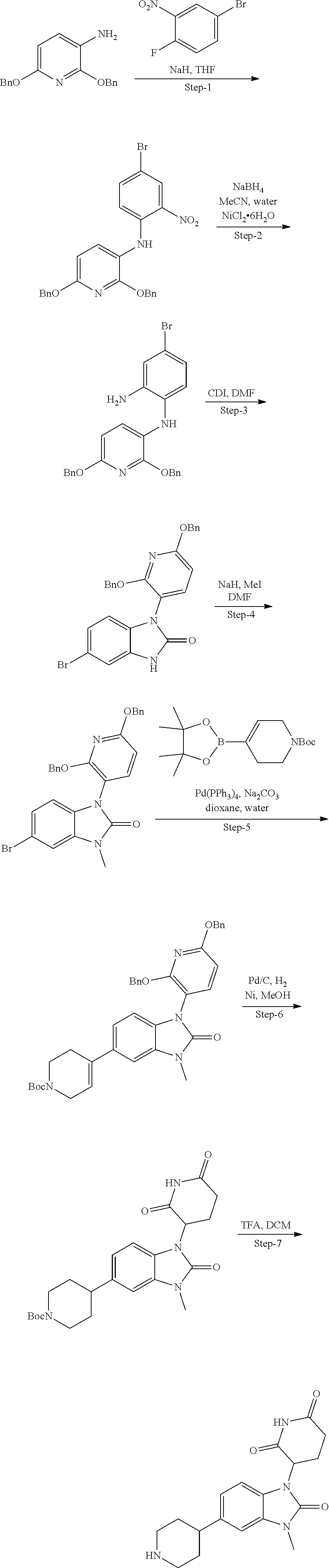

### Step-1

To sodium hydride (60% dispersion in mineral oil) (53.51 g, 2.33 mol) was added THF (2300 mL) and the suspension was cooled to 5-10° C. A solution of 2,6-dibenzyloxypyridin-3-amine (230 g, 750.76 mmol) in THF (1400 mL) was added at 5-10° C. over 20 minutes with exothermicity observed. The temperature was maintained for 30 minutes. To this solution was added 4-bromo-1-fluoro-2-nitrobenzene, 98% (247.75 g, 1.13 mol, 138.41 mL) in THF (1600 mL) at 5-10° C. over 20 minutes. The solution was warmed to room temperature and the temperature was maintained for 16 hours. TLC (20% EtOAc in pet ether) confirmed the formation of product. The reaction mass was quenched with 10% water in THF (5 V) at below 10° C., with observed exothermicity. Saturated NaCl solution (10 V) was added at below 15° C. and warmed to room temperature. The layers were separated, and the organic layer was concentrated under vacuum. The aqueous layer was taken and extracted with DCM (15 V) and kept aside. The organic layer was combined with crude and washed with water (5 V) and concentrated completely under vacuum at 45° C. The crude was charged into DCM (2.5 V) at 45° C. and maintained for 15 min until dissolution, then added pet ether (10 V) at 45° C. and maintained for 1 hr at 45° C. Cooled to room temperature and maintained for 30 min. Filtered and washed with pet ether (2×3 V) to afford 2,6-dibenzyloxy-N-(4-bromo-2-nitro-phenyl)pyridin-3-amine (400 g, 686 mmol, 91% yield). LC-MS (ES⁺): m/z 506.32 [M+H]⁺.

### Step-2

A solution of 2,6-dibenzyloxy-N-(4-bromo-2-nitro-phenyl)pyridin-3-amine (50 g, 98.75 mmol) in ACN (450 mL) and water (50 mL) was cooled to 0-5° C. and sodium borohydride (7.47 g, 197.49 mmol, 6.98 mL) was added portionwise for 60 hours, during which room temperature was maintained for 4 hours. TLC was used to monitor the progress of the reaction. Sodium borohydride (7.47 g, 197.49 mmol, 6.98 mL) was added at 0-5° C. and temperature maintained for 2 hours. Then the reaction was quenched with 10% NH₄Cl solution (5 V), water added (5 V), followed by DCM (10 V), then stirred at room temperature for 15 min. The aqueous layer was extracted with DCM (10 V) and the combined organic layers were washed with water (10 V) and concentrated completely under vacuum at 40° C. Pet ether was used to strip the residue (3 V), then charged into 10% EtOAc in pet ether (5 V) into a crude residue and heated to 45° C. The temperature was maintained at 45° C. for 30 min, cooled to room temperature, and maintained for 30 min. The pure product was filtered and washed with pet ether (3 V). LC-MS (ES⁺): m/z 476.33 [M+H]⁺.

### Step-3

To the stirred solution of 4-bromo-N1-(2,6-dibenzyloxy-3-pyridyl)benzene-1,2-diamine (200 g, 419.85 mmol) in DMF (800 mL) was added di(imidazol-1-yl)methanone (177.00 g, 1.09 mol) at 25-35° C. with observed exothermicity. CDI was charged as a single lot. Initial temperature 25° C. was monitored with the final temperature of 35° C. noted at 15 minutes. The reaction was stirred for 14 hours at room temperature. TLC showed the consumption of starting material. The reaction was charged into water (420 mL) at room temperature. Precipitation was formed (Note: Slow addition required a minimum of 1 h for bulk scale) and the mixture was stirred for 3 hours. The solid was filtered and washed with water and pet ether (2×35 ml). The product was dried under vacuum for 7 hours at 50° C. to afford 6-bromo-3-(2,6-dibenzyloxy-3-pyridyl)-1H-benzimidazol-2-one (200 g, 391.43 mmol, 93.23% yield). LC-MS (ES⁺): m/z 502.1 [M+H]⁺.

### Step-4

To a stirred solution of 6-bromo-3-(2,6-dibenzyloxy-3-pyridyl)-1H-benzimidazol-2-one (108 g, 214.99 mmol) in DMF (1000 mL) was added sodium hydride (60% dispersion in mineral oil) (14.83 g, 644.96 mmol) portionwise at 0-28° C. The reaction mixture was stirred for 1 hour, followed by dropwise addition of methyl iodide (stored over copper) (31.16 g, 214.99 mmol, 13.37 mL) over half an hour. Progress of the reaction was monitored by TLC and LC-MS. The reaction mixture was diluted with ice cold water, and the resulting solid was obtained, filtered, and dried over vacuum. The solid was extracted with ethyl acetate, then washed with brine, dried over sodium sulfate, and concentrated to dryness. The crude compound was washed with pentane to afford the product 5-bromo-1-(2,6-dibenzyloxy-3-pyridyl)-3-methyl-benzimidazol-2-one (95 g, 183.81 mmol, 85.50% yield) as a light brown solid. LC-MS (ES⁺): m/z 516.14 [M+H]⁺.

### Step-5

To a solution of 5-bromo-1-(2,6-dibenzyloxy-3-pyridyl)-3-methyl-benzimidazol-2-one (20 g, 38.73 mmol) in 1,4-dioxane (160 mL) and water (40 mL) was added sodium carbonate (12.32 g, 116.19 mmol) and tert-butyl 4-(4,4,5,5-tetramethyl-1,3,2-dioxaborolan-2-yl)-3,6-dihydro-2H-pyridine-1-carboxylate (15.57 g, 50.35 mmol). The reaction was purged with nitrogen for 20 minutes, then charged with palladium (0) tetrakis(triphenylphosphine) (2.24 g, 1.94 mmol) and heated to 90-100° C. for 5 hours. TLC confirmed the formation of product. The reaction was cooled to room temperature and filtered through a celite bed and washed with EtOAc. The filtrate was taken and distilled completely under vacuum at 45° C. The crude product was dissolved in EtOAc (15 V) and separated with water (10 V). The organic layer was washed with water (5 V), brine (5 V), then dried over anhydrous Na₂SO₄. The organic layer was concentrated in vacuo at 45° C. then purified by column chromatography (100-200 mesh silica gel, 0-30% ethyl acetate in pet ether) to afford tert-butyl 4-[1-(2,6-dibenzyloxy-3-pyridyl)-3-methyl-2-oxo-benzimidazol-5-yl]-3,6-dihydro-2H-pyridine-1-carboxylate (21 g, 33.06 mmol, 99% yield). LC-MS (ES⁺): m/z 619.41 [M+H]⁺.

### Step-6

To a solution of tert-butyl 4-[1-(2,6-dibenzyloxy-3-pyridyl)-3-methyl-2-oxo-benzimidazol-5-yl]-3,6-dihydro-2H-pyridine-1-carboxylate (40 g, 64.65 mmol) in methanol (1600 mL) was added palladium, 10% on carbon, type 487, dry (12.00 g, 112.76 mmol) and nickel (12.00 g, 204.45 mmol). Hydrogen gas (10 kg) was applied, and the reaction was maintained at 60-65° C. for 16 hours. The reaction mass was cooled to room temperature then filtered and washed with DCM and MeOH. The filtrate was taken and distilled completely under vacuum at 45° C. To the crude residue was added IPA (3 V) and heated to 60° C. for 15 minutes. Pet ether (3 V) was added and the mixture cooled to room temperature, and stirred at this temperature for 1 hour. The solid was filtered and washed with pet ether to afford tert-butyl 4-[1-(2,6-dioxo-3-piperidyl)-3-methyl-2-oxo-benzimidazol-5-yl]piperidine-1-carboxylate (21 g, 44 mmol, 69% yield). LC-MS (ES⁻): m/z 441.18 [M−H]⁻.

### Step-7

To a solution of tert-butyl 4-[1-(2,6-dioxo-3-piperidyl)-3-methyl-2-oxo-benzimidazol-5-yl]piperidine-1-carboxylate (7.5 g, 16.95 mmol) in DCM (75 mL) was added trifluoroacetic acid (55.87 g, 490.03 mmol, 37.75 mL) at 0-5° C. slowly and the temperature was maintained for 15 minutes. The reaction was warmed to room temperature and maintained for 3 hours. LC-MS complied with the formation of product. DCM and TFA were removed under vacuum at 40° C. and the crude stripped off with toluene (2×5 V) and diethyl ether added with the formation of solid observed. The reaction was decanted after adding diethyl ether (3×5 V), then dried at 45° C. The crude was dissolved in MeOH (10 V), stirred for 10 minutes, and filtered through a sintered funnel and washed with MeOH with slight undissolved particles observed. The distilled filtrate was completely evaporated under vacuum at 45° C. to afford 3-[3-methyl-2-oxo-5-(4-piperidyl)benzimidazol-1-yl]piperidine-2,6-dione (7.72 g, 16.5 mmol, 97% yield). LC-MS (ES⁻): m/z 343.35 [M−H]⁻.

### Synthesis of 3-[3-methyl-2-oxo-4-(4-piperidyl)benzimidazol-1-yl]piperidine-2,6-dione

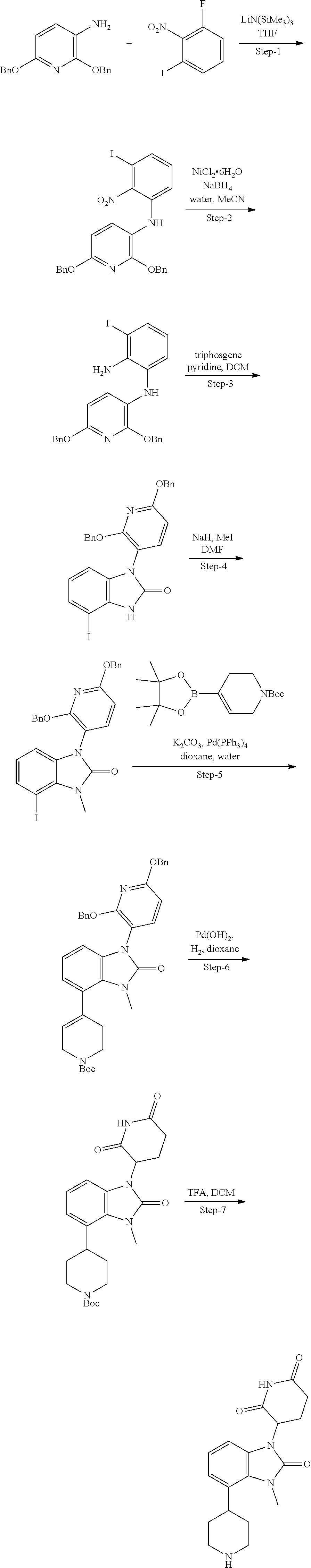

### Step-1

2,6-dibenzyloxypyridin-3-amine (50 g, 163.21 mmol) was dissolved with THF (500 mL) and cooled to −78° C. Lithium bis(trimethylsilyl)amide (40.96 g, 244.81 mmol) was added dropwise, then stirred for 1 hour at −78° C. 1-fluoro-3-iodo-2-nitro-benzene (43.58 g, 163.21 mmol) was added dropwise as a solution in THF (500 mL) at −78° C., then stirred for 1 hour at −78° C. After the reaction was complete as confirmed by TLC, the reaction was then quenched with 10% ammonium chloride solution (150 mL). The solvent was evaporated to a black gummy solid. Pet ether was added and stirred well for 15 minutes until formation of a brown solid, which was filtered through a Buchner funnel and washed with pet ether (2×300 mL). The filter cake was dried under vacuum to afford 2,6-dibenzyloxy-N-(3-iodo-2-nitro-phenyl)pyridin-3-amine (80 g, 144.57 mmol, 83% yield). LC-MS (ES⁺): m/z 554.20 [M+H]⁺.

### Step-2

A solution of 2,6-dibenzyloxy-N-(3-iodo-2-nitro-phenyl)pyridin-3-amine (80 g, 144.57 mmol) in acetonitrile (720 mL) and water (80 mL) was added nickel(II) chloride hexahydrate, 98% (8.22 g, 28.91 mmol). The reaction was cooled to 0° C. and sodium borohydride (13.67 g, 361.44 mmol) was added portionwise over 1 hour. The reaction mixture was stirred for 30 minutes at room temperature. Upon completion of the reaction as confirmed by TLC, the reaction was filtered through celite, and washed with ethyl acetate.

The organic layers were separated and washed with brine solution and dried over anhydrous Na₂SO₄. The organic layer was evaporated to obtain a black gummy solid. To this crude residue, pet ether was added and stirred until a brown solid was obtained. The solid was filtered through a Buchner funnel, then washed with pet ether and dried under vacuum to afford N1-(2,6-dibenzyloxy-3-pyridyl)-3-iodo-benzene-1,2-diamine (36 g, 66 mmol, 45% yield). LC-MS (ES⁺): m/z 524.23 [M+H]⁺.

### Step-3

A solution of N1-(2,6-dibenzyloxy-3-pyridyl)-3-iodo-benzene-1,2-diamine (5.58 g, 10.66 mmol) in DCM (120 mL) was cooled to 0° C. Pyridine (8.43 g, 106.62 mmol, 8.62 mL) was added dropwise and the solution stirred for 30 minutes at 0° C. Triphosgene (4.75 g, 15.99 mmol) was added dropwise at 0° C. as a solution. The reaction mixture was stirred for 1 hour at room temperature while monitoring by TLC. Upon completion, the reaction was quenched with saturated NaHCO₃ solution, which was added slowly at 0° C. with observed effervescence. The reaction mass was extracted with DCM, then washed with brine solution and dried over anhydrous Na₂SO₄. The organic layers were evaporated to obtain a pale brown solid. To this crude solid, diethyl ether was added and stirred well, before filtering through a Buchner funnel. The product was washed with diethyl ether and dried under vacuum to afford 3-(2,6-dibenzyloxy-3-pyridyl)-7-iodo-1H-benzimidazol-2-one (5.1 g, 8.9 mmol, 83% yield) LC-MS (ES⁺): m/z 550.55 [M+H]⁺.

### Step-4

A solution of 3-(2,6-dibenzyloxy-3-pyridyl)-7-iodo-1H-benzimidazol-2-one (47.82 g, 87.06 mmol) in DMF (410 mL) and cooled to 0° C. Sodium hydride (60% dispersion in mineral oil) (5.60 g, 243.75 mmol) was added portion-wise, then the reaction mixture stirred for 30 minutes at room temperature. Iodomethane (18.53 g, 130.58 mmol, 8.13 mL) was added dropwise at 0° C. and the reaction mixture stirred for 1 hour at room temperature. Upon completion of the reaction as confirmed by TLC, the reaction was decanted slowly into ice cold water. An off-white solid precipitated and was filtered through a Buchner funnel, then washed with ice cold water and dried under vacuum. The solid was azeotroped with toluene (2×200 mL) to obtain a pale brown solid. Pet ether was added and stirred well for 10 minutes before filtering the solid through Buchner funnel and washing with pet ether (3×100 mL). The product was dried under vacuum to afford 1-(2,6-dibenzyloxy-3-pyridyl)-4-iodo-3-methyl-benzimidazol-2-one as a light brown solid (47 g, 83 mmol, 95% yield). LC-MS (ES⁺): m/z 564.03 [M+H]⁺.

### Step-5

To a stirred solution of 1-(2,6-dibenzyloxy-3-pyridyl)-4-iodo-3-methyl-benzimidazol-2-one (25 g, 44.37 mmol) in dioxane (210 mL) and water (90 mL) were added potassium carbonate (18.40 g, 133.12 mmol) and tert-butyl 4-(4,4,5,5-tetramethyl-1,3,2-dioxaborolan-2-yl)-3,6-dihydro-2H-pyridine-1-carboxylate (20.58 g, 66.56 mmol). The reaction mixture was degassed with nitrogen for 10 minutes before palladium triphenylphosphine (5.13 g, 4.44 mmol) was added. The reaction was stirred at 100° C. for 4 hours and monitored by TLC and LC-MS. The reaction mass was filtered and concentrated under vacuum, then purified by column chromatography (100-200 mesh silica gel, 10-20% EtOAc in pet ether) to afford tert-butyl 4-[1-(2,6-dibenzyloxy-3-pyridyl)-3-methyl-2-oxo-benzimidazol-4-yl]-3,6-dihydro-2H-pyridine-1-carboxylate (20 g, 31.63 mmol, 71.27% yield) as a yellow solid. LC-MS (ES⁺): m/z 619.19 [M+H]⁺.

### Step-6

To a stirred solution of tert-butyl 4-[1-(2,6-dibenzyloxy-3-pyridyl)-3-methyl-2-oxo-benzimidazol-4-yl]-3,6-dihydro-2H-pyridine-1-carboxylate (21.00 g, 33.94 mmol) in 1,4-dioxane (600 mL) was added dihydroxypalladium (5.72 g, 40.73 mmol). The reaction mixture was stirred for 12 hours at 60-65° C. under hydrogen gas at 150 psi. TLC and LC-MS were checked to confirm reaction completion (10% methanol in DCM, R_{f} value: 0.4). Upon completion, the reaction was filtered through celite and washed with ethyl acetate. The filtrate was concentrated under reduced pressure to give the crude product, which was triturated with diethyl ether to afford tert-butyl 4-[1-(2,6-dibenzyloxy-3-pyridyl)-3-methyl-2-oxo-benzimidazol-4-yl]-3,6-dihydro-2H-pyridine-1-carboxylate (10 g, 19.98 mmol, 58.86% yield). LC-MS (ES⁺): m/z 441.54 [M+H]⁺.

### Step-7

A solution of tert-butyl 4-[1-(2,6-dioxo-3-piperidyl)-3-methyl-2-oxo-benzimidazol-4-yl]piperidine-1-carboxylate (10 g, 22.60 mmol) in DCM (150 mL) was cooled to 0° C. Trifluoroacetic acid (25.77 g, 225.99 mmol, 17.41 mL) was added and the reaction mixture stirred for 12 hours at room temperature. TLC confirmed reaction completion (10% methanol in DCM, R_{f} value: 0.2). Upon completion the reaction solvent was evaporated, and diethyl ether (2×100 mL) added to the crude mixture. Diethyl ether was removed and the product was dried under vacuum to afford 3-[3-methyl-2-oxo-4-(4-piperidyl)benzimidazol-1-yl]piperidine-2,6-dione TFA salt (10.71 g, 22.39 mmol, 99.06% yield) as an off-white solid. LC-MS (ES⁺): m/z 343.33 [M+H]⁺.

### Synthesis of 3-[3-methyl-4-[4-(methylamino)-1-piperidyl]-2-oxo-benzimidazol-1-yl]piperidine-2,6-dione

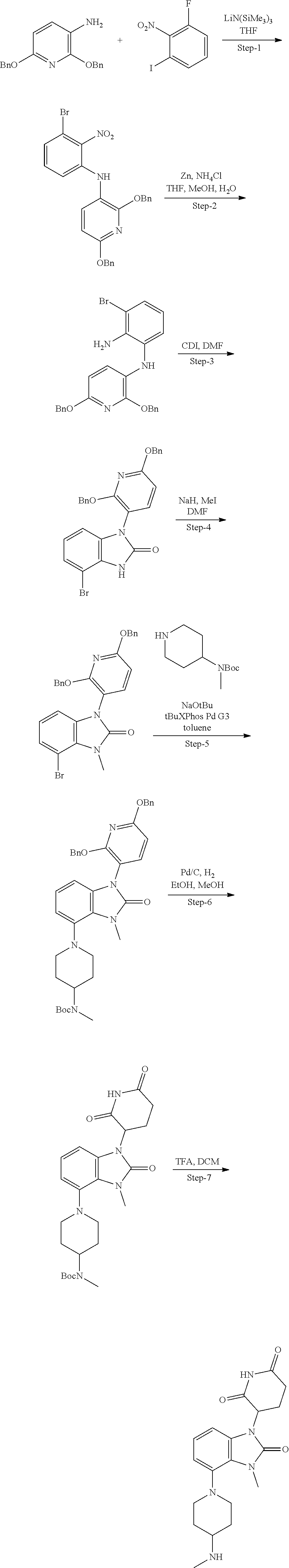

### Step-1

A solution of 2,6-dibenzyloxypyridin-3-amine (2 g, 6.53 mmol) in THF (50 mL) was cooled to −78° C. To this was added lithium bis(trimethylsilyl)azanide (1.09 g, 6.53 mmol, 6.5 mL) dropwise over 15 minutes at −78° C. The reaction was maintained at −78° C. for 1 hour, followed by the dropwise addition of 1-bromo-3-fluoro-2-nitro-benzene (1.44 g, 6.53 mmol). The reaction mixture was stirred for another 2 hours. Completion of the reaction was confirmed by TLC (20% EtOAc/Pet ether) and LC-MS. The reaction mixture was diluted with 10% ammonium chloride solution and concentrated under reduced pressure. The crude material was purified by column chromatography (pet ether and ethyl acetate) to afford 2,6-dibenzyloxy-N-(3-bromo-2-nitro-phenyl)pyridin-3-amine (2.5 g, 4.08 mmol, 62.42% yield) as a yellow solid. LC-MS (ES⁺): m/z 506.32 [M+H]⁺.

### Step-2

To a stirred solution of 2,6-dibenzyloxy-N-(3-bromo-2-nitro-phenyl)pyridin-3-amine (20 g, 39.50 mmol) in THF (65 mL) and methanol (65 mL) was added zinc (25.83 g, 394.99 mmol, 3.62 mL) followed by the addition of a suspension of ammonia hydrochloride (31.69 g, 592.48 mmol) in water (65 mL). The reaction mixture was stirred at room temperature for 2 h and the progress of the reaction monitored by TLC. Upon completion of the reaction, the contents were passed through a celite bed. The filtrate was concentrated under vacuum and extracted by EtOAc (250 ml). The organic layers were separated and dried over anhydrous Na₂SO₄, then evaporated under vacuum. The crude material was purified by column chromatography using Davisil silica (eluting solvent 0-70% EtOAc in hexane) to afford 3-bromo-N1-(2,6-dibenzyloxy-3-pyridyl)benzene-1,2-diamine (15 g, 27.56 mmol, 69.78% yield) as a brown solid. LC-MS (ES⁺): m/z 398.46 [M-Br+H]⁺.

### Step-3

A solution of 3-bromo-N1-(2,6-dibenzyloxy-3-pyridyl)benzene-1,2-diamine (210 g, 440.84 mmol) in DMF (1.17 L) was added di(imidazol-1-yl)methanone (200.15 g, 1.23 mol) at room temperature. The reaction mixture was stirred for 16 hours at room temperature. TLC confirmed the consumption of starting material (40% ethyl acetate in pet ether, R_{f} value: 0.4). Upon completion the reaction, the mixture was poured into ice cold water. An off-white solid was precipitated and filtered through Buchner funnel. The wet solid was washed with water and dried under vacuum to afford 7-bromo-3-(2,6-dibenzyloxy-3-pyridyl)-1H-benzimidazol-2-one (220 g, 378.33 mmol, 85.82% yield). LC-MS (ES⁻): m/z 500.41 [M−H]⁻.

### Step-4

To a stirred solution of 7-bromo-3-(2,6-dibenzyloxy-3-pyridyl)-1H-benzimidazol-2-one (220 g, 437.93 mmol) in DMF (2200 mL) was added sodium hydride, 60% dispersion in mineral oil, (28.19 g, 1.23 mol) at 0° C. The reaction mixture was warmed to room temperature and maintained for 1 h. The reaction was cooled again to 0° C. and iodomethane (93.24 g, 656.90 mmol, 40.89 mL) added dropwise at 0-5° C. The reaction mass was allowed to warm to room temperature and maintained for 1 h. The progress of the reaction was followed and confirmed by TLC (20% ethyl acetate: pet ether R_{f} value: 0.3). Upon completion, the reaction was quenched into ice cold water and an off-white solid precipitated that was isolated by vacuum filtration & Buchner funnel, and washed with water (1000 mL). The wet solid obtained was dried under vacuum to afford 4-bromo-1-(2,6-dibenzyloxy-3-pyridyl)-3-methyl-benzimidazol-2-one as an off-white solid (221 g, 420.66 mmol, 96.05% yield). LC-MS (ES⁺): m/z 516.09 [M+H]⁺.

### Step-5

In a sealed tube, a solution of 4-bromo-1-(2,6-dibenzyloxy-3-pyridyl)-3-methyl-benzimidazol-2-one (0.5 g, 968.27 μmol), tert-butyl N-methyl-N-(4-piperidyl)carbamate (207.50 mg, 968.27 μmol) in toluene (10 mL) was added sodium 2-methylpropan-2-olate (279.16 mg, 2.90 mmol). The reaction was degassed with argon for 15 minutes, then tBuXPhos Pd G3 (76.88 mg, 96.83 μmol) was added to the reaction mixture and degassed for another 5 minutes. The reaction mixture was then heated at 90° C. for 5 hours. The progress of the reaction was monitored by LC-MS. The reaction mixture was filtered through a celite bed and the filtrate concentrated to give the crude compound which was purified by column chromatography (100-200 mesh silica gel, 0-70% ethyl acetate in pet ether) to afford tert-butyl N-[1-[1-(2,6-dibenzyloxy-3-pyridyl)-3-methyl-2-oxo-benzimidazol-4-yl]-4-piperidyl]-N-methyl-carbamate (0.25 g, 307.80 μmol, 31.79% yield) as a yellow liquid. LC-MS (ES⁺): m/z 672.41 [M+Na]⁺.

### Step-6

Tert-butyl N-[1-[1-(2,6-dibenzyloxy-3-pyridyl)-3-methyl-2-oxo-benzimidazol-4-yl]-4-piperidyl]-N-methyl-carbamate (0.415 g, 638.68 μmol) was solvated in ethanol (3 mL) and methanol (3 mL), and purged with nitrogen for 10 minutes. To this solution was added palladium, 10% on carbon, Type 487, dry (67.97 mg, 638.68 μmol) and the reaction mixture stirred under a hydrogen atmosphere (rubber bladder) at room temperature for 5 hr. The progress of reaction was monitored by TLC (10% methanol DCM; R_{f} value: 0.3). After completion the reaction mixture was filtered through a celite bed and washed with methanol (50 mL×2) and the organic layer concentrated to furnish the product tert-butyl N-[1-[1-(2,6-dioxo-3-piperidyl)-3-methyl-2-oxo-benzimidazol-4-yl]-4-piperidyl]-N-methyl-carbamate (0.3 g, 610.75 μmol, 95.63% yield) as a brown solid. LC-MS (ES⁺): m/z 672.41 [M+H]⁺.

### Step-7

To a stirred solution of tert-butyl N-[1-[1-(2,6-dioxo-3-piperidyl)-3-methyl-2-oxo-benzimidazol-4-yl]-4-piperidyl]-N-methyl-carbamate (0.3 g, 636.20 μmol) in DCM (50 mL) at 0° C. was added TFA (72.54 mg, 636.20 μmol, 49.01 μL) over 10 minutes. The reaction mixture was stirred at 25° C. for 4 hours and the reaction was monitored by TLC (10% methanol in DCM, R_{f} value: 0.2). After reaction completion, the mixture was concentrated and co-distilled with toluene (10 ml) and diethyl ether (2×50 ml) to afford the product 3-[3-methyl-4-[4-(methylamino)-1-piperidyl]-2-oxo-benzimidazol-1-yl]piperidine-2,6-dione TFA salt (0.23 g, 447.87 μmol, 70.40% yield) as a brown solid. LC-MS (ES⁺): m/z 372.28 [M+H]⁺.

### Synthesis of 3-[3-methyl-5-[4-(methylamino)-1-piperidyl]-2-oxo-benzimidazol-1-yl]piperidine-2,6-dione

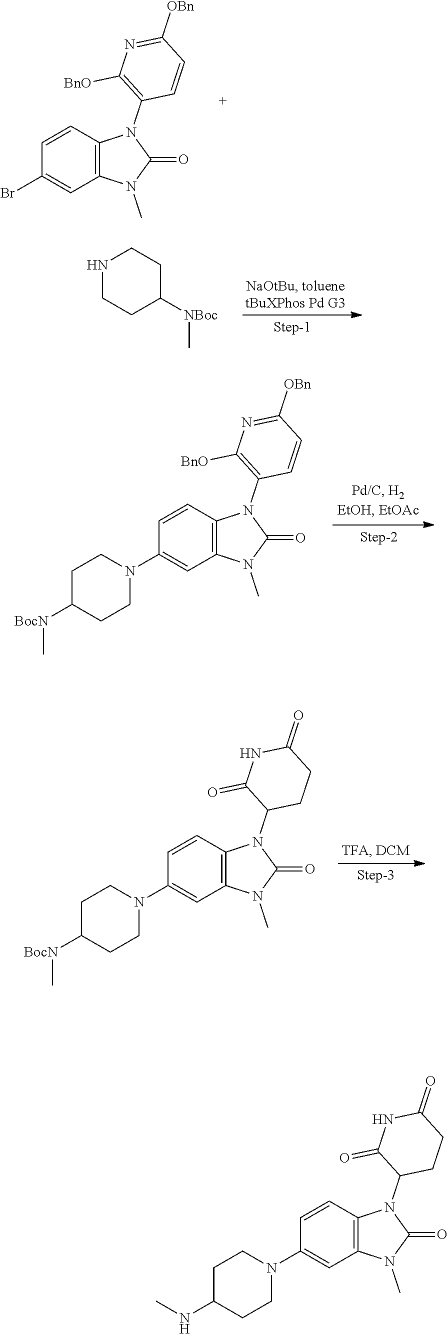

### Step-1

In a sealed tube, a solution of 5-bromo-1-(2,6-dibenzyloxy-3-pyridyl)-3-methyl-benzimidazol-2-one (1 g, 1.94 mmol) and tert-butyl N-methyl-N-(4-piperidyl)carbamate (622.52 mg, 2.90 mmol) in toluene (60 mL) was added sodium 2-methylpropan-2-olate (558.30 mg, 5.81 mmol). The reaction was degassed with argon for 15 minutes, then tBuXPhos Pd G3 (153.76 mg, 193.65 μmol) was added to the reaction mixture and degassed again for 5 minutes. The reaction mixture was then heated at 90° C. for 16 hours, and the progress of the reaction monitored by LC-MS. The reaction mixture was filtered through celite bed and the filtrate was concentrated in vacuo and then purified by column chromatography (100-200 mesh silica gel, 0-70% ethyl acetate in pet ether) to afford tert-butyl N-[1-[1-(2,6-dibenzyloxy-3-pyridyl)-3-methyl-2-oxo-benzimidazol-5-yl]-4-piperidyl]-N-methyl-carbamate (0.7 g, 1.02 mmol, 52.85% yield) as a yellow liquid. LC-MS (ES⁺): m/z 650.97 [M+H]⁺.

### Step-2

A stirring solution of tert-butyl N-[1-[1-(2,6-dibenzyloxy-3-pyridyl)-3-methyl-2-oxo-benzimidazol-5-yl]-4-piperidyl]-N-methyl-carbamate (0.6 g, 923.39 μmol) in a mixture of ethanol (50 mL) and ethyl acetate (50 mL) was purged with hydrogen gas followed by addition of palladium, 10% on carbon, type 487, dry (523.21 mg, 4.92 mmol) and concentrated HCl (254.14 mg, 7.06 mmol, 2 mL). The reaction mixture was stirred under hydrogen atmosphere (1 atm pressure) at room temperature for 5 hours. The progress of the reaction monitored by LC-MS. After complete consumption of the starting material, the reaction mixture was filtered through a celite bed and washed with methanol (50 mL×2). The filtrate was concentrated to furnish the tert-butyl N-[1-[1-(2,6-dioxo-3-piperidyl)-3-methyl-2-oxo-benzimidazol-5-yl]-4-piperidyl]-N-methyl-carbamate (0.25 g, 334.01 μmol, 36.17% yield) as a yellow solid. LC-MS (ES⁺): m/z 472.93 [M+H]⁺.

### Step-3

To a stirred solution of tert-butyl N-[1-[1-(2,6-dioxo-3-piperidyl)-3-methyl-2-oxo-benzimidazol-5-yl]-4-piperidyl]-N-methyl-carbamate (0.32 g, 678.62 μmol) at 0° C. was added TFA (77.38 mg, 678.62 μmol, 52.28 μL) over 10 minutes. The reaction mixture was stirred at 25° C. for 4 hours and the reaction monitored by TLC (10% methanol in DCM R_{f} value: 0.2). After completion, the reaction mixture was concentrated and co-distilled with toluene (10 ml) and diethyl ether (2×50 ml) to afford the product 3-[3-methyl-5-[4-(methylamino)-1-piperidyl]-2-oxo-benzimidazol-1-yl]piperidine-2,6-dione TFA salt (0.25 g, 475.69 μmol, 70.10% yield) as an off-white solid. LC-MS (ES⁺): m/z 372.28 [M+H]⁺.

### Synthesis of 3-[4-[4-(methylamino)-1-piperidyl]anilino]piperidine-2,6-dione

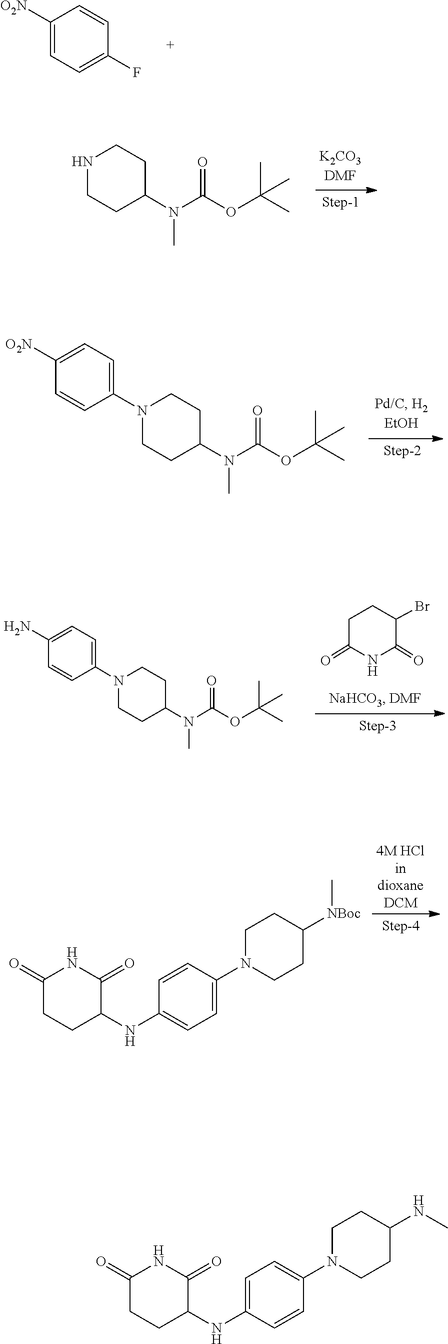

### Step-1

To a stirred solution of 1-fluoro-4-nitro-benzene (2 g, 14.17 mmol, 1.50 mL) in DMF (10 mL) was added tert-butyl N-methyl-N-(4-piperidyl)carbamate (3.04 g, 14.17 mmol) and potassium carbonate granular (3.92 g, 28.35 mmol) and the reaction was heated to 80° C. for 4 hours. TLC (R_{f}: 0.4 in 10% Ethyl acetate in pet ether) and LC-MS were checked for completion of the reaction. After completion, the reaction was concentrated under vacuum to get the crude product which was purified by flash column chromatography (silica gel) to afford tert-butyl N-methyl-N-[1-(4-nitrophenyl)-4-piperidyl]carbamate (2 g, 5.84 mmol, 41.23% yield). LC-MS (ES⁺): m/z 336.28 [M+H]⁺.

### Step-2

To the stirred solution of tert-butyl N-methyl-N-[1-(4-nitrophenyl)-4-piperidyl]carbamate (2 g, 5.96 mmol) in ethanol (20 mL) was added palladium, 10% on carbon, type 487, dry (634.59 mg, 5.96 mmol) and the reaction was stirred under H₂ atmosphere for 4 hours. TLC (R. 0.4 in 50% ethyl acetate in pet ether) and LC-MS were checked for completion of reaction. The reaction was filtered through a celite bed and washed with methanol. The solvent was evaporated under vacuum, and the residue was washed with pentane to afford tert-butyl N-[1-(4-aminophenyl)-4-piperidyl]-N-methyl-carbamate (1.5 g, 4.67 mmol, 78.24% yield). LC-MS (ES⁺): m/z 303.31 [M+H]⁺.

### Step-3

Tert-butyl N-[1-(4-aminophenyl)-4-piperidyl]-N-methyl-carbamate (1 g, 3.27 mmol) was dissolved in DMF (10 mL), and 3-bromopiperidine-2,6-dione (1.26 g, 6.55 mmol) and sodium bicarbonate (1.10 g, 13.10 mmol) were added. The reaction was heated to 100° C. for 16 hours. The reaction progress was monitored by TLC (R. 0.4 in 50% ethyl acetate in pet ether) which showed consumption of starting material. The reaction mixture was then quenched with water, extracted with ethyl acetate, and the organic layers were washed with brine, then dried over anhydrous Na₂SO₄ and concentrated under reduced pressure. The crude compound was purified by column chromatography (100-200 mesh silica gel, ethyl acetate and pet ether) to obtain tert-butyl N-[1-[4-[(2,6-dioxo-3-piperidyl)amino]phenyl]-4-piperidyl]-N-methyl-carbamate (0.6 g, 1.35 mmol, 41.36% yield) as a pale yellow solid. LC-MS (ES⁺): m/z 417.56 [M+H]⁺.

### Step-4

To a stirred solution of tert-butyl N-[1-[4-[(2,6-dioxo-3-piperidyl)amino]phenyl]-4-piperidyl]-N-methyl-carbamate (0.5 g, 1.20 mmol) in DCM (5 mL), was added 4 M HCl in dioxane (43.77 mg, 1.20 mmol) slowly at 0° C. and the reaction was stirred at 0-25° C. for 2 hours. TLC (R_{f}: 0.4 in 50% ethyl acetate in pet ether) and LC-MS were checked for completion of reaction. After completion, the reaction was concentrated under reduced pressure and washed with pentane and diethyl ether to afford 3-[4-[4-(methylamino)-1-piperidyl]anilino]piperidine-2,6-dione HCl salt (0.35 g, 823.28 μmol, 68.58% yield). LC-MS (ES⁺): m/z 317.37 [M+H]⁺.

### Synthesis of 3-[3-[[4-(methylamino)-1-piperidyl]methyl]anilino]piperidine-2,6-dione

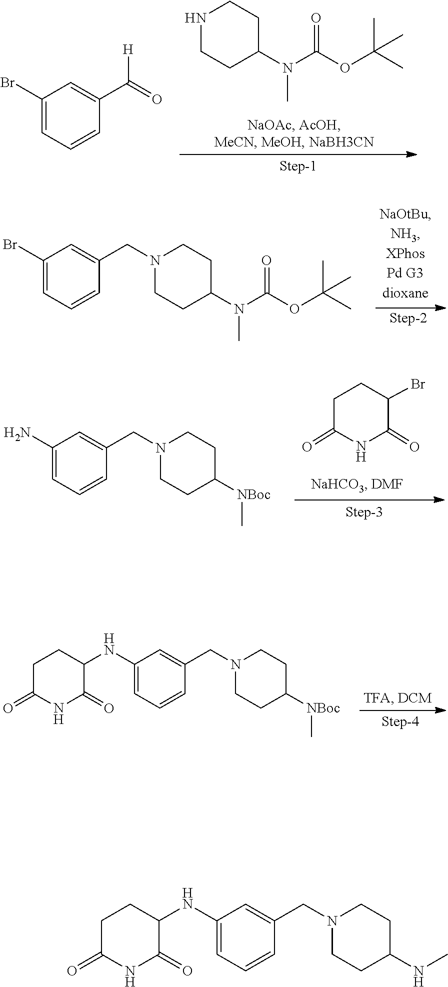

### Step-1

To a stirred solution of 3-bromo benzaldehyde (5 g, 27.15 mmol) in a mixture of MeCN:MeOH (1:1 ratio, 20 mL) was added tert-butyl methyl(piperidin-4-yl)carbamate (6.95 g, 32.58 mmol) followed by sodium acetate (6.68 g, 81.45 mmol), and catalytic acetic acid (0.1 mL). The reaction was stirred at 100° C. for 3 hours. After 3 hours, the reaction mixture was cooled to 0° C. and sodium cyanobohydride (1.68 g, 27.15 mmol) added portion-wise and allowed to stir at room temperature for 16 hours. After complete consumption of the starting material, the reaction mixture was quenched with cold water. The solvent was evaporated under reduced pressure, diluted with water, and extracted with ethyl acetate. The combined organic layer was washed with brine solution, dried over anhydrous sodium sulfate, filtered, and concentrated to get crude compound which was purified using column chromatography (silica gel 100-200 mesh, EtOAc and pet ether) to afford tert-butyl (1-(3-bromobenzyl)piperidin-4-yl)(methyl)carbamate formic acid salt (5.5 g, 10.91 mmol, 40.20% yield). LC-MS (ES⁺): m/z 385.4 [M+H]⁺.

### Step-2

In a sealed tube, a solution of tert-butyl (1-(3-bromobenzyl)piperidin-4-yl)(methyl)carbamate (3 g, 7.85 mmol) in 1,4-dioxane (20 mL) was added NaO^{t}Bu (2.26 g, 23.54 mmol). It was purged with ammonia gas for 20 minutes, followed by the addition of XPhos Pd G3 (1.25 g, 1.57 mmol). The resulting reaction mixture was heated and stirred at 90° C. for 16 hours. The progress of reaction was monitored by LC-MS and thin layer chromatography. After complete consumption of the starting material, the reaction mixture was filtered and the filtrate was concentrated to dryness. The crude compound was diluted with water and extracted with ethyl acetate. The combined organic layer was washed with brine solution, dried over anhydrous sodium sulfate, filtered, and concentrated to get tert-butyl 2-[1-[(3-aminophenyl)methyl]-4-piperidyl]propanoate (2 g, 1.24 mmol, 15.82% yield). LC-MS (ES⁺): m/z 320.38 [M+H]⁺.

### Step-3

To a stirred solution of tert-butyl N-[1-[(3-aminophenyl)methyl]-4-piperidyl]-N-methyl-carbamate (2.0 g, 6.28 mmol) in DMF (20 mL) was added NaHCO₃ (1.58 g, 18.84 mmol) and the solution was purged with argon gas for 15 minutes. Then 3-bromopiperidine-2,6-dione (3.62 g, 18.84 mmol) was added and the resulting reaction mixture was heated at 90° C. with stirring for 16 hours. The progress of reaction was monitored by LC-MS and thin layer chromatography. After complete consumption of the starting material, the reaction mixture was concentrated to dryness and purified by prep-HPLC to afford tert-butyl (1-(3-((2,6-dioxopiperidin-3-yl)amino)benzyl)piperidin-4-yl)(methyl) carbamate formic acid salt (0.4 g, 792.17 μmol, 12.61% yield). LC-MS (ES⁺): m/z 320.38 [M+H]⁺.

### Step-4

A stirred solution of tert-butyl (1-(3-((2,6-dioxopiperidin-3-yl)amino)benzyl) piperidin-4-yl)(methyl) carbamate TFA salt (0.03 g, 55.09 μmol) in DCM (3 mL) was cooled to 0° C. and TFA (444.00 mg, 3.89 mmol, 0.3 mL) was added. The reaction mixture was stirred at room temperature for 2 hours. The progress of reaction was monitored by LC-MS and thin layer chromatography (10% MeOH in DCM. R_{f} value: 0.3). After complete consumption of the starting material, the reaction mixture was concentrated to dryness to afford 3-[3-[[4-(methylamino)-1-piperidyl]methyl]anilino]piperidine-2,6-dione TFA salt (0.023 g, 42.36 μmol, 76.89% yield) as a light red solid. LC-MS (ES⁺): m/z 331.51 [M+H]⁺. Synthesis of 3-[3-[4-(methylamino)-1-piperidyl]anilino]piperidine-2,6-dione

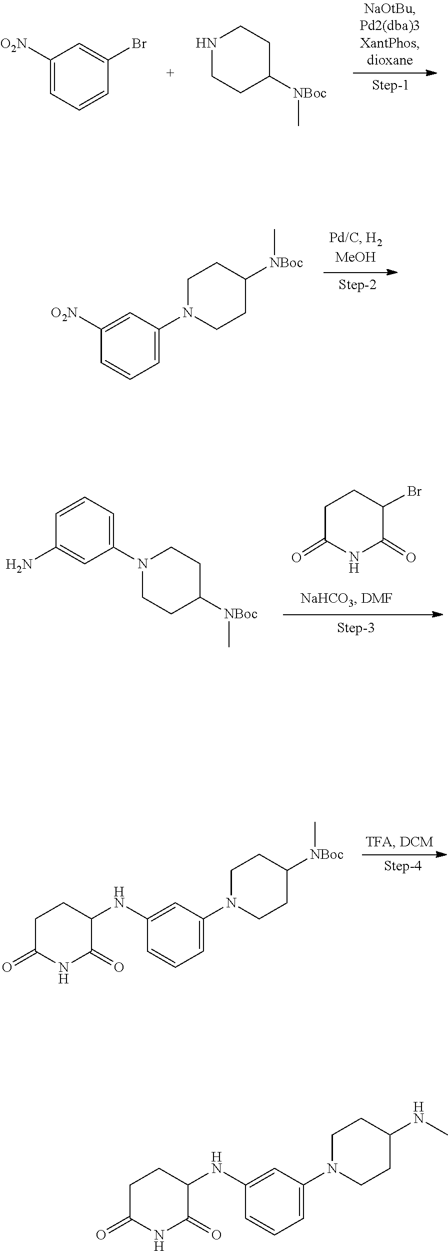

### Step-1

To a stirred solution of tert-butyl N-methyl-N-(4-piperidyl)carbamate (5.30 g, 24.75 mmol) in dioxane (30 mL) were added sodium tert-butoxide (4.76 g, 49.50 mmol), tris(dibenzylideneacetone)dipalladium(0) (1.13 g, 1.12 mmol), 4,5-Bis(diphenylphosphino)-9,9-dimethylxanthene (286.44 mg, 495.04 μmol). The reaction was stirred for 15 minutes before adding 1-bromo-3-nitro-benzene (5.0 g, 24.75 mmol, 52.52 μL). The reaction mixture stirred at 100° C. for 16 hours while monitoring by TLC (Mobile phase: 50% EtOAc: Pet ether; R_{f} (Product): 0.5). After completion, the reaction mixture was quenched with ice and the precipitated solid was filtered and dried under vacuum to afford tert-butyl N-methyl-N-[1-(3-nitrophenyl)-4-piperidyl]carbamate (5.0 g, 14.61 mmol, 59.02% yield) as a yellow solid. LC-MS (ES⁺): m/z 336.2 [M+H]⁺.

### Step-2

To a stirred solution of tert-butyl N-methyl-N-[1-(3-nitrophenyl)-4-piperidyl]carbamate (5.0 g, 14.91 mmol) in methanol (50 mL) was added palladium on carbon (5.00 g, 46.98 mmol) and the reaction mixture stirred at room temperature for 16 hours while monitoring by TLC (Mobile phase: 50% EtOAc: Pet ether. R_{f} (Product): 0.5). After completion, the reaction mixture was filtered through celite, and the organic layer was concentrated under reduced pressure to get the crude product which was purified by column chromatography (100-200 mesh silica gel, 0 to 50% EtOAc in pet ether) to afford tert-butyl N-[1-(3-aminophenyl)-4-piperidyl]-N-methyl-carbamate (3.0 g, 9.53 mmol, 63.91% yield) as a yellow solid. LC-MS (ES⁺): m/z 306.18 [M+H]⁺.

### Step-3

To a stirred solution of tert-butyl N-[1-(3-aminophenyl)-4-piperidyl]-N-methyl-carbamate (2.5 g, 8.19 mmol) and 3-bromopiperidine-2,6-dione (3.14 g, 16.37 mmol) in DMF (3 mL) was added sodium bicarbonate (2.75 g, 32.74 mmol). The reaction mixture was stirred at 80° C. for 16 hours while monitoring by TLC. The reaction mixture was cooled to room temperature, diluted with water, and extracted with ethyl acetate. The organic phase was dried over sodium sulfate, filtered, and concentrated under reduced pressure to get crude product which was purified by column chromatography (100 to 200 mesh silica gel, 0 to 50% EtOAc in pet ether) to afford tert-butyl N-[1-[3-[(2,6-dioxo-3-piperidyl)amino]phenyl]-4-piperidyl]-N-methyl-carbamate (1.3 g, 2.90 mmol, 35.46% yield) as a yellow colored gummy liquid. LC-MS (ES⁺): m/z 417.53 [M+H]⁺.

### Step-4

To a stirred solution of tert-butyl N-[1-[3-[(2,6-dioxo-3-piperidyl)amino]phenyl]-4-piperidyl]-N-methyl-carbamate (80 mg, 192.07 μmol) in DCM (5 mL) was added TFA (131.40 mg, 1.15 mmol, 88.79 p L) at 0° C. and stirred at room temperature for 2 hours, while monitoring by TLC. The reaction mixture was concentrated under reduced pressure and the residue triturated with diethyl ether (2×100 mL). The precipitated solid was filtered and dried under vacuum to afford 3-[3-[4-(methylamino)-1-piperidyl]anilino]piperidine-2,6-dione TFA salt (72 mg, 159.15 μmol, 82.86% yield) as a blue solid. LC-MS (ES⁺): m/z 317.52 [M+H]⁺.

### Synthesis of 3-[3-[4-(methylamino)-1-piperidyl]phenyl]piperidine-2,6-dione

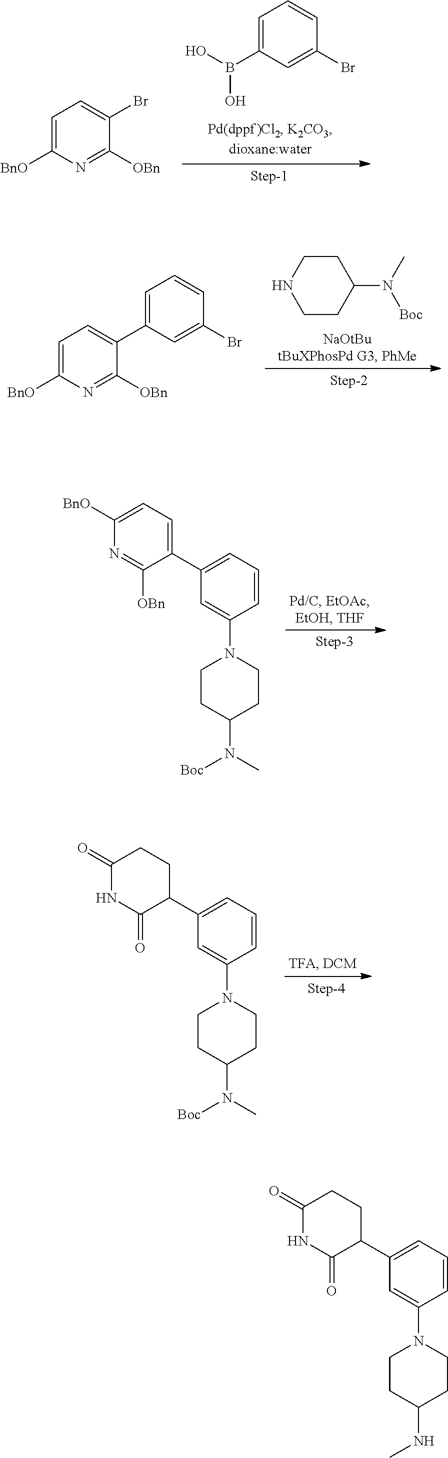

### Step-1

A solution of (3-bromophenyl)boronic acid (1 g, 4.98 mmol), 2,6-dibenzyloxy-3-iodo-pyridine (2.08 g, 4.98 mmol), potassium carbonate (2.06 g, 14.94 mmol), cyclopentyl(diphenyl)phosphane; dichloropalladium; iron (364.35 mg, 497.94 μmol) and 2,6-dibenzyloxy-3-iodo-pyridine (2.08 g, 4.98 mmol) in dioxane:water (4:1 ratio 5 mL) was stirred for 16 hours at 90° C. The progress of the reaction was monitored by LC-MS. After completion of the reaction, the reaction mixture was diluted with cold water and extracted with ethyl acetate. The combined organic layer was washed with water, brine, dried over anhydrous Na₂SO₄, filtered, and concentrated under reduced pressure to give compound 2,6-dibenzyloxy-3-(3-bromophenyl)pyridine (1.500 g, 1.41 mmol, 28.35% yield). LC-MS (ES⁺): m/z 446.2 [M+H]⁺.

### Step-2

A solution of 2,6-dibenzyloxy-3-(3-bromophenyl)pyridine (1.3 g, 2.91 mmol), tert-butyl N-methyl-N-(4-piperidyl)carbamate (749.02 mg, 3.50 mmol), sodium tert-butoxide (559.82 mg, 5.83 mmol) and tBuXPhos Pd G3 (462.52 mg, 582.52 μmol) in toluene (15 mL) was stirred for 16 hours at 100° C. The reaction mixture was concentrated under reduced pressure, diluted with cold water and extracted with ethyl acetate. The combined organic layer was washed with water, brine, dried over anhydrous Na₂SO₄, filtered, and concentrated under reduced pressure to afford tert-butyl N-[1-[3-(2,6-dibenzyloxy-3-pyridyl)phenyl]-4-piperidyl]-N-methyl-carbamate (0.9 g, 947.00 μmol, 32.51% yield). LC-MS (ES⁺): m/z 580.3 [M+H]⁺.

### Step-3

To a stirred solution of tert-butyl N-[1-[3-(2,6-dibenzyloxy-3-pyridyl)phenyl]-4-piperidyl]-N-methyl-carbamate (1.8 g, 3.10 mmol) in a mixture of ethyl acetate:ethanol:THF=1:5:4 (30 mL) was added 10% palladium on carbon (type 487, 1.8 g). Then the reaction mixture was stirred under H₂ (1 atm pressure) for 16 hours. The reaction mixture was passed through celite bed, then washed with methanol and concentrated under reduced pressure to obtain the desired crude compound. The crude material was purified by reverse phase column chromatography (Column/dimensions: X-SELECT C18 (19×250×5 um) Mobile phase A: 0.1% FA in water (aq.) Mobile phase B: ACN (org) Gradient (Time/% B): 0/20, 2/20, 10/50, 15/50, 15.1/98, 18/98, 18.1/20, 21/20. Flow rate: 16 ml/min. Solubility: ACN+THF+WATER) to afford compound tert-butyl N-[1-[3-(2,6-dioxo-3-piperidyl)phenyl]-4-piperidyl]-N-methyl-carbamate (1.00 g, 2.48 mmol, 79.92% yield). LC-MS (ES⁻): m/z 400.3 [M−H]⁻.

### Step-4

A solution of tert-butyl N-[1-[3-(2,6-dioxo-3-piperidyl)phenyl]-4-piperidyl]-N-methyl-carbamate (0.040 g, 99.63 μmol) and 20% 2,2,2-trifluoroacetic acid (11.36 mg, 99.63 μmol, 7.68 μL) in DCM (1 mL) was stirred for 4 hours at 0° C., then at room temperature. The progress of the reaction was monitored by LC-MS. After completion of the reaction, the reaction mixture was concentrated under reduced pressure and triturated with diethyl ether to afford 3-[3-[4-(methylamino)-1-piperidyl]phenyl]piperidine-2,6-dione TFA salt (27 mg, 64.16 μmol, 64.40% yield). LC-MS (ES⁺): m/z 302.3 [M+H]⁺.

### Synthesis of 3-[4-[4-(methylamino)-1-piperidyl]phenyl]piperidine-2,6-dione

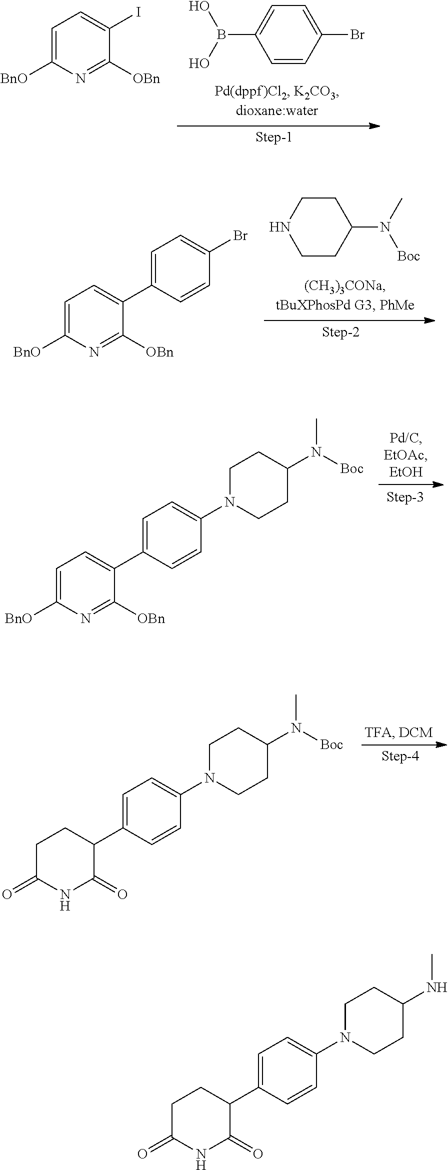

### Step-1

In a seal tube, a solution of (4-bromophenyl)boronic acid (4 g, 19.92 mmol) and 2,6-dibenzyloxy-3-iodo-pyridine (8.31 g, 19.92 mmol) in dioxane and water (20 mL) was added potassium carbonate (8.25 g, 59.75 mmol). The reaction mixture was purged with argon for 20 minutes before Pd(dppf)Cl₂ (1.46 g, 1.99 mmol) was added and the reaction was stirred for 16 hours at 90° C., The reaction progress was monitored by LC-MS. After completion of the reaction, the reaction mixture was filtered and concentrated. It was then diluted with water and extracted with ethyl acetate. The organic layer was dried over anhydrous sodium sulfate and concentrated under reduced pressure to obtain the crude product, which was purified by column chromatography (60-120 mesh silica gel, 0-4% ethyl acetate in pet ether) to afford 2,6-dibenzyloxy-3-(4-bromophenyl)pyridine formic acid salt (7 g, 9.93 mmol, 49.83% yield) as an off-white solid. LC-MS (ES⁺): m/z 446.1 [M+H]⁺.

### Step-2

To a stirred solution of tert-butyl N-methyl-N-(4-piperidyl)carbamate TFA salt (1.47 g, 4.48 mmol) in toluene (20 mL) was added (CH₃)₃CONa (861.24 mg). After 10 minutes, 2,6-dibenzyloxy-3-(4-bromophenyl)pyridine (2 g, 4.48 mmol) was added and the resulting reaction mixture was stirred for 16 hours with heating. Progress of the reaction was monitored by LC-MS. The reaction crude was filtered and concentrated. The crude mixture was diluted in ethyl acetate and washed with water. The organic layer was dried over sodium sulfate and concentrated under reduced pressure. The crude product was purified by column chromatography to afford tert-butyl N-[1-[4-(2,6-dibenzyloxy-3-pyridyl)phenyl]-4-piperidyl]-N-methyl-carbamate (3 g, 3.83 mmol, 85.45% yield). LC-MS (ES⁺): m/z 581.00 [M+H]⁺.

### Step-3

To a stirred solution of tert-butyl N-[1-[4-(2,6-dibenzyloxy-3-pyridyl)phenyl]-4-piperidyl]-N-methyl-carbamate (3 g, 5.17 mmol) in EtOAc (10 mL), and EtOH (10 mL) was added Pd/C (3.14 g, 25.87 mmol) under hydrogen atmosphere. The resulting reaction mixture was stirred at room temperature for 16 hours. Progress of the reaction was monitored by LC-MS. After consumption of the starting material, the resulted crude was filtered and concentrated in vacuo. The crude product was purified by prep-HPLC (Column/dimensions: X-BRIDGE PHENYL-C18 (19*250*5 um), Mobile phase A: 5 mM ammonium acetate in water (aqueous), Mobile phase B: ACN (org)) to afford the compound tert-butyl N-[1-[4-(2,6-dioxo-3-piperidyl)phenyl]-4-piperidyl]-N-methyl-carbamate (1.7 g, 4.21 mmol, 81.37% yield). LC-MS (ES⁺): m/z 402.5 [M+H]⁺.

### Step-4

To a solution of tert-butyl N-[1-[4-(2,6-dioxo-3-piperidyl)phenyl]-4-piperidyl]-N-methyl-carbamate formic acid salt (0.05 g, 111.73 μmol) in DCM (2 mL) at 0° C. was added TFA (12.74 mg, 111.73 μmol, 8.61 μL) and the reaction mixture was stirred at room temperature for 2 hours. The reaction mixture was concentrated in vacuo to get the crude product, which was triturated with diethyl ether (5 mL) to afford 3-[4-[4-(methylamino)-1-piperidyl]phenyl]piperidine-2,6-dione formic acid salt (0.03 g, 84.15 μmol, 75.32% yield) as a light brown solid. LC-MS (ES⁺): m/z 302.5 [M+H]⁺.

### Synthesis of 3-[2-fluoro-3-(4-piperidyl)anilino]piperidine-2,6-dione

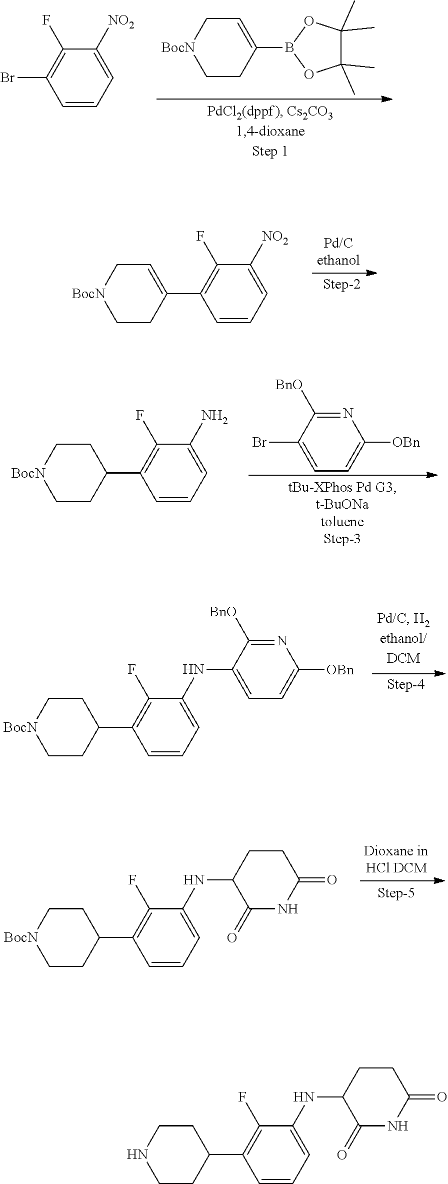

### Step-1

A solution of 1-bromo-2-fluoro-3-nitro-benzene (4 g, 18.18 mmol) in 1,4 dioxane (40 mL) were added tert-butyl 4-(4,4,5,5-tetramethyl-1,3,2-dioxaborolan-2-yl)-3,6-dihydro-2H-pyridine-1-carboxylate (6.18 g, 20.00 mmol) and cesium carbonate (14.81 g, 45.46 mmol). The reaction mixture was purged with dry argon gas for about 15 minutes before Pd(dppf)Cl₂ (1.33 g, 1.82 mmol) was added. The reaction mixture was heated to 80° C. for 16 hours. Upon completion, the mixture was cooled to room temperature and solvent was removed to give crude mixture, which was further purified by column purification (ethyl acetate/petroleum ether) to afford tert-butyl 4-(2-fluoro-3-nitro-phenyl)-3,6-dihydro-2H-pyridine-1-carboxylate (4 g, 6.20 mmol, 34.13% yield). LC-MS (ES⁺): m/z 223.4 [M+H-Boc]⁺.

### Step-2

A solution of tert-butyl 4-(2-fluoro-3-nitro-phenyl)-3,6-dihydro-2H-pyridine-1-carboxylate (4.3 g, 13.34 mmol) in ethanol (60 mL) was added Pd/C (4.3 g). The reaction mixture was stirred at 28° C. for 16 hours under hydrogen atmosphere. Upon completion of the reaction, the reaction mixture was filtered through a celite bed, then the filtrate was concentrated to get crude compound tert-butyl 4-(3-amino-2-fluoro-phenyl)piperidine-1-carboxylate (3.5 g, 9.27 mmol, 69.52% yield), which was used for the next step without further purifications. LC-MS (ES⁺): m/z 295.4 [M+H]⁺.

### Step-3

A solution of tert-butyl 4-(3-amino-2-fluoro-phenyl)piperidine-1-carboxylate (2.0 g, 6.79 mmol) in toluene (30 mL) were added 2,6-dibenzyloxy-3-bromo-pyridine (2.77 g, 7.47 mmol) and sodium tert butoxide (1.96 g, 20.38 mmol). The reaction mixture was purged with dry argon gas before t-Butyl XPhos Pd G3 (539.47 mg, 679.43 μmol) was added. Reaction was heated to 90° C. for 16 hours. Upon completion of the reaction, the crude mixture was concentrated and purified by column chromatography over 100-200 silica mesh using Ethyl acetate-Pet ether as eluent to obtain tert-butyl 4-[3-[(2,6-dibenzyloxy-3-pyridyl)amino]-2-fluoro-phenyl]piperidine-1-carboxylate (1.8 g, 2.16 mmol, 31.77% yield). LC-MS (ES⁻): m/z 583.6 [M+H]⁻.

### Step-4

A solution of tert-butyl 4-[3-[(2,6-dibenzyloxy-3-pyridyl)amino]-2-fluoro-phenyl]piperidine-1-carboxylate (3.0 g, 5.14 mmol) in ethanol (20 mL) and DCM (20 mL) was added Pd/C (3.0 g, 24.70 mmol) and the reaction mixture was stirred at 28° C. for 16 hours under hydrogen atmosphere. Upon completion of the reaction, the reaction mixture was filtered through a celite bed, and concentrated under reduced pressure to get tert-butyl 4-[3-[(2,6-dioxo-3-piperidyl)amino]-2-fluoro-phenyl]piperidine-1-carboxylate (2 g, 4.20 mmol, 81.68% yield), which was used for the next step without further purification. LC-MS (ES⁻): m/z 404.3 [M+H]⁻.

### Step-5

A stirred solution of tert-butyl 4-[3-[(2,6-dioxo-3-piperidyl)amino]-2-fluoro-phenyl]piperidine-1-carboxylate (1.1 g, 2.71 mmol) in DCM was cooled to 0° C., then HCl in dioxane (1.98 g, 54.26 mmol, 2.47 mL) was added. The reaction mixture was stirred at 28° C. for 2 hours. Upon completion of the reaction, the mixture was concentrated under reduced pressure and the resulting crude material was washed with diethyl ether and pentane to give 3-[2-fluoro-3-(4-piperidyl)anilino]piperidine-2,6-dione HCl salt (1 g, 2.37 mmol, 87.35% yield). LC-MS (ES⁺): m/z 306.3 [M+H]⁺.

### Synthesis of 3-[4-(4-piperidyl)-3-(trifluoromethyl)anilino]piperidine-2,6-dione

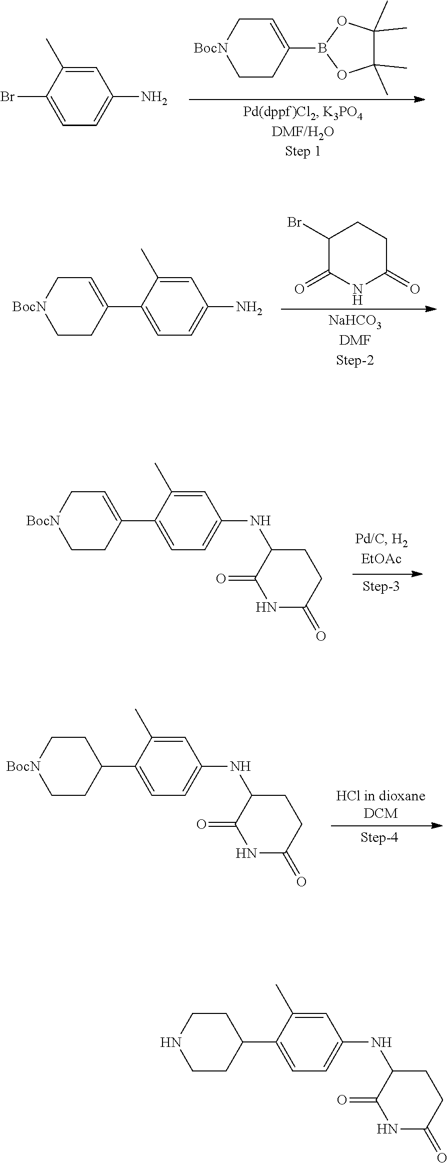

### Step-1

To a stirred solution of 4-bromo-3-methyl-aniline (1.3 g, 6.99 mmol) and tert-butyl 4-(4,4,5,5-tetramethyl-1,3,2-dioxaborolan-2-yl)-3,6-dihydro-2H-pyridine-1-carboxylate (2.16 g, 6.99 mmol) in DMF (20 mL) was added potassium phosphate tribasic anhydrous (3.71 g, 17.47 mmol) and the mixture was purged with nitrogen for 5 min. To the mixture was added cyclopentyl(diphenyl)phosphane; dichloromethane; dichloropalladium; iron (285.31 mg, 349.37 μmol) and stirred at 110° C. for 3 h. The progress of the reaction was monitored by TLC and UPLC. The reaction mixture was diluted with water, extracted with ethyl acetate, dried over sodium sulfate, and concentrated to get crude. The crude was purified by column chromatography (230-400 mesh silica, 30% ethyl acetate in pet ether) to get product tert-butyl 4-(4-amino-2-methyl-phenyl)-3,6-dihydro-2H-pyridine-1-carboxylate (1.5 g, 5.16 mmol, 73.84% yield) as a pale yellow solid. LC-MS (ES⁺): m/z 289.2 [M+H]⁺.

### Step-2

To a stirred solution of tert-butyl 4-(4-amino-2-methyl-phenyl)-3,6-dihydro-2H-pyridine-1-carboxylate (800 mg, 2.77 mmol) and 3-bromopiperidine-2,6-dione (2.66 g, 13.87 mmol) in DMF (15 mL) was added Sodium bicarbonate (1.17 g, 13.87 mmol, 539.45 μL) and the reaction mixture stirred at 80° C. for 16 h. The progress of the reaction was monitored by TLC and LC-MS. The reaction mixture was diluted with water, extracted with ethyl acetate, dried over sodium sulfate, and concentrated to get crude. The crude was purified by column chromatography (Silica 230-400, 50% ethyl acetate in pet ether) to get tert-butyl 4-[4-[(2,6-dioxo-3-piperidyl)amino]-2-methyl-phenyl]-3,6-dihydro-2H-pyridine-1-carboxylate (800 mg, 1.70 mmol, 61.14% yield) as pale green solid. LC-MS (ES⁺): m/z 400.3 [M+H]⁺.

### Step-3

To a stirred solution of tert-butyl 4-[4-[(2,6-dioxo-3-piperidyl)amino]-2-methyl-phenyl]-3,6-dihydro-2H-pyridine-1-carboxylate (500 mg, 1.25 mmol) in Ethyl Acetate (20 mL) was added Palladium, 10% on carbon, Type 487, dry (133.20 mg, 1.25 mmol) and stirred at room temperature for 16 h. The progress of the reaction was monitored by LC-MS. The reaction mixture was filtered through celite and concentrated to get crude product tert-butyl 4-[4-[(2,6-dioxo-3-piperidyl)amino]-2-methyl-phenyl]piperidine-1-carboxylate (400 mg, 898.34 μmol, 71.77% yield) as pale green solid. LC-MS (ES⁺): m/z 346.2 [M−(C(CH₃)₃+H]⁺.

### Step-4

To a stirred solution of tert-butyl 4-[4-[(2,6-dioxo-3-piperidyl)amino]-2-methyl-phenyl]piperidine-1-carboxylate (200 mg, 498.13 μmol) in DCM (5 mL) was added HCl in Dioxane (498.13 μmol, 5 mL) and the reaction mixture stirred at room temperature for 4 hr. The progress of the reaction was monitored by UPLC. The reaction mixture was concentrated to get crude product 3-[3-methyl-4-(4-piperidyl)anilino]piperidine-2,6-dione HCl salt (150 mg, 372.07 μmol, 74.69% yield) as off-white solid.

LC-MS (ES⁺): m/z 302.2 [M+H]⁺.

### Synthesis of 3-[4-(2-piperazin-1-ylethyl)anilino]piperidine-2,6-dione

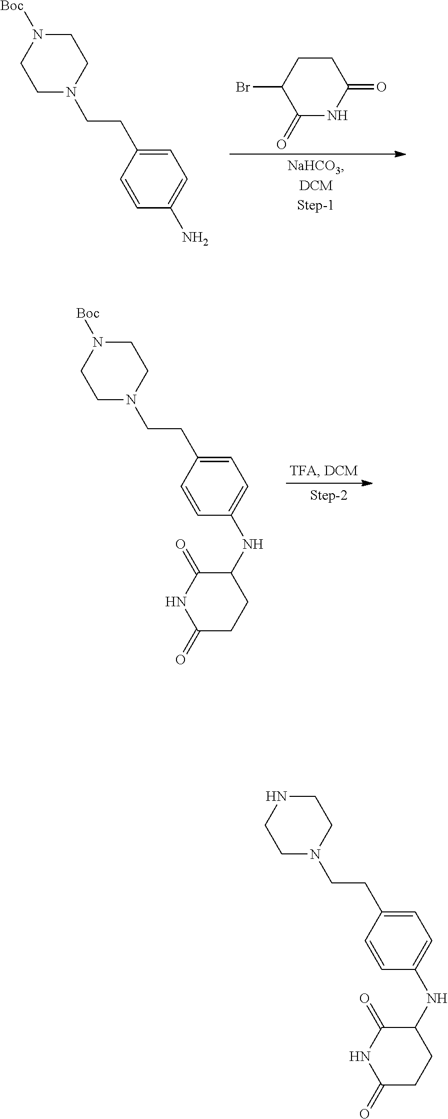 [SMILES: CC(C)(C)OC(=O)N1CCN(CCc2ccc(N)cc2)CC1 ; O=C1CCC(Br)C(=O)N1 ; CC(C)(C)OC(=O)N1CCN(CCc2ccc(NC3CCC(=O)NC3=O)cc2)CC1 ; O=C1CCC(Nc2ccc(CCN3CCNCC3)cc2)C(=O)N1]

### Step-1

To stirred a solution of tert-butyl 4-[2-(4-aminophenyl)ethyl]piperazine-1-carboxylate (1 g, 3.27 mmol) in DMF (10 mL) was added sodium bicarbonate (687.65 mg, 8.19 mmol, 318.35 μL) and the reaction mixture was stirred at 0° C. for 10 minutes. To the suspension was added 3-bromopiperidine-2,6-dione (943.03 mg, 4.91 mmol) and the reaction mixture stirred at 80° C. for 16 hrs. The reaction mixture was poured into water, extracted with ethyl acetate, layers separated, and the organic layer was concentrated under reduced pressure. The crude material was washed with diethyl ether and desiccated to give tert-butyl 4-[2-[4-[(2,6-dioxo-3-piperidyl)amino]phenyl]ethyl]piperazine-1-carboxylate (1 g, 1.70 mmol, 52.06% yield) as light yellow semi solid. Crude compound taken for next step without purification. LC-MS (ES⁻): m/z 415.3 [M−H]⁻.

### Step-2

To a stirred solution of tert-butyl 4-[2-[4-[(2,6-dioxo-3-piperidyl)amino]phenyl]ethyl]piperazine-1-carboxylate (150 mg, 360.13 μmol) in DCM (5 mL) was cooled to 0° C. then added trifluoroacetic acid (821.27 mg, 7.20 mmol, 554.91 μL) and the resultant mixture was stirred for 16 hr at 25° C. The reaction mixture was concentrated, triturated with diethyl ether, filtered and desiccated to afford 3-[4-(2-piperazin-1-ylethyl)anilino]piperidine-2,6-dione (110 mg, 253.79 μmol, 70.47% yield). LC-MS (ES⁺): m/z 317.5 [M+H]⁺.

### Synthesis of 3-(4-piperazin-1-ylphenyl)piperidine-2,6-dione

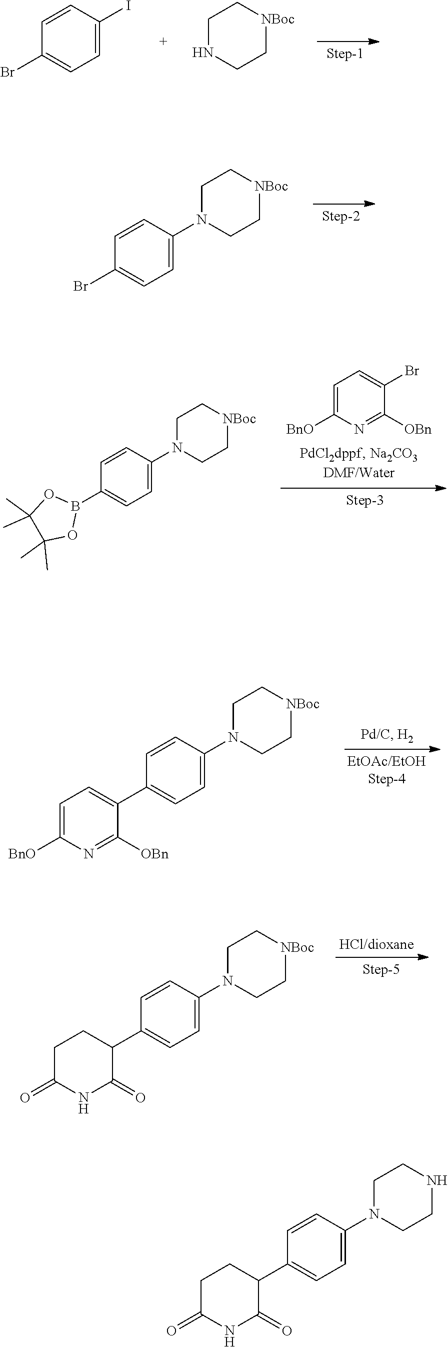

### Step-1

To a mixture of 1-bromo-4-iodo-benzene (1 g, 3.53 mmol) and tert-butyl piperazine-1-carboxylate (724.19 mg, 3.89 mmol) in toluene (30 ml) was added sodium; 2-methylpropan-2-olate (339.70 mg, 3.53 mmol) and the entire solution was degassed with nitrogen for 15 minutes followed by the addition of (1E,4E)-1,5-diphenylpenta-1,4-dien-3-one; palladium (3.24 g, 3.53 mmol) and [1-(2-diphenylphosphanyl-1-naphthyl)-2-naphthyl]-diphenyl-phosphane (2.20 g, 3.53 mmol). After addition, the reaction mixture was heated at 100° C. for 12 hr. The reaction mixture was diluted with ethyl acetate (60 mL) and was washed with water/brine and separated. After evaporation of the organic layer the residue was purified by column chromatography to afford tert-butyl 4-(4-bromophenyl)piperazine-1-carboxylate (460 mg, 1.19 mmol, 33.56% yield). LC-MS (ES⁺): m/z 341.1 [M+H]⁺.

### Step-2

To a stirred solution of tert-butyl 4-(4-bromophenyl)piperazine-1-carboxylate (20 g, 58.61 mmol) and 4,4,5,5-tetramethyl-2-(4,4,5,5-tetramethyl-1,3,2-dioxaborolan-2-yl)-1,3,2-dioxaborolane (29.77 g, 117.22 mmol) in dioxane (100 mL), potassium acetate (11.50 g, 117.22 mmol, 7.33 mL) was added. The reaction mixture was degassed with nitrogen for 15 minutes before cyclopentyl(diphenyl)phosphane; dichloromethane; dichloropalladium; iron (4.79 g, 5.86 mmol) was added. After addition, the reaction mixture was stirred at 100° C. for 12 hours in a sealed tube. Upon completion of the reaction, the reaction mixture was filtered through a celite bed which was washed with ethyl acetate several times. The combined organic layers were washed with water/brine and separated. It was then concentrated in vacuo to afford tert-butyl 4-[4-(4,4,5,5-tetramethyl-1,3,2-dioxaborolan-2-yl)phenyl]piperazine-1-carboxylate (18 g, 38.94 mmol, 66.44% yield) The resulting crude material was directly used for the next step. LC-MS (ES⁺): m/z 389.2 [M+H]⁺.

### Step-3

To a stirred solution of tert-butyl 4-[4-(4,4,5,5-tetramethyl-1,3,2-dioxaborolan-2-yl)phenyl]piperazine-1-carboxylate (12.3 g, 31.68 mmol) in DMF (80 mL) and water (10 mL) was added sodium carbonate (6.71 g, 63.35 mmol) and the resulting solution was degassed with argon for 15 minutes followed by the addition of 1,1′-bis(diphenylphosphino) ferrocene]dichloropalladium(II) (2.32 g, 3.17 mmol). The reaction mixture was then heated at 100° C. for 5 hours. After complete consumption of the starting material, the reaction mixture was filtered through a celite bed and ice cooled water was added to the filtrate. The aqueous layer was extracted with ethyl acetate. The combined organic layer was washed with water/brine and separated, dried over sodium sulfate, and concentrated under reduced pressure. The crude residue was purified by column chromatography eluting at 1% methanol in dichloromethane to afford tert-butyl 4-[4-(2,6-dibenzyloxy-3-pyridyl)phenyl]piperazine-1-carboxylate (11.5 g, 13.55 mmol, 42.78% yield) as an off white solid. LC-MS (ES⁺): m/z 552.7 [M+H]⁺.

### Step-4

A stirred solution of tert-butyl 4-[4-(2,6-dibenzyloxy-3-pyridyl)phenyl]piperazine-1-carboxylate (22 g, 39.88 mmol) in ethyl acetate (120 mL) and ethanol (120 mL) was degassed with argon for 20 minutes before 10% palladium on carbon (8.49 g, 7.98 mmol) was added and the reaction mixture was stirred under an atmosphere of hydrogen (balloon) at room temperature for 16 h. The reaction mixture was filtered through celite, concentrated under reduced pressure and purified by column chromatography eluting at 2% methanol in dichloromethane to afford tert-butyl 4-[4-(2,6-dioxo-3-piperidyl)phenyl]piperazine-1-carboxylate (13.2 g, 33.58 mmol, 84.20% yield) as an off white solid. LC-MS (ES⁺): m/z 374.2 [M+H]⁺.

### Step-5

To a stirred solution of tert-butyl 4-[4-(2,6-dioxo-3-piperidyl)phenyl]piperazine-1-carboxylate (13.1 g, 35.08 mmol) in DCM (50 mL) was added 4 M HCl in dioxane (35.08 mmol, 20 mL) at 0° C. and the reaction mixture stirred at room temperature for 1 h. Evaporation followed by lyophilization gave 3-(4-piperazin-1-ylphenyl)piperidine-2,6-dione hydrochloride (10.8 g, 34.39 mmol, 98.03% yield) as off white solid. LC-MS (ES⁺): m/z 274.4 [M+H]⁺.

### Synthesis of 3-(4-piperazin-1-ylphenoxy)piperidine-2,6-dione

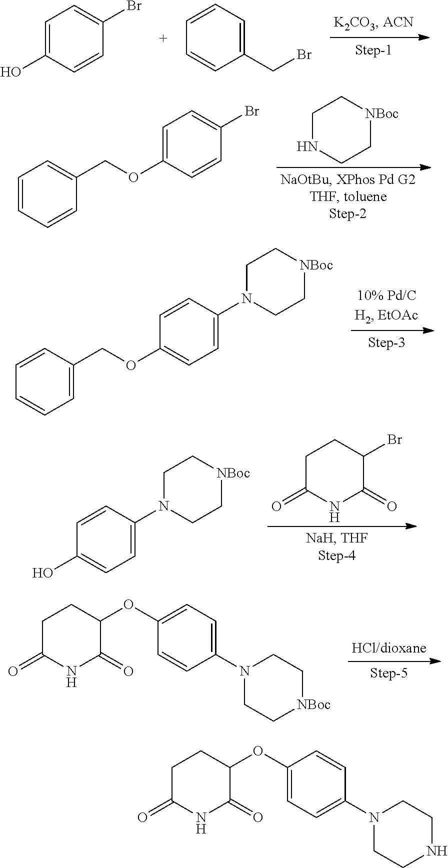

### Step-1

To a stirred solution of 4-bromophenol (8 g, 46.24 mmol) in ACN (100 mL) was added bromomethylbenzene (9.49 g, 55.49 mmol, 6.59 mL) and potassium carbonate, anhydrous powder 325 mesh (15.98 g, 115.60 mmol, 6.98 mL) at room temperature under a N₂ atmosphere and then stirred reaction mixture at room temperature for 16 hours. The reaction mixture was quenched with cold water and then extracted with EtOAc. The organic layer was washed with a brine solution and then dried over anhydrous Na₂SO₄, filtered and evaporated under reduced pressure. The obtained crude was purified by column chromatography using 100-200 silica gel, product eluted at 0-30% EtOAc in pet ether to give 1-benzyloxy-4-bromo-benzene (10 g, 36.86 mmol, 79.72% yield) as off-white solid. LC-MS (ES⁺): m/z 263.3 [M+H]⁺.

### Step-2

In a microwave vial, a stirred solution of sodium tert-butoxide (456.52 mg, 4.75 mmol) in mixture of THF (2.5 mL) and toluene (2.5 mL) was purged with argon for 5 minutes. To this solution was added 1-benzyloxy-4-bromo-benzene (0.5 g, 1.90 mmol) and tert-butyl piperazine-1-carboxylate (460.09 mg, 2.47 mmol) and purged with argon for 10 minutes. To this reaction mixture added tBuXPhos Pd G2 (149.51 mg, 190.02 μmol) and microwaved at 100° C. for 30 minutes. Evaporation followed by column chromatography (230-400 silica gel, 0 to 30% ethyl acetate and pet ether as an eluent) gave tert-butyl 4-(4-benzyloxyphenyl)piperazine-1-carboxylate (0.650 g, 1.61 mmol, 84.48% yield) as a yellow solid. LC-MS (ES⁺): m/z 369.2 [M+H]⁺.

### Step-3

To a stirred solution of tert-butyl 4-(4-benzyloxyphenyl)piperazine-1-carboxylate (0.65 g, 1.76 mmol) in ethyl acetate (10 mL) was added palladium, 10% on carbon, Type 487, dry (0.65 g, 6.11 mmol) and the reaction mixture was stirred overnight. The reaction mixture was filtered through celite using ethyl acetate (50 mL), the filtrate was concentrated under reduced pressure, and dried to obtain tert-butyl 4-[1-(2,6-dioxo-3-piperidyl)-3-methyl-2-oxo-benzimidazol-5-yl]-3,3-difluoro-piperidine-1-carboxylate (0.460 g, 922.91 μmol, 46.48% yield) as a colorless thick liquid. LC-MS (ES⁻): m/z 277.3 [M−H]⁻.

### Step-4

To a stirred solution of tert-butyl 4-(4-hydroxyphenyl)piperazine-1-carboxylate (0.4 g, 1.44 mmol) in THF (20 mL) was added sodium hydride (60% dispersion in mineral oil) (330.38 mg, 14.37 mmol) (NaH washed with hexane) under argon gas at room temperature. Then reaction mixture stirred at 70° C. for 30 minutes. After that, 3-bromopiperidine-2,6-dione (1.38 g, 7.19 mmol) in THF was added and refluxed at 70° C. for 4 hours under argon. The reaction mixture was poured on to ice-cold water and extracted with ethyl acetate. The organic layer was washed with brine solution, dried over Na₂SO₄, and concentrated at reduced pressure. The crude product was purified by silica gel column chromatography using 0-40% ethyl acetate in pet ether to afford tert-butyl 4-[4-[(2,6-dioxo-3-piperidyl)oxy]phenyl]piperazine-1-carboxylate (0.25 g, 618.96 μmol, 43.07% yield) as an off white solid. LC-MS (ES⁻): m/z 388.3 [M−H]⁻.

### Step-5

To cold hydrochloric acid in dioxane (808.84 μmol, 5.0 mL) was added tert-butyl 4-[4-[(2,6-dioxo-3-piperidyl)oxy]phenyl]piperazine-1-carboxylate (315.0 mg, 808.84 μmol) and the reaction mixture was stirred at room temperature for 2 hours. The solvent was evaporated under reduced pressure to obtain a white solid which was then washed with pentane and ether to give 3-(4-piperazin-1-ylphenoxy)piperidine-2,6-dione hydrochloride (265.0 mg, 793.07 μmol, 98.05% yield) as white solid in the form of HCl salt. LC-MS (ES⁺): m/z 290.1 [M+H]⁺. ¹H NMR (400 MHz, DMSO-d₆) δ 10.90 (s, 1H), 8.97 (br s, 2H), 6.94 (m, 4H), 5.06 (m, 1H), 3.23 (m, 8H), 2.66-2.61 (m, 2H), 2.15-2.10 (m, 2H);

### Synthesis of 3-[4-(4-piperidyloxy)anilino]piperidine-2,6-dione

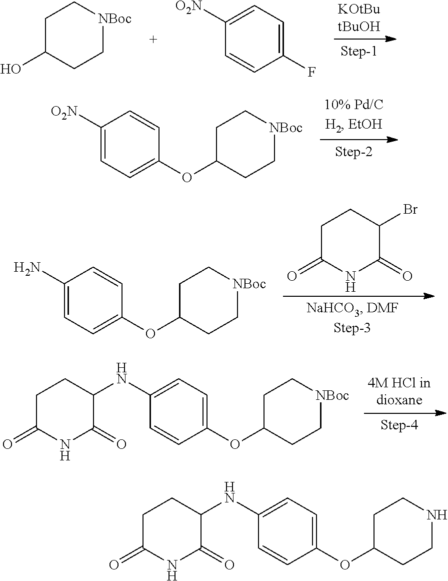

### Step-1

Into a 100 mL seal tube vessel containing a well-stirred solution of tert-butyl 4-hydroxypiperidine-1-carboxylate (2 g, 9.94 mmol) and 1-fluoro-4-nitro-benzene (1.40 g, 9.94 mmol, 1.05 mL) in tertiary butanol (30 mL) was added potassium tert-butoxide (1.23 g, 10.93 mmol) and stirred at 90° C. for 1 hour. After completion of the reaction, the reaction mixture was poured into ice cold water, the solid precipitate was filtered and dried to give tert-butyl 4-(4-nitrophenoxy)piperidine-1-carboxylate (2.5 g, 7.06 mmol, 71.02% yield). LC-MS (ES⁺): m/z 223.0 [M-COOtBu+H]⁺. ¹H NMR (300 MHz, CDCl₃): δ 8.22 (d, J=9.0 Hz, 2H), 6.97 (d, J=9.3 Hz, 2H), 4.65-4.60 (m, 1H), 3.76-3.68 (m, 2H), 3.44-3.36 (m, 2H), 2.03-1.95 (m, 2H), 1.86-1.70 (m, 2H), 1.43 (s, 9H) ppm.

### Step-2

To a solution of tert-butyl 4-(4-nitrophenoxy)piperidine-1-carboxylate (2.5 g, 7.76 mmol) in ethanol (50 mL) was added palladium, 10% on carbon, Type 487, dry (500 mg, 4.70 mmol) and the solution was degassed, then the reaction was stirred under an atmosphere of hydrogen (hydrogen bladder) at room temperature for 16 hours. After completion of the reaction, the catalyst was filtered through a celite bed and the obtained filtrate was evaporated under reduced pressure and dried to give tert-butyl 4-(4-aminophenoxy)piperidine-1-carboxylate (2 g), which was used without further purification. LC-MS (ES⁺): m/z 193.1 [M+H-Boc]⁺.

### Step-3

At room temperature 3-bromopiperidine-2,6-dione (3.94 g, 20.52 mmol), tert-butyl 4-(4-aminophenoxy)piperidine-1-carboxylate (5 g, 17.10 mmol), NaHCO₃ (5.93 g, 34.20 mmol) and DMF (50 mL) was added to a 150 mL sealed tube. The mixture purged with nitrogen, the stirred reaction sealed and heated at 40° C. overnight. The reaction mixture was diluted with water (100 ml) and extracted with ethyl acetate (3×100 ml). The combined organic layer was washed with brine (2×100 ml), dried over sodium sulfate, filtered, and evaporated under reduced pressure to give tert-butyl 4-[4-[(2,6-dioxo-3-piperidyl)amino]phenoxy]piperidine-1-carboxylate (8.2 g). Used without further purification. ¹H NMR (400 MHz, DMSO-d₆) δ 10.74 (s, 1H), 6.77 (d, J=8.9 Hz, 2H), 6.63 (d, J=8.8 Hz, 2H), 5.47 (d, J=7.3 Hz, 1H), 4.24 (ddt, J=35.6, 11.7, 5.6 Hz, 2H), 3.66-3.58 (m, 2H), 3.30 (s, 1H), 3.15 (t, J=10.6 Hz, 2H), 2.73 (ddd, J=17.3, 12.8, 5.4 Hz, 1H), 2.63-2.53 (m, 1H), 2.16-2.07 (m, 1H), 1.83 (dd, J=12.7, 7.0 Hz, 3H), 1.49 (ddd, J=12.8, 8.3, 3.9 Hz, 2H), 1.41 (s, 8H).

### Step-4

To an oven dried round bottom flask with a solution of tert-butyl 4-[4-[(2,6-dioxo-3-piperidyl)amino]phenoxy]piperidine-1-carboxylate (600 mg, 1.49 mmol) in 1,4-dioxane (3 mL) was added hydrogen chloride (4.0 M in dioxane) (10 mL). The reaction mixture was stirred for an hour at room temperature and the reaction mixture was concentrated under reduced pressure to yield 3-[4-(4-piperidyloxy)anilino]piperidine-2,6-dione (510 mg, 78.43% yield) as brown gummy solid, which was used without further purification. LC-MS (ES⁺): m/z 304.0 [M+H]⁺.

### Synthesis of 2-[4-[4-(2,6-dioxo-3-piperidyl)phenyl]-1-piperidyl]acetic acid

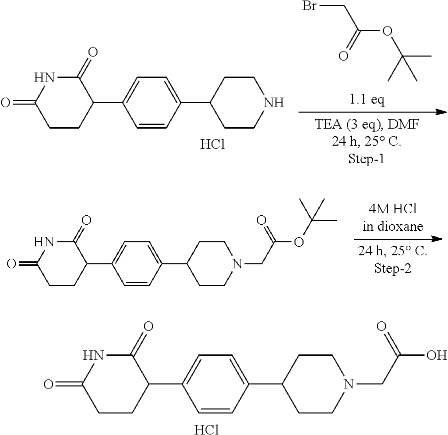

### Step-1

To a solution of 3-[4-(4-piperidyl)phenyl]piperidine-2,6-dione (2.5 g, 8.10 mmol, HCl salt) in DMF (30 mL) were added tert-butyl 2-bromoacetate (1.74 g, 8.91 mmol, 1.31 mL), and TEA (2.46 g, 24.29 mmol, 3.39 mL) under inert atmosphere at room temperature and stirred for 24 h. The reaction mixture was quenched with water (100 mL) and the solid was filtered. The solid was air dried to give tert-butyl 2-[4-[4-(2,6-dioxo-3-piperidyl)phenyl]-1-piperidyl]acetate (2.37 g, 5.83 mmol, 71.96% yield) as a white solid. LC-MS (ES⁺): m/z 387.2 [M+H]⁺.

### Step-2

tert-butyl 2-[4-[4-(2,6-dioxo-3-piperidyl)phenyl]-1-piperidyl]acetate (2.43 g, 6.29 mmol) was added to a solution of 4M HCl in dioxane (4 M in dioxane, 50.00 mL) under inert atmosphere at room temperature and was stirred for 24 h. The resulting mixture was evaporated to dryness and dried under vacuum to provide 2-[4-[4-(2,6-dioxo-3-piperidyl) phenyl]-1-piperidyl]acetic acid (2.15 g, 5.51 mmol, 87.62% yield, HCl salt). LC-MS (ES⁺): m/z 331.0 [M+H]⁺.

### Synthesis of 4-[(2,6-dioxo-3-piperidyl)oxy]benzoic acid

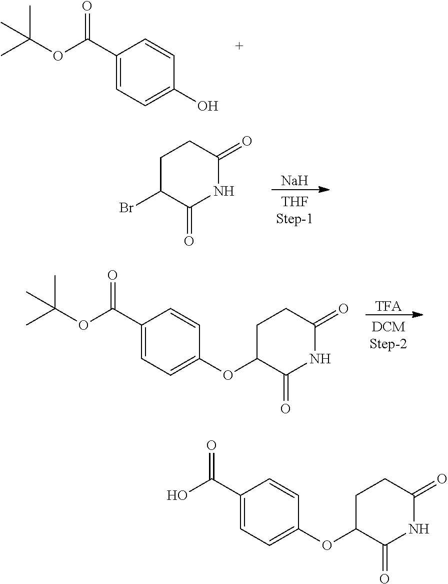

### Step-1

To a solution of tert-butyl 4-hydroxybenzoate (8.09 g, 41.66 mmol) in dry THF (150 mL) was added sodium hydride (4.17 g, 104.16 mmol) (60% dispersion in mineral oil) portionwise under nitrogen atmosphere at 0° C. The resulting reaction mixture was stirred for 30 minutes at room temperature. Then, 3-bromopiperidine-2,6-dione (10 g, 52.08 mmol) was added at 0° C., and the reaction was stirred at room temperature for 24 h. The reaction mixture was evaporated in vacuo and quenched with saturated ammonium chloride solution (50 mL) at 0° C. followed by extraction with ethyl acetate (3×150 mL). The combined organic layers were washed with ice-cooled water (2×10 mL), dried over anhydrous Na₂SO₄, filtered and concentrated. The residue was triturated with MTBE (2×30 mL) and filtered to provide tert-butyl 4-[(2,6-dioxo-3-piperidyl)oxy]benzoate (5.65 g, 18.50 mmol, 44.41% yield) as a grey crystals. LC-MS (ES⁻): m/z 304.0 [M−H]⁻.

### Step-2

To a solution of tert-butyl 4-[(2,6-dioxo-3-piperidyl)oxy]benzoate (2.1 g, 6.88 mmol) in DCM (40 mL) was added TFA (29.78 g, 261.18 mmol, 20 mL) at room temperature and stirred overnight. The resulting mixture was evaporated to dryness and triturated with MTBE (5 ml) and filtered to provide 4-[(2,6-dioxo-3-piperidyl)oxy]benzoic acid (1.6 g, 6.10 mmol, 88.68% yield) as a grey solid. LC-MS (ES⁻): m/z 248.2 [M−H]⁻.

### F. Synthesis of Representative Compounds

### Synthesis of tert-butyl 4-(2-bromoacetyl)piperidine-1-carboxylate

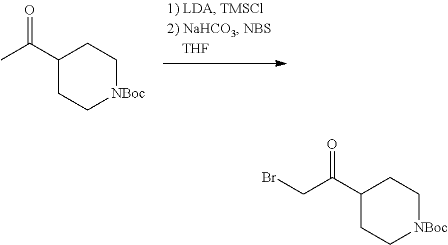 [SMILES: CC(=O)C1CCN(C(=O)OC(C)(C)C)CC1 ; CC(C)(C)OC(=O)N1CCC(C(=O)CBr)CC1]

To a solution of tert-butyl 4-acetylpiperidine-1-carboxylate (50 g, 219.97 mmol) in THF (500 mL), LDA (2 M, 131.98 mL) was added dropwise at −78° C. The solution was stirred at −78° C. for 1 hours before chlorotrimethylsilane (47.80 g, 439.95 mmol, 55.84 mL) was added at this temperature with. The reaction was stirred at −78° C. for an additional hour. After consumption of the reactant as shown by TLC, the reaction mixture was poured into aqueous sodium bicarbonate solution (200 mL) and the aqueous phase was extracted with ethyl acetate (80 mL×3). The combined organic layers were washed with brine (50 mL), dried with anhydrous Na₂SO₄, filtered, and concentrated in vacuo. The resulting residue was then dissolved in THF (500 mL), sodium bicarbonate (27.72 g, 329.96 mmol) and N-bromosuccinimide (58.73 g, 329.96 mmol) were added at 0° C. and the solution was stirred at 25° C. for 2 hours. After TLC showed complete conversion, the reaction mixture was poured into aqueous sodium bicarbonate solution (2 L) and the aqueous phase was extracted with ethyl acetate (500 mL×3). The combined organic layers were washed with brine (500 mL), dried with anhydrous Na₂SO₄, filtered, and concentrated in vacuo. The crude product was purified by column chromatography (silica gel, petroleum ether/ethyl acetate=20/1 to 5/1) to give tert-butyl 4-(2-bromoacetyl)piperidine-1-carboxylate (28 g, 64.31 mmol, 29.24% yield) as a yellow oil. LC-MS (ES⁺): m/z 249.9 [M-tBu+H]⁺.

### Synthesis of 5-bromo-4-isopropoxy-pyridin-2-amine

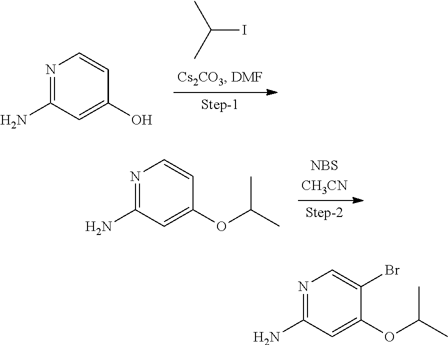

### Step-1

To a suspension of 2-aminopyridin-4-ol (40 g, 363.26 mmol) in DMF (500 mL) was added cesium carbonate (118.36 g, 363.26 mmol) and 2-iodopropane (61.75 g, 363.26 mmol, 36.32 mL). The mixture was stirred at 120° C. for 16 hours. After consumption of the reactant as demonstrated by TLC, the reaction mixture was diluted with water (500 mL) and extracted with ethyl acetate (300 mL×4). The combined organic layers were washed with brine 300 (150 mL×2), dried over Na₂SO₄, filtered, and concentrated under reduced pressure to furnish the product 4-isopropoxypyridin-2-amine (29 g, 183.73 mmol, 50.58% yield) as a yellow solid. LC-MS (ES⁺): m/z 153.1 [M+H]⁺.

### Step-2

To a solution of 4-isopropoxypyridin-2-amine (29 g, 190.55 mmol) in acetonitrile (300 mL) was added 1-bromopyrrolidine-2,5-dione (30.52 g, 171.49 mmol). The mixture was stirred at 25° C. for 2 hours. After consumption of the reactant as shown by TLC, the reaction mixture was diluted with water (500 mL) and extracted with ethyl acetate (200 mL×5). The combined organic layers were washed with brine (150 mL×2), dried over Na₂SO₄, filtered, and concentrated under reduced pressure. The crude product was purified by flash column chromatography (250 g Silica, 10-50% ethyl acetate in pet ether as eluent at 100 mL/min). Compound 5-bromo-4-isopropoxy-pyridin-2-amine (42 g, 181.75 mmol, 95.38% yield) was obtained as an orange solid. LC-MS (ES⁺): m/z 230.9 [M+H]⁺.

### Synthesis of N-[7-isopropoxy-2-(4-piperidyl)imidazo[1,2-a]pyridin-6-yl]-6-(trifluoromethyl)pyridine-2-carboxamide

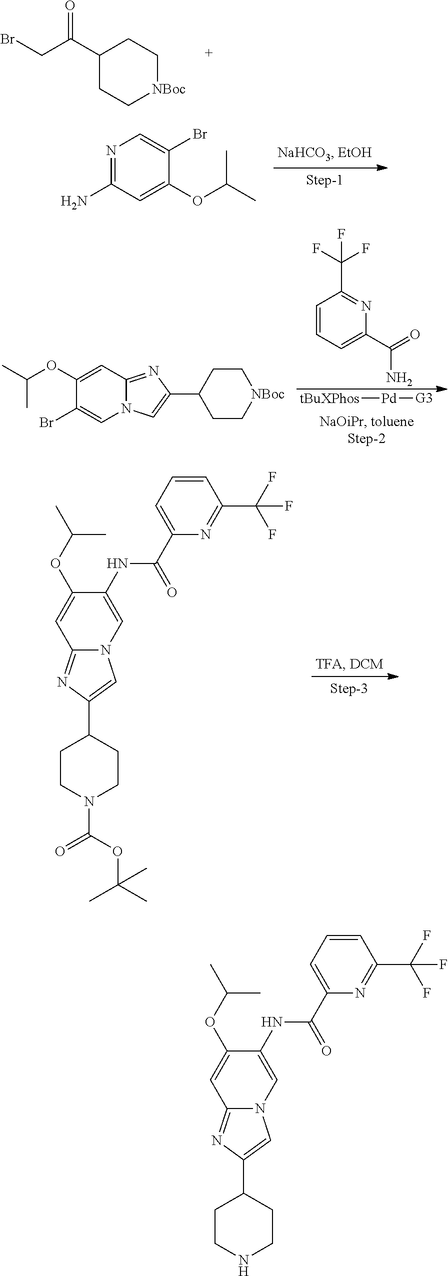

### Step-1

A solution of 5-bromo-4-isopropoxy-pyridin-2-amine (2.5 g, 10.82 mmol) and tert-butyl 4-(2-bromoacetyl)piperidine-1-carboxylate (4.97 g, 16.23 mmol) in ethanol (30 mL) was stirred for 16 hours at 90° C. The solvent was concentrated under reduced pressure and the material was purified by reverse phase column chromatography (0-40%, Acetonitrile in 0.1% formic acid in water) to afford tert-butyl 4-(6-bromo-7-isopropoxy-imidazo[1,2-a]pyridin-2-yl)piperidine-1-carboxylate (3.4 g, 6.52 mmol, 60.22% yield) as brown solid. LC-MS (ES⁺): m/z 438.8 [M+H]⁺

### Step-2

To a stirred solution of tert-butyl 4-(6-bromo-7-isopropoxy-imidazo[1,2-a]pyridin-2-yl)piperidine-1-carboxylate (5.0 g, 11.41 mmol) in toluene (50 mL) was added 6-(trifluoromethyl)pyridine-2-carboxamide (3.25 g, 17.11 mmol). The reaction mixture was purged with argon gas for 10 min. To the reaction mixture was added sodium; 2-methylpropan-2-olate (1.53 g, 15.97 mmol) and purged with argon gas for 10 minutes. Then, tBuXPhos Pd G3 (906.08 mg, 1.14 mmol) was added to the reaction mixture and purged with argon gas for another 10 minutes. The reaction mixture was stirred for 32 h at 100° C. After completion of the reaction, the reaction mixture was diluted with water (500 mL) and extracted with EtOAc (2×500 mL). The organic layer dried over anhydrous sodium Na₂SO₄ and concentrated under reduced pressure. The crude product was purified by reverse phase column chromatography to yield tert-butyl 4-[7-isopropoxy-6-[[6-(trifluoromethyl)pyridine-2-carbonyl]amino]imidazo[1,2-a]pyridin-2-yl]piperidine-1-carboxylate (3.9 g, 6.62 mmol, 58.07% yield) as an yellow solid. LC-MS (ES⁺): m/z 548.32 [M+H]⁺

### Step-3

To a stirred solution of tert-butyl 4-[7-isopropoxy-6-[[6-(trifluoromethyl)pyridine-2-carbonyl]amino]imidazo[1,2-a]pyridin-2-yl]piperidine-1-carboxylate (5.0 g, 9.13 mmol) in DCM (50 mL) was added trifluoroacetic acid (10.41 g, 91.31 mmol, 7.03 mL) dropwise at 0° C. and the reaction mixture was stirred for 16 h. After completion of reaction, the reaction mixture was concentrated under reduced pressure and trituration with diethyl ether gave N-[7-isopropoxy-2-(4-piperidyl)imidazo[1,2-a]pyridin-6-yl]-6-(trifluoromethyl)pyridine-2-carboxamide trifluoroacetic acid salt (6.15 g, 8.56 mmol, 93.71% yield) as an off white solid. LC-MS (ES⁺): m/z 448.33 [M+H]⁺

### Synthesis of N-[2-(azetidin-3-yl)-7-isopropoxy-imidazo[1,2-a]pyridin-6-yl]-6-(trifluoromethyl)pyridine-2-carboxamide

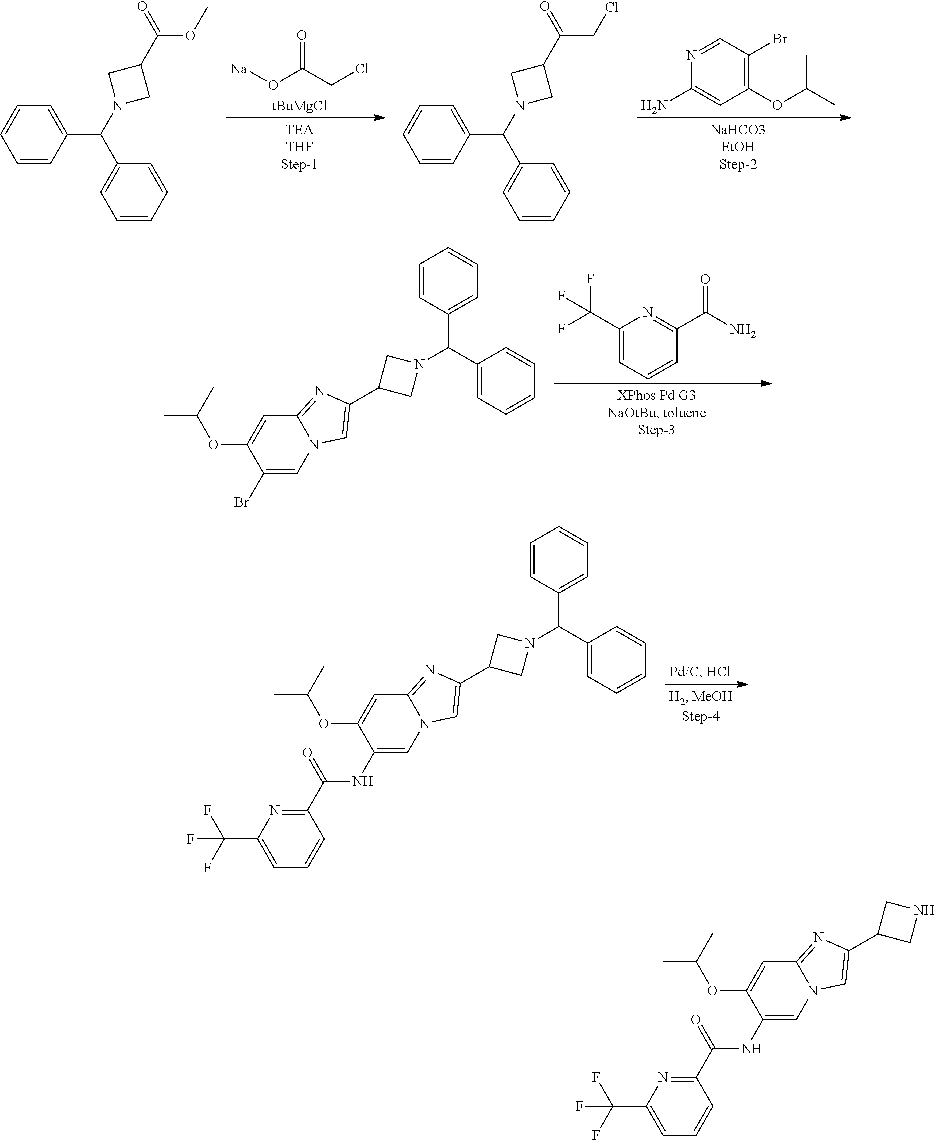

### Step-1

To a stirred solution of methyl 1-benzhydrylazetidine-3-carboxylate (40 g, 142.17 mmol) in THF was added sodium 2-chloroacetate (24.84 g, 213.26 mmol) and TEA (21.58 g, 213.26 mmol, 29.72 mL) at 0° C. and the resulting reaction mixture was stirred at room temperature for 30 minutes. Then tertbutyl magnesium chloride (33.23 g, 284.35 mmol) was added at −5° C., the cooling bath removed and stirred for 1 hr. The reaction mixture was quenched with ammonium chloride, extracted with ethyl acetate, and concentrated to give 1-(1-benzhydrylazetidin-3-yl)-2-chloro-ethanone (38 g, 50.70 mmol, 35.66% yield) as off-white solid. LC-MS (ES⁺): m/z 300.10 [M+H]⁺

### Step-2

To a stirred solution of 1-(1-benzhydrylazetidin-3-yl)-2-chloro-ethanone (25 g, 83.39 mmol) and 5-bromo-4-isopropoxypyridin-2-amine (9.64 g, 41.70 mmol) in ethanol at 0° C. was added sodium bicarbonate (4.20 g, 50.03 mmol, 1.95 mL) and the reaction mixture was stirred at 90° C. for 16 h. The reaction mixture was concentrated and purified by prep HPLC to give 2-(1-benzhydrylazetidin-3-yl)-6-bromo-7-isopropoxy-imidazo[1,2-a]pyridine (7 g, 11.75 mmol, 28.19% yield). LC-MS (ES⁺): m/z 476.21 [M+H]⁺

### Step-3

A stirred solution of 2-(1-benzhydrylazetidin-3-yl)-6-bromo-7-isopropoxy-imidazo[1,2-a]pyridine (6.0 g, 12.59 mmol), 6-(trifluoromethyl)picolinamide (3.59 g, 18.89 mmol) and sodium tert-butoxide (1.69 g, 17.63 mmol) in toluene was degassed for 30 minutes. To this mixture was added XPhos Pd G3 (1.01 mmol) and again degassed for 20 minutes followed by stirring the reaction mixture 100° C. for 16 h. After consumption of the starting material, a standard workup followed by reverse phase purification gave N-[2-(1-benzhydrylazetidin-3-yl)-7-isopropoxy-imidazo[1,2-a]pyridin-6-yl]-6-(trifluoromethyl) pyridine-2-carboxamide (2 g, 2.83 mmol, 22.51% yield). LC-MS (ES⁺): m/z 586.88 [M+H]⁺

### Step-4

To a stirred solution of N-[2-(1-benzhydrylazetidin-3-yl)-7-isopropoxy-imidazo[1,2-a]pyridin-6-yl]-6-(trifluoromethyl)pyridine-2-carboxamide (1.5 g, 2.56 mmol) in methanol was added palladium hydroxide on carbon, 20 wt. % 50% water (359.72 mg, 2.56 mmol) followed by hydrochloric acid, 36% w/w aq. soln. (93.39 mg, 2.56 mmol, 116.74 p L) and the resulting reaction mixture was stirred at room temperature for 24 hours under hydrogen balloon pressure. The reaction mixture was filtered through a celite bed and concentrated to give N-[2-(azetidin-3-yl)-7-isopropoxy-imidazo[1,2-a]pyridin-6-yl]-6-(trifluoromethyl) pyridine-2-carboxamide (0.9 g, 1.87 mmol, 72.89% yield). LC-MS (ES⁺): m/z 420.32 [M+H]⁺.

### Example 1

### Synthesis of N-[2-[1-[2-[4-[4-[(2,6-dioxo-3-piperidyl)oxy]phenyl]-1-piperidyl]acetyl]-4-piperidyl]-7-isopropoxy-imidazo[1,2-a]pyridin-6-yl]-6-(trifluoromethyl) pyridine-2-carboxamide

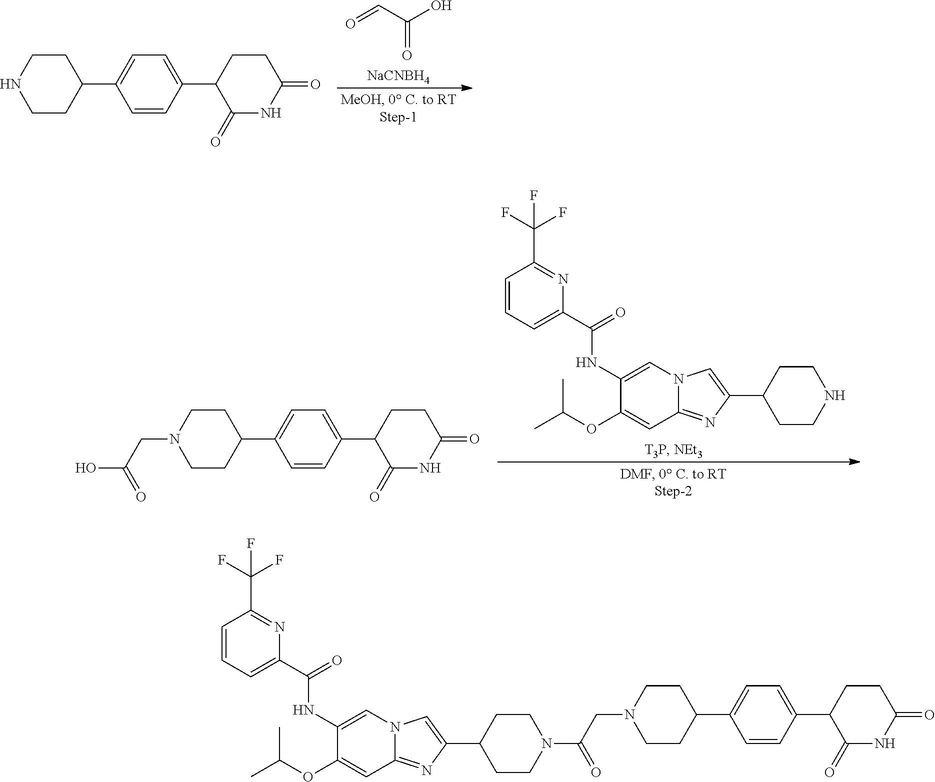

### Step-1

A stirred solution of glyoxylic acid, 50% w/w aq. soln (0.031 g, 418.72 μmol, 23.13 μL) and 3-[4-(4-piperidyl)phenoxy]piperidine-2,6-dione (100.61 mg, 348.93 μmol) in methanol was stirred for 10 min at 0° C. Then sodium cyanoborohydride (43.85 mg, 697.86 μmol) was added at 0° C. and the resulting reaction mixture was stirred at room temperature for 16 hr.

After consumption of the starting material, the reaction mixture was concentrated to give 2-[4-[4-[(2,6-dioxo-3-piperidyl)oxy]phenyl]-1-piperidyl]acetic acid (0.09 g, 148.10 μmol, 42.45% yield), which was used without further purification. LC-MS (ES⁺): m/z 345.41 [M−H]⁻

### Step-2

To a stirred solution of N-[7-isopropoxy-2-(4-piperidyl)imidazo[1,2-a]pyridin-6-yl]-6-(trifluoromethyl)pyridine-2-carboxamide (0.05 g, 111.74 μmol) and 2-[4-[4-[(2,6-dioxo-3-piperidyl)oxy]phenyl]-1-piperidyl]acetic acid (38.71 mg, 111.74 μmol) in THF was added TEA (11.31 mg, 111.74 μmol, 15.57 μL) and T3P (53.30 mg, 167.62 μmol) at 0° C. and resulting reaction mixture was stirred at room temperature for 16 hr. After consumption of the starting material, the reaction mixture was concentrated and purification by prep HPLC gave N-[2-[1-[2-[4-[4-[(2,6-dioxo-3-piperidyl)oxy]phenyl]-1-piperidyl]acetyl]-4-piperidyl]-7-isopropoxy-imidazo[1,2-a]pyridin-6-yl]-6-(trifluoromethyl)pyridine-2-carboxamide (37 mg, 46.60 μmol, 41.70% yield). LC-MS (ES⁺): m/z 776.59 [M+H]⁺. ¹H NMR (400 MHz, DMSO-d₆) δ 10.90 (s, 1H), 10.45 (s, 1H), 9.44 (s, 1H), 8.43 (q, J=8.1 Hz, 2H), 8.24 (d, J=7.3 Hz, 1H), 7.70 (s, 1H), 7.14 (d, J=10.3 Hz, 3H), 6.92 (d, J=8.5 Hz, 2H), 5.13 (q, J=5.3 Hz, 1H), 4.88 (m, 1H), 4.38 (d, J=12.8 Hz, 1H), 4.18 (d, J=12.9 Hz, 1H), 3.14 (m, 1H), 2.92 (m, 3H), 2.75 (d, J=10.9 Hz, 2H), 2.64 (m, 1H), 2.38 (t, J=21.6 Hz, 2H), 2.07 (m, 6H), 1.74 (m, 6H), 1.63 (m, 6H), 1.40 (d, J=5.9 Hz, 1H).

TLC R_{f}: 0.1 (10% methanol in DCM)

### Prep HPLC Condition:

Column/dimensions: X-Bridge C18 (19*250*5 um)
Mobile phase A: 5 mM AA in water (aq.) Mobile phase B: 100% Acetonitrile
Gradient (Time/% B): 0/10, 2/10, 2.5/25, 22/63, 22.1/95, 24/95, 24/95, 24.1/10, 26.5/10
Flow rate: 17 ml/min solubility: CAN+THF+DMSO

### Example 2

Compound of Example 2 was prepared substantially following the synthesis of Example 1

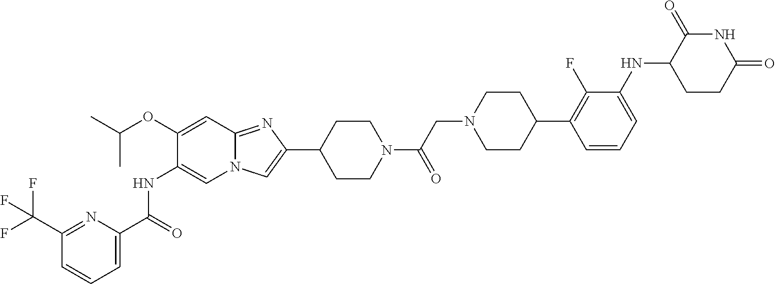 [SMILES: CC(C)Oc1cc2nc(C3CCN(C(=O)CN4CCC(c5cccc(NC6CCC(=O)NC6=O)c5F)CC4)CC3)cn2cc1NC(=O)c1cccc(C(F)(F)F)n1]

N-[2-[1-[2-[4-[3-[(2,6-dioxo-3-piperidyl)amino]-2-fluoro-phenyl]-1-piperidyl]acetyl]-4-piperidyl]-7-isopropoxy-imidazo[1,2-a]pyridin-6-yl]-6-(trifluoromethyl)pyridine-2-carboxamide. LC-MS (ES⁺): m/z 776.59 [M+H]⁺. ¹H NMR (400 MHz, DMSO-d₆) δ 10.82 (s, 1H), 10.45 (s, 1H), 9.45 (s, 1H), 8.43 (q, J=8.0 Hz, 1H), 8.25 (d, J=7.0 Hz, 1H), 8.14 (s, 1H), 7.70 (s, 1H), 7.13 (s, 1H), 6.90 (t, J=7.8 Hz, 1H), 6.68 (t, J=8.1 Hz, 1H), 6.52 (t, J=6.5 Hz, 1H), 5.51 (d, J=6.2 Hz, 1H), 4.88 (m, 1H), 4.38 (t, J=12.3 Hz, 2H), 4.06 (s, 1H), 3.16 (m, 5H), 2.83 (m, 4H), 2.03 (m, 2H), 1.71 (m, 5H), 1.52 (m, 5H), 1.40 (m, 6H), 1.25 (t, J=5.4 Hz, 1H).

### Example 3

Compound of Example 3 was prepared substantially following the synthesis of Example 1

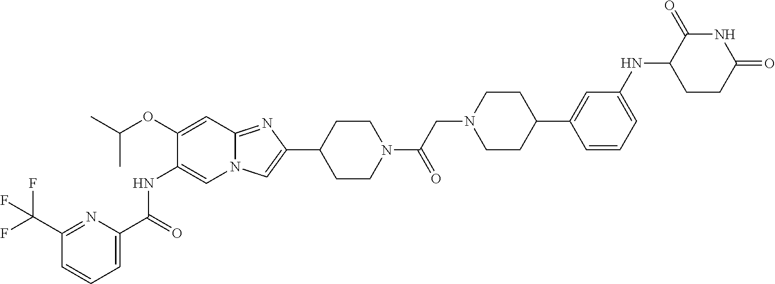 [SMILES: CC(C)Oc1cc2nc(C3CCN(C(=O)CN4CCC(c5cccc(NC6CCC(=O)NC6=O)c5)CC4)CC3)cn2cc1NC(=O)c1cccc(C(F)(F)F)n1]

N-[2-[1-[2-[4-[3-[(2,6-dioxo-3-piperidyl)amino]phenyl]-1-piperidyl]acetyl]-4-piperidyl]-7-isopropoxy-imidazo[1,2-a]pyridin-6-yl]-6-(trifluoromethyl)pyridine-2-carboxamide. LC-MS (ES⁺): m/z 775.12 [M+H]⁺. ¹H NMR (400 MHz, DMSO-d₆) δ 10.75 (s, 1H), 10.44 (s, 1H), 9.44 (s, 1H), 8.43 (q, J=8.1 Hz, 2H), 8.24 (d, J=7.1 Hz, 1H), 8.17 (s, 1H), 7.70 (s, 1H), 7.13 (s, 1H), 6.98 (t, J=7.7 Hz, 1H), 6.56 (s, 1H), 6.47 (q, J=7.1 Hz, 2H), 5.74 (d, J=7.5 Hz, 1H), 4.88-4.34 (m, 2H), 4.17 (d, J=12.8 Hz, 1H), 3.32 (s, 3H), 2.93 (s, 3H), 2.73 (m, 2H), 2.50 (s, 1H), 2.35 (t, J=9.9 Hz, 1H), 2.00-1.86 (m, 6H), 1.67 (m, 5H), 1.40 (m, 7H).

### Example 4

Compound of Example 4 was prepared substantially following the synthesis of Example 1

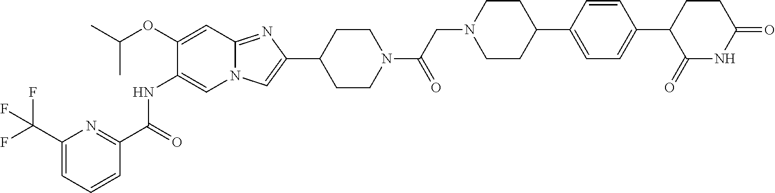 [SMILES: CC(C)Oc1cc2nc(C3CCN(C(=O)CN4CCC(c5ccc(C6CCC(=O)NC6=O)cc5)CC4)CC3)cn2cc1NC(=O)c1cccc(C(F)(F)F)n1]

N-[2-[1-[2-[4-[4-(2,6-dioxo-3-piperidyl)phenyl]-1-piperidyl]acetyl]-4-piperidyl]-7-isopropoxy-imidazo[1,2-a]pyridin-6-yl]-6-(trifluoromethyl)pyridine-2-carboxamide. LC-MS (ES⁺): m/z 760.09 [M+H]⁺. ¹H NMR (400 MHz, DMSO-d₆) δ 10.80 (s, 1H), 10.45 (s, 1H), 9.45 (s, 1H), 8.43 (m, 5H), 8.24 (d, J=7.0 Hz, 1H), 7.70 (s, 1H), 7.34-7.12 (m, 5H), 4.88 (t, J=5.9 Hz, 1H), 4.39 (d, J=14.2 Hz, 1H), 4.18 (d, J=12.7 Hz, 1H), 3.80 (q, J=5.4 Hz, 1H), 3.10-2.67 (m, 11H), 2.06 (q, J=19.3 Hz, 6H), 1.70 (d, J=40.3 Hz, 2H), 1.40 (d, J=5.9 Hz, 6H).

### Example 5

Compound of Example 5 was prepared substantially following the synthesis of Example 1

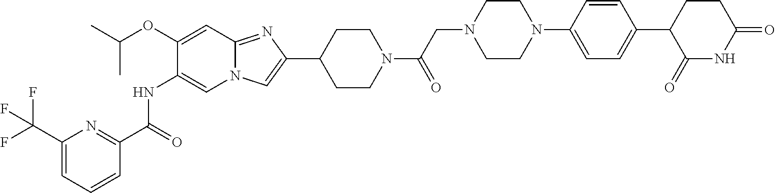 [SMILES: CC(C)Oc1cc2nc(C3CCN(C(=O)CN4CCN(c5ccc(C6CCC(=O)NC6=O)cc5)CC4)CC3)cn2cc1NC(=O)c1cccc(C(F)(F)F)n1]

N-[2-[1-[2-[4-[4-(2,6-dioxo-3-piperidyl)phenyl]piperazin-1-yl]acetyl]-4-piperidyl]-7-isopropoxy-imidazo[1,2-a]pyridin-6-yl]-6-(trifluoromethyl)pyridine-2-carboxamide. LC-MS (ES⁺): m/z 761.55 [M+H]⁺. ¹H NMR (400 MHz, DMSO-d₆) δ 10.76 (s, 1H), 10.44 (s, 1H), 9.44 (s, 1H), 8.45 (t, J=14.1 Hz, 1H), 8.39 (m, 2H), 8.24 (d, J=7.1 Hz, 1H), 7.69 (s, 1H), 7.34 (q, J=8.0 Hz, 1H), 7.08 (t, J=17.8 Hz, 1H), 6.90 (d, J=8.4 Hz, 1H), 6.71 (s, 1H), 4.88 (t, J=6.0 Hz, 1H), 4.43 (t, J=21.5 Hz, 1H), 4.15 (d, J=13.9 Hz, 1H), 3.72 (q, J=5.3 Hz, 1H), 3.58 (q, J=19.3 Hz, 1H), 3.15 (m, 8H), 2.90 (s, 1H), 2.76 (s, 1H), 2.57 (m, 5H), 2.07 (m, 8H), 1.40 (d, J=5.9 Hz, 3H).

### Example 6

Compound of Example 6 was prepared substantially following the synthesis of Example 1

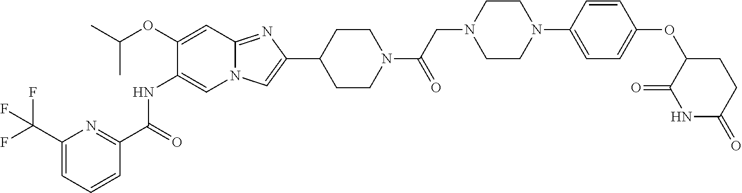 [SMILES: CC(C)Oc1cc2nc(C3CCN(C(=O)CN4CCN(c5ccc(OC6CCC(=O)NC6=O)cc5)CC4)CC3)cn2cc1NC(=O)c1cccc(C(F)(F)F)n1]

N-[2-[1-[2-[4-[4-[(2,6-dioxo-3-piperidyl)oxy]phenyl]piperazin-1-yl]acetyl]-4-piperidyl]-7-isopropoxy-imidazo[1,2-a]pyridin-6-yl]-6-(trifluoromethyl)pyridine-2-carboxamide. LC-MS (ES⁺): m/z 777.69 [M+H]⁺. ¹H NMR (400 MHz, DMSO-d₆) δ 10.87 (s, 1H), 10.44 (s, 1H), 9.44 (s, 1H), 8.44 (t, J=8.0 Hz, 2H), 8.24 (d, J=7.1 Hz, 1H), 7.69 (s, 1H), 7.13 (s, 1H), 6.89 (m, 4H), 5.02 (m, 1H), 4.94 (m, 1H), 4.40 (d, J=13.1 Hz, 1H), 4.15 (d, J=12.2 Hz, 1H), 3.11 (m, 5H), 2.91 (s, 1H), 2.67 (m, 6H), 2.08 (m, 4H), 1.43 (m, 5H), 0.94 (m, 6H).

### Example 7

Compound of Example 7 was prepared substantially following the synthesis of Example 1

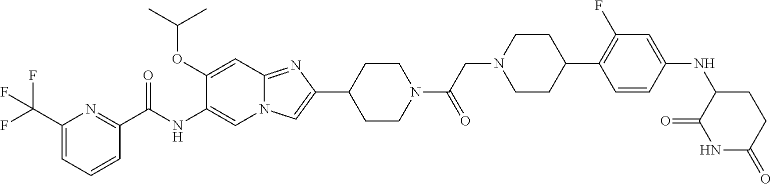 [SMILES: CC(C)Oc1cc2nc(C3CCN(C(=O)CN4CCC(c5ccc(NC6CCC(=O)NC6=O)cc5F)CC4)CC3)cn2cc1NC(=O)c1cccc(C(F)(F)F)n1]

N-[2-[1-[2-[4-[4-[(2,6-dioxo-3-piperidyl)amino]-2-fluoro-phenyl]-1-piperidyl]acetyl]-4-piperidyl]-7-isopropoxy-imidazo[1,2-a]pyridin-6-yl]-6-(trifluoromethyl)pyridine-2-carboxamide. LC-MS (ES⁺): m/z 793.23 [M+H]⁺. ¹H NMR (400 MHz, DMSO-d₆) δ 10.80 (s, 1H), 10.54 (s, 1H), 9.63 (d, J=100.7 Hz, 1H), 8.47 (t, J=7.8 Hz, 2H), 8.30 (d, J=7.1 Hz, 1H), 8.10 (s, 1H), 7.37 (s, 1H), 7.21-6.95 (m, 3H), 6.49 (t, J=9.9 Hz, 3H), 6.12 (d, J=6.5 Hz, 1H), 5.09 (s, 1H), 4.40 (m, 4H), 3.77-3.34 (m, 3H), 3.16 (m, 4H), 2.93 (t, J=11.8 Hz, 2H), 2.74 (m, 1H), 2.60 (m, 1H), 2.09 (m, 5H), 1.89 (d, J=14.3 Hz, 2H), 1.46 (d, J=5.8 Hz, 6H).

### Example 8

Compound of Example 8 was prepared substantially following the synthesis of Example 1

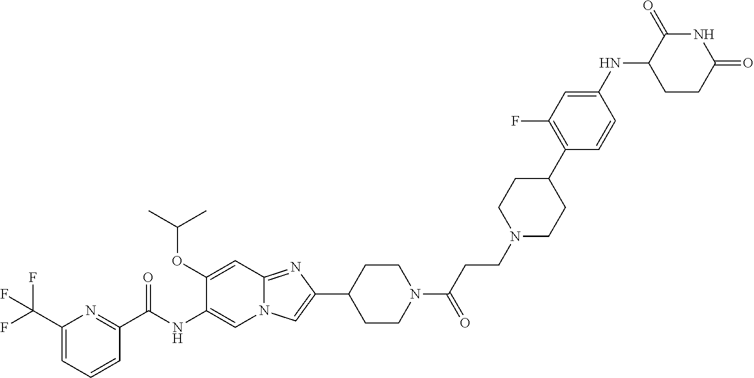 [SMILES: CC(C)Oc1cc2nc(C3CCN(C(=O)CCN4CCC(c5ccc(NC6CCC(=O)NC6=O)cc5F)CC4)CC3)cn2cc1NC(=O)c1cccc(C(F)(F)F)n1]

N-[2-[1-[3-[4-[4-[(2,6-dioxo-3-piperidyl)amino]-2-fluoro-phenyl]-1-piperidyl]propanoyl]-4-piperidyl]-7-isopropoxy-imidazo[1,2-a]pyridin-6-yl]-6-(trifluoromethyl)pyridine-2-carboxamide. LC-MS (ES⁺): m/z 807.42 [M+H]⁺. ¹H NMR (400 MHz, DMSO-d₆) δ 10.78 (s, 1H), 10.45 (s, 1H), 9.44 (s, 1H), 8.40 (m, 3H), 8.24 (d, J=7.2 Hz, 1H), 7.69 (s, 1H), 7.12 (s, 1H), 6.97 (t, J=8.7 Hz, 1H), 6.43 (t, J=6.9 Hz, 2H), 5.98 (d, J=7.7 Hz, 1H), 4.88 (m, J=5.9 Hz, 1H), 4.41 (d, J=12.7 Hz, 1H), 4.29 (m, J=6.0 Hz, 1H), 3.96 (m, 4H), 3.18 (m, 3H), 2.93 (m, 2H), 2.71 (m, 5H), 2.50 (m, 4H), 1.96 (m, J=9.8 Hz, 1H), 1.51 (m, 11H).

### Example 9

Compound of Example 9 was prepared substantially following the synthesis of Example 1

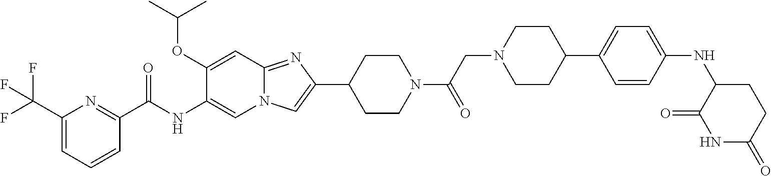 [SMILES: CC(C)Oc1cc2nc(C3CCN(C(=O)CN4CCC(c5ccc(NC6CCC(=O)NC6=O)cc5)CC4)CC3)cn2cc1NC(=O)c1cccc(C(F)(F)F)n1]

N-[2-[1-[2-[4-[4-[(2,6-dioxo-3-piperidyl)amino]phenyl]-1-piperidyl]acetyl]-4-piperidyl]-7-isopropoxy-imidazo[1,2-a]pyridin-6-yl]-6-(trifluoromethyl)pyridine-2-carboxamide. LC-MS (ES⁺): m/z 775.48 [M+H]⁺. ¹H NMR (400 MHz, DMSO-d₆) δ 10.76 (s, 1H), 10.45 (s, 1H), 9.44 (s, 1H), 8.43 (q, 2H), 8.26 (t, 2H), 7.70 (s, 1H), 7.13 (s, 1H), 6.95 (d, J=8.4 Hz, 2H), 6.60 (d, J=8.4 Hz, 2H), 5.64 (d, J=7.5 Hz, 1H), 4.88 (m, 1H), 4.38 (d, J=12.8 Hz, 1H), 4.22 (m, J=7.5 Hz, 2H), 3.17 (m, 5H), 2.91 (s, 3H), 2.73 (m, 2H), 2.56 (t, 3H), 2.32 (m, 1H), 2.04 (m, 4H), 1.85-1.4 (m, 6H).

### Example 10

Compound of Example 10 was prepared substantially following the synthesis of Example 1

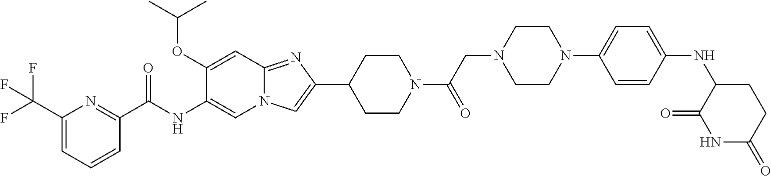 [SMILES: CC(C)Oc1cc2nc(C3CCN(C(=O)CN4CCN(c5ccc(NC6CCC(=O)NC6=O)cc5)CC4)CC3)cn2cc1NC(=O)c1cccc(C(F)(F)F)n1]

N-[2-[1-[2-[4-[4-[(2,6-dioxo-3-piperidyl)amino]phenyl]piperazin-1-yl]acetyl]-4-piperidyl]-7-isopropoxy-imidazo[1,2-a]pyridin-6-yl]-6-(trifluoromethyl)pyridine-2-carboxamide. LC-MS (ES⁺): m/z 776.56 [M+H]⁺. ¹H NMR (400 MHz, DMSO-d₆) δ 14.20 (s, 1H), 10.77 (s, 1H), 10.56 (s, 1H), 9.79 (s, 1H), 8.47 (q, J=8.1 Hz, 2H), 8.31 (t, J=4.2 Hz, 1H), 8.15 (s, 1H), 7.45 (s, 1H), 7.22 (s, 1H), 7.10 (s, 1H), 6.97 (s, 1H), 6.82 (d, J=8.6 Hz, 1H), 6.65 (d, J=8.8 Hz, 1H), 5.12 (t, J=6.0 Hz, 1H), 4.44 (q, J=16.6 Hz, 3H), 4.23 (q, J=5.3 Hz, 1H), 3.47 (m, 4H), 3.30 (m, 5H), 3.05 (m, 2H), 2.95 (d, J=12.3 Hz, 1H), 2.72 (m, 1H), 2.50 (d, J=1.6 Hz, 1H), 2.11 (q, J=9.3 Hz, 3H), 1.87 (m, 1H), 1.69 (m, 6H), 1.48 (d, J=6.0 Hz, 1H).

### Example 11 Synthesis of N-[2-[1-[2-[4-[5-[(2,6-dioxo-3-piperidyl)amino]-2-pyridyl]-1-piperidyl]-2-oxo-ethyl]-4-piperidyl]-7-isopropoxy-imidazo[1,2-a]pyridin-6-yl]-6-(trifluoromethyl)pyridine-2-carboxamide

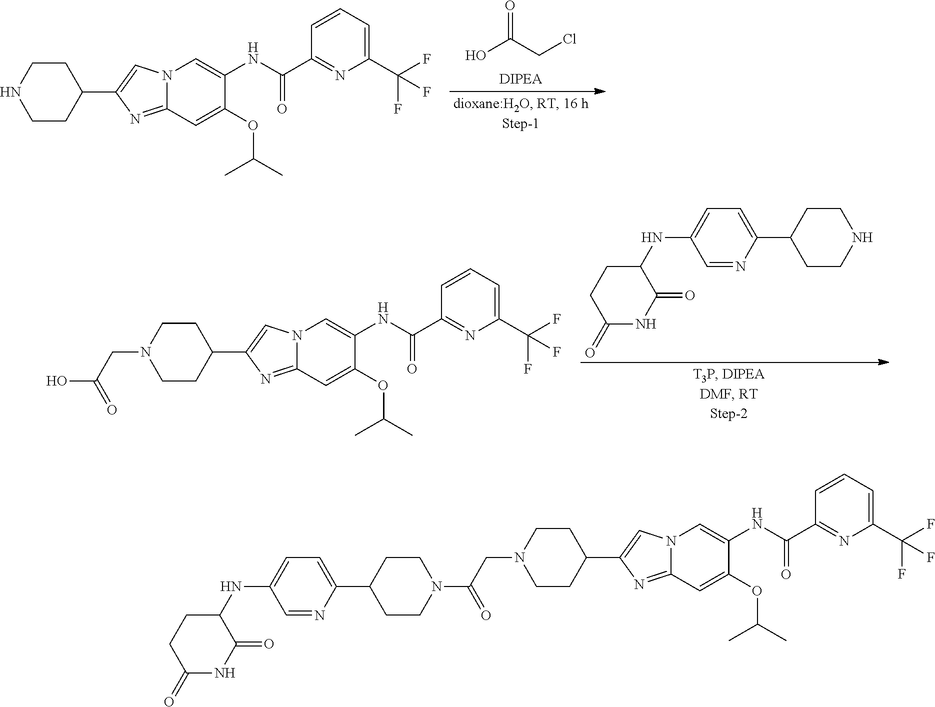

### Step-1

To a stirred solution of N-[7-isopropoxy-2-(4-piperidyl)imidazo[1,2-a]pyridin-6-yl]-6-(trifluoromethyl)pyridine-2-carboxamide trifluoroacetic acid salt (0.5 g, 890.51 μmol) in mixture of dioxane (2 mL) and water (8 mL) was added N-ethyl-N-isopropyl-propan-2-amine (230.18 mg, 1.78 mmol, 310.22 p L) followed by 2-chloroacetic acid (120.2 μL, 1.8 mmol) at room temperature. The reaction mixture was stirred for 16 h at 65° C. After completion of the reaction, the reaction mixture was concentrated under reduced pressure and purified by prep HPLC to afford 2-[4-[7-isopropoxy-6-[[6-(trifluoromethyl)pyridine-2-carbonyl]amino]imidazo[1,2-a]pyridin-2-yl]-1-piperidyl]acetic acid (170 mg, 332.95 μmol, 37.39% yield) as an off white solid. LC-MS (ES⁺): m/z 506.37 [M+H]⁺

### Step-2

To a stirred solution of 2-[4-[7-isopropoxy-6-[[6-(trifluoromethyl)pyridine-2-carbonyl]amino]imidazo[1,2-a]pyridin-2-yl]-1-piperidyl]acetic acid (20.0 mg, 39.57 μmol) in DMF (2.0 mL) was added N-ethyl-N-isopropyl-propan-2-amine (25.57 mg, 197.83 μmol, 34.46 μL) drop wise at 25° C. followed by 2,4,6-Tripropyl-1,3,5,2,4,6-trioxatriphosphorinane-2,4,6-trioxide (25.18 mg, 79.13 μmol). After 5 min, a solution of 3-[[6-(4-piperidyl)-3-pyridyl]amino]piperidine-2,6-dione (15.92 mg, 39.57 μmol) in DMF (1.0 mL) was added at room temperature and stirred for 16 h. After consumption of the starting material, the mixture was concentrated under reduced pressure. The residue was purified by Prep-HPLC to give N-[2-[1-[2-[4-[5-[(2,6-dioxo-3-piperidyl)amino]-2-pyridyl]-1-piperidyl]-2-oxo-ethyl]-4-piperidyl]-7-isopropoxy-imidazo[1,2-a]pyridin-6-yl]-6-(trifluoromethyl) pyridine-2-carboxamide trifluoroacetic acid salt (15.9 mg, 17.85 μmol, 45.11% yield) as an off white solid. LC-MS (ES⁺): m/z 776.47 [M+H]⁺. 1H NMR (401 MHz, DMSO) δ 10.87 (s, 1H), 10.55 (s, 1H), 9.74 (s, 1H), 8.47 (q, J=8.3 Hz, 1H), 8.30 (d, J=7.2 Hz, 1H), 8.15 (s, 1H), 8.02 (s, 1H), 7.44-6.97 (m, 7H), 5.09 (d, J=5.6 Hz, 1H), 4.44 (m, 4H), 3.71 (m, 4H), 3.20 (m, 4H), 2.91 (d, J=58.7 Hz, 1H), 2.75 (m, 3H), 2.28-2.00 (m, 7H), 1.73 (d, J=15.8 Hz, 1H), 1.47 (d, J=5.9 Hz, 6H).

### Example 12

Compound of Example 12 was prepared substantially following the synthesis of Example 11

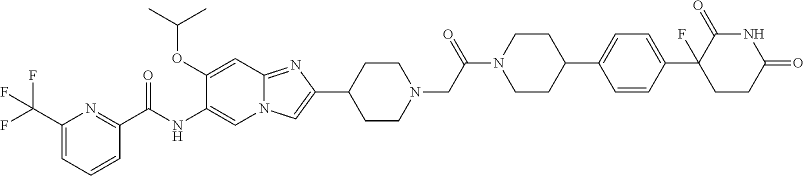 [SMILES: CC(C)Oc1cc2nc(C3CCN(CC(=O)N4CCC(c5ccc(C6(F)CCC(=O)NC6=O)cc5)CC4)CC3)cn2cc1NC(=O)c1cccc(C(F)(F)F)n1]

N-[2-[1-[2-[4-[4-(3-fluoro-2,6-dioxo-3-piperidyl)phenyl]-1-piperidyl]-2-oxo-ethyl]-4-piperidyl]-7-isopropoxy-imidazo[1,2-a]pyridin-6-yl]-6-(trifluoromethyl)pyridine-2-carboxamide. LC-MS (ES⁺): m/z 778.14 [M+H]⁺. ¹H NMR (400 MHz, DMSO-d₆) δ 11.39 (s, 1H), 10.52 (s, 1H), 9.62 (s, 1H), 8.45 (m, 2H), 8.20 (q, J=25.6 Hz, 1H), 7.54 (m, 4H), 7.18-6.52 (m, 2H), 4.96 (m, 1H), 4.55 (d, J=11.9 Hz, 1H), 4.36 (d, J=13.9 Hz, 1H), 3.77 (s, 1H), 3.62 (s, 1H), 2.26 (m, 3H), 2.03 (m, 2H), 1.89-1.45 (m, 12H), 1.28 (m, 7H), 0.89 (m, 2H).

### Example 13

Compound of Example 13 was prepared substantially following the synthesis of Example 11

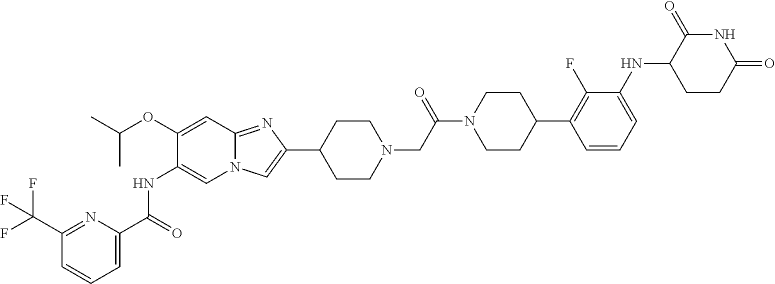 [SMILES: CC(C)Oc1cc2nc(C3CCN(CC(=O)N4CCC(c5cccc(NC6CCC(=O)NC6=O)c5F)CC4)CC3)cn2cc1NC(=O)c1cccc(C(F)(F)F)n1]

N-[2-[1-[2-[4-[3-[(2,6-dioxo-3-piperidyl)amino]-2-fluoro-phenyl]-1-piperidyl]-2-oxo-ethyl]-4-piperidyl]-7-isopropoxy-imidazo[1,2-a]pyridin-6-yl]-6-(trifluoromethyl)pyridine-2-carboxamide. LC-MS (ES⁺): m/z 792.97 [M+H]⁺. ¹H NMR (400 MHz, DMSO-d₆) δ 10.80 (d, J=5.9 Hz, 1H), 10.44 (s, 1H), 9.43 (d, J=5.1 Hz, 1H), 8.44 (m, 2H), 8.24 (t, J=4.2 Hz, 2H), 7.67 (s, 1H), 7.13 (s, 1H), 6.90 (t, J=7.8 Hz, 1H), 6.68 (t, J=8.2 Hz, 1H), 6.49 (t, J=6.7 Hz, 1H), 5.51 (d, J=7.1 Hz, 1H), 4.87 (m, 1H), 4.47 (t, J=24.9 Hz, 1H), 4.37 (q, J=5.7 Hz, 1H), 4.23 (s, 1H), 3.09 (m, 5H), 2.87-2.50 (m, 3H), 2.03 (m, 6H), 1.72 (m, 5H), 1.40-1.14 (m, 8H).

### Example 14

Compound of Example 14 was prepared substantially following the synthesis of Example 11.

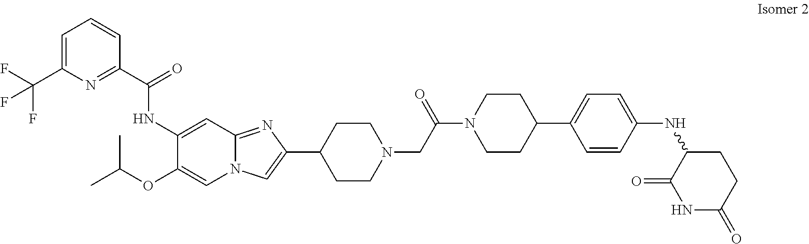 [SMILES: CC(C)Oc1cn2cc(C3CCN(CC(=O)N4CCC(c5ccc(NC6CCC(=O)NC6=O)cc5)CC4)CC3)nc2cc1NC(=O)c1cccc(C(F)(F)F)n1]

N-[2-[1-[2-[4-[4-[[2,6-dioxo-3-piperidyl]amino]phenyl]-1-piperidyl]-2-oxo-ethyl]-4-piperidyl]-6-isopropoxy-imidazo[1,2-a]pyridin-7-yl]-6-(trifluoromethyl)pyridine-2-carboxamide Isomer 2. Example 14 was prepared substantially following the synthesis of Example 11 using 3-[4-(4-piperidyl)anilino]piperidine-2,6-dione Isomer 2. LC-MS (ES⁺): m/z 775.74 [M+H]⁺. ¹H NMR (400 MHz, DMSO-d₆) δ 10.77 (s, 1H), 10.47 (s, 1H), 9.50 (s, 1H), 8.45 (t, J=7.8 Hz, 1H), 8.26 (d, J=7.1 Hz, 1H), 7.78 (s, 1H), 7.15 (t, J=25.5 Hz, 3H), 6.96 (d, J=6.7 Hz, 1H), 6.62 (d, J=7.9 Hz, 2H), 5.71 (d, J=7.2 Hz, 1H), 4.92 (s, 1H), 4.51 (d, J=11.1 Hz, 1H), 4.27 (s, 3H), 3.76 (s, 1H), 3.54 (d, J=26.8 Hz, 1H), 3.04 (d, J=98.4 Hz, 3H), 2.72 (m, 1H), 2.20 (d, J=12.8 Hz, 2H), 2.05 (q, J=14.0 Hz, 3H), 1.85 (m, 4H), 1.57 (d, J=7.3 Hz, 4H), 1.41 (d, J=5.9 Hz, 1H), 1.24 (s, 6H), 1.24 (s, 1H).

### Example 15

Compound of Example 15 was prepared substantially following the synthesis of Example 11.

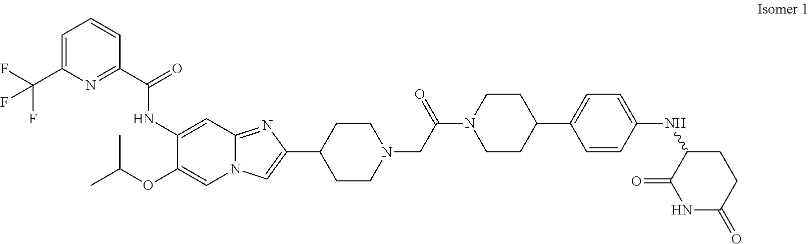 [SMILES: CC(C)Oc1cn2cc(C3CCN(CC(=O)N4CCC(c5ccc(NC6CCC(=O)NC6=O)cc5)CC4)CC3)nc2cc1NC(=O)c1cccc(C(F)(F)F)n1]

N-[2-[1-[2-[4-[4-[[2,6-dioxo-3-piperidyl]amino]phenyl]-1-piperidyl]-2-oxo-ethyl]-4-piperidyl]-6-isopropoxy-imidazo[1,2-a]pyridin-7-yl]-6-(trifluoromethyl)pyridine-2-carboxamide Isomer 1. Example 15 was prepared substantially following the synthesis of Example 11, using 3-[4-(4-piperidyl)anilino]piperidine-2,6-dione Isomer 1. LC-MS (ES⁺): m/z 775.12 [M+H]⁺. ¹H NMR (400 MHz, DMSO-d₆) δ 10.47 (s, 1H), 10.44 (s, 1H), 9.43 (s, 1H), 8.44 (q, J=7.3 Hz, 2H), 8.26 (t, J=16.6 Hz, 3H), 7.67 (s, 1H), 7.07 (s, 3H), 6.97 (d, J=8.1 Hz, 2H), 6.63 (d, J=8.1 Hz, 2H), 5.67 (d, J=8.1 Hz, 1H), 4.86 (d, J=5.9 Hz, 1H), 4.47 (s, 1H), 4.25 (t, J=5.6 Hz, 1H), 4.02 (s, 2H), 3.47 (m, 6H), 3.12 (d, J=11.7 Hz, 1H), 2.71 (m, 3H), 2.08 (m, 3H), 1.81 (m, 2H), 1.65-1.74 (m, 5H), 1.42 (m, 3H).

### Example 16

Compound of Example 16 was prepared substantially following the synthesis of Example 11.

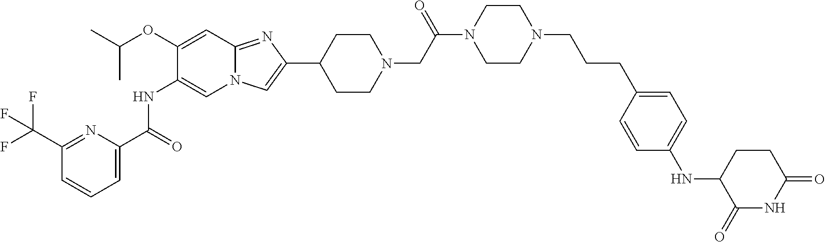 [SMILES: CC(C)Oc1cc2nc(C3CCN(CC(=O)N4CCN(CCCc5ccc(NC6CCC(=O)NC6=O)cc5)CC4)CC3)cn2cc1NC(=O)c1cccc(C(F)(F)F)n1]

N-[2-[1-[2-[4-[3-[4-[(2,6-dioxo-3-piperidyl)amino]phenyl]propyl]piperazin-1-yl]-2-oxo-ethyl]-4-piperidyl]-7-isopropoxy-imidazo[1,2-a]pyridin-6-yl]-6-(trifluoromethyl) pyridine-2-carboxamide. LC-MS (ES⁺): m/z 818.26 [M+H]⁺. ¹H NMR (400 MHz, DMSO-d₆) δ 10.76 (s, 1H), 10.44 (s, 1H), 9.43 (s, 1H), 8.43 (q, J=8.4 Hz, 2H), 8.24 (d, J=7.3 Hz, 1H), 7.67 (s, 1H), 7.12 (s, 1H), 6.92 (d, J=8.4 Hz, 2H), 6.59 (d, J=8.5 Hz, 2H), 5.62 (d, J=7.2 Hz, 1H), 4.87 (m, 1H), 4.24 (t, J=7.6 Hz, 1H), 3.58-3.13 (m, 4H), 3.13 (s, 2H), 2.81 (m, 2H), 2.59-2.50 (m, 2H), 2.43-2.31 (m, 9H), 2.11 (m, 3H), 1.94 (d, J=11.6 Hz, 3H), 1.65 (m, 3H), 1.40 (m, 7H).

### Example 17

Compound of Example 17 was prepared substantially following the synthesis of Example 11.

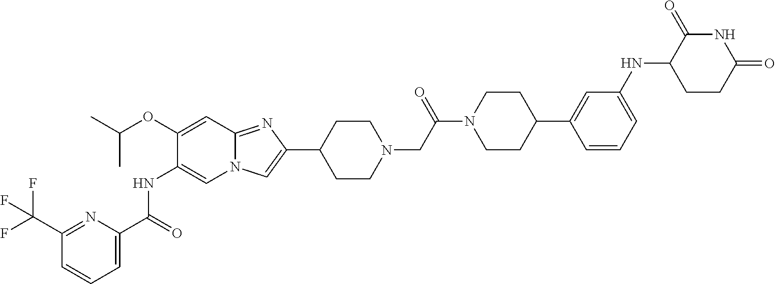 [SMILES: CC(C)Oc1cc2nc(C3CCN(CC(=O)N4CCC(c5cccc(NC6CCC(=O)NC6=O)c5)CC4)CC3)cn2cc1NC(=O)c1cccc(C(F)(F)F)n1]

N-[2-[1-[2-[4-[3-[(2,6-dioxo-3-piperidyl)amino]phenyl]-1-piperidyl]-2-oxo-ethyl]-4-piperidyl]-7-isopropoxy-imidazo[1,2-a]pyridin-6-yl]-6-(trifluoromethyl)pyridine-2-carboxamide. LC-MS (ES⁻): m/z 773.31 [M−H]⁻. ¹H NMR (400 MHz, DMSO-d₆) δ 10.76 (s, 1H), 10.44 (s, 1H), 9.42 (d, J=3.8 Hz, 1H), 8.43 (m, J=6.0 Hz, 2H), 8.24 (t, J=4.2 Hz, 1H), 7.66 (s, 1H), 7.12 (s, 1H), 7.00 (t, J=7.7 Hz, 1H), 6.48 (m, 3H), 5.76 (d, J=7.5 Hz, 1H), 4.87 (m, J=5.8 Hz, 1H), 4.50 (d, J=12.6 Hz, 1H), 4.22 (m, 1H), 3.04 (t, J=6.3 Hz, 1H), 2.91 (s, 1H), 2.63 (m, J=7.1 Hz, 1H), 2.14 (d, J=10.9 Hz, 1H), 1.95 (d, J=14.0 Hz, 1H), 1.66 (m, 14H), 1.40 (d, J=5.9 Hz, 7H), 1.26 (m, 2H), 0.87 (m, 1H).

### Example 18

Compound of Example 18 was prepared substantially following the synthesis of Example 11.

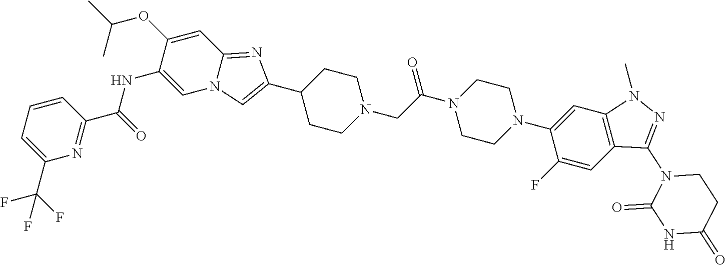 [SMILES: CC(C)Oc1cc2nc(C3CCN(CC(=O)N4CCN(c5cc6c(cc5F)c(N5CCC(=O)NC5=O)nn6C)CC4)CC3)cn2cc1NC(=O)c1cccc(C(F)(F)F)n1]

N-(2-(1-(2-(4-(3-(2,4-dioxotetrahydropyrimidin-1(2H)-yl)-5-fluoro-1-methyl-1H-indazol-6-yl)piperazin-1-yl)-2-oxoethyl)piperidin-4-yl)-7-isopropoxyimidazo[1,2-a]pyridin-6-yl)-6-(trifluoromethyl)picolinamide. LC-MS (ES⁺): m/z 833.83 [M+H]⁺. ¹H NMR (400 MHz, DMSO-d₆) δ ppm 10.54 (s, 1H) 10.44 (s, 1H) 9.44-9.40 (m, 1H) 8.47-8.36 (m, 3H) 8.26-8.22 (m, 1H) 7.39 (s, 1H) 7.16 (d, J=7.2 Hz, 1H) 7.17-7.09 (m, 2H) 4.91-4.82 (m, 1H) 3.95 (s, 3H) 3.92-3.87 (m, 2H) 3.82-3.78 (m, 2H) 3.70-3.65 (m, 2H) 3.25-3.20 (m, 4H) 3.18-3.12 (m, 4H) 3.10-2.95 (m, 2H) 2.16-2.14 (m, 2H) 2.17-2.14 (m, 2H) 1.98-1.94 (m, 2H) 1.75-1.62 (m, 2H) 1.40 (d, J=6.0 Hz, 6H).

### Example 19

Compound of Example 19 was prepared substantially following the synthesis of Example 11.

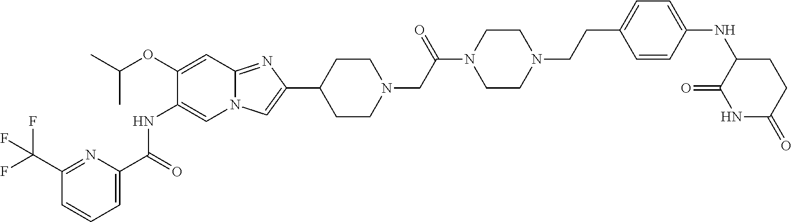 [SMILES: CC(C)Oc1cc2nc(C3CCN(CC(=O)N4CCN(CCc5ccc(NC6CCC(=O)NC6=O)cc5)CC4)CC3)cn2cc1NC(=O)c1cccc(C(F)(F)F)n1]

N-[2-[1-[2-[4-[2-[4-[(2,6-dioxo-3-piperidyl)amino]phenyl]ethyl]piperazin-1-yl]-2-oxo-ethyl]-4-piperidyl]-7-isopropoxy-imidazo[1,2-a]pyridin-6-yl]-6-(trifluoromethyl) pyridine-2-carboxamide. LC-MS (ES⁺): m/z 804.61 [M+H]⁺. ¹H NMR (400 MHz, DMSO-d₆) δ 10.77 (s, 1H), 10.46 (s, 1H), 9.47 (s, 1H), 8.45 (m, 1H), 8.25 (d, J=7.3 Hz, 1H), 8.14 (s, 2H), 7.75 (s, 1H), 7.14 (s, 1H), 6.94 (d, J=8.2 Hz, 2H), 6.60 (d, J=8.3 Hz, 2H), 5.66 (d, J=7.4 Hz, 1H), 4.90 (m, 1H), 4.26 (m, J=5.8 Hz, 1H), 3.51 (m, 10H), 2.49-2.78 (m, 9H), 2.10 (q, J=5.9 Hz, 3H), 1.87 (m, 3H), 1.41 (m, 6H), 1.26 (d, J=6.5 Hz, 1H).

### Example 20

Compound of Example 20 was prepared substantially following the synthesis of Example 11.

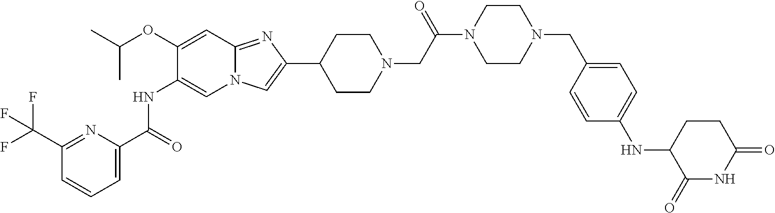 [SMILES: CC(C)Oc1cc2nc(C3CCN(CC(=O)N4CCN(Cc5ccc(NC6CCC(=O)NC6=O)cc5)CC4)CC3)cn2cc1NC(=O)c1cccc(C(F)(F)F)n1]

N-[2-[1-[2-[4-[[4-[(2,6-dioxo-3-piperidyl)amino]phenyl]methyl]piperazin-1-yl]-2-oxo-ethyl]-4-piperidyl]-7-isopropoxy-imidazo[1,2-a]pyridin-6-yl]-6-(trifluoromethyl) pyridine-2-carboxamide. LC-MS (ES⁺): m/z 792.97 [M+H]⁺. ¹H NMR (400 MHz, DMSO-d₆) δ 10.77 (s, 1H), 10.44 (s, 1H), 9.44 (s, 1H), 8.43 (q, J=8.4 Hz, 2H), 8.24 (d, J=7.2 Hz, 1H), 8.19 (s, 1H), 7.67 (s, 1H), 7.13 (s, 1H), 7.01 (d, J=8.2 Hz, 2H), 6.63 (d, J=8.4 Hz, 2H), 5.77 (d, J=7.3 Hz, 1H), 4.88 (t, J=6.1 Hz, 1H), 4.29 (d, J=4.5 Hz, 1H), 3.56 (m, 2H), 3.33 (s, 2H), 3.15 (d, J=12.7 Hz, 2H), 2.86 (d, J=10.9 Hz, 2H), 2.70-2.50 (m, 3H), 2.40 (m, 2H), 2.25 (s, 1H), 2.12 (t, J=11.1 Hz, 3H), 1.92 (d, J=7.8 Hz, 3H), 1.65 (d, J=10.8 Hz, 2H), 1.40 (m, 8H).

### Example 21

Compound of Example 21 was prepared substantially following the synthesis of Example 11.

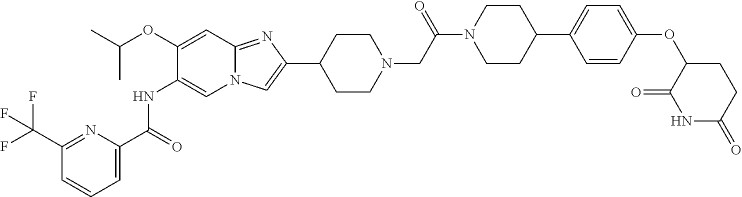 [SMILES: CC(C)Oc1cc2nc(C3CCN(CC(=O)N4CCC(c5ccc(OC6CCC(=O)NC6=O)cc5)CC4)CC3)cn2cc1NC(=O)c1cccc(C(F)(F)F)n1]

N-[2-[1-[2-[4-[4-[(2,6-dioxo-3-piperidyl)oxy]phenyl]-1-piperidyl]-2-oxo-ethyl]-4-piperidyl]-7-isopropoxy-imidazo[1,2-a]pyridin-6-yl]-6-(trifluoromethyl)pyridine-2-carboxamide. LC-MS (ES⁺): m/z 776.01 [M+H]⁺. ¹H NMR (400 MHz, DMSO-d₆) δ 14.23 (s, 1H), 10.92 (s, 1H), 10.55 (s, 1H), 9.63 (d, J=41.6 Hz, 1H), 8.47 (q, J=8.4 Hz, 1H), 8.30 (d, J=7.2 Hz, 1H), 8.14 (s, 1H), 7.42 (s, 1H), 7.15 (q, J=17.1 Hz, 3H), 6.97 (d, J=8.3 Hz, 1H), 5.14 (m, 2H), 4.43 (m, 1H), 3.75 (d, J=13.6 Hz, 2H), 3.65 (d, J=10.9 Hz, 3H), 3.20 (t, J=12.2 Hz, 4H), 2.71 (m, 4H), 2.18 (m, 6H), 1.86 (d, J=11.8 Hz, 2H), 1.62 (d, J=9.9 Hz, 1H), 1.47 (d, J=5.9 Hz, 6H), 1.19 (t, J=17.8 Hz, 1H).

### Example 22

Compound of Example 22 was prepared substantially following the synthesis of Example 11.

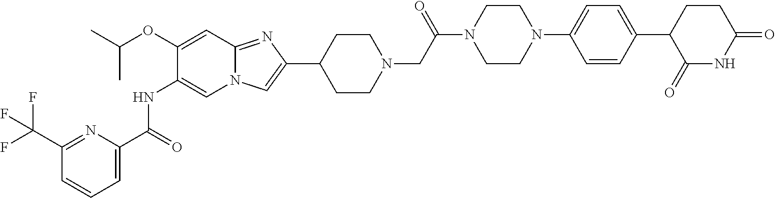 [SMILES: CC(C)Oc1cc2nc(C3CCN(CC(=O)N4CCN(c5ccc(C6CCC(=O)NC6=O)cc5)CC4)CC3)cn2cc1NC(=O)c1cccc(C(F)(F)F)n1]

N-[2-[1-[2-[4-[4-(2,6-dioxo-3-piperidyl)phenyl]piperazin-1-yl]-2-oxo-ethyl]-4-piperidyl]-7-isopropoxy-imidazo[1,2-a]pyridin-6-yl]-6-(trifluoromethyl)pyridine-2-carboxamide. LC-MS (ES⁺): m/z 761.05 [M+H]⁺. ¹H NMR (400 MHz, DMSO-d₆) δ 10.77 (s, 1H), 10.44 (s, 1H), 9.42 (s, 1H), 8.44 (t, J=8.1 Hz, 2H), 8.24 (d, J=7.1 Hz, 1H), 8.16 (s, 1H), 7.67 (s, 1H), 7.06-7.12 (t, J=11.5 Hz, 3H), 6.93 (d, J=8.6 Hz, 2H), 4.87 (m, 1H), 3.74 (q, J=5.1 Hz, 3H), 3.60 (s, 2H), 3.15 (t, J=26.8 Hz, 2H), 2.91 (d, J=10.9 Hz, 2H), 2.63 (m, 2H), 2.50 (m, 2H), 2.43 (m, 1H), 2.15 (t, J=11.7 Hz, 3H), 1.99 (m, 3H), 1.66 (d, J=10.0 Hz, 2H), 1.40 (d, J=6.0 Hz, 6H), 1.23 (s, 1H).

### Example 23

Compound of Example 23 was prepared substantially following the synthesis of Example 11.

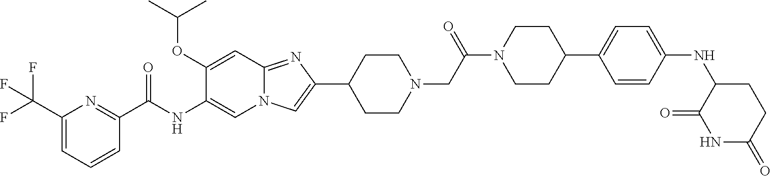 [SMILES: CC(C)Oc1cc2nc(C3CCN(CC(=O)N4CCC(c5ccc(NC6CCC(=O)NC6=O)cc5)CC4)CC3)cn2cc1NC(=O)c1cccc(C(F)(F)F)n1]

N-[2-[1-[2-[4-[4-[(2,6-dioxo-3-piperidyl)amino]phenyl]-1-piperidyl]-2-oxo-ethyl]-4-piperidyl]-7-isopropoxy-imidazo[1,2-a]pyridin-6-yl]-6-(trifluoromethyl)pyridine-2-carboxamide. LC-MS (ES⁺): m/z 775.46 [M+H]⁺. ¹H NMR (400 MHz, DMSO-d₆) δ 10.75 (s, 1H), 10.44 (s, 1H), 9.43 (s, 1H), 8.41 (m, J=11.2 Hz, 2H), 8.23-8.46 (d, J=7.1 Hz, 2H), 7.66 (s, 1H), 7.14 (s, 1H), 6.94 (d, J=8.3 Hz, 1H), 6.62 (d, J=8.2 Hz, 2H), 5.66 (d, J=7.3 Hz, 1H), 4.87 (m, J=5.9 Hz, 1H), 4.49 (d, J=11.8 Hz, 1H), 4.25 (q, J=10.5 Hz, 2H), 2.83 (m, J=24.4 Hz, 9H), 2.13 (q, J=10.3 Hz, 10H), 1.96 (t, J=13.6 Hz, 1H), 1.76 (m, J=10.5 Hz, 7H), 1.40 (d, J=5.9 Hz, 1H).

### Example 24

Compound of Example 24 was prepared substantially following the synthesis of Example 11.

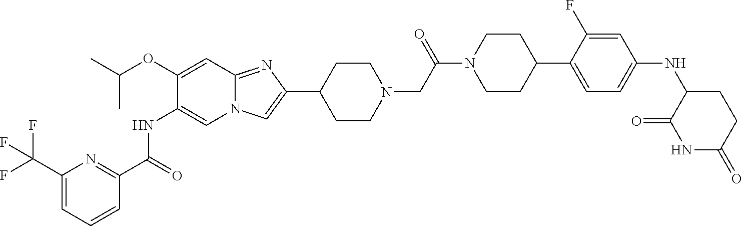 [SMILES: CC(C)Oc1cc2nc(C3CCN(CC(=O)N4CCC(c5ccc(NC6CCC(=O)NC6=O)cc5F)CC4)CC3)cn2cc1NC(=O)c1cccc(C(F)(F)F)n1]

N-[2-[1-[2-[4-[4-[(2,6-dioxo-3-piperidyl)amino]-2-fluoro-phenyl]-1-piperidyl]-2-oxo-ethyl]-4-piperidyl]-7-isopropoxy-imidazo[1,2-a]pyridin-6-yl]-6-(trifluoromethyl)pyridine-2-carboxamide. LC-MS (ES⁺): m/z 793.44 [M+H]⁺. ¹H NMR (400 MHz, DMSO-d₆) δ 14.11 (s, 1H), 10.80 (s, 1H), 10.52 (s, 1H), 9.65 (s, 1H), 8.46 (q, J=8.1 Hz, 1H), 8.29 (d, J=6.7 Hz, 1H), 7.99 (s, 2H), 7.08 (m, J=27.8 Hz, 2H), 6.46 (t, J=6.6 Hz, 1H), 6.06 (s, 1H), 5.04 (s, 4H), 4.43 (q, J=29.9 Hz, 2H), 3.69 (m, 8H), 3.08 (m, J=24.6 Hz, 1H), 2.74 (m, J=8.3 Hz, 2H), 2.60 (s, 7H), 1.44-1.50 (m, 7H), 1.24 (s, 1H).

### Example 25

Compound of Example 25 was prepared substantially following the synthesis of Example 11.

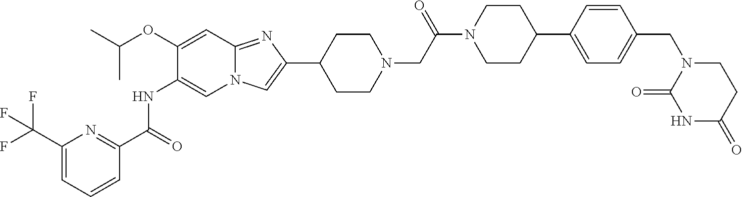 [SMILES: CC(C)Oc1cc2nc(C3CCN(CC(=O)N4CCC(c5ccc(CN6CCC(=O)NC6=O)cc5)CC4)CC3)cn2cc1NC(=O)c1cccc(C(F)(F)F)n1]

N-[2-[1-[2-[4-[4-[(2,4-dioxohexahydropyrimidin-1-yl)methyl]phenyl]-1-piperidyl]-2-oxo-ethyl]-4-piperidyl]-7-isopropoxy-imidazo[1,2-a]pyridin-6-yl]-6-(trifluoromethyl) pyridine-2-carboxamide. LC-MS (ES⁺): m/z 775.46 [M+H]⁺. ¹H NMR (400 MHz, DMSO-d₆) δ 10.53 (s, 1H), 10.20 (s, 1H), 9.68 (s, 1H), 8.46 (q, J=8.3 Hz, 1H), 8.29 (d, J=7.1 Hz, 1H), 8.06 (s, 1H), 7.36-6.96 (m, 7H), 5.05 (s, 1H), 4.55-4.33 (m, 5H), 3.70 (q, J=20.2 Hz, 1H), 3.64 (q, J=20.2 Hz, 2H), 3.36 (m, 6H), 2.82 (t, J=11.4 Hz, 2H), 2.50 (s, 2H), 2.27 (d, J=13.9 Hz, 2H), 2.03 (t, J=17.1 Hz, 2H), 1.88 (t, J=11.3 Hz, 2H), 1.64 (d, J=10.4 Hz, 1H), 1.46 (d, J=5.7 Hz, 6H).

### Example 26

Compound of Example 26 was prepared substantially following the synthesis of Example 11.

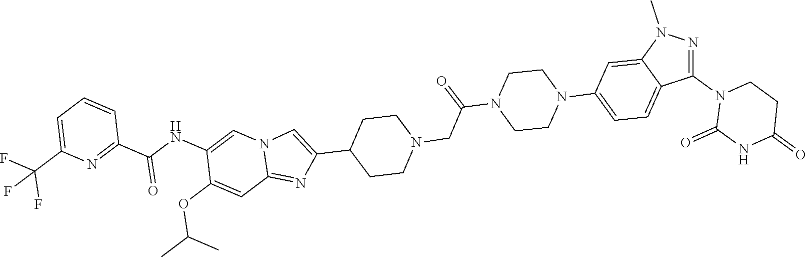 [SMILES: CC(C)Oc1cc2nc(C3CCN(CC(=O)N4CCN(c5ccc6c(N7CCC(=O)NC7=O)nn(C)c6c5)CC4)CC3)cn2cc1NC(=O)c1cccc(C(F)(F)F)n1]

N-[2-[1-[2-[4-[3-(2,4-dioxohexahydropyrimidin-1-yl)-1-methyl-indazol-6-yl]piperazin-1-yl]-2-oxo-ethyl]-4-piperidyl]-7-isopropoxy-imidazo[1,2-a]pyridin-6-yl]-6-(trifluoromethyl)pyridine-2-carboxamide. LC-MS (ES⁺): m/z 816.28 [M+H]⁺. ¹H NMR (400 MHz, DMSO-d₆) δ 10.53 (d, J=6.7 Hz, 2H), 9.70 (s, 1H), 8.47 (q, J=8.4 Hz, 2H), 8.29 (d, J=7.2 Hz, 1H), 8.09 (s, 1H), 7.51 (d, J=9.0 Hz, 1H), 7.39 (s, 1H), 7.23-6.90 (m, 5H), 5.06 (s, 1H), 4.40 (s, 2H), 3.91 (m, 5H), 3.73 (s, 2H), 3.62 (t, J=13.0 Hz, 4H), 3.36 (m, 4H), 2.74 (t, J=6.5 Hz, 2H), 2.29 (t, J=14.0 Hz, 2H), 2.04 (t, J=13.6 Hz, 2H), 1.46 (d, J=5.9 Hz, 6H).

### Example 27

Compound of Example 27 was prepared substantially following the synthesis of Example 11.

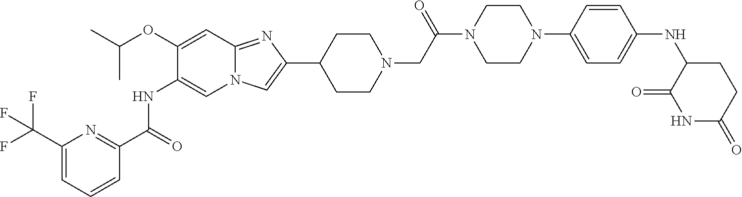 [SMILES: CC(C)Oc1cc2nc(C3CCN(CC(=O)N4CCN(c5ccc(NC6CCC(=O)NC6=O)cc5)CC4)CC3)cn2cc1NC(=O)c1cccc(C(F)(F)F)n1]

N-[2-[1-[2-[4-[4-[(2,6-dioxo-3-piperidyl)amino]phenyl]piperazin-1-yl]-2-oxo-ethyl]-4-piperidyl]-7-isopropoxy-imidazo[1,2-a]pyridin-6-yl]-6-(trifluoromethyl)pyridine-2-carboxamide. LC-MS (ES⁺): m/z 776.34 [M+H]⁺. ¹H NMR (400 MHz, DMSO-d₆) δ 10.77 (s, 1H), 10.56 (s, 1H), 9.77 (s, 1H), 8.47 (m, 2H), 8.30 (q, J=2.9 Hz, 1H), 8.19 (d, J=11.7 Hz, 1H), 7.47 (s, 1H), 7.23 (s, 1H), 7.10 (s, 1H), 6.97 (s, 1H), 6.84 (d, J=8.6 Hz, 2H), 6.65 (d, J=8.7 Hz, 2H), 5.11 (t, J=5.9 Hz, 1H), 4.39 (s, 2H), 4.23 (q, J=5.2 Hz, 1H), 3.53 (m, 3H), 3.18 (m, 4H), 3.01 (m, 4H), 2.70 (m, 1H), 2.58 (q, J=7.3 Hz, 1H), 2.27 (m, 3H), 2.06 (m, 2H), 1.87 (m, 1H), 1.47 (d, J=6.0 Hz, 6H).

### Example 28

Compound of Example 28 was prepared substantially following the synthesis of Example 11.

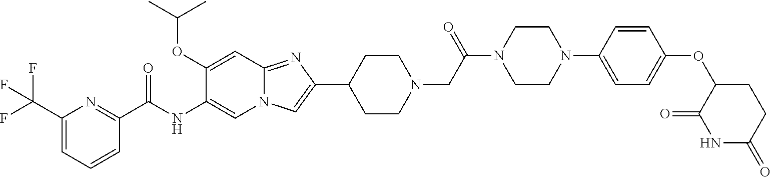 [SMILES: CC(C)Oc1cc2nc(C3CCN(CC(=O)N4CCN(c5ccc(OC6CCC(=O)NC6=O)cc5)CC4)CC3)cn2cc1NC(=O)c1cccc(C(F)(F)F)n1]

N-[2-[1-[2-[4-[4-[(2,6-dioxo-3-piperidyl)oxy]phenyl]piperazin-1-yl]-2-oxo-ethyl]-4-piperidyl]-7-isopropoxy-imidazo[1,2-a]pyridin-6-yl]-6-(trifluoromethyl)pyridine-2-carboxamide. LC-MS (ES⁺): m/z 777.13 [M+H]⁺. ¹H NMR (400 MHz, DMSO-d₆) δ 10.88 (s, 1H), 10.44 (s, 1H), 9.42 (s, 1H), 8.43 (m, 2H), 8.24 (q, J=2.9 Hz, 1H), 7.66 (s, 1H), 7.12 (s, 1H), 6.92 (m, 3H), 5.03 (q, J=5.1 Hz, 1H), 4.87 (m, 1H), 3.74 (s, 2H), 3.60 (s, 2H), 3.31 (s, 1H), 3.20 (s, 2H), 3.02 (d, J=35.1 Hz, 2H), 2.90 (d, J=11.2 Hz, 2H), 2.63 (m, 3H), 2.13 (m, 4H), 1.90 (m, 6H), 1.66 (m, 2H), 1.40 (m, 4H).

### Example 29

Compound of Example 29 was prepared substantially following the synthesis of Example 11.

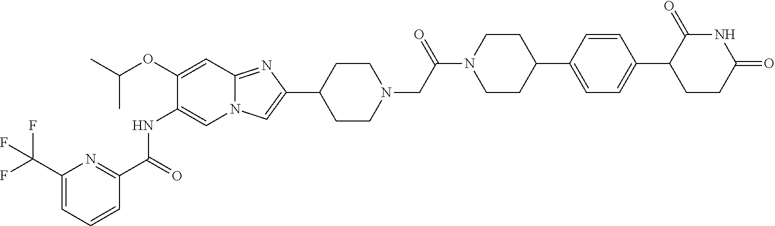 [SMILES: CC(C)Oc1cc2nc(C3CCN(CC(=O)N4CCC(c5ccc(C6CCC(=O)NC6=O)cc5)CC4)CC3)cn2cc1NC(=O)c1cccc(C(F)(F)F)n1]

N-[2-[1-[2-[4-[4-(2,6-dioxo-3-piperidyl)phenyl]-1-piperidyl]-2-oxo-ethyl]-4-piperidyl]-7-isopropoxy-imidazo[1,2-a]pyridin-6-yl]-6-(trifluoromethyl)pyridine-2-carboxamide. LC-MS (ES⁺): m/z 760.00 [M+H]⁺. 1H NMR (401 MHz, DMSO-d₆) δ 10.83 (s, 1H), 10.55 (s, 1H), 9.70 (d, J=37.2 Hz, 1H), 8.47 (q, J=8.5 Hz, 2H), 8.30 (d, J=7.1 Hz, 1H), 8.15 (s, 1H), 7.44 (s, 1H), 7.10 (m, 6H), 5.10 (t, J=5.7 Hz, 1H), 4.54 (d, J=13.0 Hz, 1H), 4.38 (q, J=17.1 Hz, 2H), 3.80 (m, 2H), 3.66 (d, J=9.9 Hz, 2H), 3.21 (q, J=10.3 Hz, 4H), 2.81 (q, J=10.8 Hz, 2H), 2.67 (m, 1H), 2.5 (m, 1H), 2.23 (m, 4H), 2.05 (m, 2H), 1.89 (t, J=8.2 Hz, 2H), 1.69 (m, 1H), 1.47 (m, 5H).

### Example 30 Synthesis of N-[2-[1-[3-[4-[4-[(2,6-dioxo-3-piperidyl)amino]phenyl]-1-piperidyl]-3-oxo-propyl]-4-piperidyl]-7-isopropoxy-imidazo[1,2-a]pyridin-6-yl]-6-(trifluoromethyl)pyridine-2-carboxamide

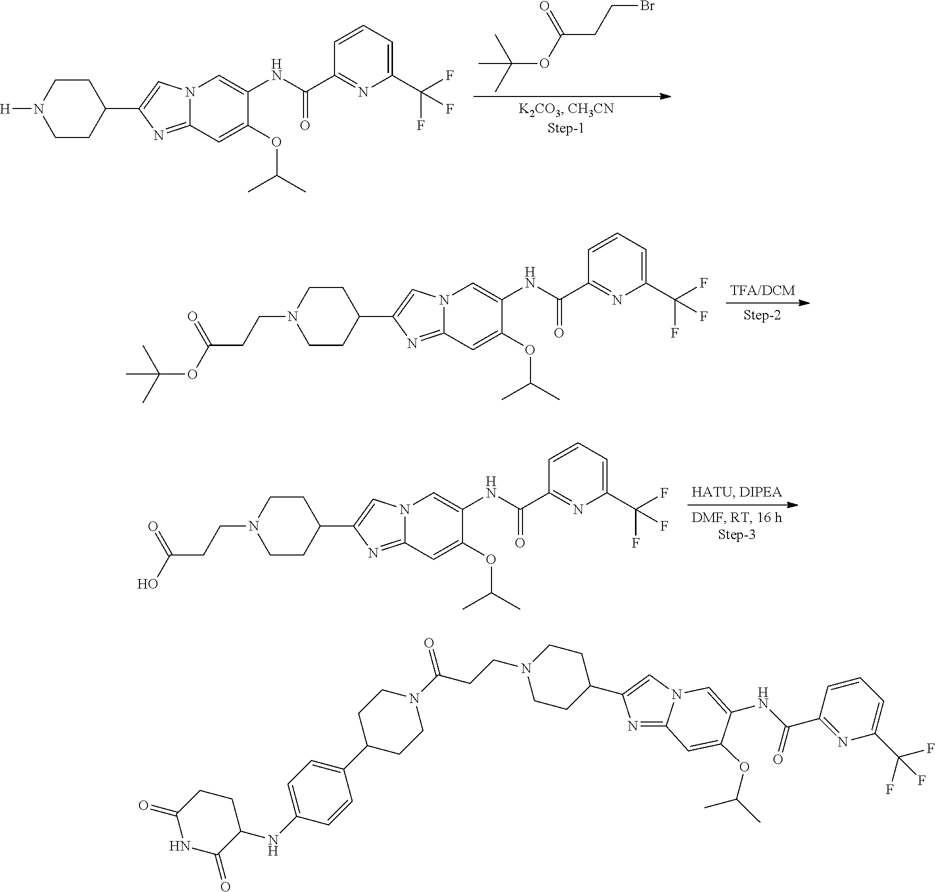

### Step-1

To stirred a solution of N-[7-isopropoxy-2-(4-piperidyl)imidazo[1,2-a]pyridin-6-yl]-6-(trifluoromethyl)pyridine-2-carboxamide trifluoroacetic acid salt (1.0 g, 1.78 mmol), potassium carbonate, anhydrous (738.44 mg, 5.34 mmol) in acetonitrile (10 mL) at 0° C. for 10 minutes, was added tert-butyl 3-bromopropanoate (446.85 mg, 2.14 mmol) and stirred for 16 hours at 80° C. The reaction mixture was poured into water and extracted with ethyl acetate. The organic layer was concentrated under reduced pressure and the residue was washed with diethyl ether then desiccated to give tert-butyl 3-[4-[7-isopropoxy-6-[[6-(trifluoromethyl)pyridine-2-carbonyl]amino]imidazo[1,2-a]pyridin-2-yl]-1-piperidyl]propanoate (0.600 g, 979.81 μmol, 55.01% yield) as light yellow semisolid. Used without further purification. LC-MS (ES⁺): m/z 576.83 [M+H]⁺.

### Step-2

To stirred a solution of tert-butyl 3-[4-[7-isopropoxy-6-[[6-(trifluoromethyl)pyridine-2-carbonyl]amino]imidazo[1,2-a]pyridin-2-yl]-1-piperidyl]propanoate (0.500 g, 868.63 μmol) in DCM (5 mL) for 10 minutes at 0° C., was added TFA (1.98 g, 17.37 mmol, 1.34 mL) and stirred for 16 hrs at room temperature. The solvent was evaporated under reduced pressure, co-distilled with toluene (twice) and washed with diethyl ether to obtain compound of 3-[4-[7-isopropoxy-6-[[6-(trifluoromethyl)pyridine-2-carbonyl]amino]imidazo[1,2-a]pyridin-2-yl]-1-piperidyl]propanoic acid trifluoroacetic acid salt (0.420 g, 643.06 μmol, 74.03% yield) as pale brown solid. LC-MS (ES⁺): m/z 520.37 [M+H]⁺.

### Step-3

To a stirred solution of 3-[4-[7-isopropoxy-6-[[6-(trifluoromethyl)pyridine-2-carbonyl]amino]imidazo[1,2-a]pyridin-2-yl]-1-piperidyl]propanoic acid trifluoroacetic acid salt (30.0 mg, 47.35 μmol) in DMF (2.0 mL) was added N-ethyl-N-isopropyl-propan-2-amine (30.60 mg, 236.77 μmol, 41.24 μL) drop wise at 25° C. followed by N,N,N′,N′-tetramethyl-1-(3-oxido-2,3-dihydrotriazolo[4,5-b]pyridin-3-ium-1-yl)methanediamine; hexafluorophosphate (36.20 mg, 94.71 μmol). After 5 min, a solution of 3-[4-(4-piperidyl)anilino]piperidine-2,6-dione HCl salt (19.01 mg, 58.70 μmol) in DMF (1.0 mL) was added at room temperature and stirred for 16 h. After consumption of starting material, the mixture was concentrated and the residue was purified by Prep-HPLC to afford N-[2-[1-[3-[4-[4-[(2,6-dioxo-3-piperidyl) amino]phenyl]-1-piperidyl]-3-oxo-propyl]-4-piperidyl]-7-isopropoxy-imidazo[1,2-a]pyridin-6-yl]-6-(trifluoromethyl)pyridine-2-carboxamide trifluoroacetic acid salt (43.0 mg, 44.46 μmol, 93.90% yield) as green solid. LC-MS (ES⁺): m/z 789.45 [M+H]⁺. ¹H NMR (400 MHz, DMSO-d₆) δ 10.77 (s, 1H), 10.55 (s, 1H), 9.75 (s, 1H), 9.33 (s, 1H), 8.47 (q, J=8.5 Hz, 2H), 8.30 (d, J=7.2 Hz, 1H), 8.16 (s, 1H), 7.47 (s, 1H), 7.24 (s, 1H), 7.11 (s, 1H), 6.96 (t, J=6.6 Hz, 2H), 6.62 (d, J=8.4 Hz, 2H), 5.09 (t, J=5.9 Hz, 1H), 4.53 (d, J=12.1 Hz, 1H), 4.27 (q, J=5.2 Hz, 1H), 3.83 (q, J=37.6 Hz, 2H), 3.44 (t, J=26.5 Hz, 2H), 3.12 (t, J=12.3 Hz, 4H), 2.92 (d, J=4.6 Hz, 2H), 2.67 (m, 4H), 2.30 (m, 4H), 2.07 (s, 1H), 1.83 (m, 4H), 1.47 (d, J=5.9 Hz, 6H).
Mobile Phase (A): 10 mM AA IN WATER
Mobile Phase (B): ACETONITRILE
Flow Rate: 17 ml/min
Column: SUNFIRE 5 μm (19×150 mm)
Gradient Time % B: 0/10, 2.5/10, 26/38.9, 26.10/100, 28.10/100, 28.20/10, 30.20/10
Solubility: ACN+THF No. of injections: 5

### Example 31

Compound of Example 31 was prepared substantially following the synthesis of Example 30.

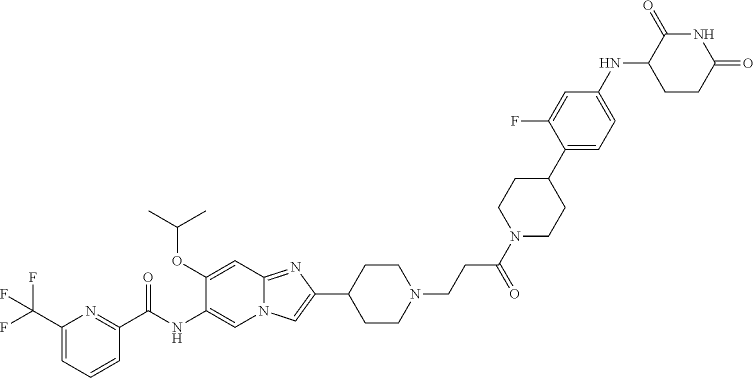 [SMILES: CC(C)Oc1cc2nc(C3CCN(CCC(=O)N4CCC(c5ccc(NC6CCC(=O)NC6=O)cc5F)CC4)CC3)cn2cc1NC(=O)c1cccc(C(F)(F)F)n1]

N-[2-[1-[3-[4-[4-[(2,6-dioxo-3-piperidyl)amino]-2-fluoro-phenyl]-1-piperidyl]-3-oxo-propyl]-4-piperidyl]-7-isopropoxy-imidazo[1,2-a]pyridin-6-yl]-6-(trifluoromethyl)pyridine-2-carboxamide. LC-MS (ES⁺): m/z 807.34 [M+H]⁺. ¹H NMR (400 MHz, DMSO-d₆) δ 10.44 (s, 1H), 9.43 (s, 1H), 8.43 (q, J=8.2 Hz, 2H), 8.24 (d, J=7.2 Hz, 1H), 7.66 (s, 1H), 7.12 (s, 1H), 6.97 (t, J=8.6 Hz, 1H), 6.45 (t, J=6.1 Hz, 2H), 6.02 (d, J=7.8 Hz, 1H), 4.88 (t, J=6.0 Hz, 1H), 4.53 (d, J=12.8 Hz, 1H), 4.31 (t, J=4.2 Hz, 1H), 4.01 (d, J=12.8 Hz, 1H), 3.09 (t, J=12.3 Hz, 1H), 2.92 (t, J=16.1 Hz, 3H), 2.70 (m, 1H), 2.50 (t, J=1.9 Hz, 7H), 2.08 (t, J=10.5 Hz, 3H), 1.92 (q, J=13.2 Hz, 2H), 1.73 (m, 8H), 1.40 (d, J=5.9 Hz, 6H).

### Example 32

Compound of Example 32 was prepared substantially following the synthesis of Example 30.

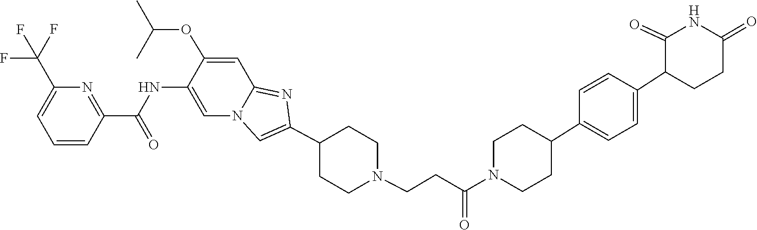 [SMILES: CC(C)Oc1cc2nc(C3CCN(CCC(=O)N4CCC(c5ccc(C6CCC(=O)NC6=O)cc5)CC4)CC3)cn2cc1NC(=O)c1cccc(C(F)(F)F)n1]

N-[2-[1-[3-[4-[4-(2,6-dioxo-3-piperidyl)phenyl]-1-piperidyl]-3-oxo-propyl]-4-piperidyl]-7-isopropoxy-imidazo[1,2-a]pyridin-6-yl]-6-(trifluoromethyl)pyridine-2-carboxamide. LC-MS (ES⁺): m/z 774.50 [M+H]⁺. ¹H NMR (400 MHz, DMSO-d₆) δ 10.82 (s, 1H), 10.56 (s, 1H), 9.77 (s, 1H), 9.30 (s, 1H), 8.47 (m, J=5.0 Hz, 2H), 8.30 (q, J=2.9 Hz, 1H), 8.19 (s, 1H), 7.48 (s, 1H), 7.22-6.97 (m, 4H), 5.11 (m, 1H), 4.56 (d, J=13.5 Hz, 1H), 3.92 (m, 1H), 3.832 (m, 1H), 3.42 (t, J=12.2 Hz, 3H), 3.16 (t, J=11.0 Hz, 4H), 2.92 (t, J=5.4 Hz, 2H), 2.81 (s, 1H), 2.67 (m, 2H), 2.31 (m, 2H), 2.17 (m, 2H), 2.04 (m, 1H), 1.86 (m, 4H), 1.62 (s, 1H), 1.47 (m, 7H).

### Example 33

Compound of Example 33 was prepared substantially following the synthesis of Example 30.

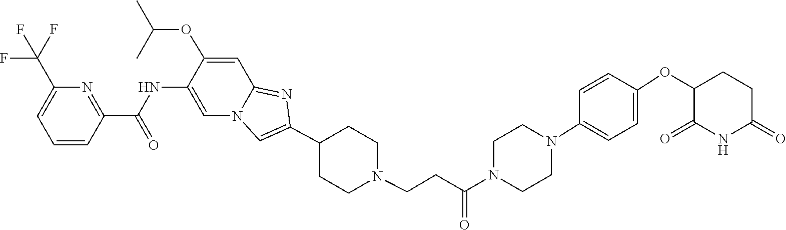 [SMILES: CC(C)Oc1cc2nc(C3CCN(CCC(=O)N4CCN(c5ccc(OC6CCC(=O)NC6=O)cc5)CC4)CC3)cn2cc1NC(=O)c1cccc(C(F)(F)F)n1]

N-[2-[1-[3-[4-[4-[(2,6-dioxo-3-piperidyl)oxy]phenyl]piperazin-1-yl]-3-oxo-propyl]-4-piperidyl]-7-isopropoxy-imidazo[1,2-a]pyridin-6-yl]-6-(trifluoromethyl)pyridine-2-carboxamide. LC-MS (ES⁺): m/z 791.45 [M+H]⁺. ¹H NMR (400 MHz, DMSO-d₆) δ 10.90 (s, 1H), 10.56 (s, 1H), 9.77 (s, 1H), 9.30 (s, 1H), 8.47 (q, J=8.3 Hz, 2H), 8.30 (d, J=7.2 Hz, 1H), 8.18 (s, 1H), 7.48 (s, 1H), 6.94 (m, 5H), 5.08 (m, 2H), 3.68 (m, 3H), 3.14 (m, 6H), 3.02 (s, 1H), 2.94 (t, J=6.9 Hz, 3H), 2.68 (m, 3H), 2.31 (t, J=8.0 Hz, 2H), 2.14 (m, 3H), 1.84 (m, 2H), 1.47 (d, J=6.0 Hz, 6H).

### Example 34

Compound of Example 34 was prepared substantially following the synthesis of Example 30, except starting with N-[2-(azetidin-3-yl)-7-isopropoxy-imidazo[1,2-a]pyridin-6-yl]-6-(trifluoromethyl)pyridine-2-carboxamide.

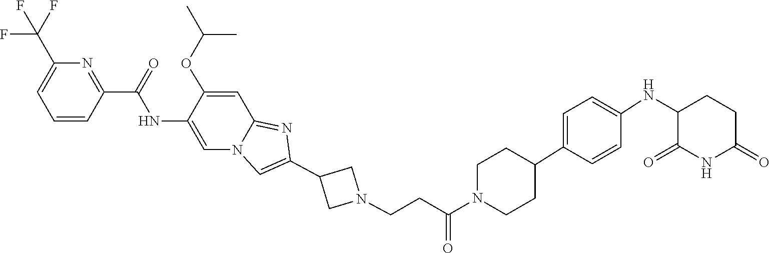 [SMILES: CC(C)Oc1cc2nc(C3CN(CCC(=O)N4CCC(c5ccc(NC6CCC(=O)NC6=O)cc5)CC4)C3)cn2cc1NC(=O)c1cccc(C(F)(F)F)n1]

N-[2-[1-[3-[4-[4-[(2,6-dioxo-3-piperidyl)amino]phenyl]-1-piperidyl]-3-oxo-propyl]azetidin-3-yl]-7-isopropoxy-imidazo[1,2-a]pyridin-6-yl]-6-(trifluoromethyl)pyridine-2-carboxamide. LC-MS (ES⁺): m/z 761.50 [M+H]⁺. ¹H NMR (401 MHz, DMSO-d₆) δ 10.76 (s, 1H), 10.45 (s, 1H), 9.44 (s, 1H), 8.43 (q, J=8.2 Hz, 2H), 8.25 (d, J=7.2 Hz, 1H), 7.75 (s, 1H), 7.16 (s, 1H), 6.95 (d, J=8.4 Hz, 2H), 6.61 (d, J=8.5 Hz, 2H), 5.66 (d, J=7.5 Hz, 1H), 4.88 (m, J=5.8 Hz, 1H), 4.52 (d, J=13.5 Hz, 1H), 4.26 (m, 1H), 3.96 (d, J=12.6 Hz, 1H), 3.61 (s, 4H), 3.18 (s, 2H), 3.07 (t, J=12.5 Hz, 1H), 2.67 (m, 5H), 2.36 (m, 2H), 2.10 (m, 2H), 1.90 (s, 4H), 1.73 (q, J=10.7 Hz, 1H), 1.41 (d, J=6.0 Hz, 6H).

### Synthesis of N-[2-[4-(hydroxymethyl)cyclohexyl]-7-isopropoxy-imidazo[1,2-a]pyridin-6-yl]-6-(trifluoromethyl)pyridine-2-carboxamide

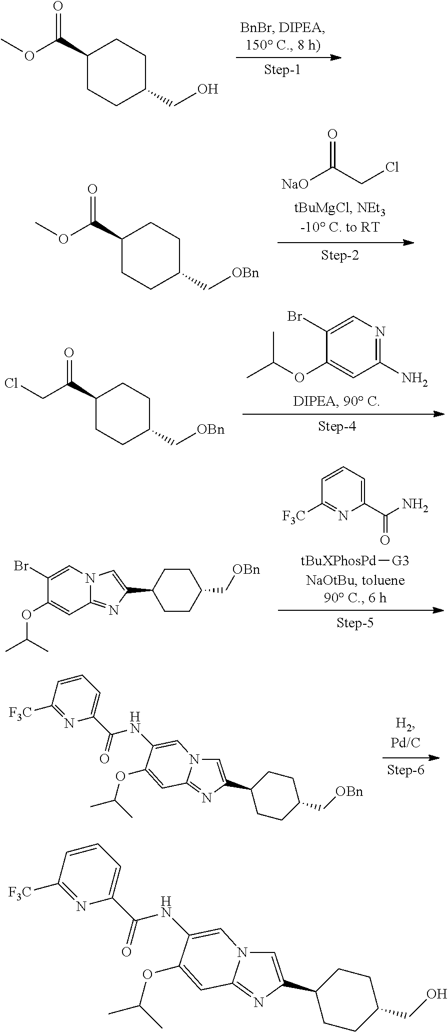

### Step-1

In a sealed tube, a stirred solution of methyl 4-(hydroxymethyl)cyclohexane carboxylate (10 g, 58.06 mmol) and bromomethylbenzene (17.28 g, 101.03 mmol, 12 mL) in DIPEA (16.32 g, 126.31 mmol, 22 mL) was stirred at 130° C. for 8 h. After complete consumption of starting material, the reaction mixture was cooled to room temperature and diluted with water and extracted with EtOAc. The combined organic extracts were washed with water, brine solution, dried over anhydrous Na₂SO₄, filtered and concentrated under reduced pressure to give the crude product which was purified by flash column chromatography, over silica (100-200 mesh) using 0-10% EtOAc in pet ether as an eluent to afford methyl 4-(benzyloxymethyl)cyclohexanecarboxylate (11.5 g, 43.84 mmol, 75.49% yield) as yellow liquid. ¹H NMR (400 MHz, DMSO-d₆) δ 7.31-7.26 (m, 5H), 4.56 (s, 2H), 3.65 (s, 3H), 3.26 (d, J=6.4 Hz 2H), 2.27-2.23 (m, 1H), 2.21-1.97 (m, 2H), 2.01-1.97 (m, 2H), 1.89 (m, 1H), 1.56 (m, 2H), 1.01 (m, 2H).

### Step-2

To a stirred solution of methyl 4-(benzyloxymethyl)cyclohexanecarboxylate (11.5 g, 43.84 mmol) in THF (170 mL) was added sodium chloroacetate (20.42 g, 175.36 mmol) and TEA (17.74 g, 175.36 mmol, 24.44 mL) then the reaction mixture was cooled to −10° C. and tert-butylmagnesium chloride solution (2.0 M in THF) (20.49 g, 175.36 mmol, 161 mL) was slowly added. The temperature was raised to 25° C. and stirred for 4 h. After complete consumption of starting material, the reaction mixture was quenched with saturated cold ammonium chloride, extracted with ethyl acetate two times, the ethyl acetate layer was washed with brine, dried over anhydrous Na₂SO₄ and concentrated to a afford 1-[4-(benzyloxymethyl)cyclohexyl]-2-chloro-ethanone (11 g, 39.18 mmol, 89.36% yield) as yellow liquid. ¹H NMR (400 MHz, DMSO-d₆) δ 7.36-7.26 (m, 5H), 4.56 (s, 2H), 3.65 (s, 3H), 3.28 (d, J=6.4 Hz 2H), 2.27-2.23 (m, 1H), 2.21-1.97 (m, 1H), 2.01-1.97 (m, 2H), 1.89 (m, 1H), 1.56 (m, 2H), 1.01 (m, 2H).

### Step-3

In a sealed tube, a stirred solution of 5-bromo-4-isopropoxy-pyridin-2-amine (6 g, 25.96 mmol) and 1-[4-(benzyloxymethyl)cyclohexyl]-2-chloro-ethanone (9.75 g, 34.72 mmol) in ethanol (5 mL) and DIPEA (15.58 g, 120.57 mmol, 21 mL) was added, then heated to 95° C. for 16 h. After complete consumption of starting material, the reaction mixture was cooled to room temperature and diluted with water and extracted with EtOAc. The combined organic extracts were washed with water, brine solution, dried over anhydrous Na₂SO₄, filtered, concentrated under reduced pressure and purification by flash column chromatography, over silica 100-200 mesh using 0-5% MeOH in DCM as an eluent afforded 2-[4-(benzyloxymethyl)cyclohexyl]-6-bromo-7-isopropoxy-imidazo[1,2-a]pyridine (5 g, 10.06 mmol, 38.73% yield) as a brown liquid. LC-MS (ES⁺): m/z 457.30 [M+H]⁺

### Step-4

In sealed tube, the stirred solution of 2-[4-(benzyloxymethyl)cyclohexyl]-6-bromo-7-isopropoxy-imidazo[1,2-a]pyridine (5 g, 10.93 mmol), 6-(trifluoromethyl)pyridine-2-carboxamide (3.25 g, 17.09 mmol) in toluene (280 mL) and sodium 2-methylpropan-2-olate (2.10 g, 21.86 mmol) was degassed with argon for 15 minutes. tBuXPhos Pd G3 (1.74 g, 2.19 mmol) was added to the reaction mixture and again degassed for 5 minutes. The reaction mixture was then heated at 90° C. for 5 h. The reaction mixture was filtered through a celite bed and the filtrate was concentrated, the crude compound was purified by silica gel column chromatography, mesh 100-200, and the product eluted with 10% methanol in DCM column chromatography to afford N-[2-[4-(benzyloxymethyl)cyclohexyl]-7-isopropoxy-imidazo[1,2-a]pyridin-6-yl]-6-(trifluoromethyl)pyridine-2-carboxamide (4 g, 6.04 mmol, 55.22% yield). LC-MS (ES⁺): m/z 565.22 [M+H]⁺.

### Step-5

To a stirred solution of N-[2-[4-(benzyloxymethyl)cyclohexyl]-7-isopropoxy-imidazo[1,2-a]pyridin-6-yl]-6-(trifluoromethyl)pyridine-2-carboxamide (4 g, 7.06 mmol) in a mixture of methanol (50 mL) and ethanol (50 mL) was purged with hydrogen gas followed by addition of palladium, 10% on carbon (4 g, 37.59 mmol) and conc. HCl (254.14 mg, 7.06 mmol, 2 mL) and the reaction mixture stirred under hydrogen atmosphere (1 atm pressure rubber bladder filled with hydrogen gas) at room temperature for 5 hr. After complete consumption of the starting material, the reaction mixture was filtered through a celite bed and washed with methanol (50 mL×2). The filtrate was concentrated to furnish N-[2-[4-(hydroxymethyl)cyclohexyl]-7-isopropoxy-imidazo[1,2-a]pyridin-6-yl]-6-(trifluoromethyl) pyridine-2-carboxamide (2.8 g, 5.35 mmol, 75.74% yield). LC-MS (ES⁺): m/z 477.30 [M+H]⁺.

### Example 35

### Synthesis of N-[2-[4-[[4-[4-(2,6-dioxo-3-piperidyl)phenyl]-1-piperidyl]methyl]cyclohexyl]-7-isopropoxy-imidazo[1,2-a]pyridin-6-yl]-6-(trifluoromethyl)pyridine-2-carboxamide

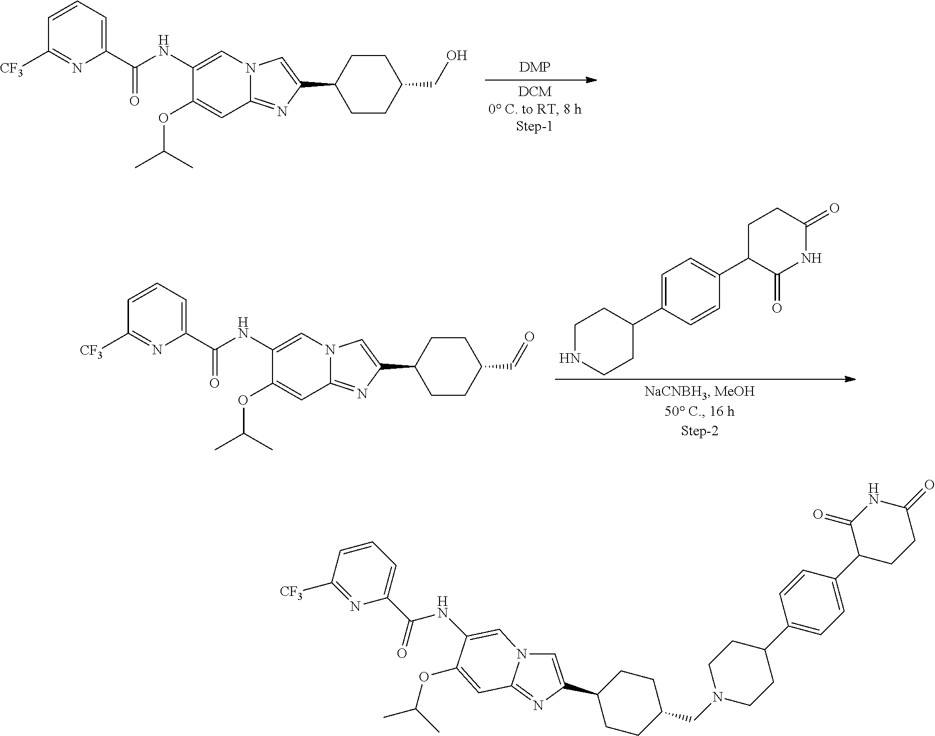

### Step-1

To a stirred solution of N-[2-[4-(hydroxymethyl)cyclohexyl]-7-isopropoxy-imidazo[1,2-a]pyridin-6-yl]-6-(trifluoromethyl)pyridine-2-carboxamide (1 g, 2.10 mmol) in DCM (25 mL) was added Dess-Martin periodinane (2.67 g, 6.30 mmol) at 0-5° C., the reaction temp was raised to 25° C. and stirred for 8 h. After completion of the reaction, the reaction was quenched with saturated cold sodium bicarbonate, extracted with DCM two times, the DCM layer was washed with brine, dried over anhydrous Na₂SO₄ and concentrated to afford N-[2-(4-formylcyclohexyl)-7-isopropoxy-imidazo[1,2-a]pyridin-6-yl]-6-(trifluoromethyl)pyridine-2-carboxamide (0.7 g, 811.42 μmol, 38.66% yield) as yellow solid. LC-MS (ES⁺): m/z 475.34 [M+H]⁺.

### Step-2

To a stirred solution of N-[2-(4-formylcyclohexyl)-7-isopropoxy-imidazo[1,2-a]pyridin-6-yl]-6-(trifluoromethyl)pyridine-2-carboxamide (0.1 g, 210.76 μmol) and 3-[4-(4-piperidyl)phenyl]piperidine-2,6-dione trifluoroacetic acid salt (52.93 mg, 136.99 μmol) in methanol (5 mL) stirred at 60° C. for 2 h, then the reaction mixture was cooled to 20° C. and sodium cyanoborohydride (35 mg, 556.95 μmol) was added, then stirred at 25° C. for 16 hours. After completion of the reaction, methanol was evaporated and the residue was purified by HPLC to afford N-[2-[4-[[4-[4-(2,6-dioxo-3-piperidyl)phenyl]-1-piperidyl]methyl]cyclohexyl]-7-isopropoxy-imidazo[1,2-a]pyridin-6-yl]-6-(trifluoromethyl)pyridine-2-carboxamide formic acid salt (18.3 mg, 22.85 μmol, 10.84% yield) as white solid. LC-MS (ES⁺): m/z 731.34 [M+H]⁺. ¹H NMR (400 MHz, DMSO-d₆) δ 10.80 (d, J=6.2 Hz, 1H), 10.47 (s, 1H), 9.50 (s, 1H), 8.44 (m, 2H), 8.26 (d, J=6.9 Hz, 1H), 8.13 (s, 1H), 7.73 (s, 1H), 7.12 (d, J=24.4 Hz, 2H), 6.52 (m, 4H), 5.90 (s, 1H), 4.93 (s, 1H), 4.31 (m, 1H), 3.60 (s, 1H), 3.07 (m, 7H), 2.50 (m, 4H), 2.10 (t, J=11.4 Hz, 3H), 1.94-1.71 (m, 9H), 1.42 (m, 8H), 1.15 (t, J=11.6 Hz, 2H).

### Preparative HPLC Conditions:

Column/dimensions: X SELECT C18 (19*250, Sum)
Mobile phase A: 0.05% TFA IN WATER
Mobile phase B: 100% Acetonitrile
Gradient (Time/% B) 0/10, 4/10, 26/38.3, 26.10/95, 28/95, 28.1/10, 30.
Flow rate: 16 ml/min
Solubility: WATER+ACN+THF

### Example 36

Compound of Example 36 was prepared substantially following the synthesis of Example 35.

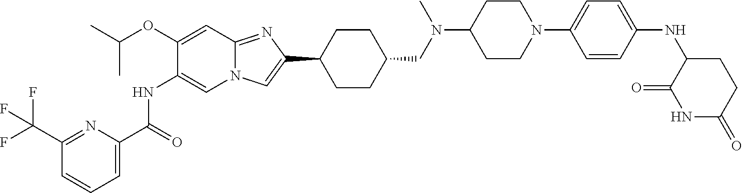 [SMILES: CC(C)Oc1cc2nc([C@H]3CC[C@H](CN(C)C4CCN(c5ccc(NC6CCC(=O)NC6=O)cc5)CC4)CC3)cn2cc1NC(=O)c1cccc(C(F)(F)F)n1]

N-[2-[4-[[[1-[4-[(2,6-dioxo-3-piperidyl)amino]phenyl]-4-piperidyl]-methyl-amino]methyl]cyclohexyl]-7-isopropoxy-imidazo[1,2-a]pyridin-6-yl]-6-(trifluoromethyl)pyridine-2-carboxamide. LC-MS (ES⁺): m/z 775.16 [M+H]⁺. ¹H NMR (400 MHz, DMSO-d₆) δ 10.75 (s, 1H), 10.44 (s, 1H), 9.43 (s, 1H), 8.43 (q, J=8.5 Hz, 1H), 8.23 (t, J=6.6 Hz, 2H), 7.66 (d, J=29.2 Hz, 1H), 7.12 (d, J=13.3 Hz, 1H), 6.76-6.53 (m, 9H), 5.36 (m, 1H), 4.87 (m, 1H), 4.18 (m, 1H), 3.34 (m, 2H), 2.67 (m, 2H), 2.58-2.38 (m, 3H), 2.23 (m, 5H), 2.08 (m, J=7.1 Hz, 2H), 1.82 (m, 5H), 1.56 (d, J=5.9 Hz, 9H), 1.00 (t, J=11.3 Hz, 2H).

### Example 37

Compound of Example 37 was prepared substantially following the synthesis of Example 35.

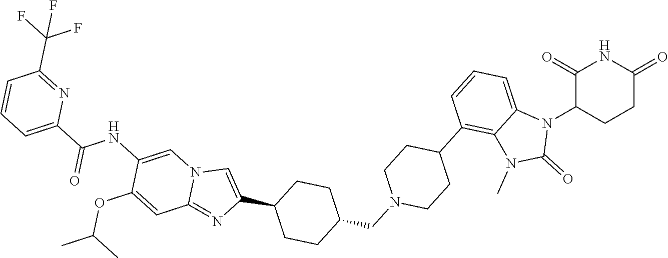 [SMILES: CC(C)Oc1cc2nc([C@H]3CC[C@H](CN4CCC(c5cccc6c5n(C)c(=O)n6C5CCC(=O)NC5=O)CC4)CC3)cn2cc1NC(=O)c1cccc(C(F)(F)F)n1]

N-[2-[4-[[4-[1-(2,6-dioxo-3-piperidyl)-3-methyl-2-oxo-benzimidazol-4-yl]-1-piperidyl]methyl]cyclohexyl]-7-isopropoxy-imidazo[1,2-a]pyridin-6-yl]-6-(trifluoromethyl)pyridine-2-carboxamide. LC-MS (ES⁺): m/z 801.45 [M+H]⁺. ¹H NMR (400 MHz, DMSO-d₆) δ 11.09 (s, 1H), 10.44 (s, 1H), 9.43 (s, 1H), 8.43 (q, J=8.2 Hz, 1H), 8.27 (t, J=14.0 Hz, 3H), 7.63 (s, 1H), 7.11 (s, 1H), 7.01 (m, 3H), 5.37 (q, J=5.9 Hz, 1H), 4.87 (m, J=6.0 Hz, 1H), 3.59 (s, 3H), 3.35 (s, 2H), 2.99 (m, 2H), 2.89 (m, 1H), 2.64 (q, J=10.9 Hz, 1H), 2.58 (q, J=10.9 Hz, 2H), 2.19 (m, 5H), 1.99 (m, 2H), 1.79 (m, 4H), 1.58 (m, 1H), 1.40 (m, 8H), 1.05 (m, 2H).

### Example 38

Compound of Example 38 was prepared substantially following the synthesis of Example 35.

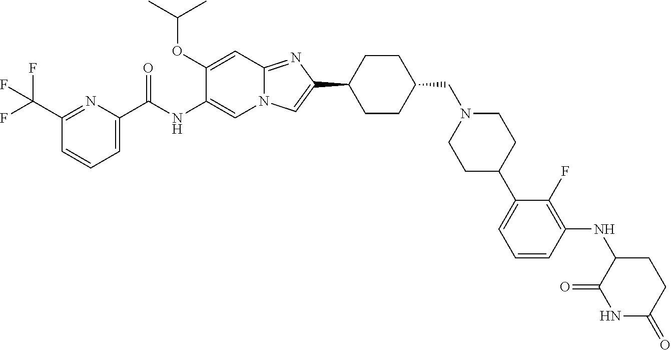 [SMILES: CC(C)Oc1cc2nc([C@H]3CC[C@H](CN4CCC(c5cccc(NC6CCC(=O)NC6=O)c5F)CC4)CC3)cn2cc1NC(=O)c1cccc(C(F)(F)F)n1]

N-[2-[4-[[4-[3-[(2,6-dioxo-3-piperidyl)amino]-2-fluoro-phenyl]-1-piperidyl]methyl]cyclohexyl]-7-isopropoxy-imidazo[1,2-a]pyridin-6-yl]-6-(trifluoromethyl)pyridine-2-carboxamide. LC-MS (ES⁺): m/z 764.39 [M+H]⁺. ¹H NMR (400 MHz, DMSO-d₆) δ 10.83 (s, 1H), 10.44 (s, 1H), 9.43 (s, 1H), 8.43 (q, J=8.2 Hz, 2H), 8.22-8.19 (m, 3H), 7.63 (s, 1H), 7.11 (s, 1H), 6.90 (t, J=7.8 Hz, 1H), 6.67-6.55 (m, 3H), 5.48 (d, J=5.4 Hz, 1H), 4.87 (m, 1H), 4.37 (t, J=8.3 Hz, 1H), 2.97 (d, J=9.7 Hz, 2H), 2.75 (q, J=5.8 Hz, 2H), 2.50 (t, J=1.6 Hz, 2H), 2.18 (m, 8H), 1.90 (d, J=11.1 Hz, 2H), 1.66 (m, 4H), 1.40 (m, 6H), 1.03 (q, J=11.1 Hz, 2H).

### Example 39

Compound of Example 39 was prepared substantially following the synthesis of Example 35.

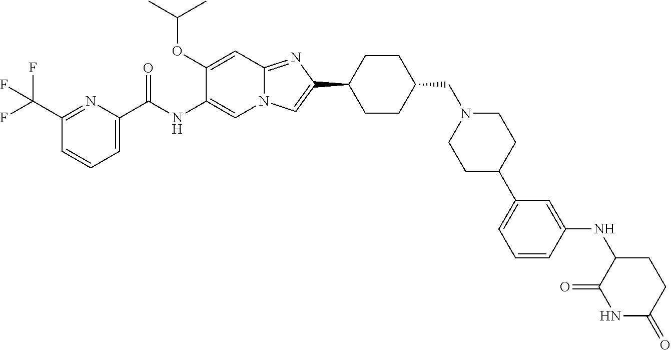 [SMILES: CC(C)Oc1cc2nc([C@H]3CC[C@H](CN4CCC(c5cccc(NC6CCC(=O)NC6=O)c5)CC4)CC3)cn2cc1NC(=O)c1cccc(C(F)(F)F)n1]

N-[2-[4-[[4-[3-[(2,6-dioxo-3-piperidyl)amino]phenyl]-1-piperidyl]methyl]cyclohexyl]-7-isopropoxy-imidazo[1,2-a]pyridin-6-yl]-6-(trifluoromethyl)pyridine-2-carboxamide. LC-MS (ES⁺): m/z 746.37 [M+H]⁺. ¹H NMR (400 MHz, DMSO-d₆) δ 10.76 (s, 1H), 10.44 (s, 1H), 9.43 (s, 1H), 8.43 (m, 2H), 8.27-8.23 (m, 3H), 7.62 (s, 1H), 7.11 (s, 1H), 6.98 (t, J=7.8 Hz, 1H), 6.57 (s, 1H), 6.47 (t, J=10.0 Hz, 3H), 5.71 (d, J=7.6 Hz, 1H), 4.87 (m, 1H), 4.34 (m, 1H), 2.94 (d, J=10.9 Hz, 2H), 2.74 (q, J=5.8 Hz, 1H), 2.50 (d, J=1.8 Hz, 1H), 2.33 (t, J=1.8 Hz, 2H), 2.10-2.07 (m, 5H), 1.90-1.55 (m, 5H), 1.63-1.55 (m, 4H), 1.40 (d, J=6.0 Hz, 6H), 1.02 (d, J=11.4 Hz, 2H).

### Example 40

Compound of Example 40 was prepared substantially following the synthesis of Example 35.

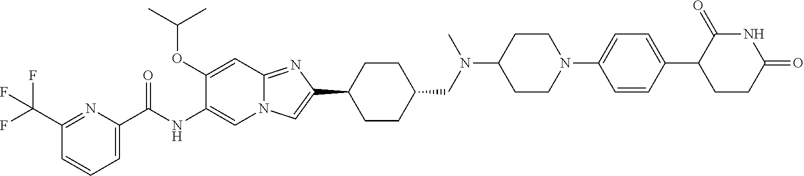 [SMILES: CC(C)Oc1cc2nc([C@H]3CC[C@H](CN(C)C4CCN(c5ccc(C6CCC(=O)NC6=O)cc5)CC4)CC3)cn2cc1NC(=O)c1cccc(C(F)(F)F)n1]

N-[2-[4-[[[1-[4-(2,6-dioxo-3-piperidyl)phenyl]-4-piperidyl]-methyl-amino]methyl]cyclohexyl]-7-isopropoxy-imidazo[1,2-a]pyridin-6-yl]-6-(trifluoromethyl)pyridine-2-carboxamide. LC-MS (ES⁺): m/z 760.29 [M+H]⁺. ¹H NMR (400 MHz, DMSO-d₆) δ 12.71 (s, 1H), 10.77 (d, J=4.9 Hz, 1H), 10.45 (s, 1H), 9.45 (s, 1H), 8.43 (q, J=8.0 Hz, 2H), 8.25 (t, J=4.3 Hz, 1H), 8.13 (s, 2H), 7.66 (s, 1H), 7.08 (m, 3H), 6.91 (t, J=8.8 Hz, 2H), 6.52 (m, 5H), 4.89 (m, 1H), 3.76 (m, 1H), 2.89 (m, 3H), 2.63 (m, 3H), 2.02-1.72 (m, 9H), 1.41 (m, 9H), 1.16 (m, 2H).

### Example 41

Compound of Example 41 was prepared substantially following the synthesis of Example 35.

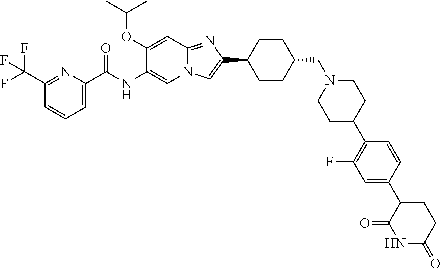 [SMILES: CC(C)Oc1cc2nc([C@H]3CC[C@H](CN4CCC(c5ccc(C6CCC(=O)NC6=O)cc5F)CC4)CC3)cn2cc1NC(=O)c1cccc(C(F)(F)F)n1]

N-[2-[4-[[4-[4-(2,6-dioxo-3-piperidyl)-2-fluoro-phenyl]-1-piperidyl]methyl]cyclohexyl]-7-isopropoxy-imidazo[1,2-a]pyridin-6-yl]-6-(trifluoromethyl)pyridine-2-carboxamide. LC-MS (ES⁺): m/z 749.36 [M+H]⁺. ¹H NMR (400 MHz, DMSO-d₆) δ 10.84 (s, 1H), 10.44 (s, 1H), 9.43 (s, 1H), 8.43 (m, 2H), 8.24 (t, J=4.2 Hz, 2H), 7.63 (s, 1H), 7.31 (t, J=8.1 Hz, 1H), 7.11 (s, 1H), 7.03 (t, J=8.2 Hz, 2H), 4.87 (m, 1H), 3.86 (q, J=5.6 Hz, 1H), 2.96 (d, J=11.0 Hz, 2H), 2.69 (m, 6H), 2.20 (m, 3H), 1.98 (m, 6H), 1.72-1.57 (m, 5H), 1.40 (m, 6H), 1.02 (m, 2H).

### Example 42

Compound of Example 42 was prepared substantially following the synthesis of Example 35.

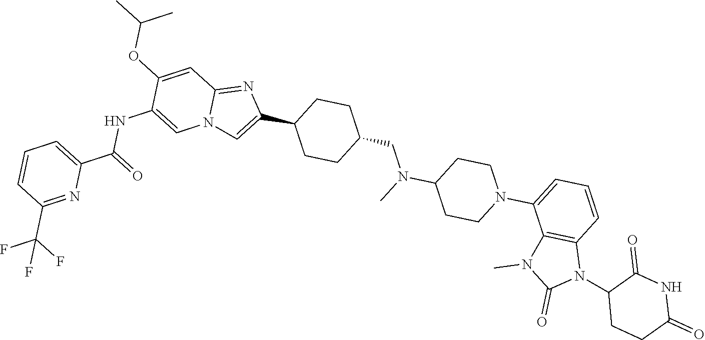 [SMILES: CC(C)Oc1cc2nc([C@H]3CC[C@H](CN(C)C4CCN(c5cccc6c5n(C)c(=O)n6C5CCC(=O)NC5=O)CC4)CC3)cn2cc1NC(=O)c1cccc(C(F)(F)F)n1]

N-[2-[4-[[[1-[1-(2,6-dioxo-3-piperidyl)-3-methyl-2-oxo-benzimidazol-4-yl]-4-piperidyl]-methyl-amino]methyl]cyclohexyl]-7-isopropoxy-imidazo[1,2-a]pyridin-6-yl]-6-(trifluoromethyl)pyridine-2-carboxamide. LC-MS (ES⁺): m/z 830.10 [M+H]⁺. ¹H NMR (400 MHz, DMSO-d₆) δ 14.04 (s, 1H), 11.10 (s, 1H), 10.56 (s, 1H), 9.78 (s, 1H), 9.01 (s, 1H), 8.47 (q, J=8.3 Hz, 2H), 8.30 (d, J=7.2 Hz, 1H), 8.12 (s, 1H), 7.41 (s, 1H), 7.06 (m, 4H), 5.37 (q, J=5.9 Hz, 1H), 5.12 (m, 1H), 3.66 (s, 3H), 3.26 (m, 4H), 2.84 (m, 8H), 2.50 (m, 2H), 2.13-1.90 (m, 8H), 1.47 (m, 6H), 1.25 (m, 3H).

### Example 43

Compound of Example 43 was prepared substantially following the synthesis of Example 35.

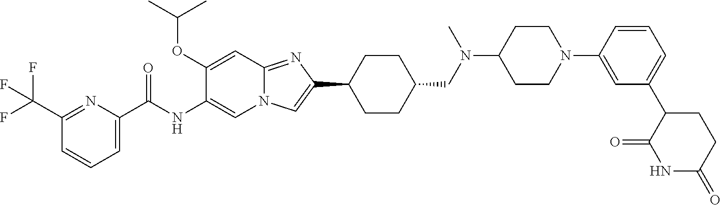 [SMILES: CC(C)Oc1cc2nc([C@H]3CC[C@H](CN(C)C4CCN(c5cccc(C6CCC(=O)NC6=O)c5)CC4)CC3)cn2cc1NC(=O)c1cccc(C(F)(F)F)n1]

N-[2-[4-[[[1-[3-(2,6-dioxo-3-piperidyl)phenyl]-4-piperidyl]-methyl-amino]methyl]cyclohexyl]-7-isopropoxy-imidazo[1,2-a]pyridin-6-yl]-6-(trifluoromethyl)pyridine-2-carboxamide. LC-MS (ES⁺): m/z 760.12 [M+H]⁺. ¹H NMR (400 MHz, DMSO-d₆) δ 10.78 (d, J=7.8 Hz, 1H), 10.44 (s, 1H), 9.42 (s, 1H), 8.42-8.24 (m, 4H), 7.66 (d, J=27.9 Hz, 1H), 7.13 (q, J=7.6 Hz, 2H), 6.81 (t, J=8.2 Hz, 3H), 6.59 (d, J=7.3 Hz, 1H), 4.87 (m, 1H), 3.73 (q, J=7.9 Hz, 3H), 2.63 (m, 4H), 2.44 (m, 2H), 2.22 (m, 6H), 2.04 (t, J=9.6 Hz, 2H), 1.88 (d, J=10.6 Hz, 2H), 1.74 (m, 2H), 1.40 (m, 11H), 0.99 (m, 2H).

### Example 44

Compound of Example 44 was prepared substantially following the synthesis of Example 35.

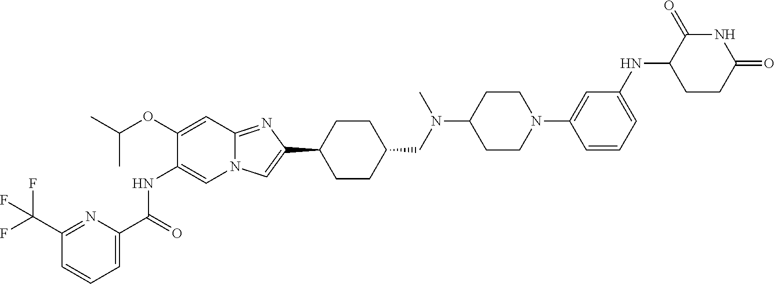 [SMILES: CC(C)Oc1cc2nc([C@H]3CC[C@H](CN(C)C4CCN(c5cccc(NC6CCC(=O)NC6=O)c5)CC4)CC3)cn2cc1NC(=O)c1cccc(C(F)(F)F)n1]

N-[2-[4-[[[1-[3-[(2,6-dioxo-3-piperidyl)amino]phenyl]-4-piperidyl]-methyl-amino]methyl]cyclohexyl]-7-isopropoxy-imidazo[1,2-a]pyridin-6-yl]-6-(trifluoromethyl) pyridine-2-carboxamide. LC-MS (ES⁺): m/z 775.16 [M+H]⁺. ¹H NMR (400 MHz, DMSO-d₆) δ 10.75 (d, J=7.7 Hz, 1H), 10.44 (s, 1H), 9.42 (s, 1H), 8.41 (m, 2H), 8.35-8.24 (m, 3H), 7.66 (d, J=28.2 Hz, 1H), 7.12 (d, J=12.4 Hz, 1H), 6.90 (t, J=7.9 Hz, 1H), 6.26-6.10 (m, 3H), 5.60 (d, J=7.5 Hz, 1H), 4.87 (t, J=6.0 Hz, 1H), 4.29 (q, J=5.5 Hz, 1H), 3.66 (d, J=10.4 Hz, 1H), 2.73 (m, 2H), 2.59 (q, J=10.0 Hz, 3H), 2.37 (d, J=34.5 Hz, 1H), 2.21 (m, 5H), 2.08 (t, J=16.5 Hz, 3H), 1.87 (t, J=11.0 Hz, 3H), 1.76 (t, J=19.1 Hz, 3H), 1.40 (m, 10H), 0.99 (m, 1H).

### Example 45

Compound of Example 45 was prepared substantially following the synthesis of Example 35.

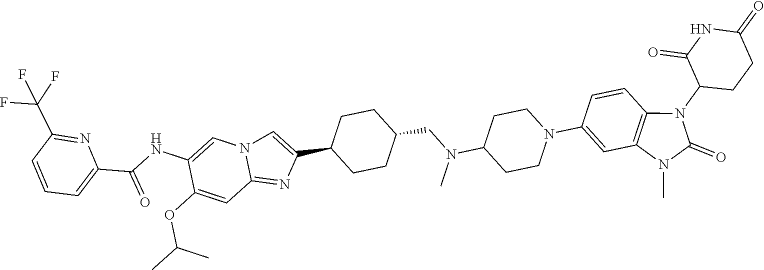 [SMILES: CC(C)Oc1cc2nc([C@H]3CC[C@H](CN(C)C4CCN(c5ccc6c(c5)n(C)c(=O)n6C5CCC(=O)NC5=O)CC4)CC3)cn2cc1NC(=O)c1cccc(C(F)(F)F)n1]

N-[2-[4-[[[1-[1-(2,6-dioxo-3-piperidyl)-3-methyl-2-oxo-benzimidazol-5-yl]-4-piperidyl]-methyl-amino]methyl]cyclohexyl]-7-isopropoxy-imidazo[1,2-a]pyridin-6-yl]-6-(trifluoromethyl)pyridine-2-carboxamide. LC-MS (ES⁺): m/z 831.10 [M+H]⁺. ¹H NMR (400 MHz, DMSO-d₆) δ 13.97 (s, 1H), 11.07 (s, 1H), 10.55 (s, 1H), 9.76 (s, 1H), 8.94 (s, 1H), 8.48 (t, J=8.2 Hz, 2H), 8.30 (d, J=7.5 Hz, 1H), 8.10 (s, 1H), 7.39 (s, 1H), 7.21-6.96 (m, 9H), 6.87 (s, 1H), 6.68 (d, J=8.2 Hz, 1H), 5.30 (t, J=6.4 Hz, 1H), 5.11 (s, 1H), 3.79 (d, J=11.3 Hz, 2H), 3.35-3.17 (m, 5H), 2.07 (m, 7H), 1.87 (t, J=13.6 Hz, 4H), 1.47 (m, 7H), 1.19 (t, J=18.0 Hz, 2H).

### Example 46

Compound of Example 46 was prepared substantially following the synthesis of Example 35.

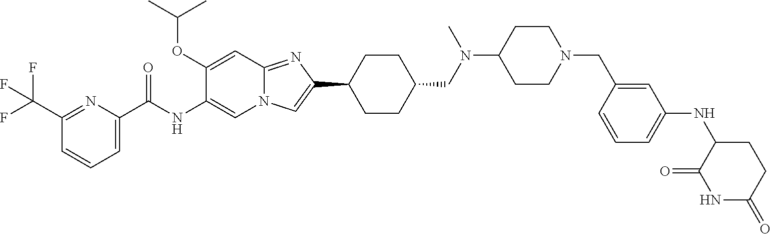 [SMILES: CC(C)Oc1cc2nc([C@H]3CC[C@H](CN(C)C4CCN(Cc5cccc(NC6CCC(=O)NC6=O)c5)CC4)CC3)cn2cc1NC(=O)c1cccc(C(F)(F)F)n1]

N-[2-[4-[[[1-[[3-[(2,6-dioxo-3-piperidyl)amino]phenyl]methyl]-4-piperidyl]-methyl-amino]methyl]cyclohexyl]-7-isopropoxy-imidazo[1,2-a]pyridin-6-yl]-6-(trifluoromethyl) pyridine-2-carboxamide. LC-MS (ES⁺): m/z 787.49 [M+H]⁺. ¹H NMR (400 MHz, DMSO-d₆) δ 10.80 (d, J=6.2 Hz, 1H), 10.47 (s, 1H), 9.50 (s, 1H), 8.44 (m, 2H), 8.26 (d, J=6.9 Hz, 1H), 8.13 (s, 1H), 7.73 (s, 1H), 7.12 (d, J=24.4 Hz, 2H), 6.52 (m, 4H), 5.90 (s, 1H), 4.93 (s, 1H), 4.31 (m, 1H), 3.60 (s, 1H), 3.07 (m, 7H), 2.50 (m, 4H), 2.10 (t, J=11.4 Hz, 3H), 1.94-1.71 (m, 9H), 1.42 (m, 8H), 1.15 (t, J=11.6 Hz, 2H).

### Example 47 Synthesis of N-[2-[4-[[4-[1-(2,6-dioxo-3-piperidyl)-3-methyl-2-oxo-benzimidazol-5-yl]-1-piperidyl]methyl]cyclohexyl]-7-isopropoxy-imidazo[1,2-a]pyridin-6-yl]-6-(trifluoromethyl)pyridine-2-carboxamide

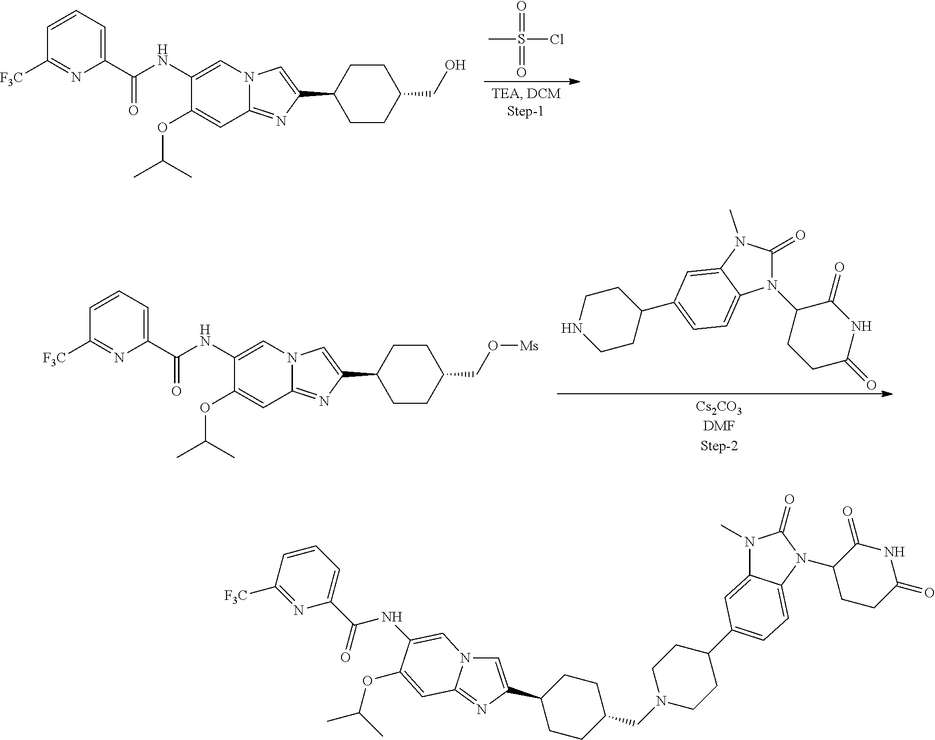

### Step-1

A stirred solution of N-[2-[4-(hydroxymethyl)cyclohexyl]-7-isopropoxy-imidazo[1,2-a]pyridin-6-yl]-6-(trifluoromethyl)pyridine-2-carboxamide (1 g, 2.10 mmol) in DCM (70 mL) was cooled to 0-5° C. and TEA (1.09 g, 10.76 mmol, 1.50 mL) was added. Methanesulfonyl chloride (2.04 g, 17.84 mmol, 1.38 mL) was added dropwise and the reaction mixture stirred at 0-5° C. for 2 hr. The reaction mixture was quenched with ice water and extracted with ethyl acetate. The organic layer was concentrated and the crude material was purified by silica gel column chromatography (0-10% methanol DCM) to afford 4-[7-isopropoxy-6-[[6-(trifluoromethyl)pyridine-2-carbonyl]amino]imidazo[1,2-a]pyridin-2-yl]cyclohexyl]methyl methanesulfonate (0.7 g, 1.19 mmol, 56.53% yield). LC-MS (ES⁺): m/z 555.63 [M+H]⁺

### Step-2

To a stirred solution of 3-[3-methyl-2-oxo-5-(4-piperidyl)benzimidazol-1-yl]piperidine-2,6-dione trifluoroacetic acid salt (123.45 mg, 270.47 μmol) in DMF (5 mL) at 25° C. was added cesium carbonate (88.13 mg, 270.47 μmol) and the mixture was stirred for 15 min before the drop wise addition of a solution of [4-[7-isopropoxy-6-[[6-(trifluoromethyl)pyridine-2-carbonyl]amino]imidazo[1,2-a]pyridin-2-yl]cyclohexyl]methyl methanesulfonate (0.1 g, 180.32 μmol) in DMF (0.5 mL) and the reaction mixture was stirred at 90° C. for 4h. DMF was evaporated and the residue was purified by HPLC to afford N-[2-[4-[[4-[1-(2,6-dioxo-3-piperidyl)-3-methyl-2-oxo-benzimidazol-5-yl]-1-piperidyl]methyl]cyclohexyl]-7-isopropoxy-imidazo[1,2-a]pyridin-6-yl]-6-(trifluoromethyl)pyridine-2-carboxamide (18.8 mg, 21.19 μmol, 11.75% yield) as white solid. LC-MS (ES⁺): m/z 801.42 [M+H]⁺. ¹H NMR (400 MHz, DMSO-d₆) δ 11.09 (s, 1H), 10.44 (s, 1H), 9.43 (s, 1H), 8.43 (q, J=8.3 Hz, 2H), 8.24 (d, J=7.2 Hz, 1H), 7.63 (s, 1H), 7.11 (s, 1H), 7.00 (m, 3H), 5.37 (q, J=5.9 Hz, 1H), 4.88 (m, 1H), 3.59 (s, 3H), 3.17 (d, J=5.2 Hz, 1H), 2.92 (m, 1H), 2.66 (m, 4H), 2.19 (d, J=6.7 Hz, 2H), 2.05 (m, 5H), 1.91 (d, J=13.6 Hz, 2H), 1.79-1.57 (m, 5H), 1.40 (m, 8H), 1.23-1.07 (m, 3H).

### Preparative HPLC Conditions:

Mobile Phase (A): 0.1% TFA in H2O
Mobile Phase (B): 100% ACETONITRILE
Flow Rate: 16 ml/min
Column: SUNFIRE C18 5 μm (19×150 mm)
Gradient Time % B: 0/15, 2/15, 10/35, 16/35, 16.1/100

### Example 48

Compound of Example 48 was prepared substantially following the synthesis of Example 47.

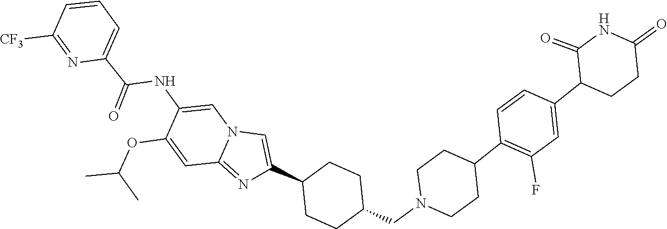 [SMILES: CC(C)Oc1cc2nc([C@H]3CC[C@H](CN4CCC(c5ccc(C6CCC(=O)NC6=O)cc5F)CC4)CC3)cn2cc1NC(=O)c1cccc(C(F)(F)F)n1]

N-[2-[4-[[4-[4-[(2,6-dioxo-3-piperidyl)amino]-2-fluoro-phenyl]-1-piperidyl]methyl]cyclohexyl]-7-isopropoxy-imidazo[1,2-a]pyridin-6-yl]-6-(trifluoromethyl)pyridine-2-carboxamide. LC-MS (ES⁺): m/z 764.12 [M+H]⁺. ¹H NMR (400 MHz, DMSO-d₆) δ 10.76 (s, 1H), 10.43 (s, 1H), 9.43 (s, 1H), 8.43 (q, J=8.2 Hz, 1H), 8.24 (d, J=7.1 Hz, 1H), 8.14 (s, 1H), 7.62 (s, 1H), 7.10 (s, 1H), 7.00 (t, J=8.8 Hz, 1H), 6.45 (t, J=8.5 Hz, 2H), 5.99 (d, J=7.4 Hz, 1H), 4.87 (t, J=5.9 Hz, 1H), 4.30 (t, J=12.0 Hz, 1H), 3.30 (s, 2H), 2.50 (m, 4H), 2.29-2.07 (m, 7H), 1.88 (q, J=9.3 Hz, 3H), 1.65-1.60 (m, 5H), 1.40 (m, 8H), 1.35-1.04 (m, 2H).

### Synthesis of methyl 2-(1-tert-butoxycarbonyl-4-piperidyl)-6-isopropoxy-indazole-5-carboxylate

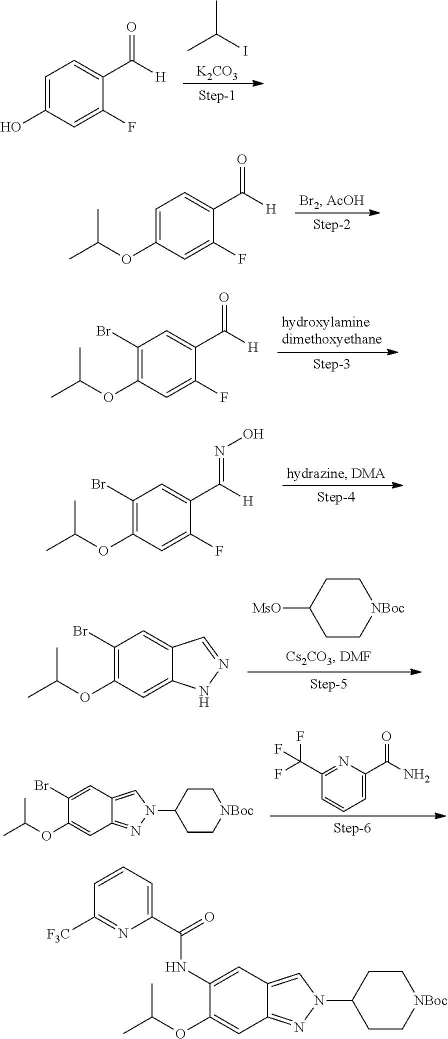

### Step-1

To a solution of 2-fluoro-4-hydroxy-benzaldehyde (20.00 g, 142.74 mmol) in DMF (200 mL) was added potassium carbonate (39.46 g, 285.49 mmol) and 2-iodopropane (26.69 g, 157.02 mmol, 15.70 mL). The reaction mixture was stirred at 80° C. for 16 hours. The reaction mixture was then diluted with water (2000 mL) and extracted with ethyl acetate (500 mL×3). The combined organic layers were washed with brine (1000 mL) and concentrated under reduced pressure. The resulting residue was purified by column chromatography (silica gel, petroleum ether/ethyl acetate=20/1 to 5/1) to afford 2-fluoro-4-isopropoxy-benzaldehyde (22 g, 120.62 mmol, 84.50% yield) as a colorless oil. LC-MS (ES⁺): m/z 183.1 [M+H]⁺.

### Step-2

To a solution of 2-fluoro-4-isopropoxy-benzaldehyde (40 g, 219.55 mmol) in acetic acid (800 mL) was added a solution of molecular bromine (38.59 g, 241.50 mmol) in acetic acid (40 mL) dropwise at 20° C. The reaction mixture was stirred at 50° C. for 16 hours. After consumption of the reactant as shown by TLC and LC-MS, the mixture was filtered and concentrated under reduced pressure to give 5-bromo-2-fluoro-4-isopropoxy-benzaldehyde (50 g, 147.46 mmol, 67.16% yield) as a yellow oil, which was used in the next step without purification. LC-MS (ES⁺): m/z 260.9 [M+H]⁺.

### Step-3

To a solution of 5-bromo-2-fluoro-4-isopropoxy-benzaldehyde (50 g, 124.48 mmol) in ethanol (500 mL) was added hydroxylamine hydrochloride (8.65 g, 124.48 mmol, 5.18 mL) and potassium carbonate (18.92 g, 136.93 mmol). The reaction mixture was stirred at 100° C. for 2 hours. After consumption of the reactant as shown by LC-MS, the reaction mixture was filtered and the filtrate was concentrated under reduce pressure to give (E)-5-bromo-2-fluoro-4-isopropoxybenzaldehyde oxime (50 g, 139.44 mmol) was obtained as a yellow oil. Used without further purification. LC-MS (ES⁺): m/z 276.0 [M+H]⁺.

### Step-4

To a solution of (E)-5-bromo-2-fluoro-4-isopropoxy-benzaldehyde oxime (50 g, 181.09 mmol) in DMA (500 mL) was added hydrazine hydrate (96.97 g, 1.94 mol, 94.15 mL). The reaction mixture was stirred at 140° C. for 16 hours. After consumption of the reactant as confirmed by LC-MS, the reaction mixture was diluted with water (1000 mL) and extracted with ethyl acetate (1000 mL×3). The combined organic layers were washed with aqueous NaCl (1000 mL×2), dried over Na₂SO₄, filtered, and the filtrate evaporated to dryness. The residue was purified by column chromatography (silica gel, petroleum ether/ethyl acetate=10/1 to 3/1) to give 5-bromo-6-isopropoxy-1H-indazole (17 g, 48.65 mmol, 26.86% yield) as a yellow oil. LC-MS (ES⁺): 254.9 m/z [M+H]⁺.

### Step-5

To a solution of 5-bromo-6-isopropoxy-1H-indazole (14 g, 54.88 mmol) in DMF (150 mL) was added dicesium carbonate (35.76 g, 109.76 mmol) and tert-butyl 4-((methylsulfonyl)oxy)piperidine-1-carboxylate (19.93 g, 71.34 mmol). The reaction mixture was stirred at 80° C. for 16 hours. The reaction mixture was diluted with water (200 mL) and extracted with ethyl acetate (200 mL*3). The combined organic layers were washed with brine (200 mL*2), dried over Na₂SO₄, filtered, and concentrated in vacuum to dryness. The residue was purified by prep-HPLC (Biotage Isolera One, I.D. 95 mm×H365 mm Welch Ultimate XB_C18 20-40 m; 120 A; Mobile phase, MeCN/H2O, Gradient B %, 30-80% 30 min; 80% 25 min). Compound tert-butyl 4-(5-bromo-6-isopropoxy-2H-indazol-2-yl)piperidine-1-carboxylate (2.1 g, 4.75 mmol, 8.66% yield) was obtained as a white solid. LC-MS (ES⁺): m/z 438.2 [M+H]⁺.

### Step-6

A vial was charged with tert-butyl 4-(5-bromo-6-isopropoxy-2H-indazol-2-yl) piperidine-1-carboxylate (7, 1.5 g, 3.42 mmol), diacetoxypalladium (153.65 mg, 684.37 μmol), Xantphos (791.98 mg, 1.37 mmol) and dicesium carbonate (2.23 g, 6.84 mmol). The vial was evacuated, backfilled with N₂, and closed with a screw cap with septa. A dioxane (45 mL) solution of 6-(trifluoromethyl)picolinamide (715.63 mg, 3.76 mmol) was added via a syringe at 20° C. The vial was sealed and heated at 100° C. for 16 h. LC-MS confirmed reactants were consumed completely and desired mass detected. The reaction mixture was filtered, and the filtrate concentrated. The residue was purified by flash silica gel chromatography (120 g Silica, Eluent of 0-70% Ethyl acetate/Petroleum ether gradient) to afford tert-butyl 4-(6-isopropoxy-5-(6-(trifluoromethyl)picolinamido)-2H-indazol-2-yl)piperidine-1-carboxylate (762.98 mg, 1.34 mmol, 39.09% yield) was obtained as a yellow solid. LC-MS (ES⁺): m/z 548.1 [M+H]⁺

### Example 49 Synthesis of N-(2-(1-(2-(4-(4-((2,6-dioxopiperidin-3-yl)amino)phenyl)piperidin-1-yl)-2-oxoethyl)piperidin-4-yl)-6-isopropoxy-2H-indazol-5-yl)-6-(trifluoromethyl) picolinamide

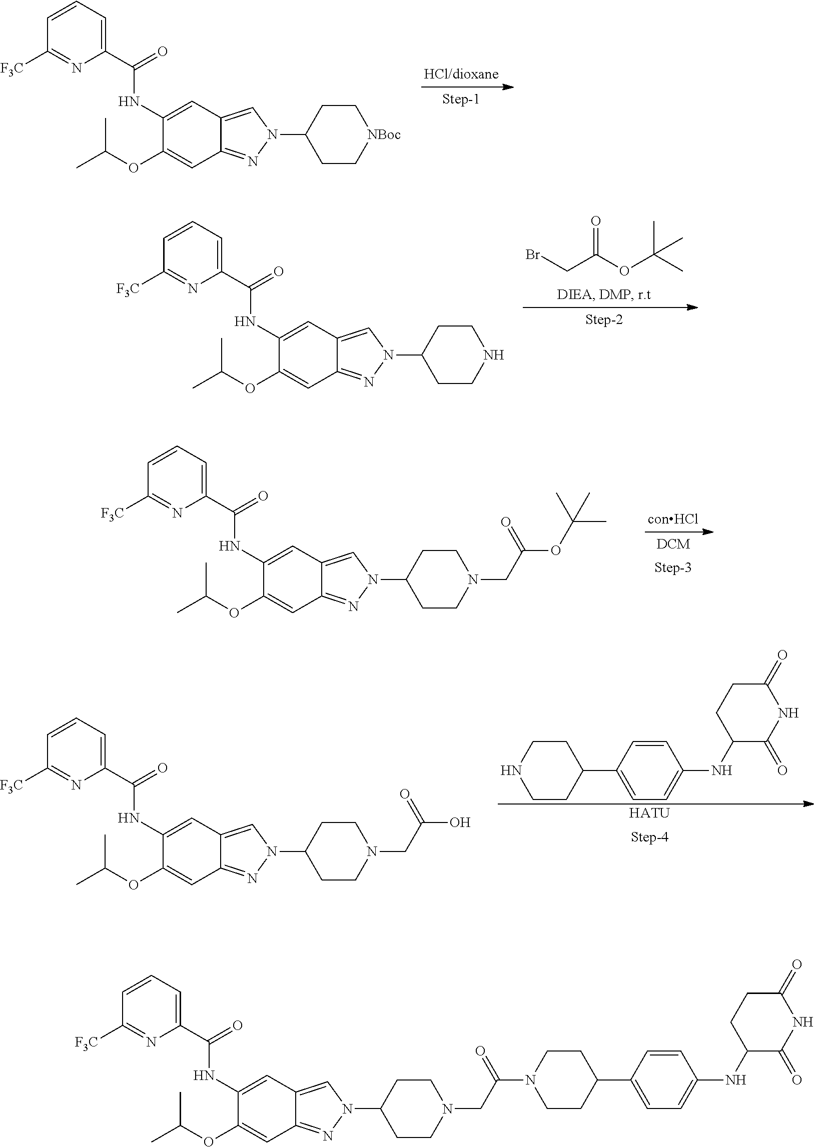

### Step 1

A solution of tert-butyl 4-[6-isopropoxy-5-[[6-(trifluoromethyl)pyridine-2-carbonyl]amino]indazol-2-yl]piperidine-1-carboxylate (650 mg, 1.19 mmol) in HCl/dioxane (10 mL) was stirred at 20° C. for 2 h. The reaction mixture was concentrated. The crude product N-[6-isopropoxy-2-(4-piperidyl)indazol-5-yl]-6-(trifluoromethyl)pyridine-2-carboxamide hydrochloride (580 mg, 1.11 mmol, 93.90% yield) was used in the next step without further purification. LC-MS (ES⁺): 448.0 [M+H]⁺

### Step 2

To a solution of N-[6-isopropoxy-2-(4-piperidyl)indazol-5-yl]-6-(trifluoromethyl)pyridine-2-carboxamide hydrochloride (180 mg, 371.97 μmol) in DMF (3 mL) was added TEA (112.92 mg, 1.12 mmol, 155.53 μL) and tert-butyl 2-bromoacetate (72.55 mg, 371.97 μmol, 54.55 μL). The mixture was stirred at 20° C. for 2 hr. The reaction was diluted with water (10 mL) and extracted with ethyl acetate (10 mL×2). The combined organic layers were dried over Na₂SO₄, filtered and concentrated under reduced pressure. The crude product tert-butyl 2-[4-[6-isopropoxy-5-[[6-(trifluoromethyl)pyridine-2-carbonyl]amino]indazol-2-yl]-1-piperidyl]acetate (3, 200 mg, 327.64 μmol, 88.08% yield) was used in the next step without further purification. LC-MS (ES⁺): 562.1 [M+H]⁺

### Step 3

To a solution of tert-butyl 2-[4-[6-isopropoxy-5-[[6-(trifluoromethyl)pyridine-2-carbonyl]amino]indazol-2-yl]-1-piperidyl]acetate (170 mg, 302.71 μmol) in DCM (3.5 mL) was added HCl (12 M, 252.26 μL). The mixture was stirred at 20° C. for 2 hours. The reaction mixture was concentrated under reduced pressure to give a residue. The crude product 2-[4-[6-isopropoxy-5-[[6-(trifluoromethyl)pyridine-2-carbonyl]amino]indazol-2-yl]-1-piperidyl]acetic acid hydrochloride (120 mg, 205.92 μmol, 68.03% yield) was used in the next step without further purification. LC-MS (ES⁺): 506.0 [M+H]⁺

### Step 4

To a solution of 2-[4-[6-isopropoxy-5-[[6-(trifluoromethyl)pyridine-2-carbonyl]amino]indazol-2-yl]-1-piperidyl]acetic acid hydrochloride (50 mg, 92.26 μmol) and 3-[4-(4-piperidyl)anilino]piperidine-2,6-dione hydrochloride (29.88 mg, 92.26 μmol) in DMF (500 μL) was added HATU (52.62 mg, 138.39 μmol) and DIPEA (71.54 mg, 553.56 μmol, 96.42 μL). The reaction mixture was stirred at 20° C. for 2 hours. The reaction mixture was directly purified by prep-HPLC (FA condition). Compound N-(2-(1-(2-(4-(4-((2,6-dioxopiperidin-3-yl)amino)phenyl)piperidin-1-yl)-2-oxoethyl)piperidin-4-yl)-6-isopropoxy-2H-indazol-5-yl)-6-(trifluoromethyl)picolinamide formic acid salt (48.41 mg, 54.26 μmol, 58.81% yield) was obtained as a white solid.

### Method for “FA Condition”

Instrument: Gilson-281
Column: Shim-pack C18 150 mm*25 mm*10 um
Mobile phase: A for H₂O (0.225% FA v/v) and B for Acetonitrile
Gradient: B 24%-64% in 13 min linearly
Flow rate: 25 ml/min
Column temperature: R.T.
Wavelength: 220 nm/254 nm

¹H NMR (400 MHz, DMSO-d₆): δ=10.77 (d, J=8.0 Hz, 2H), 9.87-9.56 (m, 1H), 8.76 (s, 1H), 8.51-8.37 (m, 3H), 8.23 (d, J=7.7 Hz, 1H), 7.22 (s, 1H), 6.97 (br d, J=8.3 Hz, 2H), 6.63 (br d, J=8.3 Hz, 2H), 5.71 (br d, J=7.4 Hz, 1H), 4.96-4.65 (m, 2H), 4.60-4.24 (m, 4H), 3.88-3.66 (m, 2H), 3.63-3.39 (m, 3H), 3.26-3.05 (m, 2H), 2.85-2.69 (m, 3H), 2.63-2.56 (m, 3H), 2.45-2.35 (m, 2H), 2.15-2.07 (m, 1H), 1.94-1.75 (m, 3H), 1.67-1.53 (m, 1H), 1.42 (d, J=6.0 Hz, 7H). LC-MS (ES⁺): m/z 775.1 [M+H]⁺

### Example 50

Compound of Example 50 was prepared substantially following the synthesis of Example 49.

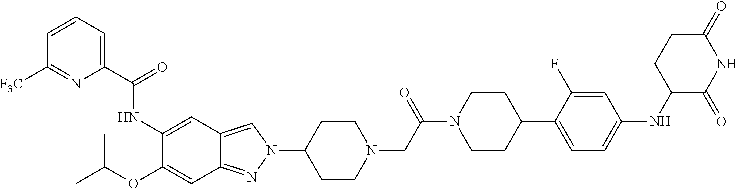 [SMILES: CC(C)Oc1cc2nn(C3CCN(CC(=O)N4CCC(c5ccc(NC6CCC(=O)NC6=O)cc5F)CC4)CC3)cc2cc1NC(=O)c1cccc(C(F)(F)F)n1]

N-[2-[1-[2-[4-[4-[(2,6-dioxo-3-piperidyl)amino]-2-fluoro-phenyl]-1-piperidyl]-2-oxo-ethyl]-4-piperidyl]-6-isopropoxy-indazol-5-yl]-6-(trifluoromethyl)pyridine-2-carboxamide ¹H NMR (400 MHz, DMSO-d₆): δ=10.78 (d, J=15.9 Hz, 2H), 9.95-9.45 (m, 1H), 8.76 (s, 1H), 8.50-8.36 (m, 3H), 8.23 (d, J=7.7 Hz, 1H), 7.22 (s, 1H), 6.97 (t, J=8.6 Hz, 1H), 6.53-6.42 (m, 2H), 6.06 (br d, J=7.8 Hz, 1H), 4.94-4.81 (m, 1H), 4.75-4.22 (m, 4H), 3.84-3.62 (m, 1H), 3.25-3.08 (m, 3H), 3.06-2.86 (m, 2H), 2.83-2.70 (m, 2H), 2.60 (br d, J=3.5 Hz, 2H), 2.44-2.36 (m, 2H), 2.14-2.02 (m, 2H), 1.95-1.60 (m, 5H), 1.49 (br dd, J=2.8, 11.8 Hz, 2H), 1.42 (d, J=6.0 Hz, 6H). LC-MS (ES⁺): m/z 793.1 [M+H]⁺

### Example 51 Synthesis of N-[2-[1-[2-[4-[4-[(2,6-dioxo-3-piperidyl) amino] phenyl]-1-piperidyl]acetyl]-4-piperidyl]-6-isopropoxy-indazol-5-yl]-6-(trifluoromethyl)pyridine-2-carboxamide

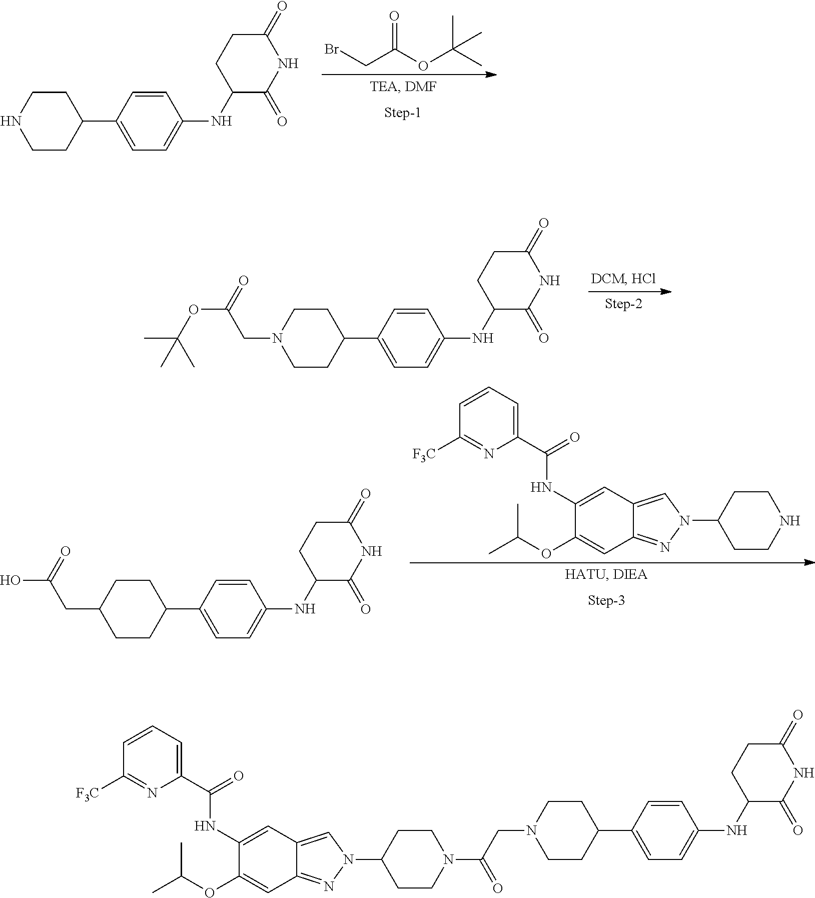

### Step-1

To a solution of 3-[4-(4-piperidyl) anilino]piperidine-2,6-dione (100 mg, 348.00 μmol), tert-butyl 2-bromoacetate (67.88 mg, 348.00 μmol, 51.04 μL) in DMF (1.5 mL) was added TEA (35.21 mg, 348.00 μmol, 48.50 μL). The mixture was stirred at 25° C. for 16 hr. The reaction mixture was poured into water (20 ml) and extracted with ethyl acetate (20 mL×3). The combined organic layers were washed with brine (15 mL), dried over Na₂SO₄, filtered and concentrated under reduced pressure to give tert-butyl 2-[4-[4-[(2,6-dioxo-3-piperidyl) amino] phenyl]-1-piperidyl]acetate (90 mg, 211.14 μmol, 60.67% yield) as a white solid. LC-MS (ESI) m/z 402.3 (M+H)+

### Step-2

To a solution of tert-butyl 2-[4-[4-[(2,6-dioxo-3-piperidyl) amino]phenyl]-1-piperidyl] acetate (100 mg, 249.07 μmol) in DCM (1 mL) was added conc. HCl, 37% aq. (90.81 mg, 2.49 mmol, 113.52 p L). The mixture was stirred at 25° C. for 1 h, then concentrated under reduced pressure. The residue was dissolved with H₂O (1 mL) and MeCN (2 mL) and the resulting solution was concentrated under reduced pressure again to afford 2-[4-[4-[(2,6-dioxo-3-piperidyl) amino]phenyl]-1-piperidyl] acetic acid HCl salt (2, 50 mg, 115.19 μmol, 46.25% yield) as a green solid which was directly used in the next step without further purification. LC-MS (ESI): m/z 297.0 (M+H)+

### Step-3

To a solution of N-[6-isopropoxy-2-(4-piperidyl) indazol-5-yl]-6-(trifluoromethyl) pyridine-2-carboxamide (90 mg, 201.14 μmol) and 2-[4-[4-[(2,6-dioxo-3-piperidyl) amino]phenyl]-1-piperidyl] acetic acid (76.81 mg, 201.14 μmol, HCl salt) in DMF (1 mL) was added HATU (114.72 mg, 301.71 μmol) and diisopropylethylamine (155.97 mg, 1.21 mmol, 210.21 μL). The mixture was stirred at 25° C. for 3 h. The reaction mixture was filtered then purified by prep-HPLC (FA condition) to give N-[2-[1-[2-[4-[4-[(2,6-dioxo-3-piperidyl) amino] phenyl]-1-piperidyl]acetyl]-4-piperidyl]-6-isopropoxy-indazol-5-yl]-6-(trifluoromethyl)pyridine-2-carboxamide formic acid salt (6.53 mg, 7.73 μmol, 3.84% yield) as a white solid.

### Method for “FA Condition”

Instrument: Gilson-281
Column: Shim-pack C18 150 mm*25 mm*10 um
Mobile phase: A for H₂O (0.225% FA v/v) and B for Acetonitrile
Gradient: B 24%-64% in 13 min linearly
Flow rate: 25 ml/min
Column temperature: R.T.
Wavelength: 220 nm/254 nm

¹H NMR (400 MHz, DMSO-d₆): δ=10.85-10.69 (m, 2H), 8.73 (s, 1H), 8.48-8.36 (m, 3H), 8.31 (s, 1H), 8.22 (dd, J=0.9, 7.6 Hz, 1H), 7.20 (s, 1H), 6.95 (d, J=8.6 Hz, 2H), 6.60 (d, J=8.4 Hz, 2H), 5.63 (d, J=7.5 Hz, 1H), 4.84 (td, J=6.0, 12.0 Hz, 1H), 4.77-4.63 (m, 1H), 4.49 (br d, J=13.1 Hz, 1H), 4.35-4.19 (m, 2H), 2.92 (br d, J=7.9 Hz, 2H), 2.83-2.66 (m, 2H), 2.61-2.53 (m, 1H), 2.39-2.28 (m, 2H), 2.23-2.02 (m, 6H), 1.98-1.78 (m, 2H), 1.77-1.52 (m, 4H), 1.40 (d, J=6.0 Hz, 6H) LC-MS (ESI): m/z 775.2 (M+H)+

### Example 52

Compound of Example 52 was prepared substantially following the synthesis of Example 41.

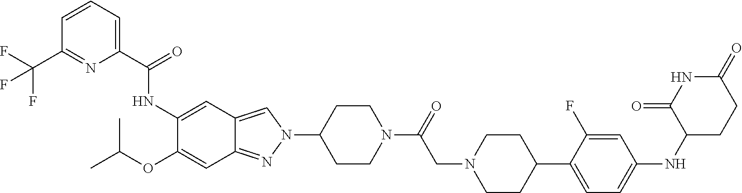 [SMILES: CC(C)Oc1cc2nn(C3CCN(C(=O)CN4CCC(c5ccc(NC6CCC(=O)NC6=O)cc5F)CC4)CC3)cc2cc1NC(=O)c1cccc(C(F)(F)F)n1]

N-(2-(1-(2-(4-(4-((2,6-dioxopiperidin-3-yl)amino)-2-fluorophenyl)piperidin-1-yl)acetyl)piperidin-4-yl)-6-isopropoxy-2H-indazol-5-yl)-6-(trifluoromethyl)picolinamide. ¹H NMR (400 MHz, DMSO-d₆) δ=10.79 (s, 1H), 10.74 (s, 1H), 8.74 (s, 1H), 8.48-8.38 (m, 2H), 8.37 (s, 1H), 8.22 (d, J=7.6 Hz, 1H), 7.18 (s, 1H), 6.99 (br s, 1H), 6.52-6.43 (m, 2H), 6.08 (br d, J=6.8 Hz, 1H), 4.84 (td, J=6.0, 12.0 Hz, 1H), 4.75 (br t, J=10.8 Hz, 1H), 4.51 (br d, J=13.6 Hz, 1H), 4.32 (m, 1H), 4.06-3.80 (m, 1H), 3.62-3.41 (m, 2H), 3.26-3.12 (m, 1H), 3.08-2.80 (m, 3H), 2.79-2.63 (m, 2H), 2.63-2.58 (m, 1H), 2.57-2.53 (m, 1H), 2.29-2.03 (m, 5H), 2.02-1.64 (m, 6H), 1.41 (d, J=5.6 Hz, 6H). LC-MS (ES⁺): m/z 793.4.

### Synthesis of tert-butyl 4-(6-amino-7-isopropoxyimidazo [1, 2-a]pyridin-2-yl) piperidine-1-carboxylate

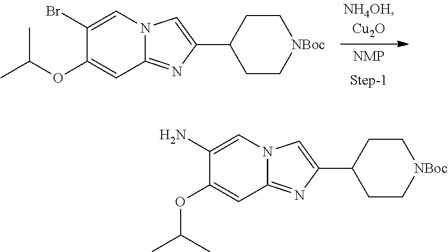 [SMILES: CC(C)Oc1cc2nc(C3CCN(C(=O)OC(C)(C)C)CC3)cn2cc1Br ; CC(C)Oc1cc2nc(C3CCN(C(=O)OC(C)(C)C)CC3)cn2cc1N]

### Step-1

To a solution of tert-butyl 4-(6-bromo-7-isopropoxy-imidazo[1,2-a]pyridin-2-yl)piperidine-1-carboxylate (6 g, 13.69 mmol) in NMP (60 mL) was added 30% ammonium hydroxide (39.97 g, 342.19 mmol, 44.42 m) and cuprous oxide (391.71 mg, 2.74 mmol). The mixture was stirred at 90° C. for 16 hours. After the complete consumption of the reactant as confirmed by LC-MS, the reaction mixture was diluted with H₂O (150 ml), and extracted with ethyl acetate (150 ml×3). The combined organic layers were washed with brine (150 ml×2), dried over Na₂SO₄, and concentrated in vacuo to give a residue. The residue was purified by column chromatography (silica gel, Petroleum ether/Ethyl acetate=10/1 to 1). Compound tert-butyl 4-(6-amino-7-isopropoxyimidazo [1, 2-a]pyridin-2-yl) piperidine-1-carboxylate (3.5 g, 8.60 mmol, 62.82% yield) was obtained as a yellow oil. LC-MS (ES⁺): m/z 375.2 [M+H]⁺.

### Example 53 Synthesis of N-[2-[1-[2-[4-[4-[(2,6-dioxo-3-piperidyl)amino]phenyl]-1-piperidyl]-2-oxo-ethyl]-4-piperidyl]-7-isopropoxy-imidazo[1,2-a]pyridin-6-yl]pyrimidine-4-carboxamide

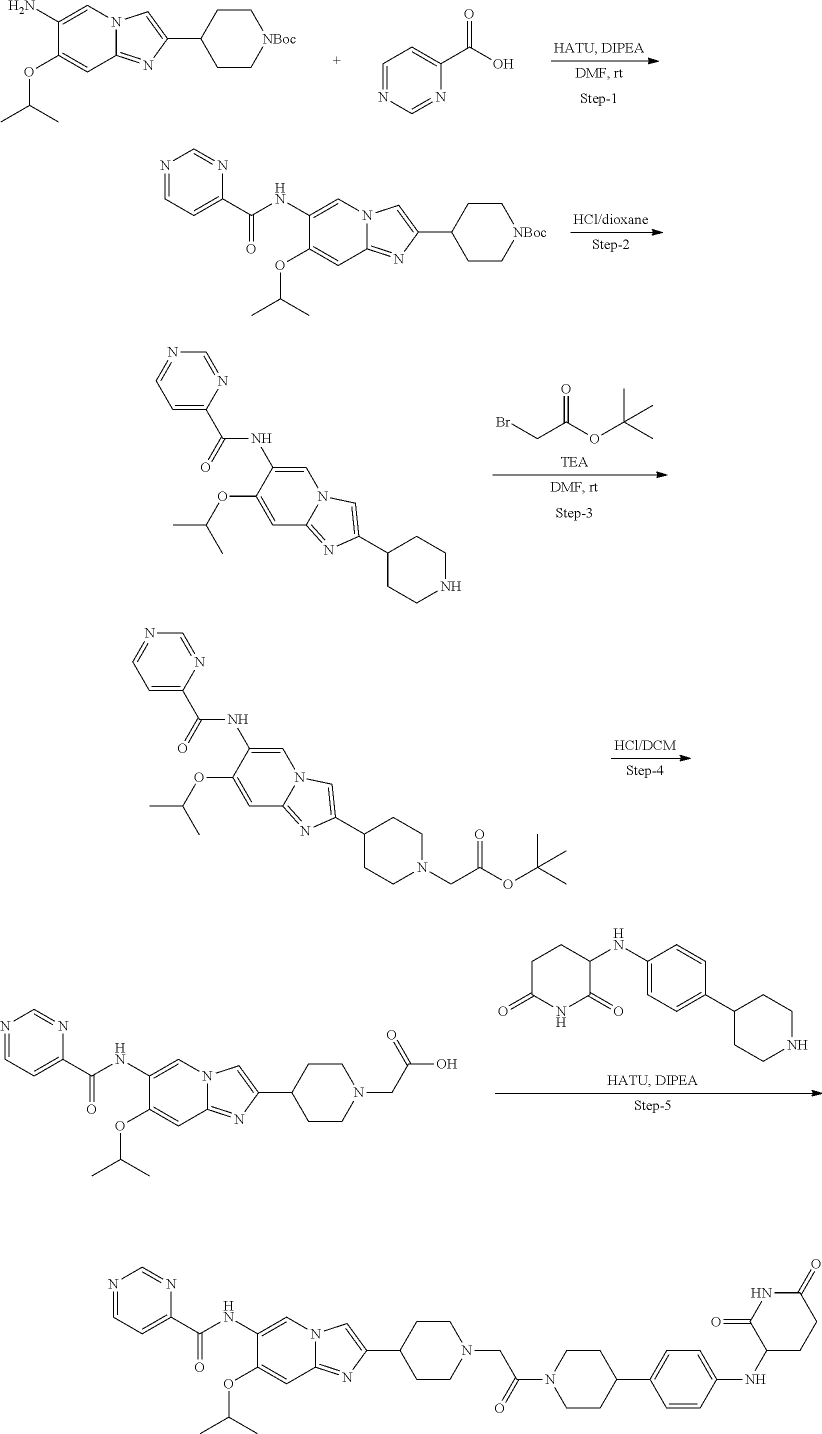

### Step-1

To a solution of tert-butyl 4-(6-amino-7-isopropoxy-imidazo[1,2-a]pyridin-2-yl) piperidine-1-carboxylate (100 mg, 267.04 μmol) and pyrimidine-4-carboxylic acid (49.71 mg, 400.56 μmol) in DMF (1 mL) was added HATU (152.30 mg, 400.56 μmol) and diisopropylethylamine (345.13 mg, 2.67 mmol, 465.14 μL). The mixture was stirred at 25° C. for 3 hours. After consumption of the reactants as shown by LC-MS, the reaction mixture was poured into water (15 ml) and extracted with ethyl acetate (20 mL×4). The combined organic layers were washed with brine (20 mL), dried over Na₂SO₄, filtered, and concentrated under reduced pressure to give an oil which was purified by prep-TLC (Pet ether: Ethyl acetate=0:1) to afford tert-butyl 4-[7-isopropoxy-6-(pyrimidine-4-carbonyl amino)imidazo[1,2-a]pyridin-2-yl]piperidine-1-carboxylate (70 mg, 121.34 μmol, 45.44% yield) as a yellow solid. LC-MS (ES⁺): m/z 481.3 [M+H]⁺.

### Step-2

A solution of tert-butyl 4-[7-isopropoxy-6-(pyrimidine-4-carbonylamino)imidazo[1,2-a]pyridin-2-yl]piperidine-1-carboxylate (60 mg, 124.85 μmol) in dioxane/HCl (4M, 1 mL) was stirred for 16 h at 25° C. After complete consumption of the reactants as shown by LC-MS, the reaction mixture was concentrated under reduced pressure to afford N-[7-isopropoxy-2-(4-piperidyl)imidazo[1,2-a]pyridin-6-yl]pyrimidine-4-carboxamide HCl salt (50 mg, 119.93 μmol, 96.06% yield) as a yellow solid, which was used in the next step without purification. LC-MS (ES⁺): m/z 381.3 [M+H]⁺

### Step-3

To a solution of N-[7-isopropoxy-2-(4-piperidyl)imidazo[1,2-a]pyridin-6-yl]pyrimidine-4-carboxamide (52 mg, 124.73 μmol, HCl salt) and tert-butyl 2-bromoacetate (24.33 mg, 124.73 μmol, 18.29 μL) in DMF (1 mL) was added TEA (100.97 mg, 997.83 μmol, 139.08 μL) and the mixture stirred at 25° C. for 3 h. After complete consumption of the reactants as shown by LC-MS, the reaction mixture was poured into water (15 mL) and extracted with ethyl acetate (20 mL×4). The combined organic layers were washed with brine (20 mL), dried over Na₂SO₄, filtered, and concentrated under reduced pressure to afford tert-butyl 2-[4-[7-isopropoxy-6-(pyrimidine-4-carbonylamino)imidazo[1,2-a]pyridin-2-yl]-1-piperidyl]acetate (60 mg, 108.21 μmol, 86.76% yield) as a yellow solid, which was used without further purification. LC-MS (ES⁺): m/z 495.3 [M+H]⁺

### Step-4

To a solution of tert-butyl 2-[4-[7-isopropoxy-6-(pyrimidine-4-carbonylamino) imidazo[1,2-a]pyridin-2-yl]-1-piperidyl]acetate (45 mg, 90.99 μmol) in DCM (1 mL) was added HCl (80.00 mg, 2.19 mmol, 0.1 mL). The mixture was stirred for 16 h. After complete consumption of the reactant as shown by LC-MS, the reaction mixture was concentrated under reduced pressure to give a residue which was purified by prep-HPLC to afford 2-[4-[7-isopropoxy-6-(pyrimidine-4-carbonylamino)imidazo[1,2-a]pyridin-2-yl]-1-piperidyl]acetic acid (25 mg, 56.21 μmol, 61.78% yield) as a white solid. LC-MS (ES⁺): m/z 439.3 [M+H]⁺

### Step-5

To a solution of 2-[4-[7-isopropoxy-6-(pyrimidine-4-carbonylamino)imidazo[1,2-a]pyridin-2-yl]-1-piperidyl]acetic acid (20 mg, 45.61 μmol) in DMF (0.5 mL) were added HATU (26.01 mg, 68.42 μmol), diisopropylethylamine (47.16 mg, 364.90 μmol, 63.56 μL), and 3-[4-(4-piperidyl)anilino]piperidine-2,6-dione (15.73 mg, 54.73 μmol). The mixture was stirred at 25° C. for 3 h. After complete consumption of the reactants as shown by LC-MS, the reaction mixture was filtered and the filtrate purified by prep-HPLC (TFA condition) to afford N-[2-[1-[2-[4-[4-[(2,6-dioxo-3-piperidyl)amino]phenyl]-1-piperidyl]-2-oxo-ethyl]-4-piperidyl]-7-isopropoxy-imidazo[1,2-a]pyridin-6-yl]pyrimidine-4-carboxamide TFA salt (6.8 mg, 7.97 μmol, 17.48% yield) as a green solid.

### Method for “TFA Condition”

Instrument: Gilson-281
Column: Phenomenex Synergi C18 150 mm*25 mm* 10 um
Mobile phase: A for H₂O (0.075% TFA v/v) and B for Acetonitrile
Gradient: B 10%-40% in 10 min linearly
Flow rate: 25 ml/min
Column temperature: R.T.
Wavelength: 220 nm/254 nm

¹H NMR (400 MHz, DMSO-d₆) δ=10.79 (s, 1H), 10.50 (s, 1H), 9.72 (s, 2H), 9.49 (d, J=0.9 Hz, 1H), 9.22 (d, J=5.0 Hz, 1H), 8.26-8.12 (m, 2H), 7.47 (s, 1H), 6.96 (br d, J=8.4 Hz, 2H), 6.62 (br d, J=8.4 Hz, 2H), 5.08 (td, J=6.0, 11.9 Hz, 1H), 4.60-4.17 (m, 4H), 3.76-3.63 (m, 2H), 3.23-3.11 (m, 4H), 2.60 (br s, 4H), 2.23 (br s, 3H), 2.14-1.95 (m, 3H), 1.94-1.73 (m, 3H), 1.65-1.54 (m, 1H), 1.47 (d, J=6.0 Hz, 6H). LC-MS (ES⁺): m/z 708.0 [M+H]⁺

### Example 54

Compound of Example 54 was prepared substantially following the synthesis of Example 53.

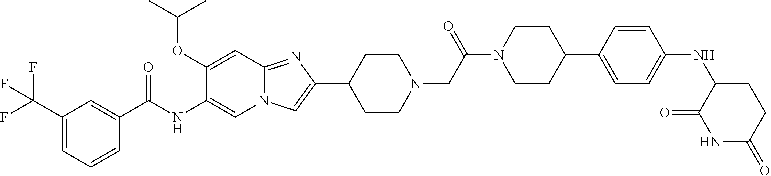 [SMILES: CC(C)Oc1cc2nc(C3CCN(CC(=O)N4CCC(c5ccc(NC6CCC(=O)NC6=O)cc5)CC4)CC3)cn2cc1NC(=O)c1cccc(C(F)(F)F)c1]

### N-[2-[1-[2-[4-[4-[(2,6-dioxo-3-piperidyl)amino]phenyl]-1-piperidyl]-2-oxo-ethyl]-4-piperidyl]-7-isopropoxy-imidazo[1,2-a]pyridin-6-yl]-3-(trifluoromethyl)benzamide

LC-MS (ES⁺): m/z 774.0 [M+H]⁺. ¹H NMR (400 MHz, DMSO-d₆)_{6=10.78} (s, 1H), 10.22 (s, 1H), 9.79-9.58 (m, 1H), 9.27 (s, 1H), 8.35-8.17 (m, 2H), 8.12-8.02 (m, 2H), 7.85 (t, J=7.8 Hz, 1H), 7.42 (s, 1H), 6.97 (br d, J=8.3 Hz, 2H), 6.64 (br d, J=8.2 Hz, 2H), 5.06-4.88 (m, 1H), 4.61-4.22 (m, 4H), 3.46-3.29 (m, 3H), 3.18 (br t, J=12.0 Hz, 4H), 2.87-2.56 (m, 4H), 2.39-2.17 (m, 3H), 2.16-1.95 (m, 3H), 1.94-1.77 (m, 3H), 1.66-1.53 (m, 1H), 1.41 (d, J=6.0 Hz, 6H).

### Example 55

Compound of Example 55 was prepared substantially following the synthesis of Example 53.

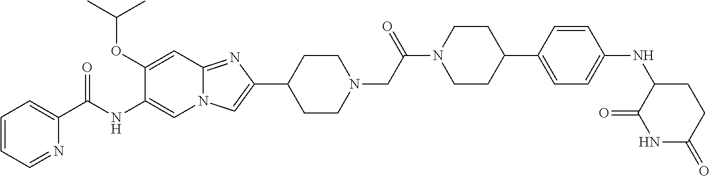 [SMILES: CC(C)Oc1cc2nc(C3CCN(CC(=O)N4CCC(c5ccc(NC6CCC(=O)NC6=O)cc5)CC4)CC3)cn2cc1NC(=O)c1ccccn1]

### N-[2-[1-[2-[4-[4-[(2,6-dioxo-3-piperidyl)amino]phenyl]-1-piperidyl]-2-oxo-ethyl]-4-piperidyl]-7-isopropoxy-imidazo[1,2-a]pyridin-6-yl]pyridine-2-carboxamide

LC-MS (ES⁺): m/z 707.0 [M+H]⁺. ¹H NMR (400 MHz, DMSO-d₆) δ=10.77 (s, 1H), 10.60 (s, 1H), 9.77 (s, 1H), 9.73-9.59 (m, 1H), 8.79 (d, J=4.5 Hz, 1H), 8.27-8.09 (m, 3H), 7.80-7.71 (m, 1H), 7.45 (s, 1H), 6.96 (br d, J=8.3 Hz, 2H), 6.63 (br d, J=8.4 Hz, 2H), 5.07 (qd, J=6.0, 11.8 Hz, 1H), 4.59-4.22 (m, 4H), 3.79-3.60 (m, 3H), 3.24-3.12 (m, 4H), 2.81-2.54 (m, 4H), 2.36-2.18 (m, 3H), 2.15-1.96 (m, 3H), 1.93-1.73 (m, 3H), 1.66-1.54 (m, 1H), 1.48 (d, J=6.0 Hz, 6H)

### Example 56

Compound of Example 56 was prepared substantially following the synthesis of Example 53.

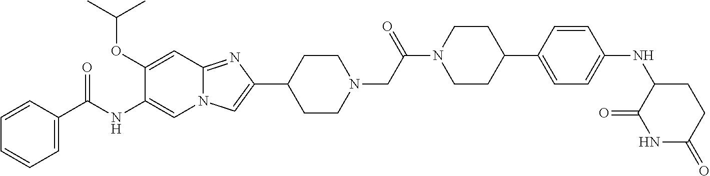 [SMILES: CC(C)Oc1cc2nc(C3CCN(CC(=O)N4CCC(c5ccc(NC6CCC(=O)NC6=O)cc5)CC4)CC3)cn2cc1NC(=O)c1ccccc1]

N-[2-[1-[2-[4-[4-[(2,6-dioxo-3-piperidyl)amino]phenyl]-1-piperidyl]-2-oxo-ethyl]-4-piperidyl]-7-isopropoxy-imidazo[1,2-a]pyridin-6-yl]benzamide. LC-MS (ES⁺): m/z 706.0 [M+H]⁺. ¹H NMR (400 MHz, DMSO-d₆)_{6=10.77} (s, 1H), 9.82 (s, 1H), 9.75-9.60 (m, 1H), 9.37 (s, 1H), 8.13-8.04 (m, 1H), 7.96 (d, J=7.3 Hz, 2H), 7.71-7.55 (m, 3H), 7.41 (s, 1H), 6.96 (br d, J=8.3 Hz, 2H), 6.63 (br d, J=8.3 Hz, 2H), 4.98 (quin, J=5.9 Hz, 1H), 4.61-4.21 (m, 4H), 3.48-3.28 (m, 3H), 3.27-3.09 (m, 4H), 2.84-2.55 (m, 4H), 2.37-2.17 (m, 3H), 2.15-1.97 (m, 3H), 1.94-1.73 (m, 3H), 1.66-1.50 (m, 1H), 1.42 (d, J=6.0 Hz, 6H)

### Example 57

Compound of Example 57 was prepared substantially following the synthesis of Example 53.

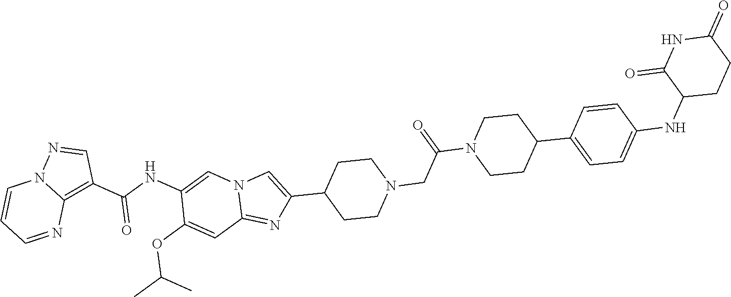 [SMILES: CC(C)Oc1cc2nc(C3CCN(CC(=O)N4CCC(c5ccc(NC6CCC(=O)NC6=O)cc5)CC4)CC3)cn2cc1NC(=O)c1cnn2cccnc12]

N-(2-(1-(2-(4-(4-((2,6-dioxopiperidin-3-yl)amino)phenyl)piperidin-1-yl)-2-oxoethyl) piperidin-4-yl)-7-isopropoxyimidazo[1,2-a]pyridin-6-yl)pyrazolo[1,5-a]pyrimidine-3-carboxamide. LC-MS (ES⁺): 747.86; [M+H]⁺: 747.4. ¹H NMR (400 MHz, DMSO-d₆) δ ppm 10.81 (s, 1H) 10.72 (s, 1H) 9.82 (s, 1H) 9.61-9.78 (m, 1H) 9.46 (dd, J=7.2, 1.51 Hz, 1H) 8.94 (dd, J=4.0, 1.51 Hz, 1H) 8.80 (s, 1H) 8.17-8.26 (m, 1H) 7.50 (s, 1H) 7.37-7.44 (m, 1H) 6.97 (br d, J=8.4 Hz, 2H) 6.63 (br d, J=8.4 Hz, 2H) 5.04-5.16 (m, 1H) 4.24-4.56 (m, 4H) 3.76-3.88 (m, 2H) 3.38-3.47 (m, 2H) 3.14-3.25 (m, 3H) 2.64-2.83 (m, 3H) 2.56-2.63 (m, 1H) 2.16-2.37 (m, 3H) 1.95-2.15 (m, 3H) 1.74-1.92 (m, 3H) 1.56 (d, J=5.6 Hz, 6H) 1.36-1.47 (m, 1H).

### Example 58

Compound of Example 58 was prepared substantially following the synthesis of Example 53.

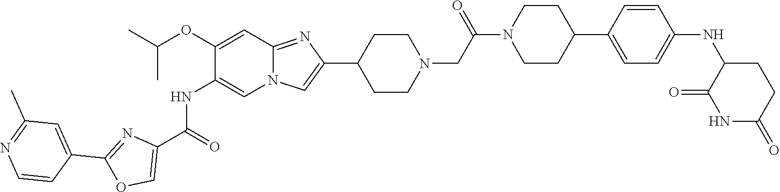 [SMILES: Cc1cc(-c2nc(C(=O)Nc3cn4cc(C5CCN(CC(=O)N6CCC(c7ccc(NC8CCC(=O)NC8=O)cc7)CC6)CC5)nc4cc3OC(C)C)co2)ccn1]

N-(2-(1-(2-(4-(4-((2,6-dioxopiperidin-3-yl)amino)phenyl)piperidin-1-yl)-2-oxoethyl)piperidin-4-yl)-7-isopropoxyimidazo[1,2-a]pyridin-6-yl)-2-(2-methylpyridin-4-yl)oxazole-4-carboxamide. LC-MS (ESI): m/z 787.90; 788.4 [M+H]⁺. ¹H NMR (400 MHz, DMSO-d₆) δ=10.81-10.74 (m, 1H), 9.68 (s, 1H), 9.61-9.57 (m, 1H), 9.19 (s, 1H), 8.75 (d, J=5.1 Hz, 1H), 8.22-8.12 (m, 1H), 7.87 (s, 1H), 7.80 (d, J=5.1 Hz, 1H), 7.45 (s, 1H), 6.97 (br d, J=8.3 Hz, 2H), 6.69-6.58 (m, 2H), 5.17-5.07 (m, 1H), 4.58-4.48 (m, 1H), 4.45-4.32 (m, 2H), 4.31-4.26 (m, 1H), 3.80-3.71 (m, 2H), 3.69-3.65 (m, 2H), 3.20-3.13 (m, 4H), 2.77-2.67 (m, 4H), 2.62 (s, 3H), 2.37-2.22 (m, 3H), 2.14-1.98 (m, 3H), 1.92-1.78 (m, 3H), 1.65-1.57 (m, 1H), 1.54-1.50 (m, 6H)

### Example 59

Compound of Example 59 was prepared substantially following the synthesis of Example 53.

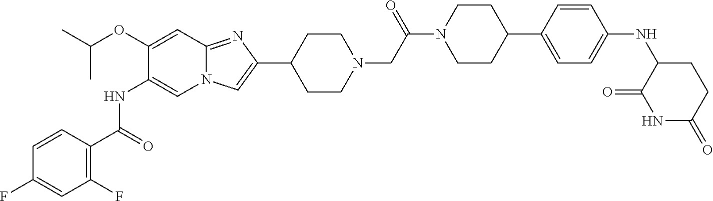 [SMILES: CC(C)Oc1cc2nc(C3CCN(CC(=O)N4CCC(c5ccc(NC6CCC(=O)NC6=O)cc5)CC4)CC3)cn2cc1NC(=O)c1ccc(F)cc1F]

N-(2-(1-(2-(4-(4-((2,6-dioxopiperidin-3-yl)amino)phenyl)piperidin-1-yl)-2-oxoethyl) piperidin-4-yl)-7-isopropoxyimidazo[1,2-a]pyridin-6-yl)-2,4-difluorobenzamide. LC-MS (ES⁺): 741.82; [M+H]⁺: 742.3. ¹H NMR (400 MHz, DMSO-d₆) δ ppm 10.78 (s, 1H) 9.79 (d, J=8.00 Hz, 1H) 9.64-9.75 (m, 1H) 9.62 (s, 1H) 8.09-8.16 (m, 1H) 7.94-8.02 (m, 1H) 7.51-7.59 (m, 1H) 7.45 (s, 1H) 7.33 (td, J=8.4, 2.4 Hz, 1H) 6.97 (br d, J=8.4 Hz, 2H) 6.64 (br d, J=8.4 Hz, 2H) 4.96-5.08 (m, 1H) 4.52 (br d, J=12.8 Hz, 1H) 4.25-4.44 (m, 3H) 3.70-3.84 (m, 3H) 3.08-3.27 (m, 4H) 2.66-2.82 (m, 3H) 2.53-2.62 (m, 1H) 2.15-2.38 (m, 3H) 1.95-2.15 (m, 3H) 1.74-1.95 (m, 3H) 1.52-1.65 (m, 1H) 1.44 (d, J=6.0 Hz, 6H).

### Example 60

Compound of Example 60 was prepared substantially following the synthesis of Example 53.

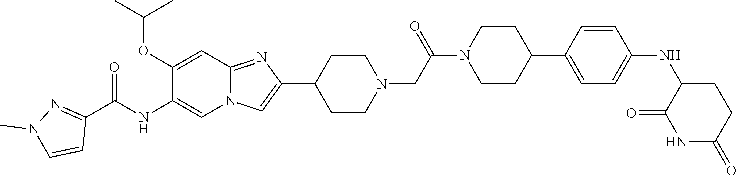 [SMILES: CC(C)Oc1cc2nc(C3CCN(CC(=O)N4CCC(c5ccc(NC6CCC(=O)NC6=O)cc5)CC4)CC3)cn2cc1NC(=O)c1ccn(C)n1]

N-[2-[1-[2-[4-[4-[(2,6-dioxo-3-piperidyl)amino]phenyl]-1-piperidyl]-2-oxo-ethyl]-4-piperidyl]-7-isopropoxy-imidazo[1,2-a]pyridin-6-yl]-1-methyl-pyrazole-3-carboxamide. LC-MS (ES⁺): m/z 710.0 [M+H]⁺. ¹H NMR (400 MHz, DMSO-d₆) δ=10.78 (s, 1H), 9.75-9.56 (m, 2H), 9.41 (s, 1H), 8.28-8.06 (m, 1H), 7.94 (d, J=2.1 Hz, 1H), 7.44 (s, 1H), 6.96 (br d, J=8.3 Hz, 2H), 6.84 (d, J=2.3 Hz, 1H), 6.63 (br d, J=8.5 Hz, 2H), 5.05 (td, J=6.0, 11.9 Hz, 1H), 4.60-4.19 (m, 4H), 3.98 (s, 3H), 3.71 (br s, 3H), 3.28-3.08 (m, 4H), 2.86-2.54 (m, 4H), 2.38-2.16 (m, 3H), 2.14-1.95 (m, 3H), 1.93-1.74 (m, 3H), 1.65-1.50 (m, 1H), 1.46 (d, J=6.0 Hz, 6H).

### Example 61

Compound of Example 61 was prepared substantially following the synthesis of Example 53.

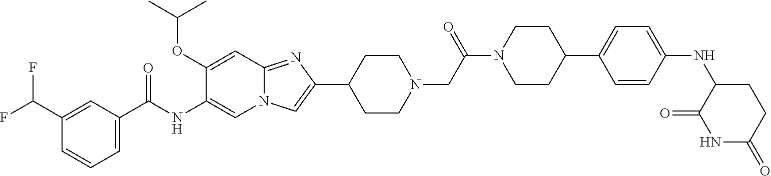 [SMILES: CC(C)Oc1cc2nc(C3CCN(CC(=O)N4CCC(c5ccc(NC6CCC(=O)NC6=O)cc5)CC4)CC3)cn2cc1NC(=O)c1cccc(C(F)F)c1]

3-(difluoromethyl)-N-(2-(1-(2-(4-(4-((2,6-dioxopiperidin-3-yl)amino) phenyl)piperidin-1-yl)-2-oxoethyl)piperidin-4-yl)-7-isopropoxyimidazo[1,2-a]pyridin-6-yl)benzamide. LC-MS (ES⁺): 755.85; [M+H]⁺: 756. ¹H NMR (400 MHz, DMSO-d₆) δ ppm 10.78 (s, 1H) 10.07 (s, 1H) 9.49-9.89 (m, 1H) 9.25 (s, 1H) 8.10-8.18 (m, 2H) 8.04 (br s, 1H) 7.86 (br d, J=7.6 Hz, 1H) 7.69-7.79 (m, 1H) 7.37 (s, 1H) 7.04-7.32 (m, 1H) 6.96 (br d, J=8.4 Hz, 2H) 6.63 (br d, J=8.4 Hz, 2H) 4.89-5.02 (m, 1H) 4.52 (br d, J=12.96 Hz, 1H) 4.24-4.46 (m, 3H) 3.62-3.81 (m, 4H) 3.17-3.25 (m, 3H) 2.66-2.81 (m, 3H) 2.55-2.62 (m, 1H) 2.16-2.40 (m, 3H) 1.98-2.15 (m, 3H) 1.75-1.93 (m, 3H) 1.52-1.65 (m, 1H) 1.42-1.50 (m, 1H) 1.40 (d, J=5.99 Hz, 6H).

### Example 62 Synthesis of N-[2-[1-[2-[4-[4-[(2,4-dioxohexahydropyrimidin-1-yl)methyl] phenyl]-1-piperidyl]ethyl]-4-piperidyl]-7-isopropoxy-imidazo[1,2-a]pyridin-6-yl]-6-(trifluoromethyl)pyridine-2-carboxamide

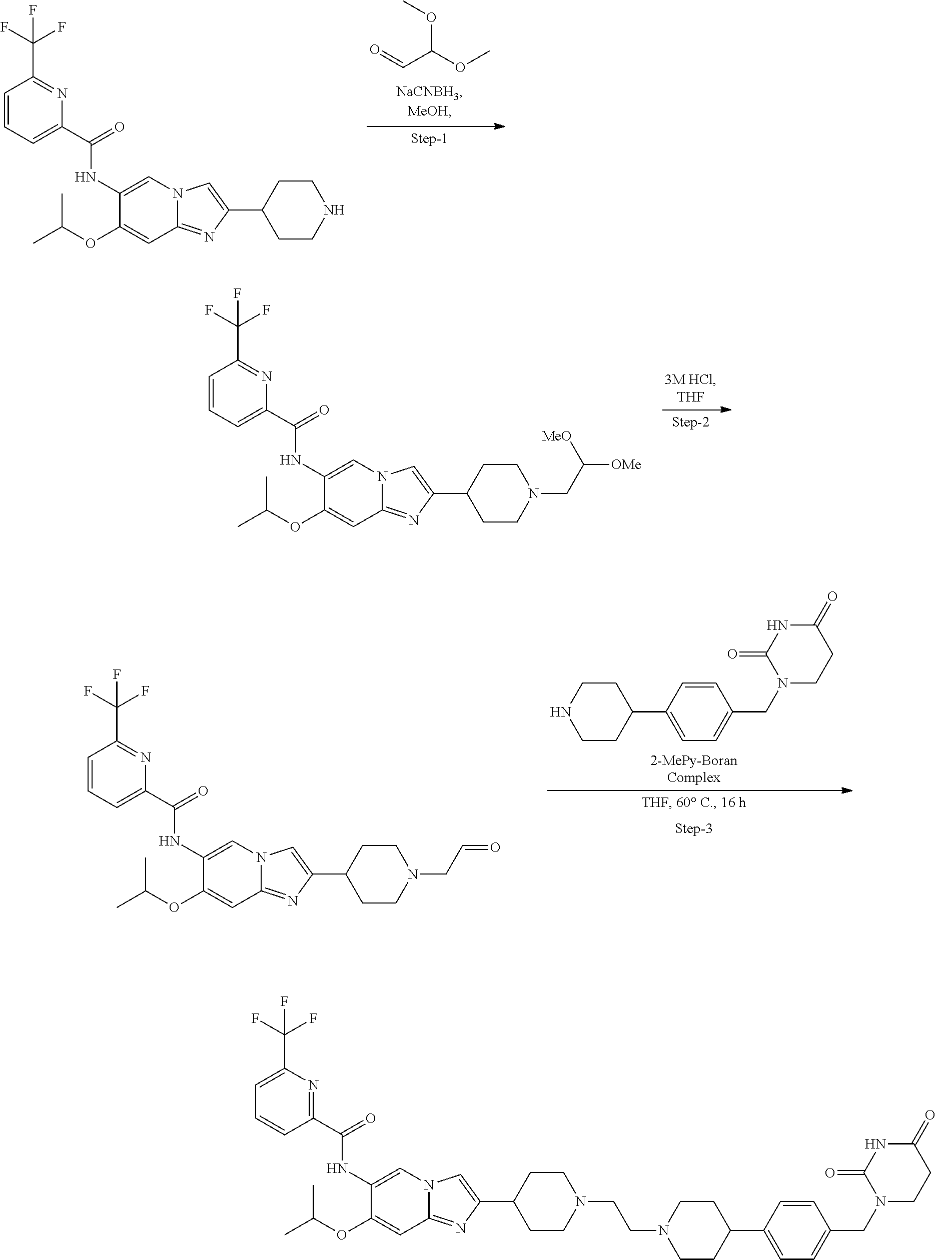

### Step-1

To a stirred solution of N-[7-isopropoxy-2-(4-piperidyl)imidazo[1,2-a]pyridin-6-yl]-6-(trifluoromethyl)pyridine-2-carboxamide (1.0 g, 2.23 mmol) in ethanol was added 2,2-dimethoxyacetaldehyde (232.66 mg, 2.23 mmol) and sodium cyanoborohydride (210.67 mg, 3.35 mmol) at 0° C. and resulting mixture was stirred at room temperature for 16 h. After completion, the reaction mixture was concentrated and dried under vacuum to give crude N-[2-[1-(2,2-dimethoxyethyl)-4-piperidyl]-7-isopropoxy-imidazo[1,2-a]pyridin-6-yl]-6-(trifluoromethyl)pyridine-2-carboxamide (1.0 g, 1.38 mmol, 61.83% yield). LC-MS (ES⁺): m/z 536.30 [M+H]⁺.

### Step-2

To a stirred solution of N-[2-[1-(2,2-dimethoxyethyl)-4-piperidyl]-7-isopropoxy-imidazo[1,2-a]pyridin-6-yl]-6-(trifluoromethyl)pyridine-2-carboxamide (1.0 g, 1.87 mmol) in THF was added 3M HCl (68.08 mg, 1.87 mmol, 3 mL) at 0° C. and resulting mixture was stirred at room temperature for 16 hours. After the completion, the reaction mixture was concentrated and dried under vacuum to give crude N-[7-isopropoxy-2-[1-(2-oxoethyl)-4-piperidyl]imidazo[1,2-a]pyridin-6-yl]-6 (trifluoromethyl)pyridine-2-carboxamide HCl salt (0.8 g, 1.03 mmol, 55.14% yield). LC-MS (ES⁺): m/z 488.28 [M−H]⁻.

### Step-3

To a stirred solution of N-[7-isopropoxy-2-[1-(2-oxoethyl)-4-piperidyl]imidazo[1,2-a]pyridin-6-yl]-6-(trifluoromethyl)pyridine-2-carboxamide (0.05 g, 102.15 μmol) and 1-[[4-(4-piperidyl)phenyl]methyl]hexahydropyrimidine-2,4-dione TFA salt (29.35 mg, 73.13 μmol) in methanol was added TEA for basification at 0° C. and stirred for 30 minutes. Then, 2-picoline borane complex (60 mg, 102.15 μmol) at 0° C. was added and the resulting mixture was stirred at 60° C. for 16 h. The reaction mixture was concentrated then purified by prep HPLC to give N-[2-[1-[2-[4-[4-[(2,4-dioxohexahydropyrimidin-1-yl)methyl]phenyl]-1-piperidyl]ethyl]-4-piperidyl]-7-isopropoxy-imidazo[1,2-a]pyridin-6-yl]-6-(trifluoromethyl) pyridine-2-carboxamide formic acid salt (11 mg, 12.77 μmol, 12.50% yield). LC-MS (ES⁺): m/z 761.18 [M+H]⁺. ¹H NMR (400 MHz, DMSO-d₆) δ 10.44 (s, 1H), 10.18 (s, 1H), 9.43 (s, 1H), 8.43 (q, J=8.4 Hz, 2H), 8.25 (t, J=7.8 Hz, 3H), 7.66 (s, 1H), 7.23 (d, J=37.7 Hz, 4H), 7.12 (s, 1H), 4.88 (t, J=6.1 Hz, 1H), 4.47 (s, 2H), 2.98 (m, 8H), 2.67 (d, J=14.5 Hz, 4H), 2.07 (m, 4H), 1.95 (d, J=12.4 Hz, 2H), 1.65 (m, 6H), 1.40 (d, J=5.9 Hz, 6H).

### Example 63

Compound of Example 63 was prepared substantially following the synthesis of Example 62.

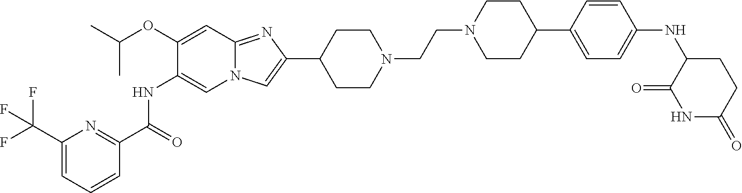 [SMILES: CC(C)Oc1cc2nc(C3CCN(CCN4CCC(c5ccc(NC6CCC(=O)NC6=O)cc5)CC4)CC3)cn2cc1NC(=O)c1cccc(C(F)(F)F)n1]

N-[2-[1-[2-[4-[4-[(2,6-dioxo-3-piperidyl)oxy]phenyl]-1-piperidyl]ethyl]-4-piperidyl]-7-isopropoxy-imidazo[1,2-a]pyridin-6-yl]-6-(trifluoromethyl)pyridine-2-carboxamide. LC-MS (ES⁺): m/z 762.11 [M+H]⁺. ¹H NMR (400 MHz, DMSO-d₆) δ 10.93 (s, 1H), 10.54 (s, 1H), 9.71 (s, 1H), 8.47 (t, J=8.1 Hz, 2H), 8.29 (d, J=7.0 Hz, 1H), 8.09 (s, 1H), 7.36 (s, 1H), 7.19 (t, J=10.3 Hz, 1H), 7.09 (s, 1H), 6.98 (t, J=6.8 Hz, 1H), 5.17 (q, J=5.3 Hz, 1H), 5.07 (s, 1H), 3.00 (m, 4H), 3.0 (m, 8H), 2.73-2.5 (m, 4H), 2.13 (m, 4H), 1.91 (m, 6H), 1.46 (d, J=5.8 Hz, 6H), 1.14 (m, 1H).

### Example 64

Compound of Example 64 was prepared substantially following the synthesis of Example 62.

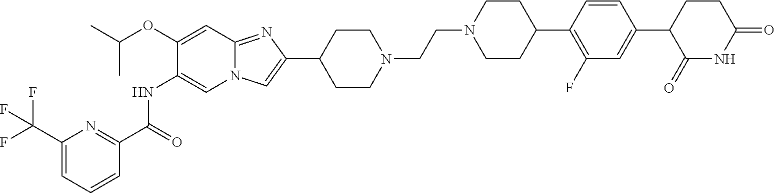 [SMILES: CC(C)Oc1cc2nc(C3CCN(CCN4CCC(c5ccc(C6CCC(=O)NC6=O)cc5F)CC4)CC3)cn2cc1NC(=O)c1cccc(C(F)(F)F)n1]

N-[2-[1-[2-[4-[4-(2,6-dioxo-3-piperidyl)-2-fluoro-phenyl]-1-piperidyl]ethyl]-4-piperidyl]-7-isopropoxy-imidazo[1,2-a]pyridin-6-yl]-6-(trifluoromethyl)pyridine-2-carboxamide. LC-MS (ES⁺): m/z 764.70 [M+H]⁺. H NMR (401 MHz, DMSO) δ 10.83 (s, 1H), 10.44 (s, 1H), 9.43 (s, 1H), 8.43 (q, J=8.5 Hz, 2H), 8.24 (d, J=7.1 Hz, 1H), 7.66 (s, 1H), 7.29 (t, J=8.0 Hz, 1H), 7.12 (s, 1H), 7.03 (t, J=8.1 Hz, 2H), 4.88 (m, 1H), 3.86 (q, J=5.5 Hz, 1H), 2.98 (m, 4H), 2.66 (m, 6H), 2.50 (m, 4H), 2.22 (q, J=5.4 Hz, 3H), 2.05 (m, 5H), 1.89 (m, 3H), 1.69 (m, 1H), 1.40 (d, J=5.9 Hz, 6H).

### Example 65

Compound of Example 65 was prepared substantially following the synthesis of Example 62.

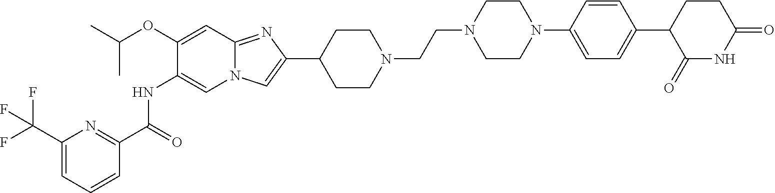 [SMILES: CC(C)Oc1cc2nc(C3CCN(CCN4CCN(c5ccc(C6CCC(=O)NC6=O)cc5)CC4)CC3)cn2cc1NC(=O)c1cccc(C(F)(F)F)n1]

N-[2-[1-[2-[4-[4-(2,6-dioxo-3-piperidyl)phenyl]piperazin-1-yl]ethyl]-4-piperidyl]-7-isopropoxy-imidazo[1,2-a]pyridin-6-yl]-6-(trifluoromethyl)pyridine-2-carboxamide. LC-MS (ES⁺): m/z 747.56 [M+H]⁺. ¹H NMR (400 MHz, DMSO-d₆) δ 10.77 (s, 1H), 10.44 (s, 1H), 9.43 (s, 1H), 8.43 (q, J=8.5 Hz, 1H), 8.24 (d, J=7.3 Hz, 1H), 7.68 (d, J=16.8 Hz, 1H), 7.08 (t, J=16.8 Hz, 2H), 6.89 (d, J=8.5 Hz, 1H), 4.88 (m, 1H), 3.72 (q, J=5.2 Hz, 4H), 3.02 (t, J=32.7 Hz, 2H), 2.58 (m, 9H), 2.01 (m, 10H), 1.62 (s, 6H), 1.40 (m, 3H), 1.24 (s, 1H).

### Example 66

Compound of Example 66 was prepared substantially following the synthesis of Example 62.

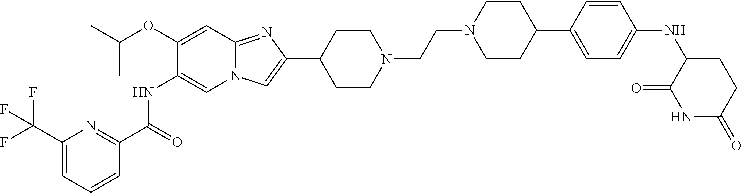 [SMILES: CC(C)Oc1cc2nc(C3CCN(CCN4CCC(c5ccc(NC6CCC(=O)NC6=O)cc5)CC4)CC3)cn2cc1NC(=O)c1cccc(C(F)(F)F)n1]

N-[2-[1-[2-[4-[4-[(2,6-dioxo-3-piperidyl)amino]phenyl]-1-piperidyl]ethyl]-4-piperidyl]-7-isopropoxy-imidazo[1,2-a]pyridin-6-yl]-6-(trifluoromethyl)pyridine-2-carboxamide. LC-MS (ES⁺): m/z 761.59 [M+H]⁺. ¹H NMR (400 MHz, DMSO-d₆) δ 10.76 (s, 1H), 10.44 (s, 1H), 9.43 (s, 1H), 8.43 (q, J=8.6 Hz, 2H), 8.24 (d, J=7.3 Hz, 1H), 7.68 (t, J=9.3 Hz, 1H), 7.12 (s, 1H), 6.95 (d, J=8.2 Hz, 1H), 6.60 (d, J=8.2 Hz, 1H), 5.63 (d, J=7.2 Hz, 1H), 4.87 (q, J=5.9 Hz, 1H), 4.26 (s, 1H), 4.13 (d, J=3.9 Hz, 1H), 2.96 (m, 4H), 2.72 (d, J=12.0 Hz, 1H), 2.59 (m, 6H), 2.45 (m, 4H), 2.30 (t, J=10.1 Hz, 1H), 1.96 (m, 7H), 1.62 (d, J=19.0 Hz, 6H), 1.40 (d, J=5.9 Hz, 1H), 1.33 (m, 3H).

### Example 67

Compound of Example 67 was prepared substantially following the synthesis of Example 62.

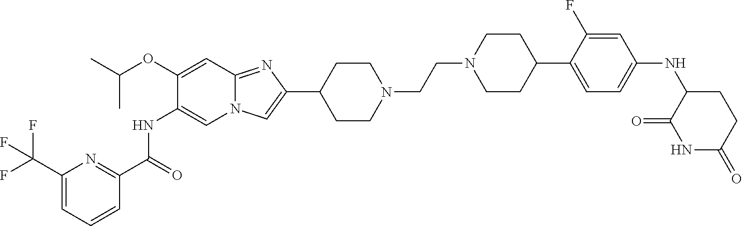 [SMILES: CC(C)Oc1cc2nc(C3CCN(CCN4CCC(c5ccc(NC6CCC(=O)NC6=O)cc5F)CC4)CC3)cn2cc1NC(=O)c1cccc(C(F)(F)F)n1]

N-[2-[1-[2-[4-[4-[(2,6-dioxo-3-piperidyl)amino]-2-fluoro-phenyl]-1-piperidyl]ethyl]-4-piperidyl]-7-isopropoxy-imidazo[1,2-a]pyridin-6-yl]-6-(trifluoromethyl)pyridine-2-carboxamide. LC-MS (ES⁺): m/z 777.53 [M+H]⁺. ¹H NMR (400 MHz, DMSO-d₆) δ 10.78 (s, 1H), 10.44 (s, 1H), 9.43 (s, 1H), 8.43 (q, J=8.5 Hz, 2H), 8.24 (d, J=7.2 Hz, 1H), 7.66 (s, 1H), 7.12 (s, 1H), 6.99 (t, J=8.9 Hz, 1H), 6.44 (t, J=8.6 Hz, 1H), 5.99 (d, J=7.6 Hz, 1H), 4.88 (m, 1H), 4.30 (t, J=11.9 Hz, 1H), 2.96 (m, 7H), 2.67 (s, 1H), 2.58 (m, 3H), 2.50 (m, 4H), 2.00 (m, 6H), 1.65 (m, 6H), 1.40 (d, J=5.9 Hz, 6H).

### Example 68 Synthesis of N-[2-[1-[2-[4-[(2,6-dioxo-3-piperidyl)amino]phenyl]ethyl]-4-piperidyl]-7-isopropoxy-imidazo[1,2-a]pyridin-6-yl]-6-(trifluoromethyl)pyridine-2-carboxamide

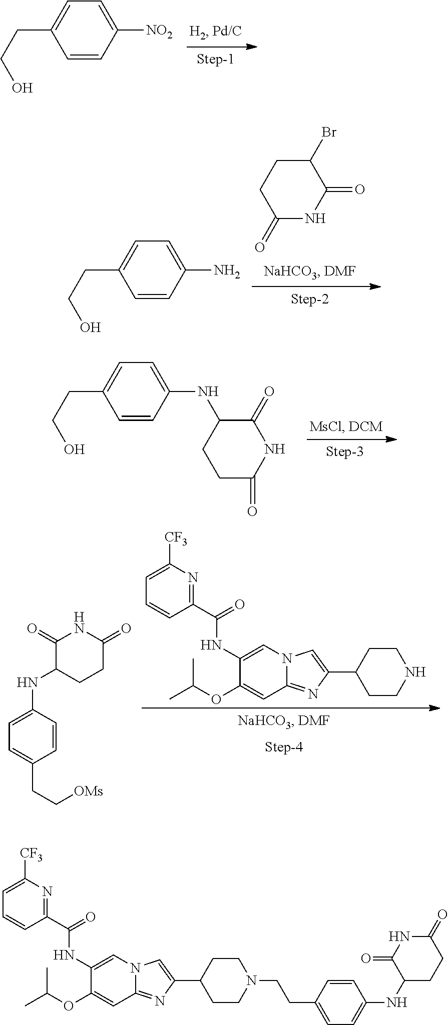

### Step-1

To a stirred solution of 2-(4-nitrophenyl)ethanol (3.00 g, 17.95 mmol) in ethanol (30 mL) placed in a 25 mL round bottom flask was added Palladium on charcoal (572.97 mg, 5.38 mmol). This resultant reaction mixture was stirred at room temperature under a hydrogen bladder atmosphere for 16 h. Upon the completion, the reaction mixture was filtered through celite bed and washed with ethyl acetate. Collected filtrate was concentrated under reduced pressure to afford 2-(4-aminophenyl)ethanol (2.3 g, 16.53 mmol, 92.13% yield) as an off white solid. LC-MS (ES⁺): m/z 137.99 [M+H]⁺.

### Step-2

To a stirred solution of 2-(4-aminophenyl)ethanol (1.5 g, 10.93 mmol) in DMF (15 mL) placed in a 50 mL round bottom flask was added sodium hydrogen carbonate (2.30 g, 27.34 mmol, 1.06 mL) and 3-bromopiperidine-2,6-dione (2.73 g, 14.22 mmol). This resultant reaction mixture was stirred at 70° C. for 16 h. Upon the completion, the reaction mixture was diluted with ethyl acetate, then washed with water and brine solution. The collected organic layer was dried over with Na₂SO₄, filtered, and then concentrated under reduced pressure to afford crude compound. The crude was purified by flash column chromatography using 100-200 silica gel, eluted with 50-60% EtOAc in pet ether to furnish 3-[4-(2-hydroxyethyl) anilino]piperidine-2,6-dione (1 g, 3.42 mmol, 31.29% yield) as an off white solid. LC-MS (ES⁺): m/z 249.35 [M+H]⁺

### Step-3

To a stirred solution of 3-[4-(2-hydroxyethyl)anilino]piperidine-2,6-dione (0.4 g, 1.61 mmol) in DCM (10 mL) placed in a 25 mL round bottom flask at 0° C., was added N,N-diethylethanamine (326.06 mg, 3.22 mmol, 449.11 μL) and methanesulfonyl chloride (239.92 mg, 2.09 mmol, 162.11 μL). This resultant reaction mixture was stirred at 70° C. for 16h. Upon the completion, the reaction mixture was diluted with dichloromethane was washed with water and brine solution. Collected organic layer was dried over with Na₂SO₄, filtered and then under reduced pressure to afford crude compound. The crude compound was purified by flash column chromatography using neutral alumina, eluted with 50-60% EtOAc in petroleum ether to furnish 2-[4-[(2,6-dioxo-3-piperidyl)amino]phenyl]ethyl methanesulfonate (0.17 g, 365.09 μmol, 22.66% yield) as an off white solid. LC-MS (ES⁺): m/z 327.12 [M+H]⁺.

### Step-4

To a stirred solution of N-[7-isopropoxy-2-(4-piperidyl)imidazo[1,2-a]pyridin-6-yl]-6-(trifluoromethyl)pyridine-2-carboxamide (274.20 mg, 612.81 μmol) in DMF (4 mL) placed in a 25 mL round bottom flask was added sodium bicarbonate (128.70 mg, 1.53 mmol, 59.58 p L) and 2-[4-[(2,6-dioxo-3-piperidyl)amino]phenyl]ethyl methanesulfonate (0.2 g, 612.81 μmol), then stirred at 75° C. for 16 h. Upon the completion, the mixture was concentrated under Genevac to remove the solvent. The residue was purified by Prep-HPLC and the fraction was lyophilized to furnish N-[2-[1-[2-[4-[(2,6-dioxo-3-piperidyl)amino]phenyl]ethyl]-4-piperidyl]-7-isopropoxy-imidazo[1,2-a]pyridin-6-yl]-6-(trifluoromethyl)pyridine-2-carboxamide formic acid salt (22 mg, 28.93 μmol, 4.72% yield) as an off white solid. LC-MS (ES⁺): m/z 678.38 [M+H]⁺. ¹H NMR (400 MHz, DMSO-d₆) δ 10.75 (s, 1H), 10.44 (s, 1H), 9.43 (s, 1H), 8.41 (m, 2H), 8.24 (d, J=7.2 Hz, 1H), 7.66 (s, 1H), 7.12 (s, 1H), 6.95 (d, J=8.2 Hz, 1H), 6.61 (d, J=8.3 Hz, 1H), 5.62 (d, J=7.3 Hz, 1H), 4.88 (m, 2H), 4.22 (m, 2H), 2.99 (d, J=10.9 Hz, 2H), 2.74 (m, 1H), 2.58 (t, J=8.7 Hz, 4H), 2.45 (m, 2H), 2.10 (m, 3H), 1.90 (m, 3H), 1.66 (q, J=10.8 Hz, 2H), 1.41 (m, 6H), 1.26 (d, J=19.3 Hz, 1H), 0.88 (t, J=6.5 Hz, 1H).

### Prep-HPLC Method:

Column/dimensions: SUNFIRE C8 (19*250*5 um)
Mobile phase A: 0.1% FA in water (aq)
Mobile phase B: 100% Acetonitrile
Gradient (Time/% B): 0/10, 2/10, 2.5/15, 17/43, 18/95, 21,/95, 21.1/10, 23/10
Flow rate: 17 ml/min
Solubility: ACN+WATER+THF
TLC Pattern: 10% methanol in DCM R_{f} value: 0.3

### Example 69

Compound of Example 69 was prepared substantially following the synthesis of Example 68.

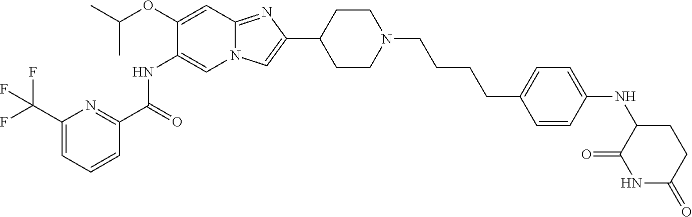 [SMILES: CC(C)Oc1cc2nc(C3CCN(CCCCc4ccc(NC5CCC(=O)NC5=O)cc4)CC3)cn2cc1NC(=O)c1cccc(C(F)(F)F)n1]

N-[2-[1-[4-[4-[(2,6-dioxo-3-piperidyl)amino]phenyl]butyl]-4-piperidyl]-7-isopropoxy-imidazo[1,2-a]pyridin-6-yl]-6-(trifluoromethyl)pyridine-2-carboxamide. LC-MS (ES⁺): m/z 706.18 [M+H]⁺. ¹H NMR (400 MHz, DMSO-d₆) δ 10.77 (s, 1H), 10.44 (s, 1H), 9.42 (s, 1H), 8.43-8.24 (m, 5H), 7.65 (s, 1H), 7.11 (s, 1H), 6.91 (d, J=8.2 Hz, 2H), 6.60 (d, J=8.3 Hz, 2H), 5.61 (d, J=7.4 Hz, 1H), 4.88 (t, J=6.0 Hz, 1H), 4.26 (m, 1H), 2.82-2.59 (m, 5H), 2.44 (m, 2H), 2.30 (q, J=7.6 Hz, 1H), 2.11 (m, 5H), 1.92 (m, 11H), 1.40 (d, J=6.0 Hz, 1H).

### Example 70

Compound of Example 70 was prepared substantially following the synthesis of Example 68.

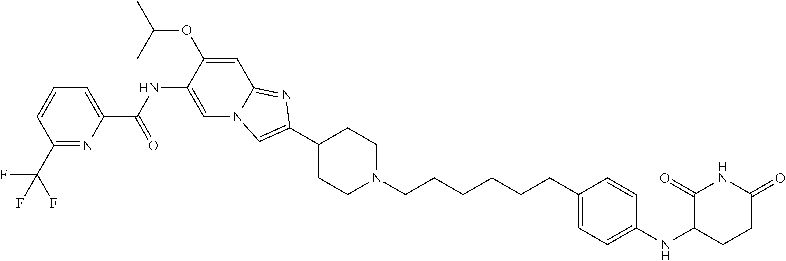 [SMILES: CC(C)Oc1cc2nc(C3CCN(CCCCCCc4ccc(NC5CCC(=O)NC5=O)cc4)CC3)cn2cc1NC(=O)c1cccc(C(F)(F)F)n1]

N-[2-[1-[6-[4-[(2,6-dioxo-3-piperidyl)amino]phenyl]hexyl]-4-piperidyl]-7-isopropoxy-imidazo[1,2-a]pyridin-6-yl]-6-(trifluoromethyl)pyridine-2-carboxamide. LC-MS (ES⁺): m/z 734.16 [M+H]⁺. ¹H NMR (400 MHz, DMSO-d₆) δ 10.77 (s, 1H), 10.44 (s, 1H), 9.43 (s, 1H), 8.45 (m, 8H), 8.24 (d, J=7.2 Hz, 1H), 7.65 (s, 1H), 7.11 (s, 1H), 6.90 (d, J=8.1 Hz, 1H), 6.59 (d, J=8.2 Hz, 1H), 5.61 (d, J=7.4 Hz, 1H), 4.88 (t, J=6.0 Hz, 1H), 4.24 (q, J=5.2 Hz, 1H), 2.90 (d, J=10.5 Hz, 2H), 2.74 (m, 1H), 2.59 (d, J=3.9 Hz, 2H), 2.50 (s, 2H), 2.26 (t, J=7.0 Hz, 2H), 2.09 (d, J=4.0 Hz, 1H), 1.92 (m, 5H), 1.63 (d, J=10.4 Hz, 2H), 1.40 (m, 5H), 1.26 (m, 5H).

### Synthesis of 2-[4-[4-[(2,6-dioxo-3-piperidyl)amino]phenyl]-1-piperidyl]acetic acid

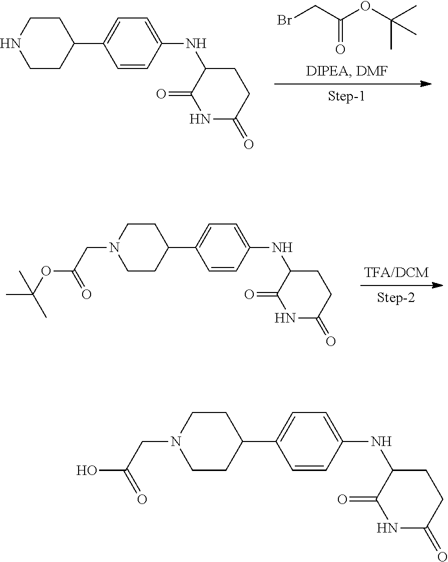

### Step-1

To a stirred solution of 3-[4-(4-piperidyl)anilino]piperidine-2,6-dione (0.15 g, 522.00 μmol) and tert-butyl 2-bromoacetate (101.82 mg, 522.00 μmol, 76.56 μL) in ACN (10 mL) was added N-ethyl-N-isopropyl-propan-2-amine (404.79 mg, 3.13 mmol, 545.54 p L) the reaction mixture was purge with nitrogen for 5 mins at room temperature, and the reaction mixture was stirred at 70° C. for 1 hour. The reaction mixture was concentrated under reduced pressure and purification by column chromatography using silica afforded tert-butyl 2-[4-[4-[(2,6-dioxo-3-piperidyl)amino]phenyl]-1-piperidyl]acetate (0.15 g, 261.52 μmol, 50.10% yield) as light green solid. LC-MS (ES⁺): m/z 402.28 [M+H]⁺.

### Step-2

To a stirred a suspension of tert-butyl 2-[4-[4-[(2,6-dioxo-3-piperidyl)amino]phenyl]-1-piperidyl]acetate (0.25 g, 622.67 μmol) in DCM (5 mL) at 0° C. was added 2,2,2-trifluoroacetic acid (851.98 mg, 7.47 mmol, 575.66 p L) dropwise. The reaction mixture was stirred for 3 h at room temperature. The reaction mixture was concentrated under vacuum and the residue was triturated with diethyl ether (10 mL) to give 2-[4-[4-[(2,6-dioxo-3-piperidyl)amino]phenyl]-1-piperidyl]acetic acid trifluoroacetic acid salt (0.2 g, 326.50 μmol, 52.44% yield). LC-MS (ES⁺): m/z 346.42 [M+H]⁺.

### Example 71 Synthesis of N-[2-[3-[[2-[4-[4-[(2,6-dioxo-3-piperidyl)amino]phenyl]-1-piperidyl]acetyl]amino]propyl]-7-isopropoxy-imidazo[1,2-a]pyridin-6-yl]-6-(trifluoromethyl)pyridine-2-carboxamide

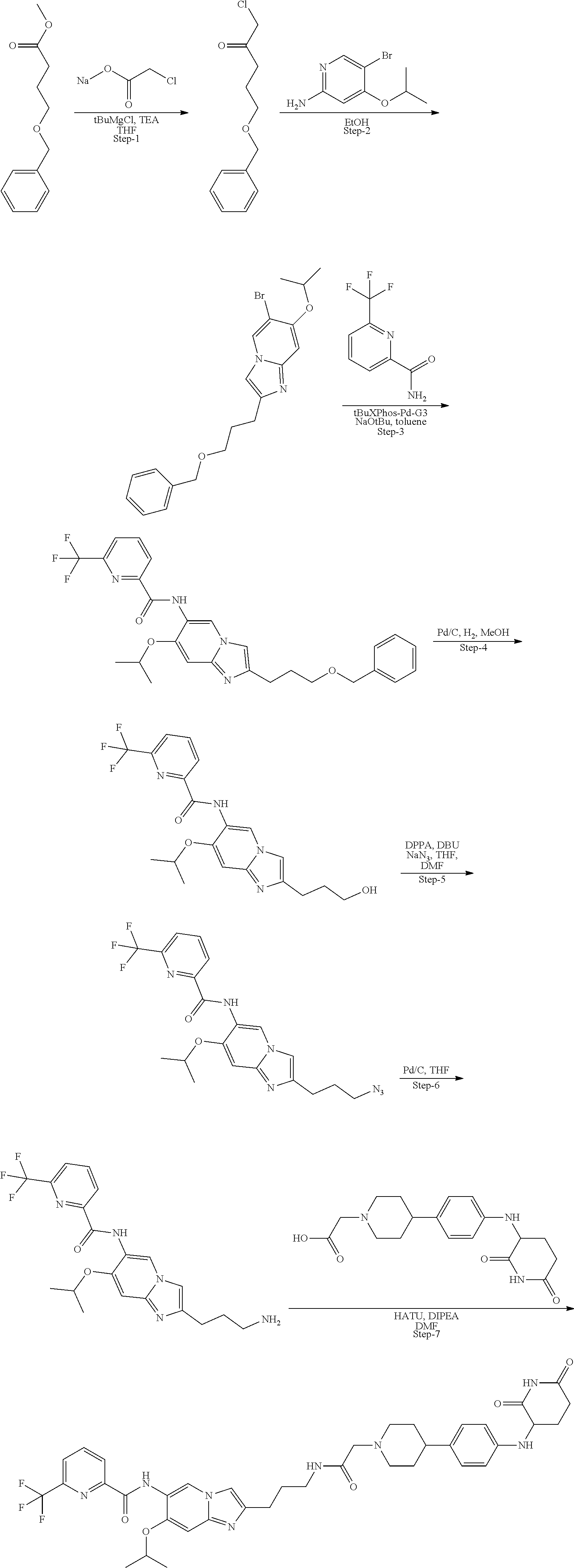

### Step-1

To a stirred solution of methyl 4-benzyloxybutanoate (27 g, 129.65 mmol), sodium; 2-chloroacetate (22.65 g, 194.47 mmol) and triethylamine (19.68 g, 194.47 mmol, 27.11 mL) in THF (200 mL) was slowly added tert-butyl(chloro)magnesium (30.30 g, 259.30 mmol) at 0° C. The reaction was brought to 25° C. and stirred for 4 hours. The reaction was quenched with cold saturated ammonium chloride solution, extracted with ethyl acetate two times, the ethyl acetate layer was washed with brine, dried over anhydrous Na₂SO₄ and concentrated.

The crude compound thus obtained was purified by silica gel column chromatography using ethyl acetate and pet ether as eluent to obtain 5-benzyloxy-1-chloro-pentan-2-one (10 g, 43.19 mmol, 33.31% yield) as pale yellow liquid. LC-MS (ES⁺): m/z 132.06 [M+H]⁺.

### Step-2

5-bromo-4-isopropoxy-pyridin-2-amine (8 g, 34.62 mmol) and 5-benzyloxy-1-chloro-pentan-2-one (8.24 g, 36.35 mmol) were dissolved in Ethanol (70 mL) and heated in a sealed tube at 100° C. for 48 hours. The ethanol was evaporated and the crude compound was subjected to reverse phase column chromatography using 0.1% in formic acid in water and acetonitrile as eluent to obtain 2-(3-benzyloxypropyl)-6-bromo-7-isopropoxy-imidazo[1,2-a]pyridine (6 g, 13.35 mmol, 42.11% yield). LC-MS (ES⁺): m/z 404.81 [M+H]⁺.

### Step-3

To a stirred solution of 2-(3-benzyloxypropyl)-6-bromo-7-isopropoxy-imidazo[1,2-a]pyridine formic acid salt (6 g, 13.35 mmol), 6-(trifluoromethyl)pyridine-2-carboxamide (3.81 g, 20.03 mmol) and sodium; 2-methylpropan-2-olate (2.57 g, 26.71 mmol) was degassed with argon for 15 minutes, tBuXPhos Pd G3 (1.06 g, 1.34 mmol) was added to the reaction mixture and again degassed for five minutes. The reaction mixture was then heated at 100° C. for 16 hours. The reaction was worked up by concentrating to remove toluene, quenched by adding water, extracted with ethyl acetate two times, the ethyl acetate layer was washed with brine, dried over anhydrous Na₂SO₄ and evaporated under reduced pressure to get crude solid. The purification of crude compound was done by reverse phase column chromatography using 10 mM Ammonium acetate in water and acetonitrile as eluent to obtain N-[2-(3-benzyloxypropyl)-7-isopropoxy-imidazo[1,2-a]pyridin-6-yl]-6-(trifluoromethyl)pyridine-2-carboxamide (1.9 g, 3.63 mmol, 27.21% yield). LC-MS (ES⁺): m/z 513.87 [M+H]⁺.

### Step-4

To a stirred solution of N-[2-(3-benzyloxypropyl)-7-isopropoxy-imidazo[1,2-a]pyridin-6-yl]-6-(trifluoromethyl)pyridine-2-carboxamide (1.9 g, 3.71 mmol) in methanol (25 mL) was added palladium, 10% on carbon, dry (394.52 mg, 3.71 mmol) and two drops of conc. HCl. The reaction mixture was then stirred under a hydrogen atmosphere for 16 hours. The reaction mixture was then passed through a celite bed, washed with methanol and concentrated under vacuum to give a residue, which was then neutralized using saturated NaHCO₃ and extracted with ethyl acetate, the ethyl acetate layer was dried over anhydrous Na₂SO₄ and subjected to evaporation to give N-[2-(3-hydroxypropyl)-7-isopropoxy-imidazo[1,2-a]pyridin-6-yl]-6-(trifluoromethyl)pyridine-2-carboxamide (1.1 g, 2.43 mmol, 65.54% yield). LC-MS (ES⁺): m/z 423.70 [M+H]⁺.

### Step-5

A stirred solution of N-[2-(3-hydroxypropyl)-7-isopropoxy-imidazo[1,2-a]pyridin-6-yl]-6-(trifluoromethyl)pyridine-2-carboxamide (0.8 g, 1.89 mmol) in THF (15 mL) was cooled in an ice bath under nitrogen and treated with [azido(phenoxy)phosphoryl]oxybenzene (1.04 g, 3.79 mmol, 820.81 p L) and 2,3,4,6,7,8,9,10-octahydropyrimido[1,2-a]azepine (576.67 mg, 3.79 mmol, 565.36 p L) in one portion. The reaction mixture was stirred at room temperature for 16 h. The reaction was monitored by TLC and LC-MS which showed consumption of starting material and generating a intermediate with a m/z 654, this intermediate (1.2 g) was isolated by quenching the reaction mixture with water, extraction with EtOAc and evaporation to give a residue. The residue obtained was dissolved DMF (15 mL), sodium azide was added (492.50 mg, 7.58 mmol, 266.22 μL) and the reaction mixture was stirred at 25° C. for 16 hours. The reaction was worked up using ethyl acetate and water. The ethyl acetate layer was separated, washed with a brine solution, dried over anhydrous Na₂SO₄ concentrated under reduced pressure and purification by silica gel chromatography using ethyl acetate pet ether as an eluent gave N-[2-(3-azidopropyl)-7-isopropoxy-imidazo[1,2-a]pyridin-6-yl]-6-(trifluoromethyl)pyridine-2-carboxamide (0.34 g, 734.85 μmol, 38.80% yield) as off white solid. LC-MS (ES⁺): m/z 448.28 [M+H]⁺.

### Step-6

To a solution of N-[2-(3-azidopropyl)-7-isopropoxy-imidazo[1,2-a]pyridin-6-yl]-6-(trifluoromethyl)pyridine-2-carboxamide (0.34 g, 759.92 μmol) in THF (15 mL) was added palladium, 10% on carbon, dry (80.87 mg, 759.92 μmol) and the reaction mixture was stirred at room temperature for 6 h. The reaction mixture was passed through celite bed and the filtrate was subjected to evaporation. The residue obtained was cooled to 0° C. and 4M HCl in 1,4-Dioxane was slowly added until the pH is 3, the reaction mixture was subjected to evaporation and the residue obtained was purified by trituration with n-pentane and diethyl ether to give N-[2-(3-aminopropyl)-7-isopropoxy-imidazo[1,2-a]pyridin-6-yl]-6-(trifluoromethyl)pyridine-2-carboxamide hydrochloride salt (0.220 g, 412.20 μmol, 54.24% yield) as off-white solid. LC-MS (ES⁺): m/z 422.26 [M+H]⁺.

### Step-7

To a stirred solution of 2-[4-[4-[(2,6-dioxo-3-piperidyl)amino]phenyl]-1-piperidyl]acetic acid formic acid salt (18.86 mg, 41.05 μmol), DIPEA (42.34 mg, 327.60 μmol, 57.06 μL) and HATU (20.76 mg, 54.60 μmol) in DMF (1 mL) at 25° C. was stirred for 20 minutes followed by the addition of N-[2-(3-aminopropyl)-7-isopropoxy-imidazo[1,2-a]pyridin-6-yl]-6-(trifluoromethyl)pyridine-2-carboxamide hydrochloride salt (0.025 g, 54.60 μmol). The reaction mixture was then stirred at 25° C. for 16 hours. The reaction mixture was evaporated to remove DMF and submitted for prep-HPLC for purification to obtain N-[2-[3-[[2-[4-[4-[(2,6-dioxo-3-piperidyl)amino]phenyl]-1-piperidyl]acetyl]amino]propyl]-7-isopropoxy-imidazo[1,2-a]pyridin-6-yl]-6-(trifluoromethyl)pyridine-2-carboxamide trifluoroacetic acid salt (15.2 mg, 16.43 μmol, 30.09% yield) as yellow solid. LC-MS (ES⁺): m/z 749.46 [M+H]⁺. ¹H NMR (400 MHz, DMSO-d₆) δ 14.05 (s, 1H), 10.78 (s, 1H), 10.56 (s, 1H), 9.79 (s, 1H), 9.66 (s, 1H), 8.68 (s, 1H), 8.47 (d, J=7.6 Hz, 2H), 8.31 (d, J=7.1 Hz, 1H), 8.11 (s, 1H), 7.46 (s, 1H), 7.08 (q, J=37.3 Hz, 1H), 6.64 (d, J=8.4 Hz, 3H), 5.12 (m, 1H), 4.28 (q, J=5.3 Hz, 1H), 3.93 (s, 2H), 3.54 (q, J=9.6 Hz, 2H), 3.27 (t, J=5.9 Hz, 2H), 3.16 (d, J=10.2 Hz, 2H), 2.83 (t, J=7.4 Hz, 1H), 2.67 (m, 4H), 2.09 (t, J=7.6 Hz, 1H), 1.89 (m, 6H), 1.47 (d, J=6.0 Hz, 6H).

### Example 72 Synthesis of N-[2-[6-[4-[4-[(2,6-dioxo-3-piperidyl)amino]phenyl]-1-piperidyl]hexyl]-7-isopropoxy-imidazo[1,2-a]pyridin-6-yl]-6-(trifluoromethyl)pyridine-2-carboxamide

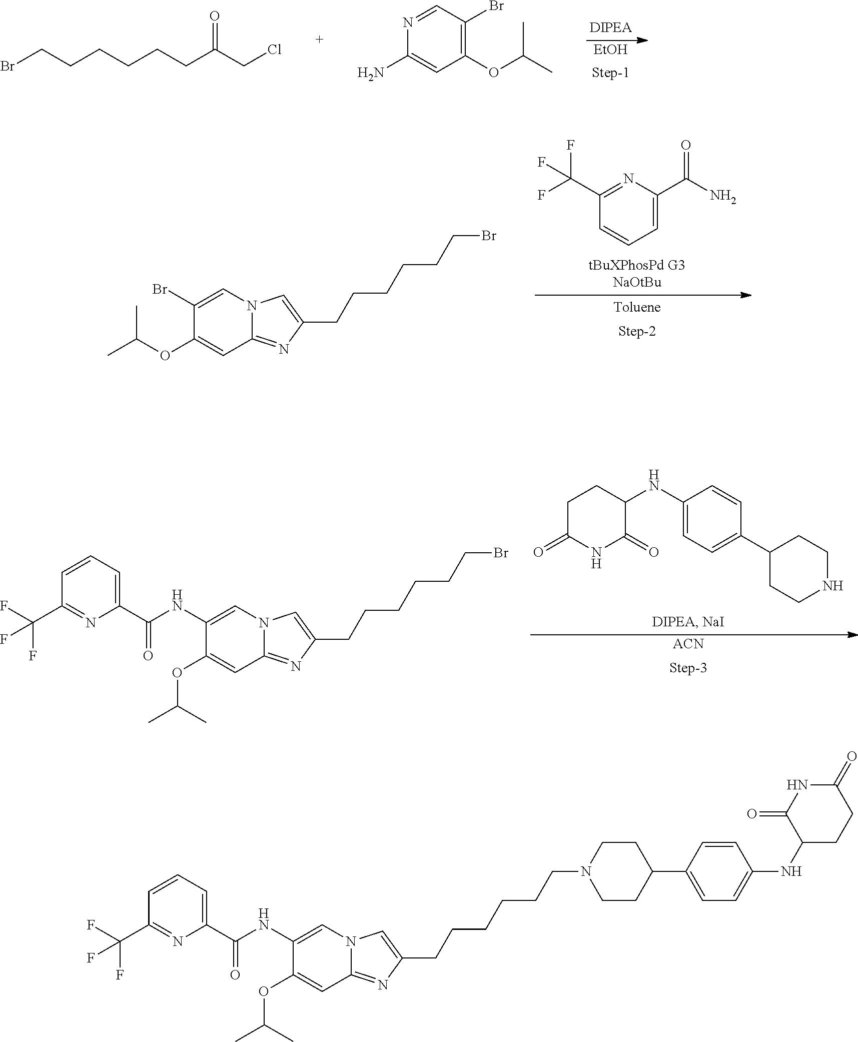

### Step-1

A stirred solution of 8-bromo-1-chloro-octan-2-one (4.1 g, 16.97 mmol) and 5-bromo-4-isopropoxy-pyridin-2-amine (3.92 g, 16.97 mmol) in ethanol (30 mL) in a sealed tube was heated at 90° C. for 16 hr. The ethanol was concentrated under reduced pressure and the crude compound was purified by silica gel column chromatography using (0-30%) pet ether/EtOAc to afford 6-bromo-2-(6-bromohexyl)-7-isopropoxy-imidazo[1,2-a]pyridine (4.1 g, 9.80 mmol, 57.76% yield). ¹H NMR (400 MHz, DMSO-d₆) δ 8.14 (s, 1H), 7.27 (s, 1H), 6.80 (s, 1H), 4.58 (m, 1H), 4.11 (m, 1H), 3.54-3.38 (m, 2H), 2.72 (m, 2H), 2.04 (m, 2H) 1.86-1.73 (m, 2H), 1.47-1.25 (m, 9H).

### Step-2

In seal tube, a stirred solution of 6-bromo-2-(6-bromohexyl)-7-isopropoxy-imidazo[1,2-a]pyridine (0.050 g, 119.57 μmol), 6-(trifluoromethyl)pyridine-2-carboxamide (27.28 mg, 143.48 μmol) in toluene (5 mL) was added sodium tert-butoxide (34.47 mg, 358.71 μmol) and degassed with argon for 15 minutes. Then tBuXPhosPd G3 (9.50 mg, 11.96 μmol) was added to the reaction mixture and again degassed for 5 minutes. The reaction mixture was then heated at 100° C. for 16 hr. The reaction mixture was filtered through a celite bed and the filtrate was concentrated to afford N-[2-(6-bromohexyl)-7-isopropoxy-imidazo[1,2-a]pyridin-6-yl]-6-(trifluoromethyl)pyridine-2-carboxamide (0.043 g, 4.08 μmol, 3.41% yield). LC-MS (ES⁺): 525.4 [M+H]⁺

### Step-3

To a stirred solution of N-[2-(6-bromohexyl)-7-isopropoxy-imidazo[1,2-a]pyridin-6-yl]-6-(trifluoromethyl)pyridine-2-carboxamide (0.2 g, 379.24 μmol) and 3-((4-(piperidin-4-yl)phenyl)amino)piperidine-2,6-dione (130.77 mg, 455.08 μmol) in acetonitrile (20 mL) was added N-ethyl-N-isopropyl-propan-2-amine (147.04 mg, 1.14 mmol, 198.17 p L) and sodium iodide (28.42 mg, 189.62 μmol, 7.74 μL). The reaction mixture was stirred at 80° C. for 16 hr. The reaction mixture was concentrated under reduced vacuum at 50° C. and purification by prep HPLC follow by lyophilization to give N-[2-[6-[4-[4-[(2,6-dioxo-3-piperidyl) amino]phenyl]-1-piperidyl]hexyl]-7-isopropoxy-imidazo[1,2-a]pyridin-6-yl]-6-(trifluoromethyl)pyridine-2-carboxamide formic acid salt (39.7 mg, 50.67 μmol, 13.36% yield). LC-MS (ES⁺): 734.20 [M+H]⁺. ¹H NMR (400 MHz, DMSO-d₆) δ 0.74 (s, 1H), 10.44 (s, 1H), 9.43 (s, 1H), 8.43 (m, 1H), 8.24 (s, 1H), 7.65 (s, 1H), 7.05 (s, 1H), 6.98 (d, J=8.5 Hz, 2H), 6.64 (d, J=8.6 Hz, 2H), 5.66 (d, J=7.6 Hz, 1H), 4.87 (t, J=6.1 Hz, 1H), 4.27 (q, J=5.4 Hz, 1H), 3.62 (m, 1H), 3.45 (d, J=10.1 Hz, 2H), 2.92 (m, 1H), 2.66 (m, 5H), 2.49-2.07 (m, 4H), 1.90 (t, J=11.8 Hz, 1H), 1.73-1.51 (m, 8H), 1.42 (m, 10H).

### Prep HPLC Purification Method:

Column/dimensions: X SELECT C18 (19*250*5 um)
Mobile phase A: 0.05% FA in water (aq.)
Mobile phase B: Acetonitrile (org)
Gradient (Time/% B): 0/10, 3/15, 17.5/31, 17.6/98, 20/98, 20.1/10, 22/10
Flow rate: 17 ml/min
Solubility: ACN+THF+WATER

### General Procedure 1 for Examples 73-79

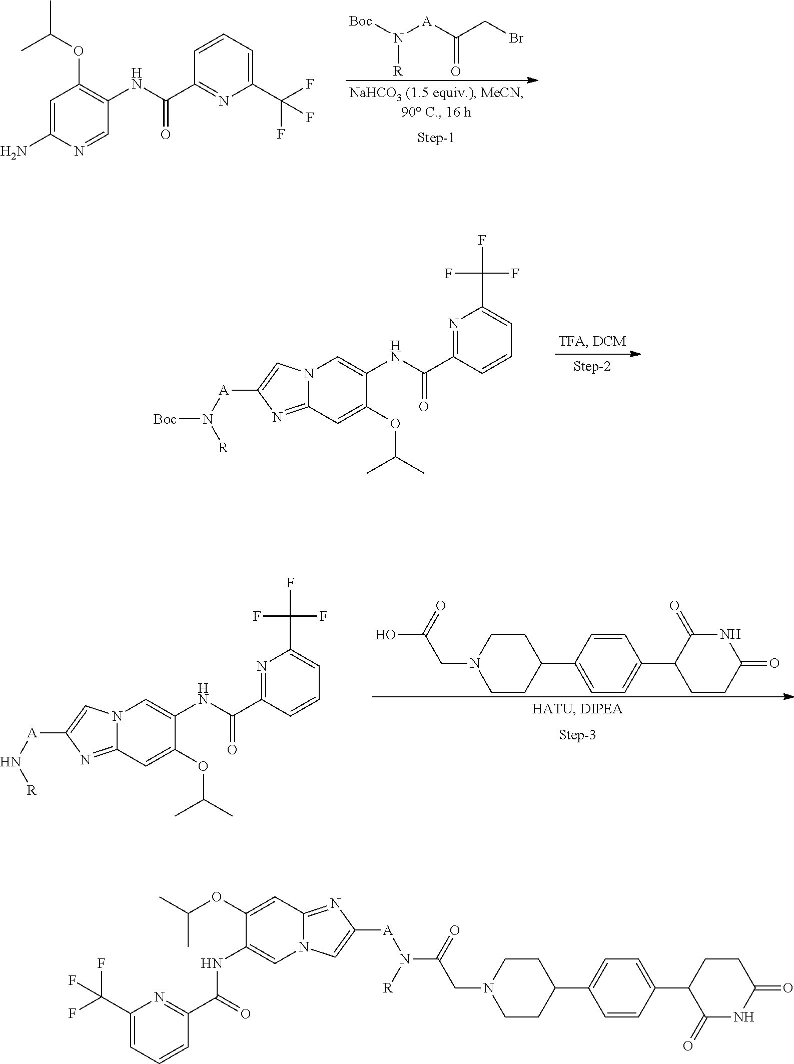

N-(6-amino-4-isopropoxypyridin-3-yl)-6-(trifluoromethyl)picolinamide was prepared following the procedure from Example 266 of WO 2020150626 A1.

### Step-1

N-(6-amino-4-isopropoxypyridin-3-yl)-6-(trifluoromethyl)picolinamide (1.0 equiv.), respective methylene bromide building block (1.3 equiv.) and NaHCO₃ (1.5 equiv.) were mixed in dry AcCN (appr. 2.5 mL per 200 mg of product). The reaction mixture was heated at 90° C. for 16 hours. The reaction mixture was cooled down, filtered and subjected to prep. HPLC (Waters SunFire C18 19*100 5 mkm column; gradient mixture H₂O-MeOH as a mobile phase) to afford the desired intermediate building block.

### Step-2 and Step-3

Respective Intermediate building block (1.0 equiv.) was dissolved in dry CH₂Cl₂ (0.5 mL) and TFA (0.5 mL) was added in one portion. The mixture was stirred for 4 hours at ambient temperature. The reacting mixture was evaporated under reduced pressure, and the residue was dissolved in dry DMSO (0.6 mL). 2-{4-[4-(2,6-Dioxopiperidin-3-yl)phenyl]piperidin-1-yl}acetic acid hydrochloride (1.0 equiv.), DIPEA (6.0 equiv.), and HATU (1.1 equiv.) were added in one portion and the reaction mixture was sealed and left at ambient temperature for 16 hours. After LCMS showed full conversion, the reaction mixture was evaporated under reduced pressure and the residue was dissolved in DMSO (0.2 mL), filtered, and subjected to prep. HPLC (Waters SunFire C18 19*100 5 mkm column; gradient mixture H₂O-CAN-0.1% TFA as a mobile phase) to afford the desired products.

### Example 73

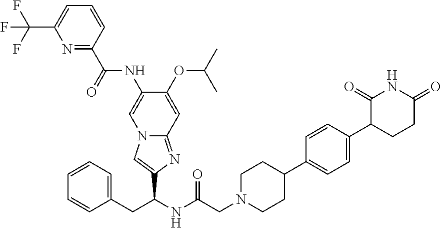 [SMILES: CC(C)Oc1cc2nc([C@H](Cc3ccccc3)NC(=O)CN3CCC(c4ccc(C5CCC(=O)NC5=O)cc4)CC3)cn2cc1NC(=O)c1cccc(C(F)(F)F)n1]

N-[2-[(1S)-1-[[2-[4-[4-(2,6-dioxo-3-piperidyl)phenyl]-1-piperidyl]acetyl]amino]-2-phenyl-ethyl]-7-isopropoxy-imidazo[1,2-a]pyridin-6-yl]-6-(trifluoromethyl)pyridine-2-carboxamide. Compound of Example 73 was prepared following the General Procedure 1, using tert-butyl (S)-(4-bromo-3-oxo-1-phenylbutan-2-yl)carbamate as the methylene bromide building block. LC-MS (ES⁻): m/z 794.0 [M−H]⁻.

### Example 74

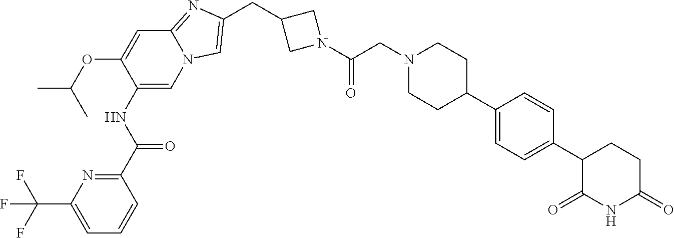 [SMILES: CC(C)Oc1cc2nc(CC3CN(C(=O)CN4CCC(c5ccc(C6CCC(=O)NC6=O)cc5)CC4)C3)cn2cc1NC(=O)c1cccc(C(F)(F)F)n1]

N-[2-[[1-[2-[4-[4-(2,6-dioxo-3-piperidyl)phenyl]-1-piperidyl]acetyl]azetidin-3-yl]methyl]-7-isopropoxy-imidazo[1,2-a]pyridin-6-yl]-6-(trifluoromethyl)pyridine-2-carboxamide. Compound of Example 74 was prepared following the General Procedure 1, using tert-butyl 3-(3-bromo-2-oxopropyl)azetidine-1-carboxylate as the methylene bromide building block. LC-MS (ES⁻): m/z 794.0 [M−H]⁻.

### Example 75

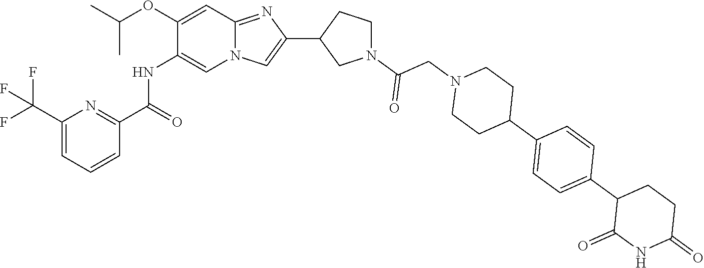 [SMILES: CC(C)Oc1cc2nc(C3CCN(C(=O)CN4CCC(c5ccc(C6CCC(=O)NC6=O)cc5)CC4)C3)cn2cc1NC(=O)c1cccc(C(F)(F)F)n1]

N-[2-[1-[2-[4-[4-(2,6-dioxo-3-piperidyl)phenyl]-1-piperidyl]acetyl]pyrrolidin-3-yl]-7-isopropoxy-imidazo[1,2-a]pyridin-6-yl]-6-(trifluoromethyl)pyridine-2-carboxamide. Compound of Example 75 was prepared following the General Procedure 1, using tert-butyl 3-(2-bromoacetyl)pyrrolidine-1-carboxylate as the methylene bromide building block. LC-MS (ES⁻): m/z 744.2 [M−H]⁻.

### Example 76

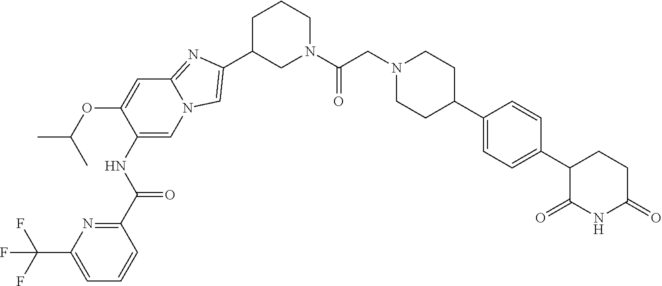 [SMILES: CC(C)Oc1cc2nc(C3CCCN(C(=O)CN4CCC(c5ccc(C6CCC(=O)NC6=O)cc5)CC4)C3)cn2cc1NC(=O)c1cccc(C(F)(F)F)n1]

N-[2-[1-[2-[4-[4-(2,6-dioxo-3-piperidyl)phenyl]-1-piperidyl]acetyl]-3-piperidyl]-7-isopropoxy-imidazo[1,2-a]pyridin-6-yl]-6-(trifluoromethyl)pyridine-2-carboxamide. Compound of Example 76 was prepared following the General Procedure 1, using tert-butyl 3-(2-bromoacetyl)piperidine-1-carboxylate as the methylene bromide building block. LC-MS (ES⁻): m/z 758.2 [M−H]⁻.

### Example 77

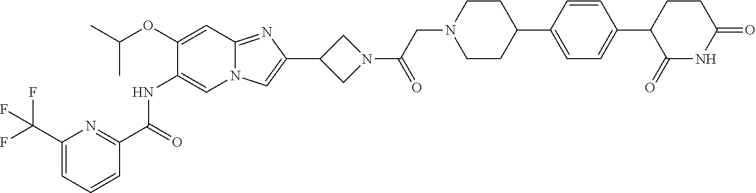 [SMILES: CC(C)Oc1cc2nc(C3CN(C(=O)CN4CCC(c5ccc(C6CCC(=O)NC6=O)cc5)CC4)C3)cn2cc1NC(=O)c1cccc(C(F)(F)F)n1]

N-[2-[1-[2-[4-[4-(2,6-dioxo-3-piperidyl)phenyl]-1-piperidyl]acetyl]azetidin-3-yl]-7-isopropoxy-imidazo[1,2-a]pyridin-6-yl]-6-(trifluoromethyl)pyridine-2-carboxamide. Compound of Example 77 was prepared following the General Procedure 1, using tert-butyl 3-(2-bromoacetyl)azetidine-1-carboxylate as the methylene bromide building block. LC-MS (ES⁻): m/z 730.4 [M−H]⁻.

### Example 78

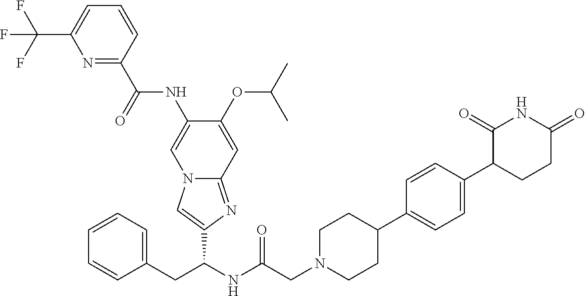 [SMILES: CC(C)Oc1cc2nc([C@@H](Cc3ccccc3)NC(=O)CN3CCC(c4ccc(C5CCC(=O)NC5=O)cc4)CC3)cn2cc1NC(=O)c1cccc(C(F)(F)F)n1]

N-[2-[(1R)-1-[[2-[4-[4-(2,6-dioxo-3-piperidyl)phenyl]-1-piperidyl]acetyl]amino]-2-phenyl-ethyl]-7-isopropoxy-imidazo[1,2-a]pyridin-6-yl]-6-(trifluoromethyl)pyridine-2-carboxamide. Compound of Example 78 was prepared following the General Procedure 1, using tert-butyl (R)-(4-bromo-3-oxo-1-phenylbutan-2-yl)carbamate as the methylene bromide building block. LC-MS (ES⁻): m/z 794.0 [M−H]⁻.

### Example 79

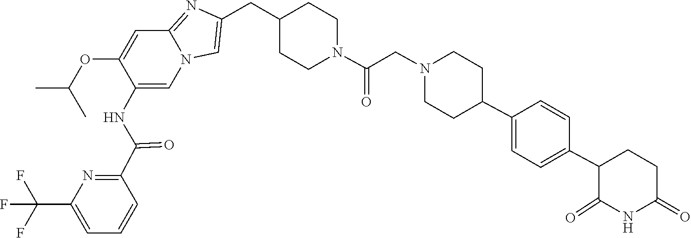 [SMILES: CC(C)Oc1cc2nc(CC3CCN(C(=O)CN4CCC(c5ccc(C6CCC(=O)NC6=O)cc5)CC4)CC3)cn2cc1NC(=O)c1cccc(C(F)(F)F)n1]

N-[2-[[1-[2-[4-[4-(2,6-dioxo-3-piperidyl)phenyl]-1-piperidyl]acetyl]-4-piperidyl]methyl]-7-isopropoxy-imidazo[1,2-a]pyridin-6-yl]-6-(trifluoromethyl)pyridine-2-carboxamide. Compound of Example 79 was prepared following the General Procedure 1, using tert-butyl 4-(3-bromo-2-oxopropyl)piperidine-1-carboxylate as the methylene bromide building block. LC-MS (ES⁻): m/z 772.0 [M−H]⁻.

### Synthesis of N-(7-isopropoxy-2-(piperidin-4-yl)imidazo[1,2-a]pyridin-6-yl)-6-(trifluoromethyl)picolinamide

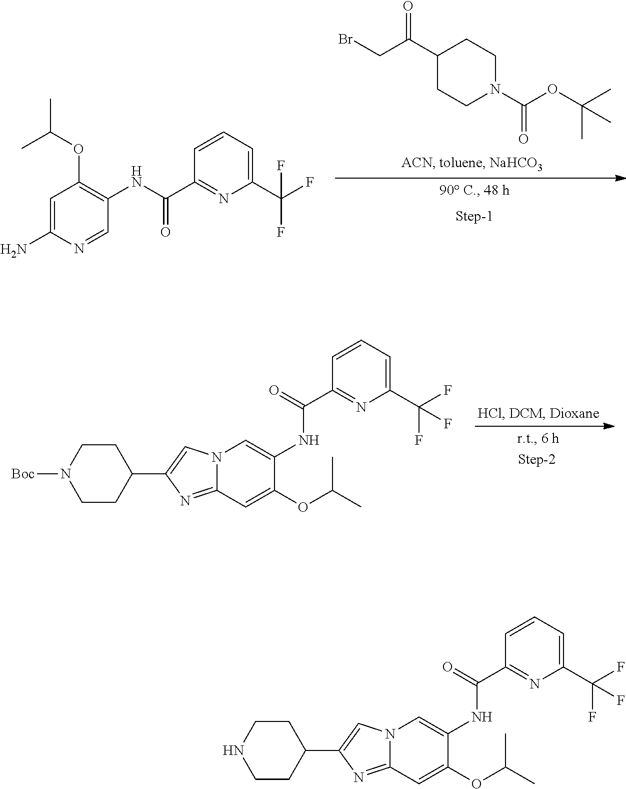 [SMILES: CC(C)Oc1cc(N)ncc1NC(=O)c1cccc(C(F)(F)F)n1 ; CC(C)(C)OC(=O)N1CCC(C(=O)CBr)CC1 ; CC(C)Oc1cc2nc(C3CCN(C(=O)OC(C)(C)C)CC3)cn2cc1NC(=O)c1cccc(C(F)(F)F)n1 ; CC(C)Oc1cc2nc(C3CCNCC3)cn2cc1NC(=O)c1cccc(C(F)(F)F)n1]

### Step-1

In a sealed tube, a mixture of N-(6-amino-4-isopropoxy-3-pyridyl)-6-(trifluoromethyl)pyridine-2-carboxamide (10 g, 29.39 mmol), tert-butyl 4-(2-bromoacetyl)piperidine-1-carboxylate (11.70 g, 38.20 mmol) and NaHCO₃ (3.21 g, 38.20 mmol, 1.49 mL) in ACN (250 mL) and Toluene (250 mL) was heated at 90° C. for 48 h. The mixture was cooled to room temperature then diluted with H₂O (100 mL) and extracted DCM (3×50 mL). The combined organic layers were washed with brine, dried over Na₂SO₄, filtered, and concentrated. The residue was purified by column chromatography on silica gel (Interchim, SiO₂ (60 g), DCM-MeOH (50-50%)) to provide tert-butyl 4-[7-isopropoxy-6-[[6-(trifluoromethyl)pyridine-2-carbonyl]amino]imidazo[1,2-a]pyridin-2-yl]piperidine-1-carboxylate (2.5 g, 4.15 mmol, 14.14% yield) as a yellow solid. LC-MS (ES⁺): m/z 548.2 [M+H]⁺.

### Step-2

To a solution of tert-butyl 4-[7-isopropoxy-6-[[6-(trifluoromethyl)pyridine-2-carbonyl]amino]imidazo[1,2-a]pyridin-2-yl]piperidine-1-carboxylate (2.5 g, 4.57 mmol) in DCM (50 mL) was added HCl (1.66 g, 45.66 mmol) in Dioxane (10 mL). The reaction mixture was stirred at room temperature for 6 h. The solvent was removed in vacuo and basified with sat. NaHCO₃ to pH=8˜9. and then extracted with DCM (50 mL×3). The combined organic layers were washed with H₂O (50 mL), brine (50 mL), dried over Na₂SO₄, filtered and concentrated. The resulting solid was purified by column chromatography on silica gel (Interchim, SiO2 (30 g), DCM-MeOH (30-70%)) to provide N-[7-isopropoxy-2-(4-piperidyl)imidazo[1,2-a]pyridin-6-yl]-6-(trifluoromethyl)pyridine-2-carboxamide (1.5 g, 3.35 mmol, 73.42% yield) as a yellow solid. LC-MS (ES⁺): m/z 448.0 [M+H]⁺.

### General Procedure 2 for the Synthesis of Examples 80-85

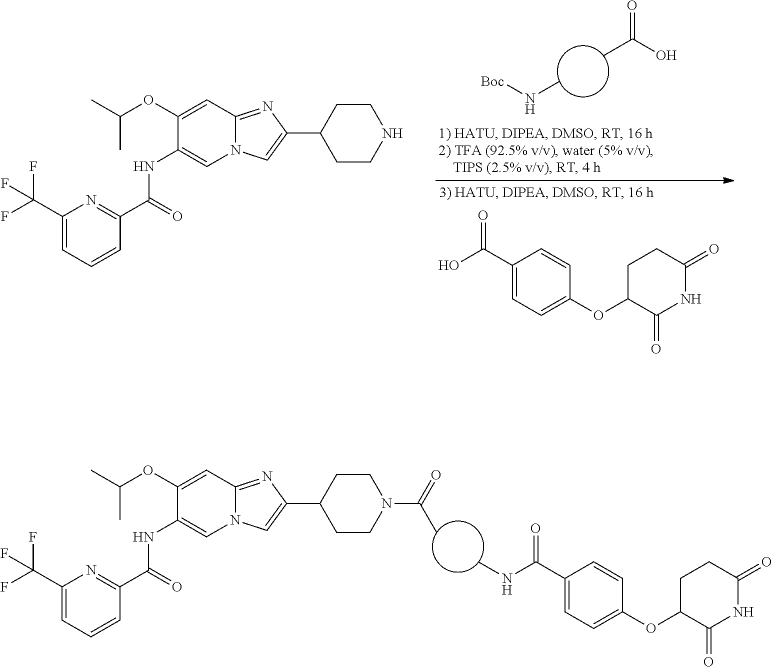

All reactions were performed on 30-50 mg scale.

A solution of N-(7-isopropoxy-2-(piperidin-4-yl)imidazo[1,2-a]pyridin-6-yl)-6-(trifluoromethyl)picolinamide (1 equiv.), respective acid building block (1.1 equiv.), HATU (1.1 equiv.) and DIPEA (2.5 equiv.) in dry DMSO (0.7 mL) was stirred at room temperature for 16 hours. The reacting mixture was washed with water (3 mL) and the obtained solution was concentrated under reduced pressure. A solution of TFA (92.5% v/v), water (5% v/v) and TIPS (2.5% v/v) was then added in one portion and stirred at room temperature for 6 hours. The reacting mixture was concentrated under reduced pressure. The residue was dissolved in dry DMSO (0.7 mL) followed by addition of 4-((2,6-dioxopiperidin-3-yl)oxy)benzoic acid (1.1 equiv.), DIPEA (5.2 equiv.), and HATU (1.1 equiv.) and was stirred at room temperature for 16 hours. The mixture was concentrated under reduced pressure and the residue was dissolved in DMSO (1 mL). The solution was filtered, analyzed by LCMS and then subjected to prep. HPLC (Waters SunFire C18 19*100 5 mkm column; gradient mixture H₂O-MeCN-0.1% TFA as a mobile phase) to afford the corresponding products.

### Example 80

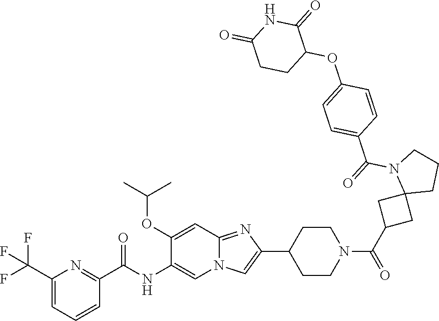 [SMILES: CC(C)Oc1cc2nc(C3CCN(C(=O)C4CC5(CCCN5C(=O)c5ccc(OC6CCC(=O)NC6=O)cc5)C4)CC3)cn2cc1NC(=O)c1cccc(C(F)(F)F)n1]

N-[2-[1-[5-[4-[(2,6-dioxo-3-piperidyl)oxy]benzoyl]-5-azaspiro[3.4]octane-2-carbonyl]-4-piperidyl]-7-isopropoxy-imidazo[1,2-a]pyridin-6-yl]-6-(trifluoromethyl)pyridine-2-carboxamide. Compound of Example 80 was prepared following the General Procedure 2, using 5-(tert-butoxycarbonyl)-5-azaspiro[3.4]octane-2-carboxylic acid as the acid building block. LC-MS (ES⁺): m/z 816.2 [M+H]⁺.

### Example 81

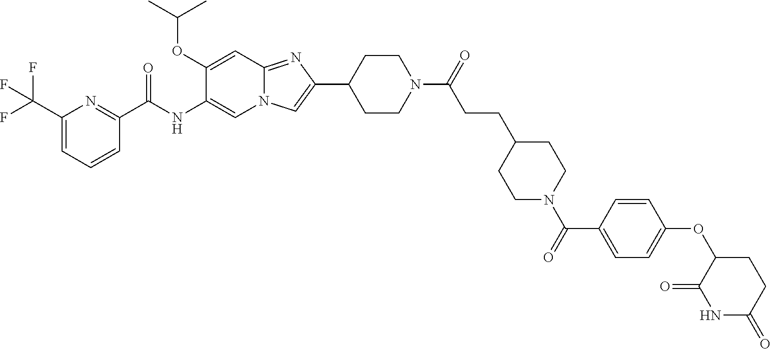 [SMILES: CC(C)Oc1cc2nc(C3CCN(C(=O)CCC4CCN(C(=O)c5ccc(OC6CCC(=O)NC6=O)cc5)CC4)CC3)cn2cc1NC(=O)c1cccc(C(F)(F)F)n1]

N-[2-[1-[3-[1-[4-[(2,6-dioxo-3-piperidyl)oxy]benzoyl]-4-piperidyl]propanoyl]-4-piperidyl]-7-isopropoxy-imidazo[1,2-a]pyridin-6-yl]-6-(trifluoromethyl)pyridine-2-carboxamide. Compound of Example 81 was prepared following the General Procedure 2, using 3-(1-(tert-butoxycarbonyl)piperidin-4-yl)propanoic acid as the acid building block. LC-MS (ES⁺): m/z 818.4 [M+H]⁺.

### Example 82

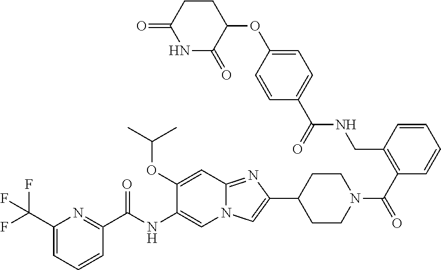 [SMILES: CC(C)Oc1cc2nc(C3CCN(C(=O)c4ccccc4CNC(=O)c4ccc(OC5CCC(=O)NC5=O)cc4)CC3)cn2cc1NC(=O)c1cccc(C(F)(F)F)n1]

N-[2-[1-[2-[[[4-[(2,6-dioxo-3-piperidyl)oxy]benzoyl]amino]methyl]benzoyl]-4-piperidyl]-7-isopropoxy-imidazo[1,2-a]pyridin-6-yl]-6-(trifluoromethyl)pyridine-2-carboxamide. Compound of Example 82 was prepared following the General Procedure 2, using 2-(((tert-butoxycarbonyl)amino)methyl)benzoic acid as the acid building block. LC-MS (ES⁺): m/z 812.2 [M+H]⁺.

### Example 83

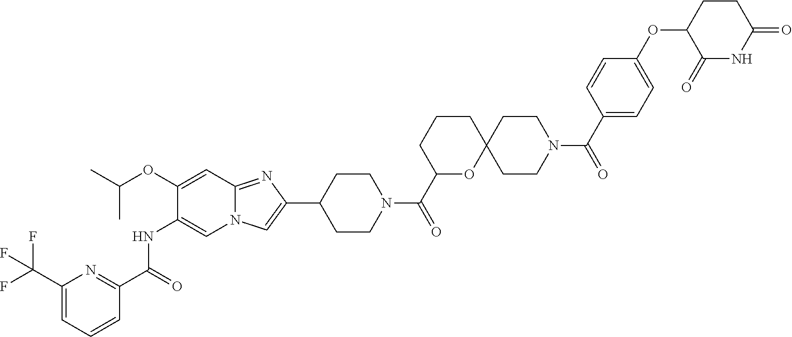 [SMILES: CC(C)Oc1cc2nc(C3CCN(C(=O)C4CCCC5(CCN(C(=O)c6ccc(OC7CCC(=O)NC7=O)cc6)CC5)O4)CC3)cn2cc1NC(=O)c1cccc(C(F)(F)F)n1]

N-[2-[1-[9-[4-[(2,6-dioxo-3-piperidyl)oxy]benzoyl]-1-oxa-9-azaspiro[5.5]undecane-2-carbonyl]-4-piperidyl]-7-isopropoxy-imidazo[1,2-a]pyridin-6-yl]-6-(trifluoromethyl) pyridine-2-carboxamide. Compound of Example 83 was prepared following the General Procedure 2, using 9-(tert-butoxycarbonyl)-1-oxa-9-azaspiro[5.5]undecane-2-carboxylic acid as the acid building block. LC-MS (ES⁺): m/z 860.2 [M+H]⁺.

### Example 84

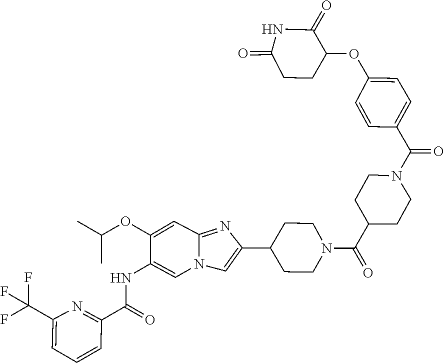 [SMILES: CC(C)Oc1cc2nc(C3CCN(C(=O)C4CCN(C(=O)c5ccc(OC6CCC(=O)NC6=O)cc5)CC4)CC3)cn2cc1NC(=O)c1cccc(C(F)(F)F)n1]

N-[2-[1-[1-[4-[(2,6-dioxo-3-piperidyl)oxy]benzoyl]piperidine-4-carbonyl]-4-piperidyl]-7-isopropoxy-imidazo[1,2-a]pyridin-6-yl]-6-(trifluoromethyl)pyridine-2-carboxamide. Compound of Example 84 was prepared following the General Procedure 2, using 1-(tert-butoxycarbonyl)piperidine-4-carboxylic acid as the acid building block. LC-MS (ES⁺): m/z 790.2 [M+H]⁺.

### Synthesis of 2-[4-[7-isopropoxy-6-[[6-(trifluoromethyl)pyridine-2-carbonyl]amino]imidazo[1,2-a]pyridin-2-yl]-1-piperidyl]acetic acid

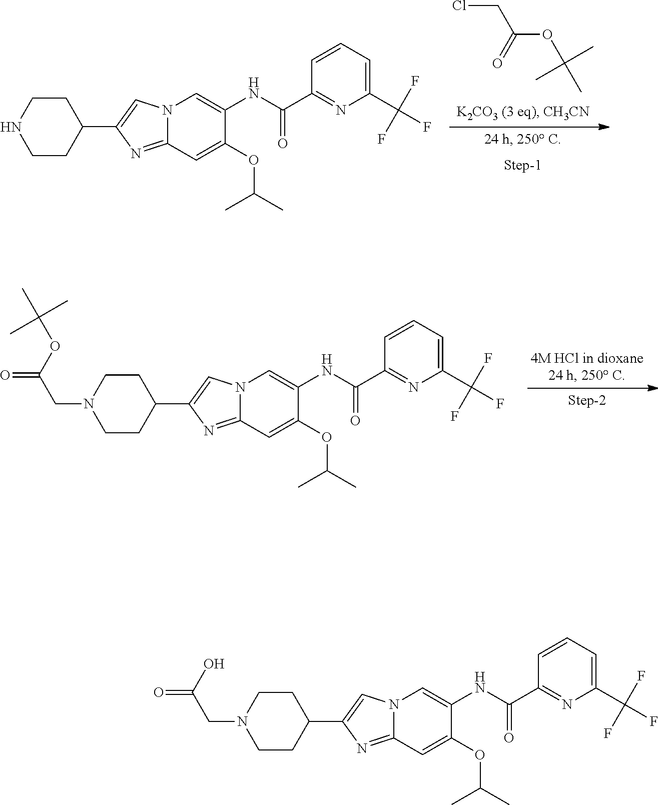

### Step-1

To a solution of N-[7-isopropoxy-2-(4-piperidyl)imidazo[1,2-a]pyridin-6-yl]-6-(trifluoromethyl)pyridine-2-carboxamide (2.7 g, 6.03 mmol) in acetonitrile (50 mL), tert-butyl 2-chloroacetate (908.76 mg, 6.03 mmol, 863.02 μL), K₂CO₃ (2.50 g, 18.10 mmol) were added under inert atmosphere. The mixture was stirred at 25° C. for 24 h. The reacting mixture was filtered and evaporated. The crude material was triturated with hexane (25 mL) to give tert-butyl 2-[4-[7-isopropoxy-6-[[6-(trifluoromethyl)pyridine-2-carbonyl]amino]imidazo[1,2-a]pyridin-2-yl]-1-piperidyl]acetate (2.95 g, 4.99 mmol, 83% yield) as a yellow solid. LC-MS (ES⁺): m/z 562.2 [M+H]⁺. ¹H NMR (600 MHz, DMSO-d₆) δ=10.43 (s, 1H), 9.41 (s, 1H), 8.51-8.34 (m, 2H), 8.22 (dd, J=1.1, 7.7 Hz, 1H), 7.65 (s, 1H), 7.10 (s, 1H), 4.86 (m, 1H), 3.10 (s, 2H), 2.88 (br d, J=11.0 Hz, 2H), 2.60-2.55 (m, 1H), 2.28 (m, 2H), 1.92 (m, 2H), 1.71-1.59 (m, 2H), 1.48-1.33 (m, 15H).

### Step-2

tert-butyl 2-[4-[7-isopropoxy-6-[[[6-(trifluoromethyl)pyridine-2-carbonyl]amino]imidazo[1,2-a]pyridin-2-yl]-1-piperidyl]acetate (2.95 g, 5.25 mmol) was added to a solution of 4 M HCl (50 mL) under inert atmosphere. The mixture was stirred at 20° C. for 24 h. The resulting mixture was evaporated to dryness and dried under vacuum to provide 2-[4-[7-isopropoxy-6-[[6-(trifluoromethyl)pyridine-2-carbonyl]amino]imidazo[1,2-a]pyridin-2-yl]-1-piperidyl]acetic acid (2.7 g, 4.73 mmol, HCl salt) as a yellow solid. LC-MS (ES⁺): m/z 506.2 [M+H]⁺.

### General Procedure 3 for the Synthesis of Examples 85-87

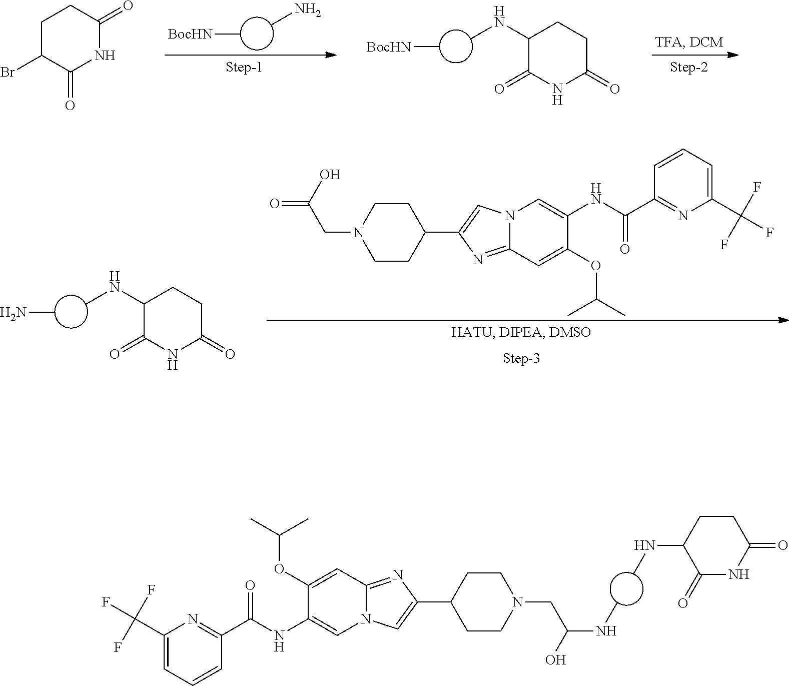

### Step-1

A solution of 3-bromopiperidine-2,6-dione (1 equiv.), the respective amine building block (1 equiv.), and DIPEA (3 equiv.) in 1,4-dioxane was stirred at 100° C. for 24 h under inert atmosphere. A second portion of 3-bromopiperidine-2,6-dione (1 equiv.) was added and the reaction mixture was stirred for another 24 h at 100° C. The reacting mixture was evaporated, and the residue was subjected to prep-HPLC. ((Waters SunFire C18 19*100 5 mkm column; gradient mixture H₂O-MeCN as a mobile phase)) to afford the desired intermediate building block.

### Step-2 and Step-3

Syntheses were performed on a ≣40 mg-scale.

The respective intermediate building block (1.0 equiv.) was stirred in dry DCM (0.5 mL) and TFA (0.5 mL) at room temperature for 4 hours, then was concentrated under reduced pressure. The residue was dissolved in dry DMSO (0.3 mL) followed by addition of 4-(4-(4-((5-(tert-butyl)-1,2,4-oxadiazole-3-carboxamido)methyl)-3-methylphenyl)pyrrolo[2,1-f][1,2,4]triazin-6-yl)benzoic acid (1.0 equiv.), DIPEA (6.0 equiv.), and HATU (1.1 equiv.) and stirred for 16 hours at room temperature. The reacting mixture was concentrated under reduced pressure and the residue was dissolved in DMSO (0.15 mL). The solution was filtered, analyzed by LC-MS, and then subjected to prep. HPLC (Waters SunFire C18 19*100 5 mkm column; gradient mixture H₂O-MeCN-0.1% TFA as a mobile phase) to afford the corresponding products.

### Example 85

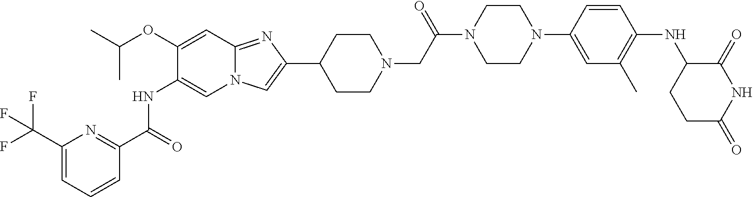 [SMILES: Cc1cc(N2CCN(C(=O)CN3CCC(c4cn5cc(NC(=O)c6cccc(C(F)(F)F)n6)c(OC(C)C)cc5n4)CC3)CC2)ccc1NC1CCC(=O)NC1=O]

N-[2-[1-[2-[4-[4-[(2,6-dioxo-3-piperidyl)amino]-3-methyl-phenyl]piperazin-1-yl]-2-oxo-ethyl]-4-piperidyl]-7-isopropoxy-imidazo[1,2-a]pyridin-6-yl]-6-(trifluoromethyl) pyridine-2-carboxamide. Compound of Example 85 was prepared was prepared following the General Procedure 3, using tert-butyl 4-(4-amino-3-methylphenyl)piperazine-1-carboxylate as the amine building block. LC-MS (ES⁻): m/z 788.2 [M−H]⁻.

### Example 86

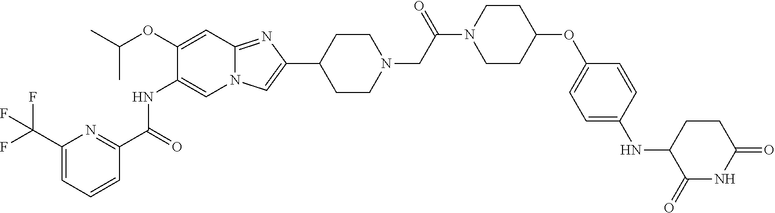 [SMILES: CC(C)Oc1cc2nc(C3CCN(CC(=O)N4CCC(Oc5ccc(NC6CCC(=O)NC6=O)cc5)CC4)CC3)cn2cc1NC(=O)c1cccc(C(F)(F)F)n1]

N-[2-[1-[2-[4-[4-[(2,6-dioxo-3-piperidyl)amino]phenoxy]-1-piperidyl]-2-oxo-ethyl]-4-piperidyl]-7-isopropoxy-imidazo[1,2-a]pyridin-6-yl]-6-(trifluoromethyl)pyridine-2-carboxamide. Compound of Example 86 was prepared was prepared following the General Procedure 3, using tert-butyl 4-(4-aminophenoxy)piperidine-1-carboxylate as the amine building block. LC-MS (ES⁺): m/z 791.2 [M+H]⁺.

### Example 87

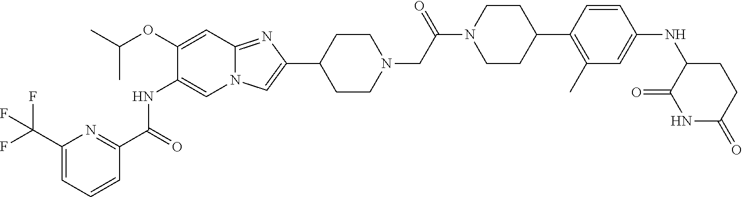 [SMILES: Cc1cc(NC2CCC(=O)NC2=O)ccc1C1CCN(C(=O)CN2CCC(c3cn4cc(NC(=O)c5cccc(C(F)(F)F)n5)c(OC(C)C)cc4n3)CC2)CC1]

N-[2-[1-[2-[4-[4-[(2,6-dioxo-3-piperidyl)amino]-2-methyl-phenyl]-1-piperidyl]-2-oxo-ethyl]-4-piperidyl]-7-isopropoxy-imidazo[1,2-a]pyridin-6-yl]-6-(trifluoromethyl) pyridine-2-carboxamide. Compound of Example 87 was prepared was prepared following the General Procedure 3, using tert-butyl 4-(4-amino-2-methylphenyl)piperidine-1-carboxylate as the amine building block. LC-MS (ES⁺): m/z 789.2 [M+H]⁺.

### V. Biological Activities

Selected compounds were tested in an IRAK4 degradation assay using the HiBiT Method. DC₅₀ values are given in Table 1.

### Materials

Phenol red-free Dulbecco's modified Eagle medium (DMEM) and fetal bovine serum (FBS) were purchased from Gibco (Grand Island, NY, USA). The Nano-Glo© HiBiT Lytic Assay System was purchased from Promega (Madison, WI, USA). Cell culture flasks and 384-well microplates were acquired from VWR (Radnor, PA, USA). The 293T cell line was engineered by knocking-in a HiBiT fusion tag into the C-terminal of the IRAK4 gene in 293T cells (Synthego, Redwood City, CA, USA).

### IRAK4 Degradation Analysis

IRAK4 degradation was measured via the quantification of luminescent signals using the Nano-Glo© HiBiT Lytic Assay kit. Test compounds were added to 384-well plates in duplicate using an 11-point half-log dilution series, with the highest dose set at 10 mM. 293T cells expressing HiBiT-tagged IRAK4 were then added into 384-well plates at a cell density of 10,000 cells per well. The plates were kept at 37° C. with 5% CO₂ for 6 hours. Cells that were treated only with DMSO served as the negative control; wells that contained only assay media served as the background control. After the 6-hour incubation, Nano-Glo® HiBiT Lytic Assay reagents were added to the cells. Luminescence was acquired using an EnVision® Multilabel Reader (PerkinElmer, Santa Clara, CA, USA).

Table 1 shows the activity of selected compounds of this disclosure in the in vitro IRAK assay, wherein each compound number corresponds to the compound numbering set forth in Examples 1-87 described herein.
“+++++” represents a DC₅₀ value of less than 10 nM.
“++++” represents a DC₅₀ value of 10-100 nM.
“+++” represents a DC₅₀ value of greater than 100 nM-500 nM.
“++” represents a DC₅₀ value of greater than 500 nM-1000 nM.
“+” represents a DC₅₀ value of greater than 1000 nM.

TABLE 1

| Example | DC50 (nM) |
|---|---|
| 1 | +++ |
| 2 | ++++ |
| 3 | +++ |
| 4 | +++ |
| 5 | +++ |
| 6 | +++ |
| 7 | +++ |
| 8 | ++++ |
| 9 | ++++ |
| 10 | +++ |
| 11 | +++ |
| 12 | +++ |
| 13 | +++ |
| 14 | ++++ |
| 15 | ++++ |
| 16 | +++ |
| 17 | ++++ |
| 18 | ++++ |
| 19 | ++++ |
| 20 | +++ |
| 21 | ++++ |
| 22 | ++++ |
| 23 | ++++ |
| 24 | ++++ |
| 25 | +++ |
| 26 | +++ |
| 27 | +++ |
| 28 | ++++ |
| 29 | ++++ |
| 30 | +++ |
| 31 | +++ |
| 32 | +++ |
| 33 | +++ |
| 34 | +++ |
| 35 | +++ |
| 36 | ++++ |
| 37 | ++++ |
| 38 | +++ |
| 39 | +++ |
| 40 | +++ |
| 41 | +++ |
| 42 | +++ |
| 43 | +++ |
| 44 | +++ |
| 45 | ++ |
| 46 | + |
| 47 | +++ |
| 48 | + |
| 49 | ++++ |
| 50 | ++++ |
| 51 | ++++ |
| 52 | ++++ |
| 53 | +++ |
| 54 | +++ |
| 55 | ++++ |
| 56 | +++ |
| 57 | +++++ |
| 58 | ++++ |
| 59 | ++++ |
| 60 | ++++ |
| 61 | ++++ |
| 62 | +++ |
| 63 | +++ |
| 64 | +++ |
| 65 | ++++ |
| 66 | +++ |
| 67 | +++ |
| 68 | ++++ |
| 69 | +++ |
| 70 | +++ |
| 71 | +++ |
| 72 | +++ |
| 73 | +++ |
| 74 | +++ |
| 75 | ++++ |
| 76 | +++ |
| 77 | +++ |
| 78 | ++ |
| 79 | ++++ |
| 80 | +++ |
| 81 | +++ |
| 82 | +++ |
| 83 | +++ |
| 84 | ++ |
| 85 | +++ |
| 86 | +++ |
| 87 | ++++ |

What is claimed is:

1. A compound of Formula (A):
IRAK-L-DSM (A)
or a pharmaceutically acceptable salt thereof, wherein:
DSM is a degradation signaling moiety that is covalently attached to the linker L,
L is a linker that covalently attaches IRAK to DSM; and
IRAK is an IRAK4 binding moiety represented by Formula (I) that is covalently attached to linker L; 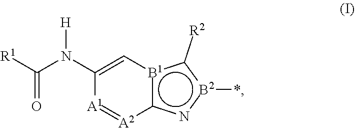
wherein:
A¹ is selected from N, CH and CR³, and A² is selected from N, CH and CR⁴, provided only one of A¹ or A² may be N;
one of B¹ and B² is N, and the other is C;
R¹ is selected from:
i. phenyl optionally substituted with 1 to 3 R⁵,
ii. a 5 or 6 membered heteroaryl having 1 to 3 heteroatoms independently selected from nitrogen, oxygen and sulfur, said heteroaryl is optionally substituted with 1 to 3 R⁵,
iii. a 5 or 6 membered partially or fully saturated heterocycle having 1 to 2 heteroatoms independently selected from oxygen and nitrogen, said heterocycle may be optionally substituted with 1 to 3 R⁵,
iv. a partially or fully saturated C₃₋₆ cycloalkyl which may be optionally substituted with 1 to 3 R⁵,
v. a 7 to 10 membered fused heterobicyclic ring system having 1, 2 or 3 heteroatoms independently selected from nitrogen and oxygen, said heterobicylic ring system is optionally substituted with 1 to 3 R⁵, and
vi. a 7 to 10 membered fused carbobicyclic ring system, said carbobicyclic ring system is optionally substituted with 1 to 3 R⁵;
R² is hydrogen, C₁₋₄ alkyl or halogen;
R³ and R⁴ are each independently selected from halogen, C₁₋₄alkyl, nitrile and —OR⁶, wherein the C₁₋₄alkyl is optionally substituted with C₁₋₄alkoxy or at least one halogen;
R⁵ for each occurrence, is independently selected from CN, hydroxyl, C₁₋₄ alkyl, oxo, halogen, —NR⁸R⁹, C₁₋₄ alkoxy, —O—C₁₋₄ alkyl, C₃₋₆cycloalkyl, —C₁₋₄alkyl-C₃₋₆cycloalkyl, C(O)NR¹⁰R¹¹, a C₄₋₇ heterocycle, and a 5 or 6 membered heteroaryl having 1 to 2 heteroatoms independently selected from nitrogen, oxygen and sulfur, said C₁₋₄ alkyl is optionally substituted with one or more substituents independently selected from CN, halo, C₁₋₄alkoxy, and hydroxyl, said C₃₋₆cycloalkyl and heteroaryl is optionally substituted with 1 to 2 substituents independently selected from the group consisting of C₁₋₄ alkyl, hydroxyl and halogen; or two R⁵ groups together with the intervening atoms can form a ring selected from phenyl, C₄₋₆ carbocycle, C₄₋₆ heterocycle, or a 7-membered bridged ring system optionally having 1 heteroatom selected from nitrogen and oxygen, wherein said phenyl, C₄₋₆ carbocycle and C₄₋₆ heterocycle are each optionally substituted with 1 to 2 C₁₋₄ alkyl, halogen or C₁₋₄ haloalkyl;
R⁶ is hydrogen, C₁₋₅alkyl, C₃₋₆cycloalkyl, a 4 to 7 membered partially or fully saturated heterocycle containing 1 or 2 heteroatoms selected from nitrogen and oxygen, a 5 to 10 membered spiro carbocyclic ring and a 4 to 10 membered heterocycle having 1 to 2 heteroatoms independently selected from nitrogen and oxygen; wherein the C₁₋₅alkyl represented by R⁶ is optionally substituted with 1 to 3 substituents R^{6a} independently selected from halogen, hydroxyl, C₁₋₅alkyl, C₁₋₄alkoxy, C₁₋₄ haloalkoxy, C₃₋₆cycloalkyl, phenyl, a 4 to 7 membered partially or fully saturated heterocycle containing 1 or 2 heteroatoms selected from nitrogen and oxygen, and a fully saturated 5 to 8 membered bridged-heterocyclic ring system having 1 to 2 heteroatoms independently selected from nitrogen and oxygen; wherein the C₃₋₆cycloalkyl represented by R⁶ is optionally substituted with 1 to 3 substituents R^{6b} independently selected from halogen, C₁₋₄alky, C₁₋₄ haloalkyl, and C₁₋₄alkoxy; wherein the 4 to 7 membered partially or fully saturated heterocycle, the 5 to 10 membered spiro carbocyclic ring and 5 to 10 membered spiro heterobicyclic ring system represented by R⁶ is optionally substituted with 1 to 3 substituents R⁶, independently selected from C₁₋₄alky and oxo; and wherein said C₃₋₆cycloalkyl, phenyl, 4 to 7 membered partially or fully saturated heterocycle represented by R^{6a} are optionally substituted with 1 to 3 R⁷;
each R⁷ is independently selected from oxo, halogen, C₁₋₄ haloalkyl and C₁₋₄ alkyl;
R⁸ and R⁹ are each independently selected from hydrogen, —C(O)C₁₋₄ alkyl and C₁₋₄ alkyl; or R⁸ and R⁹ may combine to form a 4 to 6 membered saturated ring optionally containing one additional heteroatom selected from nitrogen or oxygen wherein said additional nitrogen may be optionally substituted with C₁₋₄ alkyl;
R¹⁰ and R¹¹ are each independently selected from hydrogen and C₁₋₄ alkyl; and
—* represents a bond to the linker L.

2. The compound of claim 1, or a pharmaceutically acceptable salt thereof, wherein IRAK is an IRAK4 binding moiety represented by Formula (IA) or (IB):

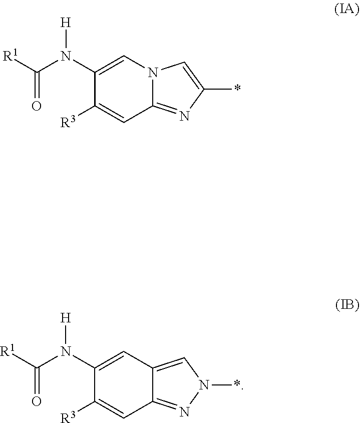

3. The compound of claim 1 or 2, or a pharmaceutically acceptable salt thereof, wherein R¹ is selected from phenyl optionally substituted with 1 to 3 R⁵; 5 or 6 membered heteroaryl having 1 to 2 nitrogen atoms, said heteroaryl is optionally substituted with 1 to 3 R⁵; and 9 to 10 membered bicyclic heteroaryl having 1, 2 or 3 nitrogen atoms, said ring system is optionally substituted with 1 to 3 R⁵.

4. The compound of claim 1 or 2, or a pharmaceutically acceptable salt thereof, wherein R¹ is selected from oxazole optionally substituted with 1 to 2 R⁵; phenyl optionally substituted with 1 to 2 R⁵; pyrazole optionally substituted with 1 to 2 R⁵; pyridine optionally substituted with 1 to 2 R⁵; pyridone optionally substituted with 1 to 2 R⁵; pyrimidine optionally substituted with 1 to 2 R⁵; and pyrazolo[1,5-a]pyrimidine optionally substituted with 1 to 2 R⁵.

5. The compound of claim 1 or 2, or a pharmaceutically acceptable salt thereof, wherein R¹ is selected from oxazole optionally substituted with 1 to 2 R⁵; phenyl optionally substituted with 1 to 2 R⁵; pyrazole optionally substituted with 1 to 2 R⁵; pyridine optionally substituted with 1 to 2 R⁵; pyrimidine optionally substituted with 1 to 2 R⁵; and pyrazolo[1,5-a]pyrimidine optionally substituted with 1 to 2 R⁵

6. The compound of claim 1 or 2, or a pharmaceutically acceptable salt thereof, wherein R¹ is represented by one of the following formulae: 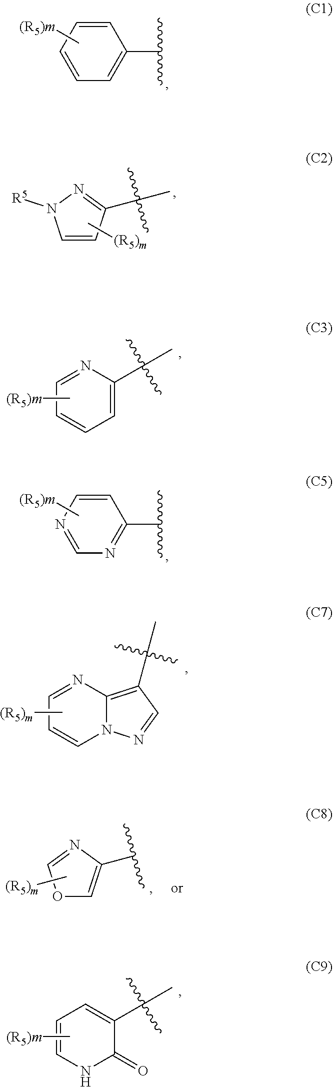
wherein m is 0, 1 or 2.

7. The compound of claim 1 or 2, or a pharmaceutically acceptable salt thereof, wherein R¹ is represented by one of the following formulae: 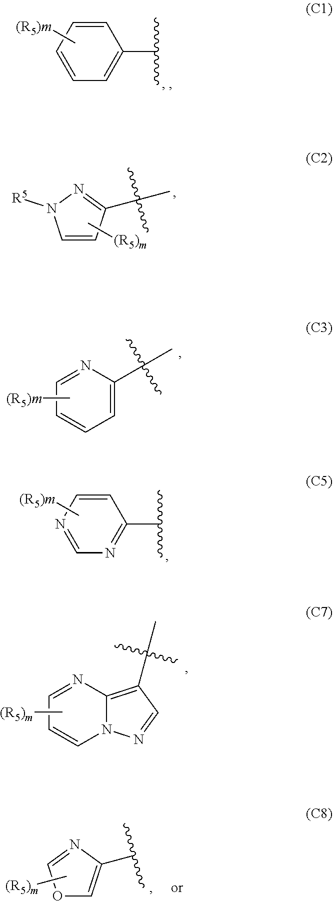
wherein m is 0, 1 or 2.

8. The compound of claim 1 or 2, or a pharmaceutically acceptable salt thereof, wherein R¹ is represented by one of the following formulae:

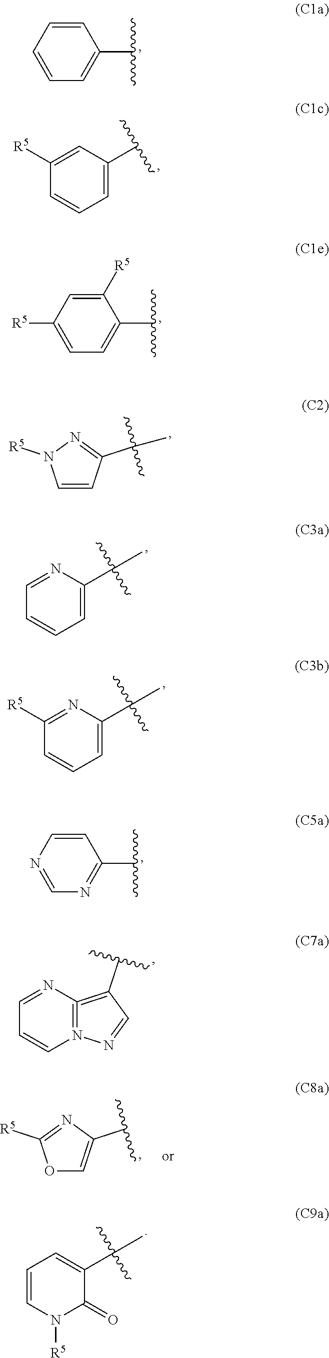

9. The compound of claim 1 or 2, or a pharmaceutically acceptable salt thereof, wherein R¹ is represented by one of the following formulae:

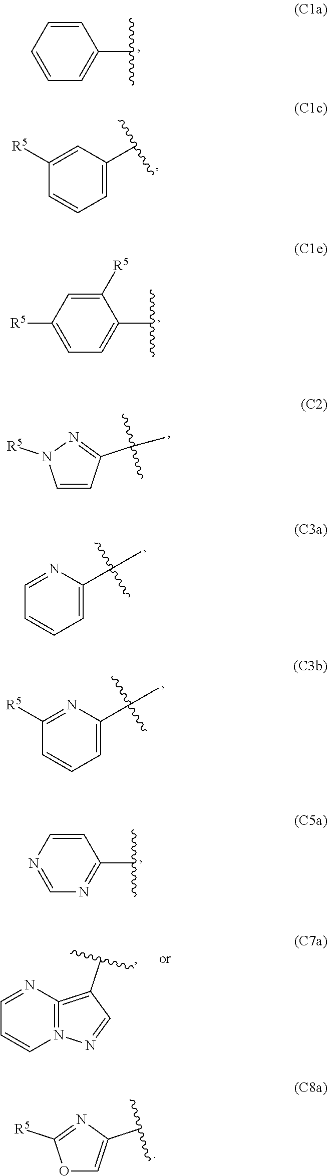

10. The compound of claim 1 or 2, or a pharmaceutically acceptable salt thereof, wherein R¹ is represented by the following formula:

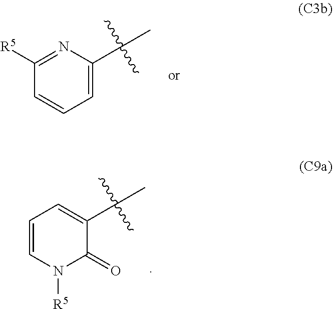

11. The compound of claim 1 or 2, or a pharmaceutically acceptable salt thereof, wherein R¹ is represented by the following formula:

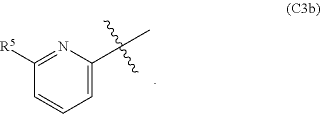

12. The compound of any one of claims 1-11, or a pharmaceutically acceptable salt thereof, wherein R² is hydrogen.

13. The compound of claim 1, or a pharmaceutically acceptable salt thereof, wherein IRAK is an IRAK4 binding moiety represented by one of the following formulae:

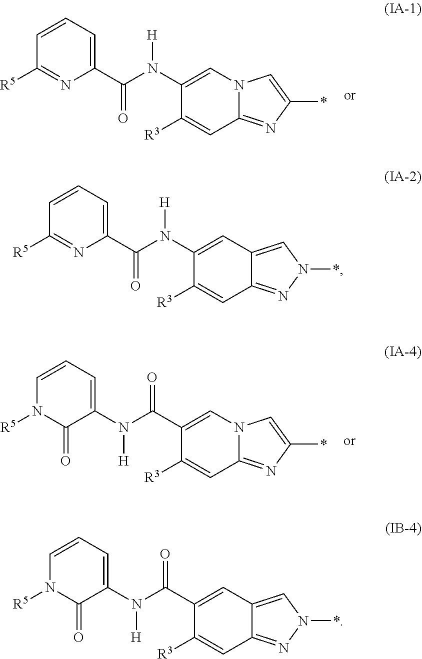

14. The compound of claim 1, or a pharmaceutically acceptable salt thereof, wherein IRAK is an IRAK4 binding moiety represented by one of the following formulae:

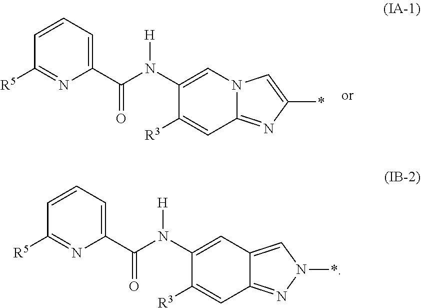

15. The compound of any one of claims 1-14, or a pharmaceutically acceptable salt thereof, wherein:
R³ is C₁₋₄alkyl or —OR⁶, wherein the C₁₋₄alkyl is optionally substituted with at least one halogen; and
R⁶ is C₁₋₅alkyl, C₃₋₆cycloalkyl or a 4 to 7 membered fully saturated heterocycle containing 1 or 2 heteroatoms selected from nitrogen and oxygen, wherein the C₁₋₅alkyl represented by R⁶ is optionally substituted with one to three halogens, and the C₃₋₆cycloalkyl represented by R⁶ is optionally substituted with 1 to 3 substituents R^{6b} independently selected from halogen, C₁₋₄alky, C₁₋₄ haloalkyl, and C₁₋₄alkoxy.

16. The compound of claim 15, or a pharmaceutically acceptable salt thereof, wherein:
R³ is C₁₋₄alkyl or —OR⁶, wherein the C₁₋₄alkyl is optionally substituted with one to three halogens; and
R⁶ is C₁₋₅alkyl.

17. The compound of claim 15, or a pharmaceutically acceptable salt thereof, wherein:
R³ is —CF₃ or —O—CH(CH₃)₂.

18. The compound of claim 15, or a pharmaceutically acceptable salt thereof, wherein R³ is —O—CH(CH₃)₂.

19. The compound of any one of claim 1-18, or a pharmaceutically acceptable salt thereof, wherein R⁵ for each occurrence, is independently selected from C₁₋₄ alkyl, halogen, C₁₋₄haloalkyl, and C₃₋₄cycloalkyl, and wherein said C₃₋₄cycloalkyl is optionally substituted with 1 halo.

20. The compound of any one of claim 1-18, or a pharmaceutically acceptable salt thereof, wherein R⁵ for each occurrence, is independently selected from C₁₋₄ alkyl, halogen, and C₁₋₄ haloalkyl.

21. The compound of claim 19, or a pharmaceutically acceptable salt thereof, wherein R⁵ for each occurrence, is independently selected from —CH₃, —CHF₂, —CF₃, F, cyclopropyl, and

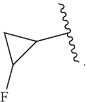 [SMILES: *C1CC1F]

22. The compound of claim 20, or a pharmaceutically acceptable salt thereof, wherein R⁵ for each occurrence, is independently selected from —CH₃, —CHF₂, —CF₃ and F.

23. The compound of claim 1, or a pharmaceutically acceptable salt thereof, wherein IRAK is an IRAK4 binding moiety represented by one of the following formulae: 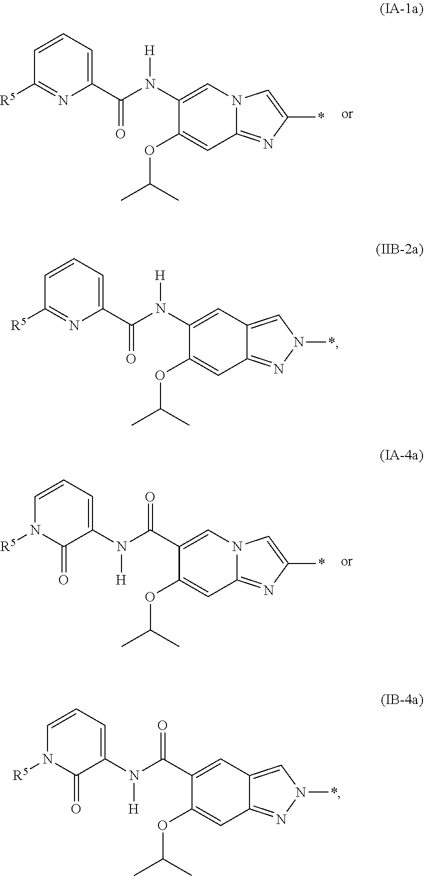
wherein R⁵ is C₁₋₃ alkyl, C₁₋₃ haloalkyl, C₃₋₄cycloalkyl, and wherein said C₃₋₄cycloalkyl is optionally substituted with 1 halo.

24. The compound of claim 1, or a pharmaceutically acceptable salt thereof, wherein IRAK is an IRAK4 binding moiety represented by one of the following formulae: 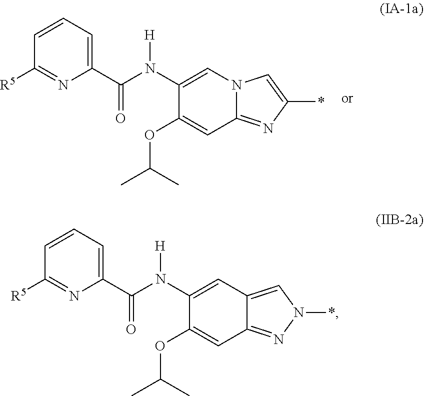
wherein R⁵ is C₁₋₃ alkyl or C₁₋₃ haloalkyl.

25. The compound of claim 23, or a pharmaceutically acceptable salt thereof, wherein R⁵ is CH₃, CHF₂, CF₃, cyclopropyl, or

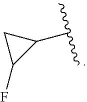 [SMILES: *C1CC1F]

26. The compound of claim 24, or a pharmaceutically acceptable salt thereof, wherein R⁵ is CH₃, CHF₂, or CF₃.

27. The compound of any one of claims 1-26, or a pharmaceutically acceptable salt thereof, wherein DSM is a degradation signaling moiety of formula (D): 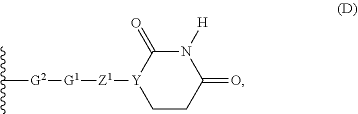
wherein: [symbol]
represents a bond to the linker L;
Y is CR^{D1} or N;
Z¹ is selected from bond, —NR^{D2}—, —O— and —CH₂—;
G¹ is selected from 6- to 10-membered aryl, 5- to 10-membered heteroaryl and partially saturated 4- to 11-membered heterocycle; wherein the 6- to 10-membered aryl, 5- to 10-membered heteroaryl and partially saturated 4- to 11-membered heterocycle represented by G¹ are each optionally substituted with one or more R^{D3};
G² is selected from Het₁, *—NR^{D4}-Het₁-_{*}^{*}, *—NR^{D4}-Het₁-C₁₋₄ alkyl-_{*}^{*}, *—C₁₋₄ alkyl-C(R^{D1})=Het₁-_{*}^{*}, *—C(O)—C₁₋₄ alkyl-Het₁-_{*}^{*}, *-Het₁-C₁₋₆ alkyl-_{*}^{*}, *-Het₁-O—_{*}^{*}, *—C(O)—C₁₋₄ alkyl-Het₁-C(O)—_{*}^{*}, *—C(O)-Het₁-C(O)—_{*}^{*}, *—C(O)-phenyl-C₁₋₄ alkyl-NHC(O)—_{*}^{*}; wherein *— represents a bond to the linker L, and _{*}^{*}— represents a bond to G¹;
Het₁ is 4- to 7-membered monocyclic heterocycle or 7- to 11-membered bicyclic heterocycle, each of which is optionally substituted with one or more R^{D5};
R^{D1} is selected from H, C₁₋₆ alkyl or halogen;
R^{D2} is H or C₁₋₃ alkyl;
R^{D3} is, for each occurrence, independently selected from H, halogen, C₁₋₄ alkyl and C₁₋₄haloalkyl;
R^{D4} is H or C₁₋₃ alkyl; and
R^{D5} is, for each occurrence, independently selected from H, halogen, hydroxyl, C₁₋₄ alkyl, C₁₋₄haloalkyl and C₁₋₄ alkoxy.

28. The compound of claim 26, or a pharmaceutically acceptable salt thereof, wherein Het₁ is a 4 to 7 membered monocyclic saturated heterocycle containing 1 or 2 nitrogen atoms or a 7 to 11 membered saturated spiro bicyclic heterocycle containing 1 or 2 heteroatoms selected from N and O, each of which is optionally substituted with 1 or 2 R^{D5}.

29. The compound of claim 27, or a pharmaceutically acceptable salt thereof, wherein Het₁ is piperidine, piperazine, 2-azaspiro[3.3]heptane, 2,6-diazaspiro[3.3]heptane, 5-azaspiro [3.4]octane or 9-oxa-9-azaspiro[5.5]undecane, each of which is optionally substituted with 1 or 2 R^{D5}.

30. The compound of any one of claims 1-27, or a pharmaceutically acceptable salt thereof, wherein DSM is a degradation signaling moiety of formula (D-I) or (D-II): 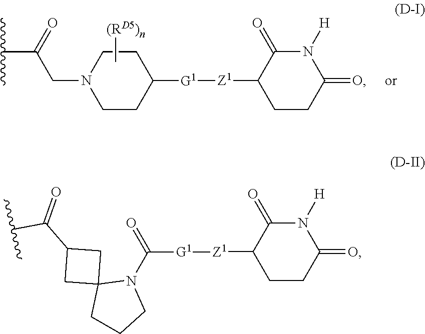
wherein: [symbol]
represents a bond to the linker L;
Z¹ is selected from bond, —NR^{D2}_and —O—;
G¹ is selected from 6- to 10-membered aryl, 5- to 10-membered heteroaryl and partially saturated 4- to 11-membered heterocycle; wherein the 6- to 10-membered aryl, 5- to 10-membered heteroaryl and partially saturated 4- to 11-membered heterocycle represented by G¹ are each optionally substituted with one or more R^{D3};
R^{D2} is H or C₁₋₃ alkyl;
R^{D3} is, for each occurrence, independently selected from H, halogen and C₁₋₄ alkyl;
R^{D4} is C₁₋₃ alkyl;
R^{D5} is halogen; and
n is 0, 1 or 2.

31. The compound of any one of claims 27-30, or a pharmaceutically acceptable salt thereof, wherein G1 is 6- to 10-membered aryl, 5- to 10-membered heteroaryl or partially saturated 4- to 11-membered heterocycle; wherein the 6- to 10-membered aryl, 5- to 10-membered heteroaryl and partially saturated 4- to 11-membered heterocycle represented by G1 are each optionally substituted with 1 or 2 R^{D3}.

32. The compound of any one of claims 27-30, or a pharmaceutically acceptable salt thereof, wherein G¹ is selected from phenyl, pyrazolyl, pyridinyl and pyrimidinyl, 1,3-dihydro-2H-benzo[d]imidazol-2-one, indazolyl, and indolyl, each of which is optionally substituted with 1 or 2 R^{D3}.

33. The compound of any one of claims 27-30, or a pharmaceutically acceptable salt thereof, wherein G¹ is represented by any one of the following formulae: 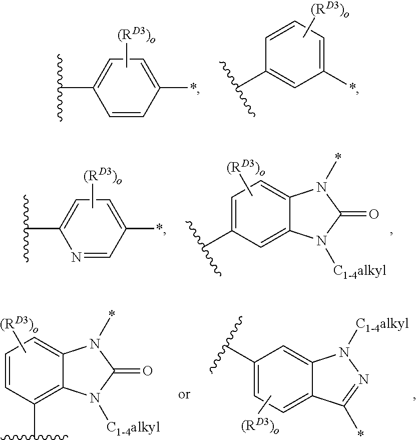
wherein:
is 0, 1 or 2, [symbol]
represents a bond to G², and
—* represents a bond to Z¹.

34. The compound of any one of claims 27-30, or a pharmaceutically acceptable salt thereof, wherein G¹ is 6- to 10-membered aryl or 5- to 10-membered heteroaryl; wherein the 6- to 10-membered aryl and 5- to 10-membered heteroaryl represented by G¹ are each optionally substituted with 1 or 2 R^{D3}.

35. The compound of any one of claims 27-30, or a pharmaceutically acceptable salt thereof, wherein G¹ is selected from phenyl, pyrazolyl, pyridinyl and pyrimidinyl, indazolyl, and indolyl, each of which is optionally substituted with 1 or 2 R^{D3}.

36. The compound of any one of claims 27-30, or a pharmaceutically acceptable salt thereof, wherein G¹ is represented by any one of the following formulae: 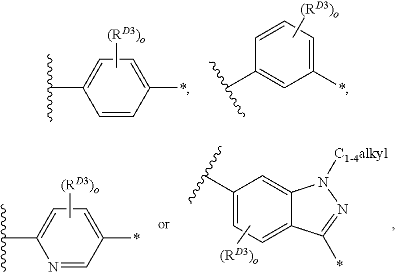
wherein:
is 0, 1 or 2, [symbol]
represents a bond to G², and
—* represents a bond to Z¹.

37. The compound of any one of claims 27-36, or a pharmaceutically acceptable salt thereof, wherein R^{D1} is H, —CH₃ or F.

38. The compound of any one of claims 27-37, or a pharmaceutically acceptable salt thereof, wherein R^{D2} is H.

39. The compound of any one of claims 27-38, or a pharmaceutically acceptable salt thereof, wherein R^{D3} is, for each occurrence, independently selected from H, Cl, F and —CH₃.

40. The compound of any one of claims 27-39, or a pharmaceutically acceptable salt thereof, wherein R^{D4} is —CH₃.

41. The compound of any one of claims 27-40, or a pharmaceutically acceptable salt thereof, wherein R^{D5} for each occurrence, is independently F or OH.

42. The compound of any one of claims 1-26, or a pharmaceutically acceptable salt thereof, wherein DSM represents any one of the following attached to L:

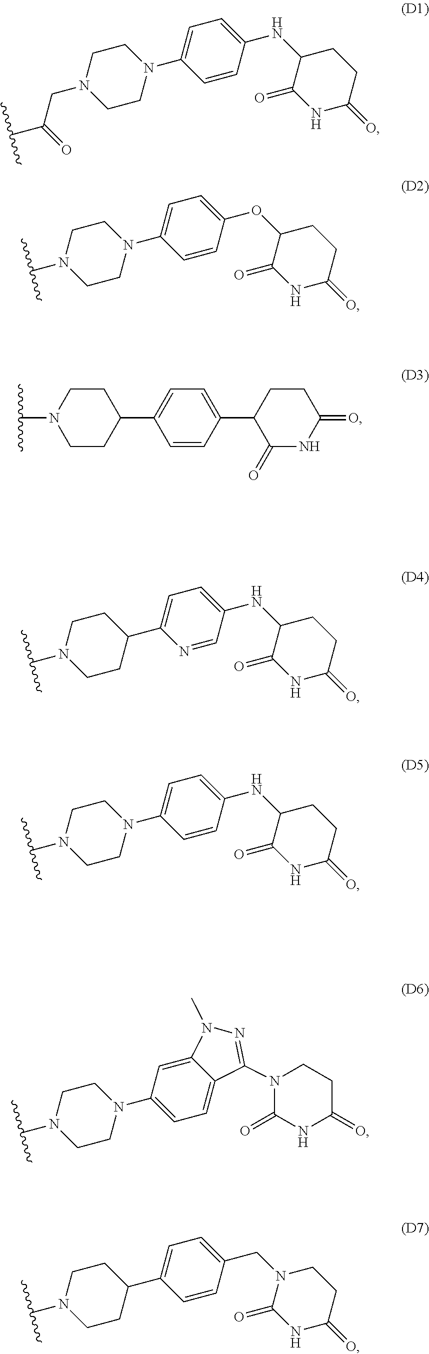

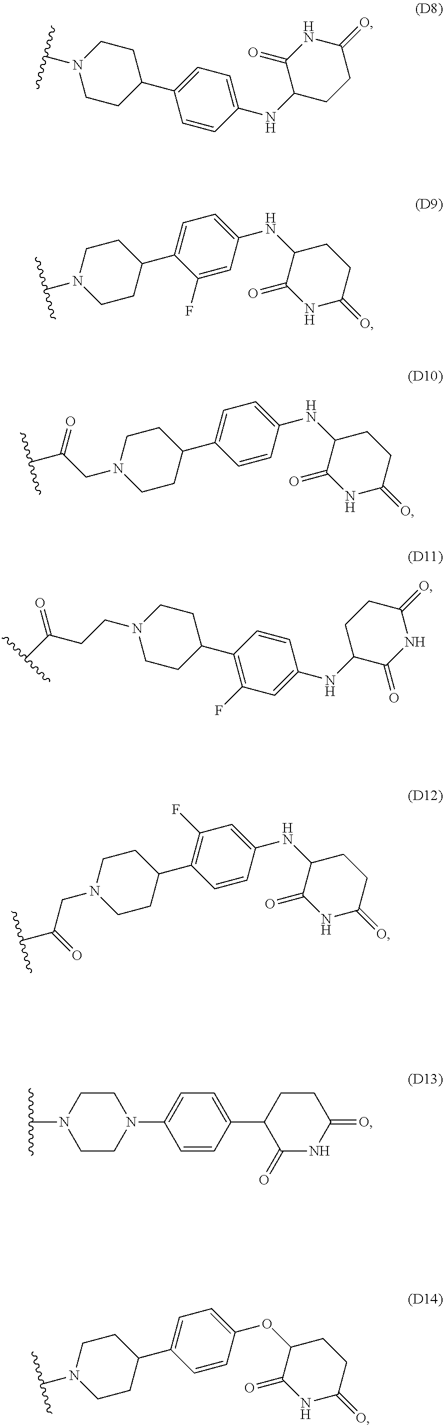

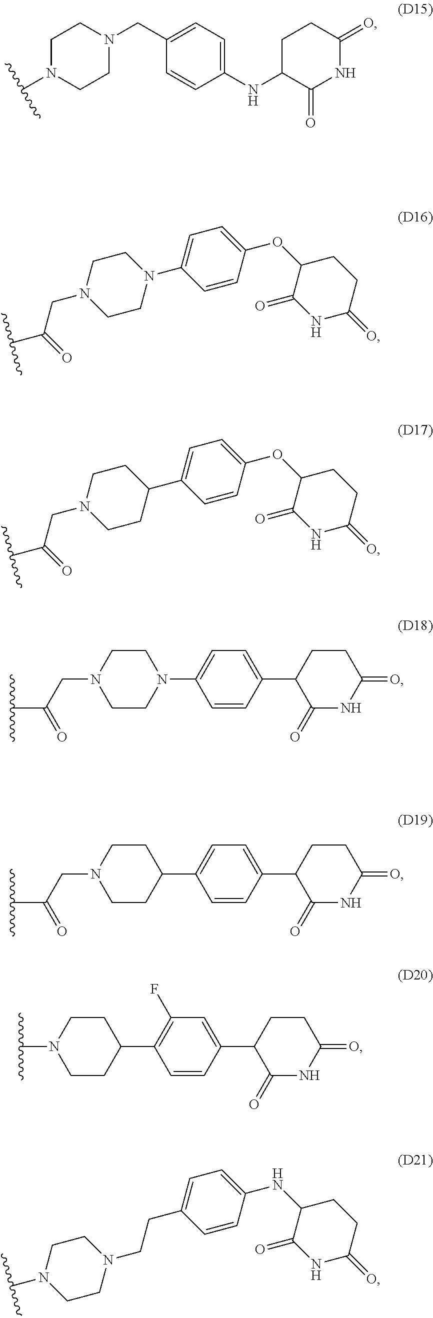

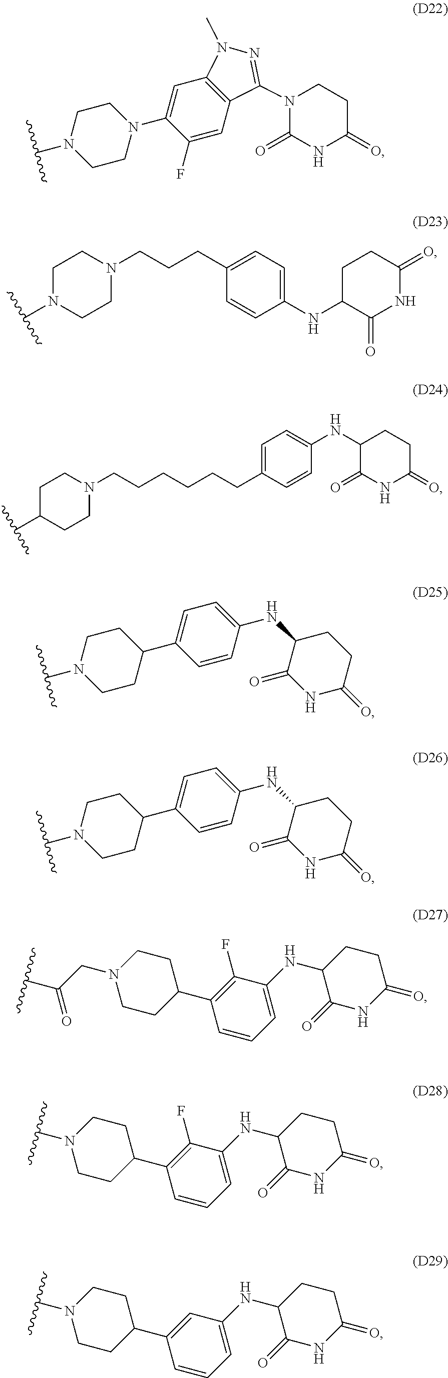

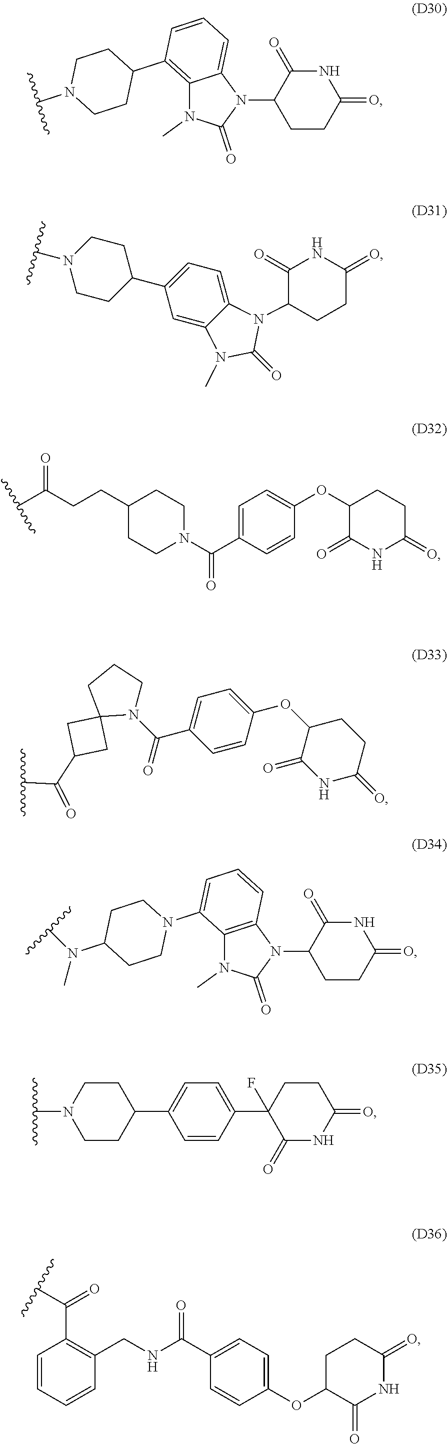

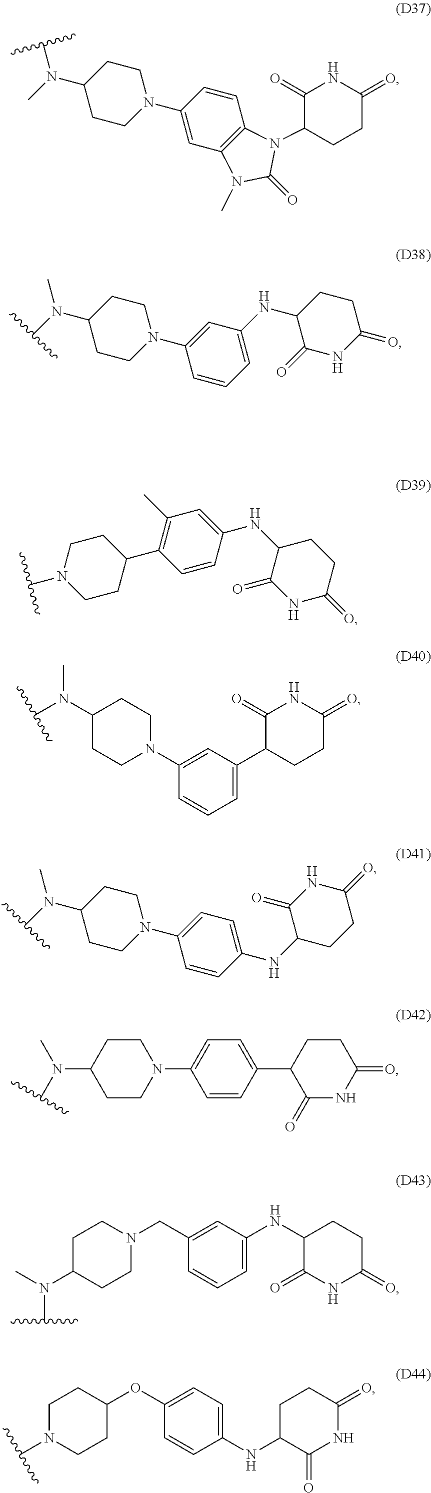

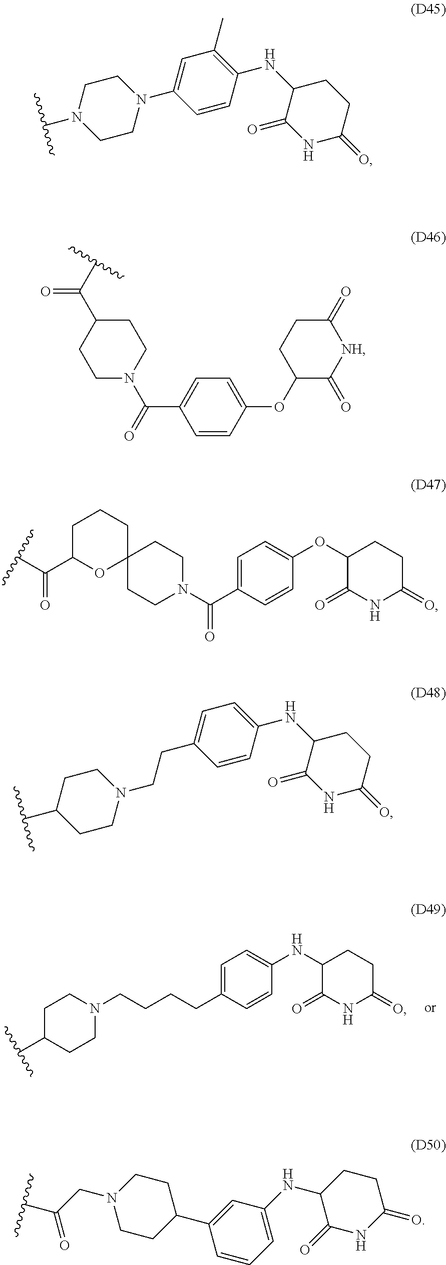

43. The compound of any one of claims 1-42, or a pharmaceutically acceptable salt thereof, wherein L is bond, C₁₋₈ alkyl, or is represented by formula (L-1), (L-2) or (L-3): 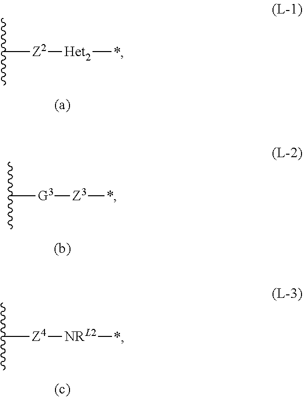
wherein:
Z² is bond or C₁₋₄ alkyl optionally substituted with one or more halogen;
Het₂ is 4- to 7-membered heterocycle optionally substituted by one or more R^{L1};
G³ is C₃₋₇ cycloalkyl or 4- to 7-membered heterocycle; wherein the C₃₋₇ cycloalkyl and 4- to 7-membered heterocycle represented by G³ are each optionally substituted with one or more R^{L3};
Z³ is C₁₋₄ alkyl or _{*}^{*}—C₁₋₄ alkyl-C(O)—*, wherein _{*}^{*}— represents a bond connected to G³; —* is a bond connected to the DSM; and the C₁₋₄ alkyl is optionally substituted with one or more halogen;
Z⁴ is C₁₋₄ alkyl optionally substituted by R^{L4};
R^{L1} is, for each occurrence, independently selected from H, halogen, C₁₋₄ alkyl and C₁₋₄haloalkyl;
R^{L2} is H or C₁₋₄ alkyl;
R^{L3} is, for each occurrence, independently selected from H, halogen, C₁₋₄ alkyl and C₁₋₄haloalkyl;
R^{L4} is halo, —OR^{L5}, or C₁₋₄ alkyl optionally substituted by halogen, C₃₋₇ cycloalkyl, phenyl, 4- to 7-membered monocyclic saturated heterocycle, or 5- to 6-membered heteroaryl, wherein the C₃₋₇ cycloalkyl, phenyl, 4- to 7-membered monocyclic saturated heterocycle, and 5- to 6-membered heteroaryl are each optionally substituted with one to three substituents independently selected from halogen, C₁₋₄ alkyl, C₁₋₄ haloalkyl, C₁₋₄ alkoxy and C₁₋₄ haloalkoxy;
R^{L5} is H, C₁₋₄ alkyl or C₁₋₄ haloalkyl; [symbol]
represents a bond to the IRAK4 binding moiety; and
—* represents a bond to the degradation signaling moiety DSM.

44. The compound of any one of claims 1-42, or a pharmaceutically acceptable salt thereof, wherein L is a bond.

45. The compound of any one of claims 1-42, or a pharmaceutically acceptable salt thereof, wherein L is C₁₋₈ alkyl.

46. The compound of claim 43, or a pharmaceutically acceptable salt thereof, wherein:
L is presented by formula (L-1), (L-2) or (L-3):
Het₂ is selected from azetidinyl, piperidinyl and pyrrolidinyl; wherein the azetidinyl, piperidinyl and pyrrolidinyl represented by Het₂ are each optionally substituted by one or more R^{L1}; and
G³ is azetidinyl, cyclohexyl or piperidinyl; wherein the cyclohexyl and piperidinyl represented by G³ are each optionally substituted with one or more R^{L3}.

47. The compound of claim 43 or 46, or a pharmaceutically acceptable salt thereof, wherein:
Z² is bond or —CH₂—;
Z³ is —CH₂—, —CH₂—CH₂—, _{*}^{*}—CH₂—C(O)—* or _{*}^{*}—CH₂—CH₂—C(O)—*; and
Z⁴ is —CH(CH₂Ph)- or —CH₂—CH₂—CH₂—.

48. The compound of any one of claims 43-47, or a pharmaceutically acceptable salt thereof, wherein:
R^{L1} is H;
R^{L2} is H;
R^{L3} is H; and
R^{L4} is benzyl.

49. The compound of claim 43, or a pharmaceutically acceptable salt thereof, wherein L is represented by formula (L-1) and Het₂ is represented by one of the formulae: 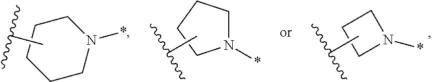
wherein: [symbol]
represents a bond to Z²; and
—* represents a bond to the degradation signaling moiety DSM.

50. The compound of claim 43, or a pharmaceutically acceptable salt thereof, wherein L is represented by formula (L-2) and G³ is represented by one of the formulae: 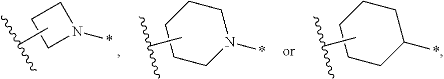
wherein: [symbol]
represents a bond to the IRAK4 binding moiety; and
—* represents a bond to Z³.

51. The compound of claim 43, or a pharmaceutically acceptable salt thereof, wherein L is represented by formula (L-1) and Het₂ is: 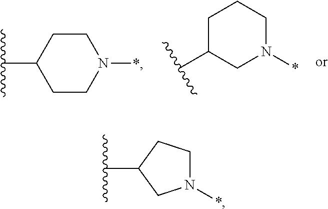
wherein: [symbol]
represents a bond to Z²; and
—* represents a bond to the degradation signaling moiety DSM.

52. The compound of claim 43, or a pharmaceutically acceptable salt thereof, wherein:
L is represented by formula (L-3);
Z⁴ is C₁₋₄ alkyl optionally substituted by benzyl; and
R^{L2} is H.

53. The compound of any one of claims 1-42, or a pharmaceutically acceptable salt thereof, wherein L is represented by any one of the following formulae: 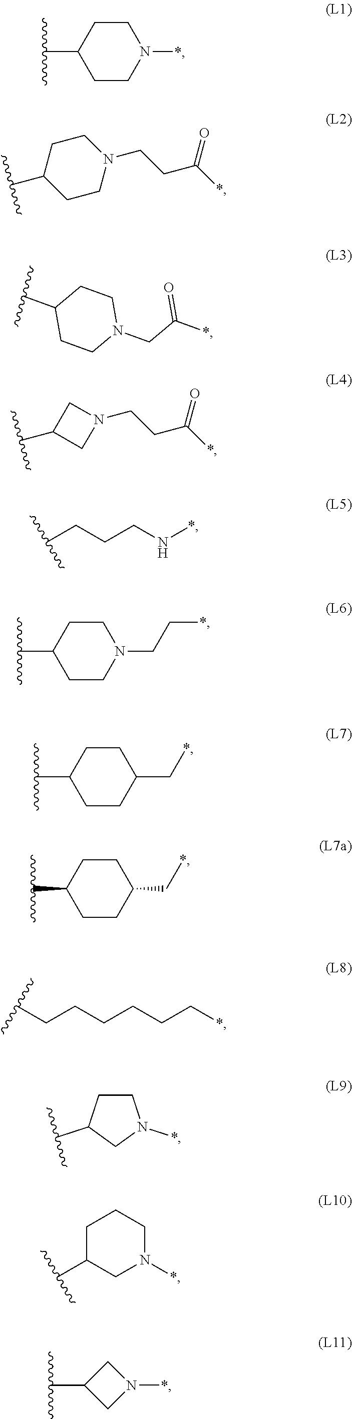 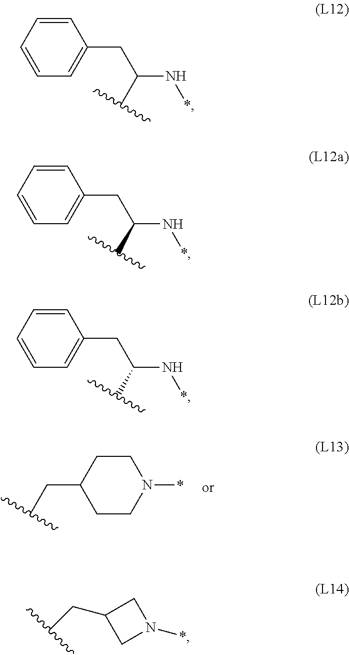
wherein: [symbol]
represents a bond to the IRAK4 binding moiety; and
—* represents a bond to the degradation signaling moiety DSM.

54. The compound of claim 1, selected from a compound of any one of Examples 1-87 or a pharmaceutically acceptable salt thereof.

55. A pharmaceutical composition comprising a compound or a pharmaceutically acceptable salt thereof of any one of claims 1-54 and a pharmaceutically acceptable carrier.

56. A method of treating an IRAK4-mediated disease in a subject comprising administering to the subject a compound or a pharmaceutically acceptable salt thereof of any one of claims 1 to 54 or a pharmaceutical composition of claim 55.

57. The method of claim 56, wherein the IRAK4-mediated disease is selected from the group consisting of Rheumatoid Arthritis, Psoriatic arthritis, Osteoarthritis, Systemic Lupus Erythematosus, Lupus nephritis, Cutaneous Lupus Erythematosus, Ankylosing Spondylitis, Osteoporosis, Neuromyelitis optica, Systemic sclerosis, Psoriasis, Dermatomyositis, Atopic Dermatitis, Hidradenitis Suppurativa, Type I diabetes, Type II diabetes, Inflammatory Bowel Disease, Crohn's Disease, Ulcerative Colitis, Hyperimmunoglobulinemia D, periodic fever syndrome, Cryopyrin-associated periodic syndromes, Schnitzler's syndrome, Systemic juvenile idiopathic arthritis, Adult's onset Still's disease, Gout, Pseudogout, SAPHO syndrome, Castleman's disease, Sepsis, Stroke, Atherosclerosis, Celiac disease, Deficiency of IL-1 Receptor Antagonist, Alzheimer's disease, Parkinson's disease, Multiple Sclerosis and Cancer.

58. The method of claim 56, wherein the IRAK4-mediated disease is selected from the group consisting of an autoimmune disease, an inflammatory disease, bone diseases, metabolic diseases, neurological and neurodegenerative diseases and/or disorders, cardiovascular diseases, allergies, asthma, hormone-related diseases, Ischemic stroke, Cerebral Ischemia, hypoxia, Traumatic Brain Injury, Chronic Traumatic Encephalopathy, epilepsy, Parkinson's disease, and Amyotrophic Lateral Sclerosis.
